SEQUENCE LISTING (85 sequences)

SEQ ID NO: 1 - DNA, 101 bp, Cucumis sp.
  source 1..101: mol_type = unassigned DNA
gtggcaatggagcaagacaacgaagaggtgaagacgtttcagagcttaggaatctgtgagcaattggttgaggcatgtgacagtttaggctggaaaaatcc

SEQ ID NO: 2 - DNA, 101 bp, Cucumis sp.
  source 1..101: mol_type = unassigned DNA
catcaactttttaattctaaaacattggtagtatagtaatgaaaaaaagggtattaattattgcatcacactaatttaataagagactaaatggagaagat

SEQ ID NO: 3 - DNA, 101 bp, Cucumis sp.
  source 1..101: mol_type = unassigned DNA
gggcatttcatgataccacatgcagtgaaagaagacacaactttggagaactatttcataccaaagaatggcatcgtgagtttctttgcagtgggaatggg

SEQ ID NO: 4 - DNA, 101 bp, Cucumis sp.
  source 1..101: mol_type = unassigned DNA
aacattcttaatctaagagcaagtaatcaccacagaaaaggagaagatgagccaatcaagggaaccatacaaatttatgataaaaaaagatacgaaaaaag

SEQ ID NO: 5 - DNA, 101 bp, Cucumis sp.
  source 1..101: mol_type = unassigned DNA
taaattatcaaattggctcatacggtgtacaattaggaaagaatgtctaaaacaggagatgaagatctgcttggatctttgacttcaaaagaaaaagggaa

SEQ ID NO: 6 - DNA, 101 bp, Cucumis sp.
  source 1..101: mol_type = unassigned DNA
ctaggaccttgagtctttcctcatttgtcccaccactcttgtccacttacaagaacttgctcttatcaaagtccgtagaaaaggtagatagagcacgacga

SEQ ID NO: 7 - DNA, 101 bp, Cucumis sp.
  source 1..101: mol_type = unassigned DNA
atccctttttcccatttttttgaaaaattaattctatcgtccttttacaacatctgataacgacaatcaagttgatttttcggtatatcatattcacttct

SEQ ID NO: 8 - DNA, 101 bp, Cucumis sp.
  source 1..101: mol_type = unassigned DNA
aaagtaagccttttttgcttattttggtttttgtaaattgggaaagggaacatttttaacgtccaccattgcaggacagcccatgctatatatagttctct

SEQ ID NO: 9 - DNA, 101 bp, Cucumis sp.
  source 1..101: mol_type = unassigned DNA
ttgctcaatcatatattcaagctaatgtattaatactttgttgctttagtattggcaaaccaaatcagcatttccaattcaccactgcatattttgctctt

SEQ ID NO: 10 - DNA, 101 bp, Cucumis sp.
  source 1..101: mol_type = unassigned DNA
tttatacgaatgaacatttaaataggtagcaagtgcagatttaaaaaccaaaacattctttcacttttcaaaacttggattttgaaagcaagaatctagct

SEQ ID NO: 11 - DNA, 101 bp, Cucumis sp.
  source 1..101: mol_type = unassigned DNA
taagtgtgaaatttgtggaaatgtgcaaaatcgtagttaagaaaatagatcttgaaatgaactttcactacaattttcttaacgcacaaattcagatgttg

SEQ ID NO: 12 - DNA, 101 bp, Cucumis sp.
  source 1..101: mol_type = unassigned DNA
aaaaacttgtacatattaatatagtcttaactcttaaccatcacaacatcgttcaaaagatgctccttttaacaccatttctccaaaacgaagctgcagtt

SEQ ID NO: 13 - DNA, 101 bp, Cucumis sp.
  source 1..101: mol_type = unassigned DNA
attgaatatttgagaagttcaatgaagatatttaaaggaagaaagaaaaaagaaacagagatacataggtttgtattggtaacataaaaaatgcaaaaaga

SEQ ID NO: 14 - DNA, 101 bp, Cucumis sp.
  source 1..101: mol_type = unassigned DNA
tagctatgttattgtatgtattgaaaattaatatagccattgtttaatttgttgcaatgattatgatatattgcatgagtcgtgcaagttatgaacaactt

SEQ ID NO: 15 - DNA, 101 bp, Cucumis sp.
  source 1..101: mol_type = unassigned DNA
ttggattgttgcattttgagcattcaattattaagtcataacagaagaacgatctctttaaataatttttaaaaggttatgttctcaataattgatctcaa

SEQ ID NO: 16 - DNA, 101 bp, Cucumis sp.
  source 1..101: mol_type = unassigned DNA
tgtaggaatatagaacaaaatcaatattgcaatagcaaaactttgagaaactatagtaaaattatttcaaatttcatcgtattaatatttatagttaaacg

SEQ ID NO: 17 - DNA, 101 bp, Cucumis sp.
  source 1..101: mol_type = unassigned DNA
taatttcaagacaacaaaatttaaanaaaaaaaaaacgaaaaaagagtttatggggtgagagaggctcgaactctcgacctcaggatcactctaagctatg

SEQ ID NO: 18 - DNA, 101 bp, Cucumis sp.
  source 1..101: mol_type = unassigned DNA
acactcaaggaaaacaactaatttactgtttgaagatccaggggaaaaaagagtaaccttgactgcagctgtaataactttttcaccaataattccaacac

SEQ ID NO: 19 - DNA, 101 bp, Cucumis sp.
  source 1..101: mol_type = unassigned DNA
ttcgaaatcgaaagtttcgaatttctctgtaaaattacaataagaaaaacgttaaactattctacttccgtggttataaataaatcttcaagccntttttt

SEQ ID NO: 20 - DNA, 103 bp, Cucumis sp.
  source 1..103: mol_type = unassigned DNA
tcaccatttacatcaacctgtgtcaaagcaataaaagtttattcaatgatgatgtcattcgccctcaaattaagatttttttcctttactaaatccactttgt

SEQ ID NO: 21 - DNA, 101 bp, Cucumis sp.
  source 1..101: mol_type = unassigned DNA
gaaattgaaattgaaattctaatttttcatttctttttcagcttttgttttaaattaaattaaattaatttgtttattgcttttgttaacccaaacgctct

SEQ ID NO: 22 - DNA, 101 bp, Cucumis sp.
  source 1..101: mol_type = unassigned DNA
gtttgagtttttggtgaagatttaaactcgttcatctgctcaaattttgcaaacacccaaacaaatccaaatccataatccataattcataatccaatcca

SEQ ID NO: 23 - DNA, 101 bp, Cucumis sp.
  source 1..101: mol_type = unassigned DNA
ttttcaaggagatacaaattttcatttttttttatatttcaaaattagcacaaagttaaattaagatagatacgagtatttttctatttgaattagcctta

SEQ ID NO: 24 - DNA, 101 bp, Cucumis sp.
  source 1..101: mol_type = unassigned DNA
atgtatgtttgcacggtcatgattttatttgatagtattcagttttgcacagccataattttatgcatgtatttgatactacttatacatttttatattgg

SEQ ID NO: 25 - DNA, 101 bp, Cucumis sp.
  source 1..101: mol_type = unassigned DNA
tagcaacttccagcgacatgaaaataaacttccgtgccaaaaaaaggtaaggtgatattaagaaaagagattttaattgacatcaccaagatcatagaata

SEQ ID NO: 26 - DNA, 101 bp, Cucumis sp.
  source 1..101: mol_type = unassigned DNA
tgtattatttgatgaaaaaatcattaaaataaactctgtatttatattaattttagcattttgaatacaaactctaataaataatcattttatatttgttt

SEQ ID NO: 27 - DNA, 101 bp, Cucumis sp.
  source 1..101: mol_type = unassigned DNA
acaaatattaaacacaaaagtcaaaaaaaaatccagaccttgatgttatccctcacctactctgtttctgagaatgaatttcaacggagttttgtgcgata

SEQ ID NO: 28 - DNA, 101 bp, Cucumis sp.
  source 1..101: mol_type = unassigned DNA
ttgaagagaagctgaagagtggatgatgagcagaagaagactcgggaatcggagagaagattgaagagatgctatggctgcagaaaaaggcgggaccggag

SEQ ID NO: 29 - DNA, 101 bp, Cucumis sp.
  source 1..101: mol_type = unassigned DNA
tatcagaaaattaatataaactctaggcaaggataattatataaagaatacatttcagtgaatcacaaaacctaagccaggtccagattacagttcaaagc

SEQ ID NO: 30 - DNA, 101 bp, Cucumis sp.
  source 1..101: mol_type = unassigned DNA
ttttactttttgataatgaaaatttacaacaaatatatcccaagttaactaatttatattatatagattttaattttgaaaaagaatataatgtcaaagct

SEQ ID NO: 31 - DNA, 101 bp, Cucumis sp.
  source 1..101: mol_type = unassigned DNA
tttaactgttgactgaccttactcttggttttcttcgggaaagtcttcgtgagttcgtttagtatatggagaaatacttgggtcaagaagaagaaattagt

SEQ ID NO: 32 - DNA, 101 bp, Cucumis sp.
  source 1..101: mol_type = unassigned DNA
tggatgcgtaaaagtggctgtagttggagtttccgtgtgaaatctgacgtgtggacttccaaaaaggtgttagggacccaccttagaaggaaataaacgac

SEQ ID NO: 33 - DNA, 101 bp, Cucumis sp.
  source 1..101: mol_type = unassigned DNA
cttatatgagtttttttttttttttttaaaaatatcttcttctaagaaatgaaattattttcctttttagaaatttctaaaatttaggaaatcaaattatt

SEQ ID NO: 34 - DNA, 101 bp, Cucumis sp.
  source 1..101: mol_type = unassigned DNA
ttgttacaatcatcttcaattattcagcattatctacatggatggattatgtataattagtgacaacacataattttataagaaattagaagcaaaattat

SEQ ID NO: 35 - DNA, 101 bp, Cucumis sp.
  source 1..101: mol_type = unassigned DNA
caatcaaagtatttggttgatataaaaatgagaaagatttagatttgggatattttcaaatataagaaatgcattaaaatatttataaaaaaatagcaaag

SEQ ID NO: 36 - DNA, 101 bp, Cucumis sp.
  source 1..101: mol_type = unassigned DNA
ggaattgtcttcaaaggtagctgttctgaggagaaatttatcatccaagttggggttgctatacctgatattgatgtaaggccatgttaggccttattcac

SEQ ID NO: 37 - DNA, 101 bp, Cucumis sp.
  source 1..101: mol_type = unassigned DNA
tcatattaactgcattctaatttaacaatgtaaaatgacttttattatacctatttggagaattttgttattttattattattgtttttttttactacaca

SEQ ID NO: 38 - DNA, 101 bp, Cucumis sp.
  source 1..101: mol_type = unassigned DNA
acataccttctagttcattgaaccttcacactaatatcatgacctagttggacagcggtagacacgtgacattttttagatatattcattcctcttaacat

SEQ ID NO: 39 - DNA, 101 bp, Cucumis sp.
  source 1..101: mol_type = unassigned DNA
gaaggagaagtgttaaaagttgttcatttgacaaggctacgttaaatcaccttttctcccaattgccctcaaaatcaaattgagatgaattttagttttga

SEQ ID NO: 40 - DNA, 101 bp, Cucumis sp.
  source 1..101: mol_type = unassigned DNA
gatatgcatctcaataaaataattcatccacaatcttttccataaaaaaatttttttttgctcactttcaaagatagaaatatgggatttgtctttaaatt

SEQ ID NO: 41 - DNA, 101 bp, Cucumis sp.
  source 1..101: mol_type = unassigned DNA
agttgggaaatgtaaagtaaaagtttgacttatgaacattatgatctacaatttcatttacattttatatataatagaaagcatatgaaacatgagttttg

SEQ ID NO: 42 - DNA, 101 bp, Cucumis sp.
  source 1..101: mol_type = unassigned DNA
ggagaggatgaaaaaacaagttaaatataaaataaaaataaaaataaaataacttagttcccatcgtacaaaaatttaaaatttctttttcttttatctaa

SEQ ID NO: 43 - DNA, 102 bp, Cucumis sp.
  source 1..102: mol_type = unassigned DNA; note = SEQ ID NO: 23 with insertion
ttttcaaggagatacaaattttcatttttttttatatttcaaaattagcacaaaagttaaattaagatagatacgagtatttttctatttgaattagcctta

SEQ ID NO: 44 - DNA, 102 bp, Cucumis sp.
  source 1..102: mol_type = unassigned DNA; note = SEQ ID NO: 36 with insertion
ggaattgtcttcaaaggtagctgttctgaggagaaatttatcatccaagttgggggttgctatacctgatattgatgtaaggccatgttaggccttattcac

SEQ ID NO: 45 - DNA, 101 bp, Cucumis sativus
  source 1..101: mol_type = unassigned DNA
gaatcttcttggaaaattcagtctgcaatgaaagaaaaacaaagaatgttaatgcaaatgaaattaaatgccaagtggtttttgctttcttaccttttaca

SEQ ID NO: 46 - DNA, 101 bp, Cucumis sativus
  source 1..101: mol_type = unassigned DNA
caaactttataaatacataacacgntcktatactatgtttgtcttgatagttttgtatctcttcacatggccatttagttacttgcattgcaatgtagata

SEQ ID NO: 47 - DNA, 101 bp, Cucumis sativus
  source 1..101: mol_type = unassigned DNA
catcgacctcgcttcgaacccagccattcccatcctggcacgccacttcctcgaaacctcgcatctttccactcggtctctaccctcacaagctaaggaat

SEQ ID NO: 48 - DNA, 101 bp, Cucumis sativus
  source 1..101: mol_type = unassigned DNA
ttattttacacaccactaaagtaatattaacataaatggcaatgttgattcttcattaaactaattttggctttaaatttagaagaaaaagaaaaggatga

SEQ ID NO: 49 - DNA, 101 bp, Cucumis sativus
  source 1..101: mol_type = unassigned DNA
acatttaaaatgctagaaaatttatcgaaaatgatacatttataattgttggaaggagtagggtttagtagatttttgaagtgattgttcttttattacat

SEQ ID NO: 50 - DNA, 101 bp, Cucumis sativus
  source 1..101: mol_type = unassigned DNA
aatcaaggatttggatttggatttggattttgttggattggatgcttggtttgatttggtgcttttgtccwtttttgtgagaaaaaratgcaataaaagta

SEQ ID NO: 51 - DNA, 101 bp, Cucumis sativus
  source 1..101: mol_type = unassigned DNA
atttcaaaaacttagcttgccttctaagcttttactatatatttttgttcaagttcaattttacttgtttctaaatgtaggactagaatgactcgaggttt

SEQ ID NO: 52 - protein, 371 aa, Cucumis sativus
  source 1..371: note = 2-OGD1 protein
MANLTPLSKLDETFDRVSELKAFDQTKAGVKGLVDSGVAEIPRIFYYPHKERSNSDKTSVTDEPHLGVPVVDLVDIDKDPFKRRKVVDKIREASESWGFFQVLNHGVPASVQDEIINGTRQFFEQDIEMKKQYYTRDNTKPFVYNSNFDLFSTSTANWRDTVFIQMAPNPPNPQDFPLVCRDILVEYSKQMEKVGEMIFGLLSEALGLQSTHLLELDCSEGHAFMCHYYPSCPQPELTIGTTQHSDSSFITVLLQNHIGGLQVLHHNKWVDVPPVPGAFAVNVGSLLQLISNDKFVSSVHRVVANREGPRISVASAFSTGTIPTSKLYGPIKQLLSQQNPPKYRQITVKEYRLYFAKKGLDGTDALTHFRL

SEQ ID NO: 53 - protein, 412 aa, Cucumis sativus
  source 1..412: note = 2-OGD2 protein
MENRRGTGSGIPDPTPPRVISKFTSSPPNNTISVQNTHQTKMANLTPFSKLDQTFHRTSELKAFDQTKAGVKGLVDSGITEIPAIFYYPHKERSNSDKTSVPDEPHFGVPVVDLEDIDKDPLKRKQVVDKIREASETWGFFQLLNHGVPVSVQEEIINGTRRFFEQDIEEKKQYYTRDNSKPFLYNSNFDLFIAPFANWRDTILTQIAPTSFGPQELPQVCRDILLDYSKHMENVGELIFGLLSEALGLKSTHLVDIGCNEGHALLCHFYPSCPQPELTIGITEHADGTFITVLLQDHIGGLQVLHDNKWVEIPPIPGALVVNVGNLLQLISNDKFVSSIHRVLATRNGPRVSVATFFSTGYAETFKLYGPIEELLSEQNPPKYKQTTVRDYRLYFSKKGLDGMDPLTHFRI

SEQ ID NO: 54 - protein, 368 aa, Cucumis sativus
  source 1..368: note = 2-OGD3 protein
MANLTPFSKLDKTFDRASELKAFDQTKAGVKGLVDSGVAEIPGIFYCPPKECSSSIPEEETHLSVPVVDLEDIDKDPFKRRQVVDKIREASETGGFFQVINHGVPVSVQEAIIDGVRRFFEQDSEVKKQYYTRDYTKPFIYNCNFDLFTAPVANWRDTIVTFMAPNPPNPQDLPQVCRDILPEYSKQIMKLGELIFGLLSEGLGLKSTHLLDLDCNEGLRVICHYYPSSPQPELCIGTTEHSDDSFITVLLQDNMGGLQVRQQNKWVDVTPVPGAFVINVGNLLQLITNDRFVSSEHKVLANREGPRVSVASFFSTGRLPTSKLYGPIKELLSEQNPPKYKEITVREYNIYFNEKGLDGTPALPHFKI

SEQ ID NO: 55 - protein, 386 aa, Cucumis sativus
  source 1..386: note = 2-OGD4 protein
MLHQGTNGVHRIDLTPAANGDEYFDRSAELKLFDDTKAGVKGLVDNGITQIPRIFYRPPDSSDYPVAGDTELSIPVIDLEGIDADSSKRRHVVNTVREASEKWGFFQLVNHGVPLSVLDEIKNRTLRFYEQDTELKKLFYTRHNTKSIVYNSNFDLFTAPAANWRDTFLCFMAPNVPNPQDLPEVCRDILFEYSKEMKKLGRVLFGMLSEALGLNTNYLSDIECDRGLAVLCHYYPACPQPELTLGTTEHADNDFLTLLLQDDQIGGLQVLHEKKWVDIPPIPGALVINIGDLLQLISNDRFKSVEHRVVANREGPRVSVASFFGIGVYPTSQVYGPIKELLSEKNRAKYRETTLKDFYFYHNMRGLNGISALQHFRLGPEDERNG

SEQ ID NO: 56 - protein, 374 aa, Cucumis sativus
  source 1..374: note = 2-OGD5 protein
MAVAASGINLTPLSKADENFHRPTELKAFDDTKAGVKGLVDAKVNEIPRIFYHPPEDVHSAQTHIHIPLIDLEGVGNDSLKRKHIIEQIRDASEELGFFQLINHGIPTSVLEEMRDSVRRFHEQDTEDKKQYYTRDFMKPFIYNSNFDLYTAPTTNWRDTFSYVCAPNPPNPQELPEICRDIMVDYSKWVMEIGKLVLELLSEALGLNPNYLDNIGCNDGLAFVYHYYPPCPQPKLTTGISEHSDTDFITVLLQDHIGGLQIRHDNKWIDVHPVAGALVVNIGDLMQLITNDRFKSVNHRVLASHEGPRISVAGVFSTMVSPSDKLYGPIKELVSEEKPAIYRETTVRDFSVQFKSDGLGTSTLKHYKLNQAHI

SEQ ID NO: 57 - protein, 393 aa, Cucumis sativus
  source 1..393: note = 2-OGD6 protein
MRWNQPALQDKIKIDIMADAGNKFILTPLSKTTENYDRSSELKAFDDTKSGVKGLVDAGVTEIPRIFYSPPEDSHSDGVSSKTQIQIPVIDLNHVGTNSLKRKYTTDRIRKASEKFGFFQLINHGIPVSVLEGAYEAVRRFNEQETEVKKQYYSRDFSKPFIYTSNFDLYFAKTTNWRDTFRCASATTSQDQQDIPEICRDILLDYSKQLMELGKLLFELLSEALGLNPNYLNDIGCSEGVSVLYHYYPPCPQPNLTIGTSEHTDNDFITVLYQDQIGGLQIRYENSWVEVAPLGGALVVNIGDFMQLTTNDKFKSVKHRVVANKEGPRVSVAGFFTEFPFSTSKLRPIEELVSEENPAVYRETTIRDFNILFRSKGIGTTTLEHFKIKPGDA

SEQ ID NO: 58 - protein, 381 aa, Cucumis sativus
  source 1..381: note = 2-OGD7 protein
MAVAPSRVDGLNLTPLSKDDQNYHRPTDLKAFDDTKAGVKGLVDAGITEIPRIFYRPPENYDSHNISAETQVQIPVIDLDQINKSSLKRKYTIDRVREASEKLGFFQLINHGIPENVLEEMKDAVRRFHEQETESKKQYYTRDLTKPLIYNSNFDLYSAAITNWRDSFGYISAPSPHSPQDLPEICRDVLVDYSKRVMEIGNLLFELLSEALDLNPNYLKDIDCNEGLALVCHYYPPCPQPNLAIGTSEHTDNDFITVLLQDHIGGLQIRYENNWVDVPPVAGALVVNIGDLMQLITNDKFKSVKHRVLANKEDARVSVAGVFSTLHFQTSKLYRPIKELISEEKPAIYRETTIRDFNIQFRSVGLGTSTLQHFKLRQADA

SEQ ID NO: 59 - protein, 374 aa, Cucumis sativus
  source 1..374: note = 2-OGD8 protein
MAVSPSDGFILTPLSKADANYHRPTELKAFDDTKAGVKGLVDAGITEIPRIFYQPVEDYYSHKLSDETHHQIPVIDLDEVHRNPLKRKDAINRVREASEKLGFFQLINHGIPVNVLEEMKDAVKRFNEQDTEVRKQYYTRNYTKPLIYNSNFDLYSASTTNWRDTVGYVSAPNPPDPQALPEIIRDNLLDYSKRVMGIGKLLFELMSEALGLNPNYLNEIGCSEGLAIGGHYYPPCPQPELTLGTSKHSDNVFITVLFQDNIGGLQIQDQKKWVDVPPVAGALVVNIGEIMQLITNDRFVSVAHRVLAKKEGPRISVASFFSTLGFPSTKMYGPLKELLSEENPAKYRETTIREVDKLYRSKGLGTSALSHLKI

SEQ ID NO: 60 - protein, 138 aa, Cucumis sativus
  source 1..138: note = HR-like protein
MGFFSALGRLLFASFFILSACQMFNNNGGPAAVELSSKVRHVVGLSLSLKGIGALLFVLGSRVGAYLLLLYLAIITPVLFDFFNLSLLKPTFGIMLSDFLEHIALFGALLFFIGMKGTSPKKHSKKKTQKQKAKAKAH

SEQ ID NO: 61 - DNA, 1116 bp, Cucumis sativus
  source 1..1116: mol_type = unassigned DNA; note = 2-OGD1 cDNA
atggcaaacctcacacctctctctaaacttgatgaaaccttcgacagagtttccgaactcaaagccttcgaccaaactaaggccggcgtcaaaggccttgtggactccggcgttgccgaaattccaagaatattctactacccgcacaaagaacgttcaaattccgacaagacttctgttaccgacgaaccccacttgggtgttccagtggtggatttggtagacatcgataaagacccattcaaacgcagaaaagtggtggacaaaatccgtgaagcttccgaaagttgggggtttttccaagtgcttaaccatggggttccggcgagtgttcaggatgagatcataaatgggacacgtcaatttttcgaacaggatattgaaatgaagaaacaatattatacaagagacaacactaagcctttcgtttacaattccaatttcgatttgtttagcacatccactgccaattggagagacaccgtcttcattcaaatggccccaaatcctcccaacccgcaagactttccactagtttgcagagacatcctagttgagtattcaaaacaaatggaaaaggttggagaaatgatatttgggctactttcagaggcccttggtttgcaatcaacccatctcctggaattggattgcagtgaaggacatgcctttatgtgccactattatccatcctgtcctcagccagaactcaccattggcaccacccagcactccgatagtagcttcatcactgtgctgcttcaaaaccatatcggaggtcttcaagtgcttcatcacaacaaatgggtggatgttccgcctgttcctggagcctttgccgtcaatgtcggcagcttattgcagttgatttcaaatgacaaatttgtaagctcggtgcatagagtggtggctaatcgtgagggtccaagaatatcagtggcaagtgccttcagcaccggcactataccaacctccaaactctatggacccatcaagcaattgttgtcccaacaaaatcctccaaagtatagacaaatcactgttaaagagtatcgtctctactttgctaaaaaagggttggatggaacggatgctctcacacattttagactttga

SEQ ID NO: 62 - DNA, 1239 bp, Cucumis sativus
  source 1..1239: mol_type = unassigned DNA; note = 2OGD2 cDNA
atggaaaataggagggggacgggaagtggcatccccgatcccacgcctccccgtgtaatctccaagttcacgtcctcacccccaaataatactatctccgttcagaacactcatcaaacaaaaatggcaaacctcacacctttctctaaacttgatcaaaccttccacagaacttccgaactcaaagccttcgaccaaactaaggccggagtcaaaggccttgtggactccggcatcaccgaaattcctgctatattttactacccacacaaagaacgttcaaattccgacaagacctctgttcccgacgaaccccattttggcgttccagtggtggatttggaagacatcgataaagatccactcaaacgcaaacaagtggtggacaaaatccgagaagcttcagagacttgggggttctttcaattgcttaaccatggagttccggtgagtgttcaggaggagatcataaatgggacacgtcgattttttgaacaggacattgaagagaagaaacaatattatacaagagacaactctaaacctttcctttacaattccaatttcgatttgtttattgcaccctttgccaattggagggataccatcctcactcaaatagctccaacttcttttggcccacaagaattgcctcaagtttgcagagacattctacttgattattcaaaacatatggagaatgttggagaattgatatttggactactttcagaggcccttggtctgaaatcaactcatctcgtggacataggttgcaatgagggacatgccttgctctgccacttttatccatcgtgccctcagccggaactcaccattggcatcaccgagcacgctgatggtaccttcatcaccgtactgctgcaagaccatatcggaggtcttcaagtgctacatgacaacaaatgggtagaaattccacccattcctggagctttagtcgtcaatgtcggcaacttactgcagttgatctcgaatgacaaatttgtgagctcgatccatagagtgttggctactcgaaatggtccaagggtatcggtggcaactttcttctccactggctatgcagaaaccttcaaactttatggaccaatcgaagaattgttgtccgaacaaaatcctcccaaatataaacaaactactgtgagagattaccgtctctacttttctaaaaaagggttggatggaatggatcctctcacacattttaggatttga

SEQ ID NO: 63 - DNA, 1107 bp, Cucumis sativus
  source 1..1107: mol_type = unassigned DNA; note = 2-OGD3 cDNA
atggccaacctcacaccattctccaaacttgacaaaaccttcgacagagcttctgaactcaaagccttcgaccaaacaaaggctggcgtcaaaggccttgttgactccggcgttgcggagattccaggaatattctattgcccacccaaagaatgttcaagttccattccggaggaggaaacccatttgagtgttccggtggtggatcttgaagacatcgataaagacccattcaaacgcagacaagtggtggacaaaatccgagaagcttcagaaactgggggtttttttcaagtgattaaccatggagttccggtgagtgttcaggaggcgatcatagatggggttcgtcgatttttcgaacaggacagtgaagtgaagaaacaatattatacaagagactacactaaacctttcatttacaattgcaatttcgatttgtttaccgcaccagtggccaattggagggataccatcgtcactttcatggccccaaatcctcctaacccacaagacttgcctcaagtttgcagagacatccttcctgaatattcaaaacaaattatgaagcttggagaattaatatttggtttactttcggagggtcttggtttgaaatcaactcatctcttagacttggactgtaacgagggacttcgcgtaatctgccactattatccgtcaagtcctcaaccggaactctgcatcggcacaactgaacactctgatgattcattcatcaccgtgctactgcaggacaatatgggaggcctccaagtgcgtcaacaaaacaaatgggtggatgttacaccagttcctggggccttcgttatcaacgtcggcaacttattgcagttgataacaaatgatagatttgtgagttcagagcataaagtattggcgaaccgtgagggcccaagagtatctgtggcaagtttctttagcaccggccgtctaccaacatctaaactttatggacctatcaaagaattgttgtctgaacaaaatcctcccaaatacaaagaaatcactgttagagagtacaatatctactttaatgaaaaagggttggatggcactcctgctcttccacatttcaagatttga

SEQ ID NO: 64 - DNA, 1161 bp, Cucumis sativus
  source 1..1161: mol_type = unassigned DNA; note = 2-OGD4 cDNA
atgttgcaccaaggcaccaatggagtccaccgaattgacttaactccggcagccaacggggacgaatatttcgacagatctgctgaactgaaactcttcgacgacaccaaagccggagtgaaaggccttgtcgacaacggcattacccaaataccgcgaatattttaccgaccgcctgattccagcgactaccctgttgccggggacaccgaactgagcattccagtaatagacctcgaaggcattgacgcagattcatccaaacgaagacatgtcgtcaatacagtcagagaagcctctgagaaatggggtttctttcaattggtcaaccatggagttccgctgagtgttctggatgaaattaaaaatagaacactaagattttacgaacaagataccgaactgaagaagctattctacacccgccacaacacaaaatccattgtttacaacagtaatttcgatctgtttactgcaccagccgctaattggagagacacattcctctgtttcatggcccctaatgttccaaacccacaagacttgcctgaagtttgccgagatatcctgttcgagtattcaaaagaaatgaagaaattggggcgagtactatttgggatgctttcggaggctcttggcttgaacacgaattatctaagcgatatcgaatgcgacaggggattagctgttctgtgtcattactatccagcatgtccacagccagagctcactttaggcacaactgaacacgccgacaatgatttcttgacgcttcttctacaagacgaccaaatcggaggtcttcaagttctccacgaaaagaagtgggttgatattcctccaattcccggagcactggtgatcaacattggggatcttcttcagctgatttccaacgatcggttcaagagcgtggagcatagggtggtggcgaatcgagaaggtccgagagtttcggttgcgagcttttttgggatcggcgtttatccaacatctcaagtttatggacccataaaggaattgttatcggagaaaaatcgtgccaagtatagagaaactacgctcaaagacttctatttctaccacaatatgagagggttgaatggaatttccgctctgcaacatttcaggctcggtcccgaggatgaaagaaatggttag

SEQ ID NO: 65 - DNA, 1125 bp, Cucumis sativus
  source 1..1125: mol_type = unassigned DNA; note = 2-OGD5 cDNA
atggcggtcgcagcttcaggaatcaaccttactccactatccaaagccgacgaaaacttccatagacccactgaactcaaggctttcgatgacactaaagctggcgttaaaggcttagtcgacgcaaaagttaatgaaatccctcgtatcttttaccacccaccggaggacgtccactccgcccaaacccatatccacattccattgatagaccttgaaggcgttggcaacgattcactcaaacgcaaacacatcatcgaacaaatccgtgacgcttcggaagaattgggcttcttccaattgattaaccatggaattccgacgagtgttcttgaagaaatgagagactctgttcggagatttcacgaacaagacacggaagataagaaacaatactatacaagagatttcatgaagccttttatttacaatagtaatttcgatttgtatactgctcccactaccaactggagggatacatttagttatgtctgtgctccaaatcctccaaatccacaagaattgccggaaatttgcagagatattatggtggattactcgaaatgggtgatggagatcggaaaattagtgttggaattgctgtcggaagctttgggtttgaatcccaactacttggacaacataggatgcaatgatgggcttgcatttgtatatcactattaccctccatgtccacagccaaaattgaccaccggcatatccgagcactctgacactgacttcatcacggtgctgttgcaagaccacatcggcggcctacagattcgtcatgacaacaagtggattgacgtgcatcctgtagctggagccttagtcgttaacatcggtgaccttatgcaactaataacaaatgacaggttcaaaagtgttaatcatagggttctagcaagtcatgaaggtccgagaatatcagtagcgggcgttttctcaacgatggtttcaccaagtgacaaactttatggacccatcaaggaattggtatcagaagaaaagcctgcaatttatagggaaactacagttagagactttagtgttcaattcaagtcggatgggcttggaacttctactttgaagcattacaagctgaatcaagcacacatttaa

SEQ ID NO: 66 - DNA, 1182 bp, Cucumis sativus
  source 1..1182: mol_type = unassigned DNA; note = 2-OGD6 cDNA
atgcgttggaaccaaccagctttgcaagataagataaagatagatataatggcggatgcaggcaacaaattcatcctcactccactgtccaaaaccacagaaaattacgatagatcctctgagctcaaagctttcgatgacactaaatccggtgtcaaaggcttagtcgacgccggagtcactgaaattccacgcatcttttacagtccaccagaggactcccattccgacggtgtttctagcaaaacccaaatccagatccccgtgatagatctcaaccacgttggcactaattcactcaaacgcaaatacaccaccgacagaatccgaaaagcttcagaaaaatttggatttttccaactgattaaccatgggattccggtgagcgttttggaaggggcgtatgaagcggttcgaagatttaacgaacaagaaacagaagtaaagaaacaatactattctcgcgacttctctaagccttttatttacaccagcaatttcgatctctactttgcaaaaaccaccaattggagggacacatttagatgcgcaagcgccacaacttcccaggatcagcaagacataccagaaatttgcagagacattctgctggattactcaaaacaattgatggaacttgggaagttactatttgaattgttatcggaagctctggggttgaatccaaattacttgaacgatataggatgtagcgaaggggtttcagttctgtatcactactacccgccatgcccacagccgaatttgaccatcggcacatcagagcacaccgacaatgattttataactgttctgtatcaagatcaaatcggcggcctacagattcgttacgagaacagttgggtggaggttgcccctttgggaggagctttagtggtgaacatcggagattttatgcagctgacaacaaacgacaaattcaaaagcgttaagcacagagttgttgcgaacaaggaaggtccaagagtatcagtggctggttttttcaccgagtttccattttcgacgtccaaattaagacccattgaggagttggtttcggaagaaaatcctgcagtatacagagaaaccacaattagagacttcaacatcctgttccgttcaaaaggtattggaactactactttggagcatttcaagataaaacctggagatgcttaa

SEQ ID NO: 67 - DNA, 1146 bp, Cucumis sativus
  source 1..1146: mol_type = unassigned DNA; note = 2-OGD7 cDNA
atggcggtcgcaccctcaagagtcgatggactcaacctcactccgctgtccaaagacgatcaaaactaccacagacccactgacctcaaagctttcgatgacaccaaagccggcgtcaaaggcttagtcgacgccggaatcaccgaaattccgcgtatcttttaccgcccaccggagaattatgactcccacaatatttccgccgaaacccaagtccagataccagtgatagacctcgatcaaatcaacaaaagttcactcaaacgcaaatacaccatcgacagagtcagagaggcgtcagagaaattgggttttttccaactgattaaccatgggattccggagaacgttcttgaggagatgaaagatgcagttcgaagatttcacgagcaagaaaccgaatccaagaaacaatattacactcgcgacctcaccaaacctttgatttacaatagtaatttcgatctgtactctgcagccatcaccaattggagggatagctttggctatatcagtgccccaagtccccacagtccgcaagacctgccggaaatttgcagagatgttcttgtggattactcaaaacgagtgatggagattgggaatttactgtttgaattgctgtcggaagctcttgacttgaatccaaattatttgaaagatatagactgcaatgaaggacttgcacttgtatgccactactacccaccatgcccacagccgaatttagccatcggcacatcagagcatactgacaatgactttatcactgtgctattgcaagaccatatcggcggccttcagattcggtatgagaacaactgggtcgacgtgcccccggtcgctggagctttagtggtgaacatcggagatcttatgcagctaataacaaatgacaaattcaaaagcgttaagcacagagttcttgcaaacaaggaggatgcgagagtatcagtggcaggggttttctcgacgcttcattttcaaacctccaaattgtatagacctatcaaggagttgatatcagaagagaaacctgcaatatacagagaaaccaccatcagagacttcaatatccagttccgttccgttggccttggaacttctactctgcagcatttcaaactgagacaagcagatgcttga

SEQ ID NO: 68 - DNA, 1125 bp, Cucumis sativus
  source 1..1125: mol_type = unassigned DNA; note = 2-OGD8 cDNA
atggccgtgtcaccctccgacggattcatcctcactcctttatccaaagccgatgccaattaccatagacccaccgaactcaaagccttcgacgatacaaaagcaggcgttaagggcttagttgacgcaggaatcaccgaaattccacgcatcttttaccaaccagtggaagattactactcccataaactttccgacgaaactcatcaccaaatcccagtgatcgatctcgacgaggtccacagaaatccactgaaacgaaaagacgccataaacagagtccgagaagcttcagagaaattgggtttttttcaactgattaatcacgggattccggtgaacgttctcgaagaaatgaaagatgcagtcaagagattcaacgaacaagacacggaagtcaggaaacaatactacacccggaactacacaaaacctttaatttacaacagcaatttcgatctgtatagtgcatcgactacgaattggagagacacggttgggtatgtaagtgcccctaatcctcccgatccacaagccttacccgaaatcatcagagataatctcttggactactcgaagagagtgatggggattgggaaattgttatttgaattaatgtcggaagctttagggctgaacccaaattacttaaacgaaataggctgcagcgagggactcgcaattgggggccattattacccaccatgtccacagcccgaattgacactaggcacatccaaacacagtgacaatgttttcatcactgttctatttcaagacaacatcggcgggcttcagattcaagaccagaaaaaatgggtggatgtgccacctgtcgccggagctttggtggtgaacatcggagagattatgcagttaataacaaacgacagattcgtaagtgtggcacatagagtgttggcgaagaaggaaggtccgagaatttcagtagctagctttttttcgacgctgggatttccgagcacgaaaatgtatggacccctaaaggaattgttatcggaagagaatcctgcgaagtacagggaaaccacaattagagaagtggataagctgtatcgttctaaagggcttgggacttccgctctgtcgcatcttaagatttga

SEQ ID NO: 69 - DNA, 417 bp, Cucumis sativus
  source 1..417: mol_type = unassigned DNA; note = HR-like cDNA
atgggattcttctccgcccttggacgcctcctctttgcctccttcttcatcctctctgcttgtcagatgtttaacaataatggtgggccagcagctgtggaattgtcttcaaaggtcagacatgtggttggtctttcattatctctcaaagggattggagctcttctatttgttttgggctcaagagttggagcttatcttttgcttctttacttggctattatcactccggttcttttcgatttcttcaaccttagtctactgaaacctacttttggtatcatgttaagtgatttcttagagcacattgcactttttggtgctctccttttcttcatagggatgaagggcacaagtccaaagaagcactcaaaaaagaagacccaaaaacaaaaagcaaaagcaaaagcacattag

SEQ ID NO: 70 - protein, 365 aa, Arabidopsis thaliana
  source 1..365: note = At1g06620 protein
MESSLPQVAALDRSTLLKAFDETKTGVKGLIDAGITEIPSIFRAPPATLTSPKPPSSSDFSIPTIDLKGGGTDSITRRSLVEKIGDAAEKWGFFQVINHGIPMDVLEKMIDGIREFHEQDTEVKKGFYSRDPASKMVYSSNFDLFSSPAANWRDTLGCYTAPDPPRPEDLPATCGEMMIEYSKEVMKLGKLLFELLSEALGLNTNHLKDMDCTNSLLLLGHYYPPCPQPDLTLGLTKHSDNSFLTILLQDHIGGLQVLHDQYWVDVPPVPGALVVNVGDLLQLITNDKFISVEHRVLANVAGPRISVACFFSSYLMANPRVYGPIKEILSEENPPNYRDTTITEYAKFYRSKGFDGTSGLLYLKI

SEQ ID NO: 71 - protein, 369 aa, Arabidopsis thaliana
  source 1..369: note = At1g06650 protein
MEMMKIDPLFDRASELKAFDETKTGVKGLVDSGVSQVPRIFHHPTVKLSTPKPLPSDLLHLKTIPTIDLGGRDFQDAIKRNNAIEEIKEAAAKWGFFQVINHGVSLELLEKMKKGVRDFHEQSQEVRKEFYSRDFSRRFLYLSNFDLFSSPAANWRDTFSCTMAPDTPKPQDLPEICRDIMMEYSKQVMNLGKFLFELLSEALGLEPNHLNDMDCSKGLLMLSHYYPPCPEPDLTLGTSQHSDNSFLTVLLPDQIEGLQVRREGHWFDVPHVSGALIINIGDLLQLITNDKFISLEHRVLANRATRARVSVACFFTTGVRPNPRMYGPIRELVSEENPPKYRETTIKDYATYFNAKGLDGTSALLHFKI

SEQ ID NO: 72 - DNA, 28180 bp, Cucumis sativus
  source 1..28180: mol_type = genomic DNA
tattttcaaatataagaaatgcattaaaatatttataaaaaaatagcaaagtttcatatttaattagtgatacacttatagattattagtatgagtcaataacattgacatttcaaccaaattctaacttcaaaaattatagagattgatgacatttttacccattcattctaaaatttatgtaatcctaatcaaattttattgatggatcactcctgatcagcttgaatcccaaacctatttaattgtctttcatcacttctacttaaaaatacaaatatgtcatatttattgttatagttaatgttaccattgtcatttgaccttaattgttaaaacaggtaaagaaacaacattgtttgggacgaactcacagttaactacagaggatacaacatacaaaacaataagtaaagagagtagaaatacacaatacattgttaatctagttcaatgacacaatatttacgtctagagggtcctttactctaagatcagagattttatttttttgatatataaatcataatcaaactattatgattctcagtagaaggtttaacactaattttcttcactcagtactctatcagtaatacaatattaaagtaacactatcactaaactgtttcaaatatagatttggtttggttaccaaaatctccctcttaatcaagtttctcaccaatcaacagattaagtagtaaactagaggaactcccctgaatcacatccttcatcaaggcgtaaacaaaagttccttatatcatacaacagaaacttgaacttcatatcttataacctcggcactaatcgtcactaaataaaagcaacaacaacaaaccacacgaatgaaagaaaacttacacgctttaggttctatttttttaacgggtggttcaatcgagaagttgttcttcacttttctatcatcgtgcattgcaacaacttttctcataacaagaataaaaattcgtacacacaaatatctactacaaagacaaatcttcttcttttaagaaaaaaaaaatatgttcacaataggacccaaataaaatctttgtctcatccaaatattcttcacaaactttcaaaacactttttgccacgtcaaccaaaaagaagaaaatagtgaacaaataaaatattcacgacctcacacaatttctaacactaacgataactatacactaacatgttttacaaattaaaagtttacacctctaacaaagaaattcaaccttaacatagttcaaacattttagtaaagcttggaatagggaaaccaaaaaagagaagagtaaatcaaagtctaaaatgtgtgagagcatccgttccatccaacccttttttagcaaagtagagacgatactctttaacagtgatttgtctatactttggaggattttgttgggacaacaattgcttgatgggtccatagagtttggaggttggtatagtgccggtgctgaaggcacttgccactgatattcttggaccctcacgattagccaccactctatgcaccgagcttacaaatttgtcatttgaaatcaactgcatgctctcataaaaggaactcaattaatcaaaccatgaaaacacaacaatatattatatagcttagcttaatgaatgcatgataatagttaataatgaaaaccttgcctgcaataagctgccgacattgacggcaaaggctccaggaacaggcggaacatccacccatttgttgtgatgaagcacttgaagacctccgatatggttttgaagcagcacagtgatgaagctactatcggagtgctgggtggtgccaatggtgagttctggctgaggacaggatggataatagtggcacataaaggcatgtccttcactgcaatccaattccaggagatgggttgattgcaaaccaagggcctctgaaagtagcccaaatatcatttctccaaccttttccatttgttttgaatactcaactaggatgtctctgcaacaaaattcacaaagtagaaagtaacaccaaaattctttactccgtgatattcaatcagttcctaacaaataacatattcaaaaggtatgctaggaggccacaagtattctaacaaaataaacatcttaataacaccttgatattttattagacaatcttgacttttttttatttatggagaaaaatagttaacttttagaatcttaaaagtaaaataaaaaatatataaacctttagtacagtaattgcacattaacagaaagatcgtttgttttctttaccaatgactcaaatccaaaatgcaccataacaagatgatctcatatctctcttcacagtacgaaaacgtttttttttccttttttgggtttgaagttcatctctatttgatccataaagtttcaaaatagacactttagtgtcgaacgacggcaaaatactttcaaataatttttaacttagctttaagttaatccaatgcttattggtagagaaaattaattaacattaattttaaaagaaccattaatgaaaatattttaaaaaagatttgatacttttattggtacattctaagatgaacccaattctttttcattgaaaatttgcatgattaaatttatttatttattttcaaaactacactaaatcagtcatttatgttaaattaaattattcttgaacttaactttgaatttgtcacaaaatttgaaatccataacatttaaaaggaatatattctgcacacatctagacatttttatacaattatcttctattctcattctcatgcatcactaatctacctttgttactcataaacaattccacacctttcaataatatatatacatccattaccgatacacaatattatagacgtaaaaatagtatactaagccctgataggagttcactcaattcacgtacgaaacgcttttacgattaatctgaccctcaaagtcacccatgcaacaaaaagatagagaatggagaggcagagagtttacctgcaaactagtggaaagtcttgcgggttgggaggatttggggccatttgaatgaagacggtgtctctccaattggcagtggatgtgctaaacaaatcgaaattggaattgtaaacgaaaggcttagtgttgtctcttgtataatattgtttcttcatttcaatatcctgttcgaaaaattgacgtgtcccatttatgatctcatcctgaacactcgccggaaccccatggttaagcacttggaaaaacccccaactttcggaagcttcacggattttgtccaccacttttctgcgtttgaatgggtctttatcgatgtctaccaaatccaccactggaacacccaagtggggttcgtcggtaacagaagtcttgtcggaatttgaacgttctttgtgcgggtagtagaatattcttggaatttcggcaacgccggagtccacaaggcctttgacgccggccttagtttggtcgaaggctttgagttcggaaactctgtcgaaggtttcatcaagtttagagagaggtgtgaggtttgccatttttgtttgaggagtgtactgtgtatggtcttttaatttaatcagtttatctgaatggagatagttatttttgctgtgagaaggacgttaatgcttgctaatccactgtcggcaaatacagacagaaaacatgagaagatagttcataactcagaccccctcatgctgcctttctctctggtcttggtttttcgtacgagtcatgattcaaaaatatcccagtgttcaaagtgttcaaagtgtttaaaaaacatattaacctaagtaattaaagatatgagattcatatccaaaatttcttttactaaattccaacttttaaaacattttgtctaattctttctctttttttaattactgaagtttaaattccaatctttgaaattgtagatactaaattaaacttagttgacatactcaaacctaattcaatcaaaggtttaacaaataatttctaaagacataacaaaccttacaacagggtaggaaaagacatttcaataaaatcaatgttatagtgtttaaaaatcacacactctaacaaagtcgaacttaaacatttaacaaaccttagaattatgaaccaaaaaaacagttcagtaaaatcaaatcctaaaatgtgtgagaggatccattccatccaacccttttttagaaaagtagagacggtaatctctcacagtagtttgtttatatttgggaggattttgttcggacaacaattcttcgattggtccataaagtttgaaggtttctgcatagccagtggagaagaaagttgccaccgatacccttggaccatttcgagtagccaacactctatggatcgagctcacaaatttgtcattcgagatcaactgcatccataaaggaacccaaacccatcattatattttctatatatgtgtatcttgttacttaatgaatgcataacaaaacattgcctgcagtaagttgccgacattgacgactaaagctccaggaatgggtggaatttctacccatttgttgtcatgtagcacttgaagacctccgatatggtcttgcagcagtacggtgatgaaggtaccatcagcgtgctcggtgatgccaatggtgagttccggctgagggcacgatggataaaagtggcagagcaaggcatgtccctcattgcaacctatgtccacgagatgagttgatttcagaccaagggcctctgaaagtagtccaaatatcaattctccaacattctccatatgttttgaataatcaagtagaatgtctctgcaacaaaaatctttatcatcaatcacaataaataacaccaattacaataatttgttatctatttagatagacaactgttagtaatttaatatatatacatatataacaagctgaaaacaaaaagaagctttaagaacaacacgtcagtaaaaataaaatggcagaatctgatgatcttcaagacacacgaacgagtgtccaaaatgtctgagcatggaacgctaaatcaagtgcaaagacgttacgattgcgattaatccaaaagaatttcctaatagaaccacttaaatggacttgtctaagtatgtatcaagctaatcctgaacctcaatcgcagagttagaattcccaaccactaatagagtaagtttccactaggtaatacccactccaagagctgagaaacatcgtttaatataataatatcctcaactacattcatggatcccagaaatgattatacgtaacaagcatagcttaaccatatgaattgcaaacaacagctccaaacaccaacacccactttaccgacgagaaaagtcttggggggcatgttagctatcgtaattaggcaaaagaacagagacagagagtggagagagtagagattacctgcaaacttgaggcaattcttgtgggccaaaagaagttggagctatttgagtgaggatggtatccctccaattggcaaagggtgcaataaacaaatcgaaattggaattgtaaaggaaaggtttagagttgtctcttgtataatattgtttcttctcttcaatgtcctgttcaaaaaatcgacgtgtcccatttatgatctcctcctgaacactcaccggaactccatggttaagcaattgaaagaacccccaagtctctgaagcttctcggattttgtccaccacttgtttgcgtttgagtggatctttatcgatgtcttccaaatccaccactggaacgccaaaatggggttcgtcgggaacagaggtcttgtcggaatttgaacgttctttgtgtgggtagtaaaatatagcaggaatttcggtgatgccggagtccacaaggcctttgactccggccttagtttggtcgaaggctttgagttcggaagttctgtggaaggtttgatcaagtttagagaaaggtgtgaggtttgccatttttgtttgatgagtgttctgaacggagatagtattatttgggggtgaggacgtgaacttggagattacacggggaggcgtgggatcggggatgccacttcccgtccccctcctattttccatccacacaaaattttccaccaacaaatatttccgcttttatttttttgtttctttgttttgattgggacgagtgatcgggtatggtatccctcttgagaaaacaataactgtatagtcagtagtaaacaaacttaaactaacataccatttttaatcaacttagattcacactctacactgatacagtttagtaaagccactttgtcactacaatttcaaagttaaattttgctaaacaaaatcctctccttgatcatgcaaatcactccttattagattaaggtggaagagtctccccttgaaaaacctcattcaaaccagtgtttatgtaatcttgtctgctaatgaagttttaccctccaacgtcaaacaaaaccagcaactaatcacaaattgttggtccaaaagtagaggatagttaagaaatgattgggtttgagggggtgaggacgtgaatggccttatatgggtccttggtttttgtcacattaaaatggcctatgcctttactttgataattattattgtttctatatattaatgatcttgacacaacaaaagatagttgatacaaaagaacatctccagactcaacagactcagttttaacacattttaatcaaatatttaataaagcattgcgatatgaaggtattgttagcatgtttagtggtgtcaatggcgaattccaagtgtggacctcaagtgccaaaaacgtccccaaccctcctttcatcacacacaaatatgaacatgtgtattagaggaccaatttataaatagtttcactccacagccaaaaaacgagaataataagtatcttcaaatgagagttaaaagaccaagaaccttagttctttattgtttagttgagttagtcacgcattcaatcactgtaggctcaaaaatcccttaaatagaggtgagtttgaacaagatcgagatcaaacaaaaggaatgtgagtttgaggtcaagaccatagagcactcacaaccaacatatttacaaatgagataagaatgctatcctatgttaacatctattaattaattgaaaatagttataattaatcaacttttcatgaccatccaaatttaatttaaagtttggagggtgactgacaacgaatagagataagtcgagggattattaaacaccaaaaccatttttctaaaagttgatttcagatatgtgtttctttgaccaactaatttcataagttcatcaactaatttcataaattcatctttttcaaaagaaactattttgagttattgctagacattccattgttattattttaaaatgatagttctttttcagaaaaaaatgaaaaaaaaaggcagtaataaaaaaagttattaaaccaaacacacaaattacaatagttgacattataggtcaggaataaattaaaagtcacactaacaaaaactcaaacttcaatacaacaatttaacaaataatctctagacatagcaaaccttacaacaaaacaataacattttaataaacccatgtttaacaaaatattagaaacaacaaaccttcttaattaaatcagtggaaggcggatttaataaacattattcaacacactgaccttaacaaactaaaaagttggggtttggaacagcgaaccaaagacgaggtgaagcaaatcaaagcctaaaatgtgtgagaacatgagttccatccaaccctttctgagcatggtgaagaacatattctctaactgtgatttgtctgtattcgggaggattttgtttcgataacaactgctcgatgggttcatagagtttggaggttggtatagaggcagtggtgaaaaagcatgtcaccgatactcttgagtgagaaactactaaacaaactctttcaacaagttaggctccccccaatggtatagatcttcgcacacactgatctactgcatcaaaacattcaccaagtgttcaatatgacatatcagaaagatgttgtaaaggagaaactcagcttcaaaaccacactctcctccacacaacaacacaccgaaaatagcccaacttttgacttgtatgcatatatatatatatatagtactatcactcgcaagctgcaatctaggttattaaaagtcggggaacaaccaaatcttgcacaatcccgagaaggactcaatctattaggacctttcagaaaaagagaaaaacatattatttagcaacagccctgaccatccgataatagcataaaataaaagaaatttgtacgccaaactcataacaactcacaacaaattatactatgattcccctagtaactgacttttcaatcatctttgtaatcaaatcttgaaatgtggaagagcaggagtgccatccaaccctttttcattaaagtagatattgtactctctaacagtgatttctttgtatttgggaggattttgttcagacaacaattctttgataggtccataaagtttagatgttggtagacggccggtgctaaagaaacttgccacagatactcttgggccctcacggttcgccaatactttatgctctgaactcacaaatctatcatttgttatcaactgcaaaaccaaccccccaaacacctttcgttaattttccctttattaatatgtatccaaatcaatcaggattgtatcaatggaaataattgtcaaatcacttgcctgcaataagttgccgacgttgataacgaaggccccaggaactggtgtaacatccacccatttgttttgttgacgcacttggaggcctcccatattgtcctgcagtagcacggtgatgaatgaatcatcagagtgttcagttgtgccgatgcagagttccggttgaggacttgacggataatagtggcagattacgcgaagtccctcgttacagtccaagtctaagagatgagttgatttcaaaccaagaccctccgaaagtaaaccaaatattaattctccaagcttcataatttgttttgaatattcaggaaggatgtctctgaaacaaaaattttaaataagttaatgcatcaaacaaaaatattatctgcttttggacagagggtgtccatttatgtgttcaattaataaaaaataaacattttacagttgaacaaatctatactgtattaaaatctaaggataagaaaaatggatttttcccaaaaatgtttgaagagatgggaagcggcaattaagaagtaagaacagagagaaataatgggcagagtatctatcacctgcaaacttgaggcaagtcttgtgggttaggaggatttggggccatgaaagtgacgatggtatccctccaattggccactggtgcggtaaacaaatcgaaattgcaattgtaaatgaaaggtttagtgtagtctcttgtataatattgtttcttcacttcactgtcctgttcgaaaaatcgacgaaccccatctatgatcgcctcctgaacactcaccggaactccatggttaatcacttgaaaaaaacccccagtttctgaagcttctcggattttgtccaccacttgtctgcgtttgaatgggtctttatcgatgtcttcaagatccaccaccggaacactcaaatgggtttcctcctccggaatggaacttgaacattctttgggtgggcaatagaatattcctggaatctccgcaacgccggagtcaacaaggcctttgacgccagcctttgtttggtcgaaggctttgagttcagaagctctgtcgaaggttttgtcaagtttggagaatggtgtgaggttggccatttttgtgtatttcgaaactccaattttgaggaggcgatgttcgattcatcttgaggggagagaaatttgatggtgacggagacttcaatagtctccgatcaaattgtgggctcaaataaaaatatatgaaaattggaatttacgactattttaacaataataataaaggatatttcaacattttaaaaaaattgtaaattattacaaaattaaggatatttcaacattttaaaaaattgtaaatattacaaaattatcatcgatagacttgtattgctgatagatttcatatggtatataagtgatagattaatatttacaacatagtcggtcagtgatagacttatatcattgatagaatttgacaaattttgttatatttgtaatttttttaaaattgtgctatatatctaattaatttgatatatatatatatatatctaaagatctcaaagaactaacagttattgattttaaaacaaatgatccataaaatctcttctcgaagaaaacaaacataaatactctctaagatatattgtcaagagagaaaaaaaataaatgaaacttattgcatttattcaatttaatggactaatatttataataaaaagaagaaaaaaaaagagtaaatgaaatagtgcttgctaaaattttggagataattaaaaaaataaataaccaaaacaataaataaaagaccaacttaattcatagcccacggcagtaacacttaccaaaaatggaaattttgtgggagtgtcctcaaaataagaaggaaagcttcaaataaaagatatgtactttttaaaattggtgtttttgtttatgtcatcatatgtaaaatcatcaactttagtcttaaatgtttctttcctcttttcttcgttttgtacttcttttgttttttttatcttcctttcttcttcttcttttataaccattcttttttcttttctttttttgtgatcctcttttgtatttcttcttccgtttttatagttctttcttttgtgtttttttctggaattcttcctctgttatttatagcattctaacctttccctttttccttttcttctttttgtgttcatttaagaagagtgacaaacatgtataacacacataaaacttcaataagctagtgtttactgttcagtataaagtatagaaaatagaggagtatcgtatcttttatctataaagcatcaagtaaatgtcacttttcatttgtcttaaaaattagatcatctttaatttagtgtttttttagggaatatctcttttccaacttgttaaaattatagaatttctatgtgtttggatgatattgatcgtgaaatggctaaacagaacaatgctatgaattatatacatcagctaattaacttgcttactagatgggaagtttattgtgagctttgttcaagggtgttaataccaaaaagttgtggaaaattgtcagaaataatagaacattaaatcttttgtcatatatggatcaacaaaaatttaacaatgctcgaaactatttgaactagcatttggaatgtagtcaagaaaacccgaaacaatcaattaagtaggaaaatatcctttaactatttggtgcgccattgtttgaaatcgacgaacagagtcaataaacttcaaataacagatctcaaatccaaccagctttcagaaataataataatcaaactaacctaaacactcccgtagatcttcctcttctacttccacccactaaaattaaaaacaatcggccactaaccatttctttcatcctcgggaccgagcctgaaatgttgcagagcggaaattccattcaaccctctcatattgtggtagaaatagaagtctttgagcgtagtttctctatacttggcacgatttttctccgataacaattcctttatgggtccataaacttgagatgttggataaacgccgatcccaaaaaagctcgcaaccgaaactctcggaccttctcgattcgccaccaccctatgctccacgctcttgaaccgatcgttggaaatcagctgcaacaatggtaagtgttaagaaatgggaaagctttcattctcaatttttatgggtaaatcttataagcttgcctgaagaagatccccaatgttgatcaccagtgctccgggaattggaggaatatcaacccacttcttttcgtggagaacttgaagacctccgatttggtcgtcttgtagaagaagcgtcaagaaatcattgtcggcgtgttcagttgtgcctaaagtgagctctggctgtggacatgctggatagtaatgacacagaacagctaatcccctgtcgcattcgatatcgcttagataattcgtgttcaagccaagagcctccgaaagcatcccaaatagtactcgccccaatttcttcatttcttttgaatactcgaacaggatatctctacaacaaacaccagctaattaacaaaagagaagaaaaaaaaagagcctgtttttttaatctgatcgaaaaccagaattttaccggcaaacttcaggcaagtcttgtgggtttggaacattaggggccatgaaacagaggaatgtgtctctccaattagcggctggtgcagtaaacagatcgaaattactgttgtaaacaatggattttgtgttgtggcgggtgtagaatagcttcttcagttcggtatcttgttcgtaaaatcttagtgttctatttttaatttcatccagaacactcagcggaactccatggttgaccaattgaaagaaaccccatttctcagaggcttctctgactgtattgacgacatgtcttcgtttggatgaatctgcgtcaatgccttcgaggtctattactggaatgctcagttcggtgtccccggcaacagggtagtcgctggaatcaggcggtcggtaaaatattcgcggtatttgggtaatgccgttgtcgacaaggcctttcactccggctttggtgtcgtcgaagagtttcagttcagcagatctgtcgaaatattcgtccccgttggctgccggagttaagtcaattcggtggactccattggtgccttggtgcaacattgtaatttgcaactaatctaatcttctctgattagattttcgtttgagaatctcaatttcagtttcaagtttctagactcccattgatataaagatttagttaaatctaacgcgactaacactcgaaatattttaacaaattcttttattttattttgttagctcatggtttcatgtcccctcctacatcgtacctcttgggtgcattacaacgacaaattacatatttgcatctttttttattattcacaaatacaaatgcatgtcatcaattatgaatgccaataaatgcgcatttacgaaatttttgcatcccgacctattatttatgttttatctatttttgttctatatccctaatttttattaaatattcttaattatttatctcagtcatgaaaaggaaattcatagggatgggtgcatcctaagtgttgcccttaatattaaactttgggtttgacttagttcctaattttgattcaaaataatcgtggcatcatttagctagcaacacctgtcatttacatttttttatattttaatattaattttaaaatgttatattataatatatattaaataatactttattattactaaattctattcccttcacaactttaacattttaaatttgaatcttatctactgcctctatttttcttctttttttgttatatagtgtttccaaattatattgaccactaattatctagaagccatattgttttgtttaatttcaacttcaacttttacaatagtagtggaagaaaatagttagaagaaagatagataaagaccgtattttatgaaaacaaacttgaggcatatctatcatttacgttcttcatcacatcgctcatccacaaaaaaaaaatatttttgtgggatggaaagatgaagaaagaaaatataagacagatggatctaaacaattccacgaattattatgggggatggtacgtcaatataattgcaatttaaatgtgtgcttgattcagcttgtaatgcttcaaagtagaagttccaagcccatccgacttgaattgaacactaaagtctctaactgtagtttccctataaattgcaggcttttcttctgataccaattccttgatgggtccataaagtttgtcacttggtgaaaccatcgttgagaaaacgcccgctactgatattctcggaccttcatgacttgctagaaccctatgattaacacttttgaacctgtcatttgttattagctgcatccaaattgttatgttatagaaaaaaattagatgagattaaaagaaaaaaaaagtgataagctcggattactataaaaacaaacgagtcttacttgcataaggtcaccgatgttaacgactaaggctccagctacaggatgcacgtcaatccacttgttgtcatgacgaatctgtaggccgccgatgtggtcttgcaacagcaccgtgatgaagtcagtgtcagagtgctcggatatgccggtggtcaattttggctgtggacatggagggtaatagtgatatacaaatgcaagcccatcattgcatcctatgttgtccaagtagttgggattcaaacccaaagcttccgacagcaattccaacactaattttccgatctccatcacccatttcgagtaatccaccataatatctctacaaaaaaaaacagaggttaataataaacccaaaattaaactaaaaagattatttgatttaggtccaaaatgatcaatttacctgcaaatttccggcaattcttgtggatttggaggatttggagcacagacataactaaatgtatccctccagttggtagtgggagcagtatacaaatcgaaattactattgtaaataaaaggcttcatgaaatctcttgtatagtattgtttcttatcttccgtgtcttgttcgtgaaatctccgaacagagtctctcatttcttcaagaacactcgtcggaattccatggttaatcaattggaagaagcccaattcttccgaagcgtcacggatttgttcgatgatgtgtttgcgtttgagtgaatcgttgccaacgccttcaaggtctatcaatggaatgtggatatgggtttgggcggagtggacgtcctccggtgggtggtaaaagatacgagggatttcattaacttttgcgtcgactaagcctttaacgccagctttagtgtcatcgaaagccttgagttcagtgggtctatggaagttttcgtcggctttggatagtggagtaaggttgattcctgaagctgcgaccgccattatggatatggatctgttgggttgatggaagagattgaatttatggaatgaagagtgtgatttcaaaattgtaaaagtggtttggtgaatggtgggaaggggtgggatataaatggtgaagtagtaggatagccagggtttgtaagttggtggtgggctctaattgtaattcaagttcaagttgtccactagaaaggaattttcaaattttgactgattggataaatagatagatgttcataattcatataggttttcctttgaattttaaactcagtcatgttgaaaagtttatattttagaatttatttattaatattttaaggaattaggggtatatttatttttgtgtttaattatctcaagggaaattgtgttaaggtgagcttggtttttttatacagactagttataaataagtttctccaaaataagaataattttttattagtttctattctttaaatccaaaattatgctatatcttataaatattttaatttattttactatttttaaaatataaataagtttggttgaaaatataaaaagaagaagaaaaaatgaaggtagagaattaaaaaagttgatttataataagggagagactcaaaataaacatcaattttctaatattttccaaaaagagtaccacgtggcacatttttatttggttgcatttatcttttatatagtttgactaacctttaatatgttgaatttaaaaaaaaaaaatgaaagaaagagaaggtggaagaagagaagaattagccttgtaaataagcatatatgaatatgacacttttgtttcatgaggtgcaagaagagttgcttttagacctttcaaaaataaattgcttttagccaaggtggtccaacgaattattgatagagtgttccacgatttcaattttcgtacatttttttcttcaatatgacaattcaaacacgtgaatttacggtggacttgagactcaatctctctcaattttatggtatttgtaatatgatatatagaaaaaacaaatgtaatattaataatagtagtcagtatcacgataaaatgcatatcctaactcattttattcctaagtttggatagaacatcatcccatctatttattcaattttacctttaaaatttaaattttatttcgttttgttctctaatttaccatatttacatttttaaccttagcttttcattaaataatcactttcattcattaaggttaatacctttttattaggttaaaataattatgaagggaaaatttaaattcatgtttctatgttgtatgttagttaatgatgtattaacatttctatgttttagcattgttgaagcaatagtcaatgaccttagccttttttttttttttttacatgattttggacgttttgtgaagaataatctaagattattaactcggaaaccttggaataattacataaacatattcttgctattatagtatgacagaaacggcatggagcaacctttaattcactttagtttttcaataatagatagaagttcaatcaaagagagaacgaacatccacaactaatcacttcttcatctactaactattaattaagcatctccaggttttatcttgaaatgctccaaagtagtagttccaataccttttgaacggaacaggatgttgaagtctctaattgtggtttctctgtatactgcaggattttcttccgaaaccaactcctcaatgggtcttaatttggacgtcgaaaatggaaactcggtgaaaaaaccagccactgatactcttggaccttccttgttcgcaacaactctgtgcttaacgcttttgaatttgtcgtttgttgtcagctgcataaaatctccgatgttcaccactaaagctcctcccaaaggggcaacctccacccaactgttctcgtaacgaatctgtaggccgccgatttgatcttgatacagaacagttataaaatcattgtcggtgtgctctgatgtgccgatggtcaaattcggctgtgggcatggcgggtagtagtgatacagaactgaaaccccttcgctacatcctatatcgttcaagtaatttggattcaaccccagagcttccgataacaattcaaatagtaacttcccaagttccatcaattgttttgagtaatccagcagaatgtctctgcaataacaaaatgttagcataaacagggatctctgttttcttttctttctttaggcttcaaagacagagctaattttacctgcaaatttctggtatgtcttgctgatcctgggaagttgtggcgcttgcgcatctaaatgtgtccctccaattggtggtttttgcaaagtagagatcgaaattgctggtgtaaataaaaggcttagagaagtcgcgagaatagtattgtttctttacttctgtttcttgttcgttaaatcttcgaaccgcttcatacgccccttccaaaacgctcaccggaatcccatggttaatcagttggaaaaatccaaatttttctgaagcttttcggattctgtcggtggtgtatttgcgtttgagtgaattagtgccaacgtggttgagatctatcacggggatctggatttgggttttgctagaaacaccgtcggaatgggagtcctctggtggactgtaaaagatgcgtggaatttcagtgactccggcgtcgactaagcctttgacaccggatttagtgtcatcgaaagctttgagctcagaggatctatcgtaattttctgtggttttggacagtggagtgaggatgaatttgttgcctgcatccgccattatatctatctttatcttatcttgcaaagctggttggttccaacgcatcaacacataataaagttaaaacttgagttagaaatattgagtatactcatatatcaatgaaacatttcttatcaaatttgtgaggtttgctaagtacaagaattcgttgaaataaatatccaacatatttacgtttgacatgtggattagtagagtgtgatatgaactctctaaggcatcacgttattaataaaattttattgaattaaatcatattttagtccataaattttcaagattgaacattagattaatcaattaattgaattgatatataaccaaaaacgatagtactaatttattctataaatattttatatagcacaaaaacataatcaataaaggtgaatcaaacgaactcaaaaactaattttattatttttccctctaaaactcaattttcttacatttcctcttcggctcccacccattctttttattttcaaattggaatcttttaagtttattttcaaattggaatcttgaagttaagaacattgagtagaatgaatgaattaataaaatagaagttgtatccatgaccacataaaataaactttaaaaaatttagttagggataaataaaggaccatagattactttttttaaaaaaaataaaattaaaaagttgttttttagaagtttataaatcaaaatagaacaacaccaatataggattcaaaccaaatttgatgtaattttagtatttcttaatttaagttaagcacggatatttttttcattttagtgttgcattaggatttttattggagagacttagttttgctatgattgttttaagaagatttggatgatttgagatttaggctttgtgattgttttcaagattagtctaagaatttttaaatttttaaatcatgtgttgaattacatgcagaacatttaagaggattgattaatttttcttgtggagtgtttcagagttaaaaaaacttattttttttcatccagttgtttgatcttaaaaacaatccgtatctaacccaatctgaaatcaagattcttaattcaaattgcaactgttattgtatcaaaattactcatccaagaatttgataggatatctctaacacgccaattttattatttattcatattcatctaacaatcttacattgacaacaacgtacgtacaccgtagaacaagtttcagtttaaagctataggattgtgcaggtcacgatcacaagttctcaatcaaaactgagaacacacagaatcaaccactctgttttctttactttcctctattaacaacaaatcaagcatctgcttgtctcagtttgaaatgctgcagagtagaagttccaaggccaacggaacggaactggatattgaagtctctgatggtggtttctctgtatattgcaggtttctcttctgatatcaactccttgataggtctatacaatttggaggtttgaaaatgaagcgtcgagaaaacccctgccactgatactctcgcatcctccttgtttgcaagaactctgtgcttaacgcttttgaatttgtcatttgttattagctgcataagatctccgatgttcaccactaaagctccagcgaccgggggcacgtcgacccagttgttctcataccgaatctgaaggccgccgatatggtcttgcaatagcacagtgataaagtcattgtcagtatgctctgatgtgccgatggctaaattcggctgtgggcatggtgggtagtagtggcatacaagtgcaagtccttcattgcagtctatatctttcaaataatttggattcaagtcaagagcttccgacagcaattcaaacagtaaattcccaatctccatcactcgttttgagtaatccacaagaacatctctgcaaaacacagaggttagttattagtaaaagggggggtgttcctttttcttcttttaataatcaaaattagtatctttacctgcaaatttccggcaggtcttgcggactgtggggacttggggcactgatatagccaaagctatccctccaattggtgatggctgcagagtacagatcgaaattactattgtaaatcaaaggtttggtgaggtcgcgagtgtaatattgtttcttggattcggtttcttgctcgtgaaatcttcgaactgcatctttcatctcctcaagaacgttctccggaatcccatggttaatcagttggaaaaaacccaatttctctgacgcctctctgactctgtcgatggtgtatttgcgtttgagtgaacttttgttgatttgatcgaggtctatcactggtatctggacttgggtttcggcggaaatattgtgggagtcataattctccggtgggcggtaaaagatacgcggaatttcggtgattccggcgtcgactaagcctttgacgccggctttggtgtcatcgaaagctttgaggtcagtgggtctgtggtagttttgatcgtctttggacagcggagtgaggttgagtccatcgactcttgagggtgcgaccgccattgtggatggaacagttgcctttgcccctaaagaacgtgttttgaaaaattgtaagtggttaggggttgtccattgtaatcgtagacatatttaataaagaaatgatgggggcatttatcggtttgtccctgctcactttaaataaataattgtgttaagactttggaatggtttgcatattaaaagttatacaactcttaaccacacactaagatcgtatagtctatatattcaccgcatatgatagatattcatttattaagtcgctaataaatctagatactgagttcaaccatgtaaacatccaactagcaataattttactatatgtatgactacctatagaataaatttgtggaccgacaccaagttatcgttattagattataataattaatagtttatggtataaaatactttagtatttttaataatagtttatgtttagagtttaaattatatttatttgatgtgaaataataaacactttaacaaacaaatataaagaagaagaaaaggaaaaaatattaaacaagattaatagttgcacatgccaataaccaatagttgattgactggttcaacttgataaaaaatatttttttctagacagcagagaagttgtcatgtttaacccaactcctcaataaataattattttagattggaatatgaatagggtaattaagcacgtgggttcttttgaataaataataggttgtgtatataaacaatatatataagttggtttgaaaatcaaacaagtggtgatcaatccgttacttgaacactttatatctccatgagctattttctttataagatgatggttatcttcttacctaggggatgtatagctagccatacttttttccgatttaactattcttacaaagagaacttaaccattaagtaaattaaaatgtatcaatcctctaaagtcaatacatcaagtcaatcagacatattggaaaaatccttaaatgcccgttttgtattaagtcgtgaatgacataaacgaggaagaaaaatagtgttgaatcaaatggtagaatttggacacttttttttccttctaatggtttttgaccaattcattcacaaaaccatgtggacattttgtcttatttgcaaacatggtggacaatttcatatgtgatgcatgccaacaatgtcacaaaaagtatatatcacttttaagattgaaataatttaatttgaaatttttaatcttttaattgagattatacattttaacgagttaaattatttaattttcaggttgtcaattgtatattattttacaattcattttttttctcataagattccnnnntctcataagattcccaaatataaacatattaatctacatcttttaaatcaatttgtaaagctaaatatgtcctaaacttagttatattttagatatcgtttctttatatatactatattaaattaataaaaataggtatagtatactgtcaaaatgactttccaaaacctctgtccttcaaggttggtttaatctctcatcgatggtataatatattcaatttttttgataagaaattgtgtcaaatgataaaaaaatttgaaaaaaaaagttaatgacacatttttttttgcactatcaaatgatattagatggttactaacttttaaatttgctacttttgcatatcattatcaaatggtattagatggttactaacttttaaatttgttacttttacaatttaaaaaaatgattgatattgacactattattattattattatttttttttttttgggtttttgcatgtgcctatttgtcttatttgaaaacaaattattgagaaagaggggaaacctatatataataagaagcacgtttcaaaatgaaaagtctcatggagataataattaataacaaaccaaaactattaaaataaaaggcaagaaaatttagccttcacatttatttttctgcctacacactttattatttaatgctttccatgtgcataaggtgcatacaagtttcttacaatacacggtcaccaatagcaaggtcaattatttgatagatgtgattttatttatttgcaaacatagtggcccattccatatgtgatcacacatgtcaccaatatatttcattctcttatactatttttaccatcaaaattcattaggaatcaatactttctaattttttctcctaaaaaacacaaaccagaaaatccaaaaaattgatagtaaaaaccaaatcaaaccatcaaccgaatttggattcaaaaggttagagaaataaagtaaaagaataaatacgtatgaatagttgtgggtggcagaggtttgaaagggtattttttttgttgaaaagcgaatgcacgcagattaatctgaagctttcattgctttctccacaaacacagattaaaggttcaaacaaacataaattacacatcaggcttttcactcaaatcttaagatgcgacagagcggaagtcccaagccctttagaacgatacagcttatccacttctctaattgtggtttccctgtacttcgcaggattctcttccgataacaattcctttaggggtccatacattttcgtgctcggaaatcccagcgtcgaaaaaaagctagctactgaaattctcggaccttccttcttcgccaacactctatgtgccacacttacgaatctgtcgtttgttattaactgcaaattcccagataatccaaatcaatactcacatacttaaagaaacagagagattacagaatcttgttatataacacacttacctgcataatctctccgatgttcaccaccaaagctccggcgacaggtggcacatccacccattttttctggtcttgaatctgaagcccgccgatgttgtcttgaaatagaacagtgatgaaaacattgtcactgtgtttggatgtgcctagtgtcaattcgggctgtggacatggtgggtaataatggcccccaattgcgagtccctcgctgcagcctatttcgtttaagtaatttgggttcagccctaaagcttccgacattaattcaaataacaatttcccaatccccatcactctcttcgagtagtccaagagattatctctgcaaaacaatcactgttaaatggaaatcagagtccaagaacatgaaatttcagtcaatttaattgtttacctgatgatttcgggtaaggcttgtggatcgggaggattaggggcacttacatacccaaccgtgtctctccaattcgtagtcgatgcactatacagatcgaaattgctgttgtaaattaaaggttttgtgtagttccgggtgtagtattgtttcctgacttccgtgtcttgttcgttgaatctcttgactgcatctttcatttcttcgagaacgttcaccggaatcccgtgattaatcagttgaaaaaaacccaatttctctgaagcttctcggactctgtttatggcgtcttttcgtttcagtggatttctgtggacctcgtcgagatcgatcactgggatttggtgatgagtttcgtcggaaagtttatgggagtagtaatcttccactggttggtaaaagatgcgtggaatttcggtgattcctgcgtcaactaagcccttaacgcctgcttttgtatcgtcgaaggctttgagttcggtgggtctatggtaattggcatcggctttggataaaggagtgaggatgaatccgtcggagggtgacacggccattttctgttatggaaatgatttattggactgtgtattattctatatgaatgaatggcttatagcttttgtattaaatggaaaggaaacttaattctttttatttttagaaatggttatatatatgtatagtgcaaaataagtatgtgtatgctctttcatttctattatgctttgtccagttccacacatgtaacacattcgtaacaaagtccttgctattcatatacattcaataaacttatatactcataacactttatacacttcataggcatgctattctctaattcatgttcgtatacatgtgatactccttgatgcactcgatacatttgatatttcttgatatcttttagattatgatacacttgatgtaaatgatattatttgatacaattaatgtgcattgttgaatgattgatgttgtcacgaccccatctagaacgccttcttcttgtattctcgatatggtcgagacttgggtacttaagatcaacttcttaatgaggtttgcttcttttttagaaaatttcaattgttatcaaacgtagggtgctttagttctttcattttctccaagaaagcgtgactttgatcatgtaaatttgtttgcaaaattaatcattgtttctttcgtgtaagaatatgtgaggtgttccaagacattgagtttttgtttgaaaattaaagttaagttatttagtttgctgatttatctttaatttaagtgaaacctattaatcttaattaactaatgttttcctagaatttgctagtttttgttgttcccagcaaaatttgaattacattaaatcatttctacataaaattaagttcaatttaagattaattctaaatatggaatgcactaatttcttaatggaaataaaagatgaattctagcttcaattttttcttcgttaaaaatgtcaatcttgccaaagttctctcataaattgtaatgtcatcttgatgttgggatgatttatgtttgaaatagaaaaagtattgtttgataattgttcctacaatttagaatgaattgcaattactcccaattcactataaatctctcaaataatgtaattgtataagctaataaaaaaataggttgagacaaacctattgaataaaatatggtagttttgagttttacaaattattattatttttatttaatatatatattgcacatccccaccccttcatttatattacaggcgtgactacactttcatatatataattaaaaaagagaaggcactaacgatcaaaactcctctatttatgtaaatggttttgcctcaaacttatttttgtgattcgtttggccattcacattattttagaaaaaaattccccaattaattgagaatcaagaatggatcttagtcaaatcaccacaagaaaaaaaaaaactaagttgatttctaaggatgacttgattagcaagaatggtaagcattattctttttcattttagatttgaagtaagaattaaatgaaattttagtttgcaaaattaatcaccctatttagtccataacaactccttttacattcttgcacataatttttaacgatttctttagacttgtagcatgtataaattttaaataactatttcttttaaacaaaaactaaactaaacttgctcctaactcaactctctaacaatttgtttcgtttgcttgaaaaagataaacttaaaacataagtttttataatgagtaacaatttagtactaatagaataggataaaagaaatgtcacattagctggtgctagtctggcctcaatgttttgtgttatataacccttgtttttggaagttttaagtacaactgggaagtttaattttaatttgaacttttattgacactaaaattaaaaaacttacacttaaatataaaatttaaataatatattaaaaatggaatggttataaaatttcaaactttttaaaattgaaaatttagggattcactttaaagataaatttattaaacaaatcctttataaatgccaaatcaaaactgaataaattagtcagccgactaaaaagatatagactcgaggatttagagatttttttagaaaaataaatttgatttgatggtttgaaatttgaaattttgggaagtggtgtgacactccaaattcctttttgtaggtcctcatttataactgtgttttgccatcaattttttttgcctctaagagttgcgagccagattacctttgtccattttcaaatatgggattcttctccgcccttggacgcctcctctttgcctccttcttcatcctctctgcttgtcagatgtaagttcacttgtattcctcactcaattctcatatttattggatctagactattgttgctattttggcttgtattccactccttttcatttcccaaatagctattttggctatttgggttttgcttccatttccaaagaaacgaaaactcaaatgaaaatggctatgctagttgttcttatgtgataatttttacttttaatgggccagatttattatgttcccatttattggtcttttatttggttattttcattgagagtggagggctcaaagcattttcactgccctaattttattatctataggccttcatttgtatatgtatctatattttacacagatatttatttattgtttttttaatatctgtgtgtttttcttaggttcagctcttttttaggtatgggtgttatatcaaggggatgaaattgcattgaaaatttccaattttcaaatttccattagctaatcctgggataacaaaaccaaggactcaatcaccagctgccttacctcattgaactaataaatgaaagtttaattatgagtgatctgtttttcaccaatttggtttcataaataaatagaagaaaagtaatctttaatctatctctgttggatgcacaatcttcctgaaaatcatttttttttggctaaaaaccaatttttgaaagttttatttagtttgaacacaggccaatctcacctgcactatgtttacatttaccttgttaggtttaacaataatggtgggccagcagctgtggaattgtcttcaaaggtagctgttctgaggagaaatttatcatccaagttggggttgctatacctgatattgatgtaaggccatgttaggccttattcaccacttcaatattgacaatattgaccatacctgtagactgaaattttgataattttctcaggtcagacatgtggttggtctttcattatctctcaaagggattggagctcttctatttgttttgggctcaagagttggagcttatcttttggtaaggattcaccatgttcaaaatttagcattgtacacatacatttgctacccacatttattcctaactaatgttactctaaatttatgcatctgcaaattgccctctctttcagcttctttacttggctattatcactccggttcttttcgatttcttcaaccttagtctactgaaacctacttttggtatcatgttaagtgatttcttagaggtttgtccatgtgctttctaactttttccatcttttcattttaagcatctccaatttaatcataagaatcctattctcttggcaatttaatcataagaaatttaatcataagaatcctattctcttggcagcacattgcactttttggtgctctccttttcttcatagggatgaagggcacaagtccaaagaagcactcaaaaaagaagacccaaaaacaaaaagcaaaagcaaaagcacattag

SEQ ID NO: 73 - DNA, 28180 bp, Cucumis sativus
  source 1..28180: mol_type = genomic DNA
ctaatgtgcttttgcttttgctttttgtttttgggtcttcttttttgagtgcttctttggacttgtgcccttcatccctatgaagaaaaggagagcaccaaaaagtgcaatgtgctgccaagagaataggattcttatgattaaatttcttatgattaaattgccaagagaataggattcttatgattaaattggagatgcttaaaatgaaaagatggaaaaagttagaaagcacatggacaaacctctaagaaatcacttaacatgataccaaaagtaggtttcagtagactaaggttgaagaaatcgaaaagaaccggagtgataatagccaagtaaagaagctgaaagagagggcaatttgcagatgcataaatttagagtaacattagttaggaataaatgtgggtagcaaatgtatgtgtacaatgctaaattttgaacatggtgaatccttaccaaaagataagctccaactcttgagcccaaaacaaatagaagagctccaatccctttgagagataatgaaagaccaaccacatgtctgacctgagaaaattatcaaaatttcagtctacaggtatggtcaatattgtcaatattgaagtggtgaataaggcctaacatggccttacatcaatatcaggtatagcaaccccaacttggatgataaatttctcctcagaacagctacctttgaagacaattccacagctgctggcccaccattattgttaaacctaacaaggtaaatgtaaacatagtgcaggtgagattggcctgtgttcaaactaaataaaactttcaaaaattggtttttagccaaaaaaaaatgattttcaggaagattgtgcatccaacagagatagattaaagattacttttcttctatttatttatgaaaccaaattggtgaaaaacagatcactcataattaaactttcatttattagttcaatgaggtaaggcagctggtgattgagtccttggttttgttatcccaggattagctaatggaaatttgaaaattggaaattttcaatgcaatttcatccccttgatataacacccatacctaaaaaagagctgaacctaagaaaaacacacagatattaaaaaaacaataaataaatatctgtgtaaaatatagatacatatacaaatgaaggcctatagataataaaattagggcagtgaaaatgctttgagccctccactctcaatgaaaataaccaaataaaagaccaataaatgggaacataataaatctggcccattaaaagtaaaaattatcacataagaacaactagcatagccattttcatttgagttttcgtttctttggaaatggaagcaaaacccaaatagccaaaatagctatttgggaaatgaaaaggagtggaatacaagccaaaatagcaacaatagtctagatccaataaatatgagaattgagtgaggaatacaagtgaacttacatctgacaagcagagaggatgaagaaggaggcaaagaggaggcgtccaagggcggagaagaatcccatatttgaaaatggacaaaggtaatctggctcgcaactcttagaggcaaaaaaaattgatggcaaaacacagttataaatgaggacctacaaaaaggaatttggagtgtcacaccacttcccaaaatttcaaatttcaaaccatcaaatcaaatttatttttctaaaaaaatctctaaatcctcgagtctatatctttttagtcggctgactaatttattcagttttgatttggcatttataaaggatttgtttaataaatttatctttaaagtgaatccctaaattttcaattttaaaaagtttgaaattttataaccattccatttttaatatattatttaaattttatatttaagtgtaagttttttaattttagtgtcaataaaagttcaaattaaaattaaacttcccagttgtacttaaaacttccaaaaacaagggttatataacacaaaacattgaggccagactagcaccagctaatgtgacatttcttttatcctattctattagtactaaattgttactcattataaaaacttatgttttaagtttatctttttcaagcaaacgaaacaaattgttagagagttgagttaggagcaagtttagtttagtttttgtttaaaagaaatagttatttaaaatttatacatgctacaagtctaaagaaatcgttaaaaattatgtgcaagaatgtaaaaggagttgttatggactaaatagggtgattaattttgcaaactaaaatttcatttaattcttacttcaaatctaaaatgaaaaagaataatgcttaccattcttgctaatcaagtcatccttagaaatcaacttagtttttttttttcttgtggtgatttgactaagatccattcttgattctcaattaattggggaatttttttctaaaataatgtgaatggccaaacgaatcacaaaaataagtttgaggcaaaaccatttacataaatagaggagttttgatcgttagtgccttctcttttttaattatatatatgaaagtgtagtcacgcctgtaatataaatgaaggggtggggatgtgcaatatatatattaaataaaaataataataatttgtaaaactcaaaactaccatattttattcaataggtttgtctcaacctatttttttattagcttatacaattacattatttgagagatttatagtgaattgggagtaattgcaattcattctaaattgtaggaacaattatcaaacaatactttttctatttcaaacataaatcatcccaacatcaagatgacattacaatttatgagagaactttggcaagattgacatttttaacgaagaaaaaattgaagctagaattcatcttttatttccattaagaaattagtgcattccatatttagaattaatcttaaattgaacttaattttatgtagaaatgatttaatgtaattcaaattttgctgggaacaacaaaaactagcaaattctaggaaaacattagttaattaagattaataggtttcacttaaattaaagataaatcagcaaactaaataacttaactttaattttcaaacaaaaactcaatgtcttggaacacctcacatattcttacacgaaagaaacaatgattaattttgcaaacaaatttacatgatcaaagtcacgctttcttggagaaaatgaaagaactaaagcaccctacgtttgataacaattgaaattttctaaaaaagaagcaaacctcattaagaagttgatcttaagtacccaagtctcgaccatatcgagaatacaagaagaaggcgttctagatggggtcgtgacaacatcaatcattcaacaatgcacattaattgtatcaaataatatcatttacatcaagtgtatcataatctaaaagatatcaagaaatatcaaatgtatcgagtgcatcaaggagtatcacatgtatacgaacatgaattagagaatagcatgcctatgaagtgtataaagtgttatgagtatataagtttattgaatgtatatgaatagcaaggactttgttacgaatgtgttacatgtgtggaactggacaaagcataatagaaatgaaagagcatacacatacttattttgcactatacatatatataaccatttctaaaaataaaaagaattaagtttcctttccatttaatacaaaagctataagccattcattcatatagaataatacacagtccaataaatcatttccataacagaaaatggccgtgtcaccctccgacggattcatcctcactcctttatccaaagccgatgccaattaccatagacccaccgaactcaaagccttcgacgatacaaaagcaggcgttaagggcttagttgacgcaggaatcaccgaaattccacgcatcttttaccaaccagtggaagattactactcccataaactttccgacgaaactcatcaccaaatcccagtgatcgatctcgacgaggtccacagaaatccactgaaacgaaaagacgccataaacagagtccgagaagcttcagagaaattgggtttttttcaactgattaatcacgggattccggtgaacgttctcgaagaaatgaaagatgcagtcaagagattcaacgaacaagacacggaagtcaggaaacaatactacacccggaactacacaaaacctttaatttacaacagcaatttcgatctgtatagtgcatcgactacgaattggagagacacggttgggtatgtaagtgcccctaatcctcccgatccacaagccttacccgaaatcatcaggtaaacaattaaattgactgaaatttcatgttcttggactctgatttccatttaacagtgattgttttgcagagataatctcttggactactcgaagagagtgatggggattgggaaattgttatttgaattaatgtcggaagctttagggctgaacccaaattacttaaacgaaataggctgcagcgagggactcgcaattgggggccattattacccaccatgtccacagcccgaattgacactaggcacatccaaacacagtgacaatgttttcatcactgttctatttcaagacaacatcggcgggcttcagattcaagaccagaaaaaatgggtggatgtgccacctgtcgccggagctttggtggtgaacatcggagagattatgcaggtaagtgtgttatataacaagattctgtaatctctctgtttctttaagtatgtgagtattgatttggattatctgggaatttgcagttaataacaaacgacagattcgtaagtgtggcacatagagtgttggcgaagaaggaaggtccgagaatttcagtagctagctttttttcgacgctgggatttccgagcacgaaaatgtatggacccctaaaggaattgttatcggaagagaatcctgcgaagtacagggaaaccacaattagagaagtggataagctgtatcgttctaaagggcttgggacttccgctctgtcgcatcttaagatttgagtgaaaagcctgatgtgtaatttatgtttgtttgaacctttaatctgtgtttgtggagaaagcaatgaaagcttcagattaatctgcgtgcattcgcttttcaacaaaaaaaataccctttcaaacctctgccacccacaactattcatacgtatttattcttttactttatttctctaaccttttgaatccaaattcggttgatggtttgatttggtttttactatcaattttttggattttctggtttgtgttttttaggagaaaaaattagaaagtattgattcctaatgaattttgatggtaaaaatagtataagagaatgaaatatattggtgacatgtgtgatcacatatggaatgggccactatgtttgcaaataaataaaatcacatctatcaaataattgaccttgctattggtgaccgtgtattgtaagaaacttgtatgcaccttatgcacatggaaagcattaaataataaagtgtgtaggcagaaaaataaatgtgaaggctaaattttcttgccttttattttaatagttttggtttgttattaattattatctccatgagacttttcattttgaaacgtgcttcttattatatataggtttcccctctttctcaataatttgttttcaaataagacaaataggcacatgcaaaaacccaaaaaaaaaaaaataataataataataatagtgtcaatatcaatcatttttttaaattgtaaaagtaacaaatttaaaagttagtaaccatctaataccatttgataatgatatgcaaaagtagcaaatttaaaagttagtaaccatctaatatcatttgatagtgcaaaaaaaaatgtgtcattaactttttttttcaaatttttttatcatttgacacaatttcttatcaaaaaaattgaatatattataccatcgatgagagattaaaccaaccttgaaggacagaggttttggaaagtcattttgacagtatactatacctatttttattaatttaatatagtatatataaagaaacgatatctaaaatataactaagtttaggacatatttagctttacaaattgatttaaaagatgtagattaatatgtttatatttgggaatcttatgagannnnggaatcttatgagaaaaaaaatgaattgtaaaataatatacaattgacaacctgaaaattaaataatttaactcgttaaaatgtataatctcaattaaaagattaaaaatttcaaattaaattatttcaatcttaaaagtgatatatactttttgtgacattgttggcatgcatcacatatgaaattgtccaccatgtttgcaaataagacaaaatgtccacatggttttgtgaatgaattggtcaaaaaccattagaaggaaaaaaaagtgtccaaattctaccatttgattcaacactatttttcttcctcgtttatgtcattcacgacttaatacaaaacgggcatttaaggatttttccaatatgtctgattgacttgatgtattgactttagaggattgatacattttaatttacttaatggttaagttctctttgtaagaatagttaaatcggaaaaaagtatggctagctatacatcccctaggtaagaagataaccatcatcttataaagaaaatagctcatggagatataaagtgttcaagtaacggattgatcaccacttgtttgattttcaaaccaacttatatatattgtttatatacacaacctattatttattcaaaagaacccacgtgcttaattaccctattcatattccaatctaaaataattatttattgaggagttgggttaaacatgacaacttctctgctgtctagaaaaaaatattttttatcaagttgaaccagtcaatcaactattggttattggcatgtgcaactattaatcttgtttaatattttttccttttcttcttctttatatttgtttgttaaagtgtttattatttcacatcaaataaatataatttaaactctaaacataaactattattaaaaatactaaagtattttataccataaactattaattattataatctaataacgataacttggtgtcggtccacaaatttattctataggtagtcatacatatagtaaaattattgctagttggatgtttacatggttgaactcagtatctagatttattagcgacttaataaatgaatatctatcatatgcggtgaatatatagactatacgatcttagtgtgtggttaagagttgtataacttttaatatgcaaaccattccaaagtcttaacacaattatttatttaaagtgagcagggacaaaccgataaatgcccccatcatttctttattaaatatgtctacgattacaatggacaacccctaaccacttacaatttttcaaaacacgttctttaggggcaaaggcaactgttccatccacaatggcggtcgcaccctcaagagtcgatggactcaacctcactccgctgtccaaagacgatcaaaactaccacagacccactgacctcaaagctttcgatgacaccaaagccggcgtcaaaggcttagtcgacgccggaatcaccgaaattccgcgtatcttttaccgcccaccggagaattatgactcccacaatatttccgccgaaacccaagtccagataccagtgatagacctcgatcaaatcaacaaaagttcactcaaacgcaaatacaccatcgacagagtcagagaggcgtcagagaaattgggttttttccaactgattaaccatgggattccggagaacgttcttgaggagatgaaagatgcagttcgaagatttcacgagcaagaaaccgaatccaagaaacaatattacactcgcgacctcaccaaacctttgatttacaatagtaatttcgatctgtactctgcagccatcaccaattggagggatagctttggctatatcagtgccccaagtccccacagtccgcaagacctgccggaaatttgcaggtaaagatactaattttgattattaaaagaagaaaaaggaacacccccccttttactaataactaacctctgtgttttgcagagatgttcttgtggattactcaaaacgagtgatggagattgggaatttactgtttgaattgctgtcggaagctcttgacttgaatccaaattatttgaaagatatagactgcaatgaaggacttgcacttgtatgccactactacccaccatgcccacagccgaatttagccatcggcacatcagagcatactgacaatgactttatcactgtgctattgcaagaccatatcggcggccttcagattcggtatgagaacaactgggtcgacgtgcccccggtcgctggagctttagtggtgaacatcggagatcttatgcagctaataacaaatgacaaattcaaaagcgttaagcacagagttcttgcaaacaaggaggatgcgagagtatcagtggcaggggttttctcgacgcttcattttcaaacctccaaattgtatagacctatcaaggagttgatatcagaagagaaacctgcaatatacagagaaaccaccatcagagacttcaatatccagttccgttccgttggccttggaacttctactctgcagcatttcaaactgagacaagcagatgcttgatttgttgttaatagaggaaagtaaagaaaacagagtggttgattctgtgtgttctcagttttgattgagaacttgtgatcgtgacctgcacaatcctatagctttaaactgaaacttgttctacggtgtacgtacgttgttgtcaatgtaagattgttagatgaatatgaataaataataaaattggcgtgttagagatatcctatcaaattcttggatgagtaattttgatacaataacagttgcaatttgaattaagaatcttgatttcagattgggttagatacggattgtttttaagatcaaacaactggatgaaaaaaaataagtttttttaactctgaaacactccacaagaaaaattaatcaatcctcttaaatgttctgcatgtaattcaacacatgatttaaaaatttaaaaattcttagactaatcttgaaaacaatcacaaagcctaaatctcaaatcatccaaatcttcttaaaacaatcatagcaaaactaagtctctccaataaaaatcctaatgcaacactaaaatgaaaaaaatatccgtgcttaacttaaattaagaaatactaaaattacatcaaatttggtttgaatcctatattggtgttgttctattttgatttataaacttctaaaaaacaactttttaattttattttttttaaaaaaagtaatctatggtcctttatttatccctaactaaattttttaaagtttattttatgtggtcatggatacaacttctattttattaattcattcattctactcaatgttcttaacttcaagattccaatttgaaaataaacttaaaagattccaatttgaaaataaaaagaatgggtgggagccgaagaggaaatgtaagaaaattgagttttagagggaaaaataataaaattagtttttgagttcgtttgattcacctttattgattatgtttttgtgctatataaaatatttatagaataaattagtactatcgtttttggttatatatcaattcaattaattgattaatctaatgttcaatcttgaaaatttatggactaaaatatgatttaattcaataaaattttattaataacgtgatgccttagagagttcatatcacactctactaatccacatgtcaaacgtaaatatgttggatatttatttcaacgaattcttgtacttagcaaacctcacaaatttgataagaaatgtttcattgatatatgagtatactcaatatttctaactcaagttttaactttattatgtgttgatgcgttggaaccaaccagctttgcaagataagataaagatagatataatggcggatgcaggcaacaaattcatcctcactccactgtccaaaaccacagaaaattacgatagatcctctgagctcaaagctttcgatgacactaaatccggtgtcaaaggcttagtcgacgccggagtcactgaaattccacgcatcttttacagtccaccagaggactcccattccgacggtgtttctagcaaaacccaaatccagatccccgtgatagatctcaaccacgttggcactaattcactcaaacgcaaatacaccaccgacagaatccgaaaagcttcagaaaaatttggatttttccaactgattaaccatgggattccggtgagcgttttggaaggggcgtatgaagcggttcgaagatttaacgaacaagaaacagaagtaaagaaacaatactattctcgcgacttctctaagccttttatttacaccagcaatttcgatctctactttgcaaaaaccaccaattggagggacacatttagatgcgcaagcgccacaacttcccaggatcagcaagacataccagaaatttgcaggtaaaattagctctgtctttgaagcctaaagaaagaaaagaaaacagagatccctgtttatgctaacattttgttattgcagagacattctgctggattactcaaaacaattgatggaacttgggaagttactatttgaattgttatcggaagctctggggttgaatccaaattacttgaacgatataggatgtagcgaaggggtttcagttctgtatcactactacccgccatgcccacagccgaatttgaccatcggcacatcagagcacaccgacaatgattttataactgttctgtatcaagatcaaatcggcggcctacagattcgttacgagaacagttgggtggaggttgcccctttgggaggagctttagtggtgaacatcggagattttatgcagctgacaacaaacgacaaattcaaaagcgttaagcacagagttgttgcgaacaaggaaggtccaagagtatcagtggctggttttttcaccgagtttccattttcgacgtccaaattaagacccattgaggagttggtttcggaagaaaatcctgcagtatacagagaaaccacaattagagacttcaacatcctgttccgttcaaaaggtattggaactactactttggagcatttcaagataaaacctggagatgcttaattaatagttagtagatgaagaagtgattagttgtggatgttcgttctctctttgattgaacttctatctattattgaaaaactaaagtgaattaaaggttgctccatgccgtttctgtcatactataatagcaagaatatgtttatgtaattattccaaggtttccgagttaataatcttagattattcttcacaaaacgtccaaaatcatgtaaaaaaaaaaaaaaaaggctaaggtcattgactattgcttcaacaatgctaaaacatagaaatgttaatacatcattaactaacatacaacatagaaacatgaatttaaattttcccttcataattattttaacctaataaaaaggtattaaccttaatgaatgaaagtgattatttaatgaaaagctaaggttaaaaatgtaaatatggtaaattagagaacaaaacgaaataaaatttaaattttaaaggtaaaattgaataaatagatgggatgatgttctatccaaacttaggaataaaatgagttaggatatgcattttatcgtgatactgactactattattaatattacatttgttttttctatatatcatattacaaataccataaaattgagagagattgagtctcaagtccaccgtaaattcacgtgtttgaattgtcatattgaagaaaaaaatgtacgaaaattgaaatcgtggaacactctatcaataattcgttggaccaccttggctaaaagcaatttatttttgaaaggtctaaaagcaactcttcttgcacctcatgaaacaaaagtgtcatattcatatatgcttatttacaaggctaattcttctcttcttccaccttctctttctttcattttttttttttaaattcaacatattaaaggttagtcaaactatataaaagataaatgcaaccaaataaaaatgtgccacgtggtactctttttggaaaatattagaaaattgatgtttattttgagtctctcccttattataaatcaacttttttaattctctaccttcattttttcttcttctttttatattttcaaccaaacttatttatattttaaaaatagtaaaataaattaaaatatttataagatatagcataattttggatttaaagaatagaaactaataaaaaattattcttattttggagaaacttatttataactagtctgtataaaaaaaccaagctcaccttaacacaatttcccttgagataattaaacacaaaaataaatatacccctaattccttaaaatattaataaataaattctaaaatataaacttttcaacatgactgagtttaaaattcaaaggaaaacctatatgaattatgaacatctatctatttatccaatcagtcaaaatttgaaaattcctttctagtggacaacttgaacttgaattacaattagagcccaccaccaacttacaaaccctggctatcctactacttcaccatttatatcccaccccttcccaccattcaccaaaccacttttacaattttgaaatcacactcttcattccataaattcaatctcttccatcaacccaacagatccatatccataatggcggtcgcagcttcaggaatcaaccttactccactatccaaagccgacgaaaacttccatagacccactgaactcaaggctttcgatgacactaaagctggcgttaaaggcttagtcgacgcaaaagttaatgaaatccctcgtatcttttaccacccaccggaggacgtccactccgcccaaacccatatccacattccattgatagaccttgaaggcgttggcaacgattcactcaaacgcaaacacatcatcgaacaaatccgtgacgcttcggaagaattgggcttcttccaattgattaaccatggaattccgacgagtgttcttgaagaaatgagagactctgttcggagatttcacgaacaagacacggaagataagaaacaatactatacaagagatttcatgaagccttttatttacaatagtaatttcgatttgtatactgctcccactaccaactggagggatacatttagttatgtctgtgctccaaatcctccaaatccacaagaattgccggaaatttgcaggtaaattgatcattttggacctaaatcaaataatctttttagtttaattttgggtttattattaacctctgtttttttttgtagagatattatggtggattactcgaaatgggtgatggagatcggaaaattagtgttggaattgctgtcggaagctttgggtttgaatcccaactacttggacaacataggatgcaatgatgggcttgcatttgtatatcactattaccctccatgtccacagccaaaattgaccaccggcatatccgagcactctgacactgacttcatcacggtgctgttgcaagaccacatcggcggcctacagattcgtcatgacaacaagtggattgacgtgcatcctgtagctggagccttagtcgttaacatcggtgaccttatgcaagtaagactcgtttgtttttatagtaatccgagcttatcactttttttttcttttaatctcatctaatttttttctataacataacaatttggatgcagctaataacaaatgacaggttcaaaagtgttaatcatagggttctagcaagtcatgaaggtccgagaatatcagtagcgggcgttttctcaacgatggtttcaccaagtgacaaactttatggacccatcaaggaattggtatcagaagaaaagcctgcaatttatagggaaactacagttagagactttagtgttcaattcaagtcggatgggcttggaacttctactttgaagcattacaagctgaatcaagcacacatttaaattgcaattatattgacgtaccatcccccataataattcgtggaattgtttagatccatctgtcttatattttctttcttcatctttccatcccacaaaaatattttttttttgtggatgagcgatgtgatgaagaacgtaaatgatagatatgcctcaagtttgttttcataaaatacggtctttatctatctttcttctaactattttcttccactactattgtaaaagttgaagttgaaattaaacaaaacaatatggcttctagataattagtggtcaatataatttggaaacactatataacaaaaaaagaagaaaaatagaggcagtagataagattcaaatttaaaatgttaaagttgtgaagggaatagaatttagtaataataaagtattatttaatatatattataatataacattttaaaattaatattaaaatataaaaaaatgtaaatgacaggtgttgctagctaaatgatgccacgattattttgaatcaaaattaggaactaagtcaaacccaaagtttaatattaagggcaacacttaggatgcacccatccctatgaatttccttttcatgactgagataaataattaagaatatttaataaaaattagggatatagaacaaaaatagataaaacataaataataggtcgggatgcaaaaatttcgtaaatgcgcatttattggcattcataattgatgacatgcatttgtatttgtgaataataaaaaaagatgcaaatatgtaatttgtcgttgtaatgcacccaagaggtacgatgtaggaggggacatgaaaccatgagctaacaaaataaaataaaagaatttgttaaaatatttcgagtgttagtcgcgttagatttaactaaatctttatatcaatgggagtctagaaacttgaaactgaaattgagattctcaaacgaaaatctaatcagagaagattagattagttgcaaattacaatgttgcaccaaggcaccaatggagtccaccgaattgacttaactccggcagccaacggggacgaatatttcgacagatctgctgaactgaaactcttcgacgacaccaaagccggagtgaaaggccttgtcgacaacggcattacccaaataccgcgaatattttaccgaccgcctgattccagcgactaccctgttgccggggacaccgaactgagcattccagtaatagacctcgaaggcattgacgcagattcatccaaacgaagacatgtcgtcaatacagtcagagaagcctctgagaaatggggtttctttcaattggtcaaccatggagttccgctgagtgttctggatgaaattaaaaatagaacactaagattttacgaacaagataccgaactgaagaagctattctacacccgccacaacacaaaatccattgtttacaacagtaatttcgatctgtttactgcaccagccgctaattggagagacacattcctctgtttcatggcccctaatgttccaaacccacaagacttgcctgaagtttgccggtaaaattctggttttcgatcagattaaaaaaacaggctctttttttttcttctcttttgttaattagctggtgtttgttgtagagatatcctgttcgagtattcaaaagaaatgaagaaattggggcgagtactatttgggatgctttcggaggctcttggcttgaacacgaattatctaagcgatatcgaatgcgacaggggattagctgttctgtgtcattactatccagcatgtccacagccagagctcactttaggcacaactgaacacgccgacaatgatttcttgacgcttcttctacaagacgaccaaatcggaggtcttcaagttctccacgaaaagaagtgggttgatattcctccaattcccggagcactggtgatcaacattggggatcttcttcaggcaagcttataagatttacccataaaaattgagaatgaaagctttcccatttcttaacacttaccattgttgcagctgatttccaacgatcggttcaagagcgtggagcatagggtggtggcgaatcgagaaggtccgagagtttcggttgcgagcttttttgggatcggcgtttatccaacatctcaagtttatggacccataaaggaattgttatcggagaaaaatcgtgccaagtatagagaaactacgctcaaagacttctatttctaccacaatatgagagggttgaatggaatttccgctctgcaacatttcaggctcggtcccgaggatgaaagaaatggttagtggccgattgtttttaattttagtgggtggaagtagaagaggaagatctacgggagtgtttaggttagtttgattattattatttctgaaagctggttggatttgagatctgttatttgaagtttattgactctgttcgtcgatttcaaacaatggcgcaccaaatagttaaaggatattttcctacttaattgattgtttcgggttttcttgactacattccaaatgctagttcaaatagtttcgagcattgttaaatttttgttgatccatatatgacaaaagatttaatgttctattatttctgacaattttccacaactttttggtattaacacccttgaacaaagctcacaataaacttcccatctagtaagcaagttaattagctgatgtatataattcatagcattgttctgtttagccatttcacgatcaatatcatccaaacacatagaaattctataattttaacaagttggaaaagagatattccctaaaaaaacactaaattaaagatgatctaatttttaagacaaatgaaaagtgacatttacttgatgctttatagataaaagatacgatactcctctattttctatactttatactgaacagtaaacactagcttattgaagttttatgtgtgttatacatgtttgtcactcttcttaaatgaacacaaaaagaagaaaaggaaaaagggaaaggttagaatgctataaataacagaggaagaattccagaaaaaaacacaaaagaaagaactataaaaacggaagaagaaatacaaaagaggatcacaaaaaaagaaaagaaaaaagaatggttataaaagaagaagaagaaaggaagataaaaaaaacaaaagaagtacaaaacgaagaaaagaggaaagaaacatttaagactaaagttgatgattttacatatgatgacataaacaaaaacaccaattttaaaaagtacatatcttttatttgaagctttccttcttattttgaggacactcccacaaaatttccatttttggtaagtgttactgccgtgggctatgaattaagttggtcttttatttattgttttggttatttatttttttaattatctccaaaattttagcaagcactatttcatttactctttttttttcttctttttattataaatattagtccattaaattgaataaatgcaataagtttcatttattttttttctctcttgacaatatatcttagagagtatttatgtttgttttcttcgagaagagattttatggatcatttgttttaaaatcaataactgttagttctttgagatctttagatatatatatatatatatcaaattaattagatatatagcacaattttaaaaaaattacaaatataacaaaatttgtcaaattctatcaatgatataagtctatcactgaccgactatgttgtaaatattaatctatcacttatataccatatgaaatctatcagcaatacaagtctatcgatgataattttgtaatatttacaattttttaaaatgttgaaatatccttaattttgtaataatttacaatttttttaaaatgttgaaatatcctttattattattgttaaaatagtcgtaaattccaattttcatatatttttatttgagcccacaatttgatcggagactattgaagtctccgtcaccatcaaatttctctcccctcaagatgaatcgaacatcgcctcctcaaaattggagtttcgaaatacacaaaaatggccaacctcacaccattctccaaacttgacaaaaccttcgacagagcttctgaactcaaagccttcgaccaaacaaaggctggcgtcaaaggccttgttgactccggcgttgcggagattccaggaatattctattgcccacccaaagaatgttcaagttccattccggaggaggaaacccatttgagtgttccggtggtggatcttgaagacatcgataaagacccattcaaacgcagacaagtggtggacaaaatccgagaagcttcagaaactgggggtttttttcaagtgattaaccatggagttccggtgagtgttcaggaggcgatcatagatggggttcgtcgatttttcgaacaggacagtgaagtgaagaaacaatattatacaagagactacactaaacctttcatttacaattgcaatttcgatttgtttaccgcaccagtggccaattggagggataccatcgtcactttcatggccccaaatcctcctaacccacaagacttgcctcaagtttgcaggtgatagatactctgcccattatttctctctgttcttacttcttaattgccgcttcccatctcttcaaacatttttgggaaaaatccatttttcttatccttagattttaatacagtatagatttgttcaactgtaaaatgtttattttttattaattgaacacataaatggacaccctctgtccaaaagcagataatatttttgtttgatgcattaacttatttaaaatttttgtttcagagacatccttcctgaatattcaaaacaaattatgaagcttggagaattaatatttggtttactttcggagggtcttggtttgaaatcaactcatctcttagacttggactgtaacgagggacttcgcgtaatctgccactattatccgtcaagtcctcaaccggaactctgcatcggcacaactgaacactctgatgattcattcatcaccgtgctactgcaggacaatatgggaggcctccaagtgcgtcaacaaaacaaatgggtggatgttacaccagttcctggggccttcgttatcaacgtcggcaacttattgcaggcaagtgatttgacaattatttccattgatacaatcctgattgatttggatacatattaataaagggaaaattaacgaaaggtgtttggggggttggttttgcagttgataacaaatgatagatttgtgagttcagagcataaagtattggcgaaccgtgagggcccaagagtatctgtggcaagtttctttagcaccggccgtctaccaacatctaaactttatggacctatcaaagaattgttgtctgaacaaaatcctcccaaatacaaagaaatcactgttagagagtacaatatctactttaatgaaaaagggttggatggcactcctgctcttccacatttcaagatttgattacaaagatgattgaaaagtcagttactaggggaatcatagtataatttgttgtgagttgttatgagtttggcgtacaaatttcttttattttatgctattatcggatggtcagggctgttgctaaataatatgtttttctctttttctgaaaggtcctaatagattgagtccttctcgggattgtgcaagatttggttgttccccgacttttaataacctagattgcagcttgcgagtgatagtactatatatatatatatatgcatacaagtcaaaagttgggctattttcggtgtgttgttgtgtggaggagagtgtggttttgaagctgagtttctcctttacaacatctttctgatatgtcatattgaacacttggtgaatgttttgatgcagtagatcagtgtgtgcgaagatctataccattggggggagcctaacttgttgaaagagtttgtttagtagtttctcactcaagagtatcggtgacatgctttttcaccactgcctctataccaacctccaaactctatgaacccatcgagcagttgttatcgaaacaaaatcctcccgaatacagacaaatcacagttagagaatatgttcttcaccatgctcagaaagggttggatggaactcatgttctcacacattttaggctttgatttgcttcacctcgtctttggttcgctgttccaaaccccaactttttagtttgttaaggtcagtgtgttgaataatgtttattaaatccgccttccactgatttaattaagaaggtttgttgtttctaatattttgttaaacatgggtttattaaaatgttattgttttgttgtaaggtttgctatgtctagagattatttgttaaattgttgtattgaagtttgagtttttgttagtgtgacttttaatttattcctgacctataatgtcaactattgtaatttgtgtgtttggtttaataactttttttattactgcctttttttttcatttttttctgaaaaagaactatcattttaaaataataacaatggaatgtctagcaataactcaaaatagtttcttttgaaaaagatgaatttatgaaattagttgatgaacttatgaaattagttggtcaaagaaacacatatctgaaatcaacttttagaaaaatggttttggtgtttaataatccctcgacttatctctattcgttgtcagtcaccctccaaactttaaattaaatttggatggtcatgaaaagttgattaattataactattttcaattaattaatagatgttaacataggatagcattcttatctcatttgtaaatatgttggttgtgagtgctctatggtcttgacctcaaactcacattccttttgtttgatctcgatcttgttcaaactcacctctatttaagggatttttgagcctacagtgattgaatgcgtgactaactcaactaaacaataaagaactaaggttcttggtcttttaactctcatttgaagatacttattattctcgttttttggctgtggagtgaaactatttataaattggtcctctaatacacatgttcatatttgtgtgtgatgaaaggagggttggggacgtttttggcacttgaggtccacacttggaattcgccattgacaccactaaacatgctaacaataccttcatatcgcaatgctttattaaatatttgattaaaatgtgttaaaactgagtctgttgagtctggagatgttcttttgtatcaactatcttttgttgtgtcaagatcattaatatatagaaacaataataattatcaaagtaaaggcataggccattttaatgtgacaaaaaccaaggacccatataaggccattcacgtcctcaccccctcaaacccaatcatttcttaactatcctctacttttggaccaacaatttgtgattagttgctggttttgtttgacgttggagggtaaaacttcattagcagacaagattacataaacactggtttgaatgaggtttttcaaggggagactcttccaccttaatctaataaggagtgatttgcatgatcaaggagaggattttgtttagcaaaatttaactttgaaattgtagtgacaaagtggctttactaaactgtatcagtgtagagtgtgaatctaagttgattaaaaatggtatgttagtttaagtttgtttactactgactatacagttattgttttctcaagagggataccatacccgatcactcgtcccaatcaaaacaaagaaacaaaaaaataaaagcggaaatatttgttggtggaaaattttgtgtggatggaaaataggagggggacgggaagtggcatccccgatcccacgcctccccgtgtaatctccaagttcacgtcctcacccccaaataatactatctccgttcagaacactcatcaaacaaaaatggcaaacctcacacctttctctaaacttgatcaaaccttccacagaacttccgaactcaaagccttcgaccaaactaaggccggagtcaaaggccttgtggactccggcatcaccgaaattcctgctatattttactacccacacaaagaacgttcaaattccgacaagacctctgttcccgacgaaccccattttggcgttccagtggtggatttggaagacatcgataaagatccactcaaacgcaaacaagtggtggacaaaatccgagaagcttcagagacttgggggttctttcaattgcttaaccatggagttccggtgagtgttcaggaggagatcataaatgggacacgtcgattttttgaacaggacattgaagagaagaaacaatattatacaagagacaactctaaacctttcctttacaattccaatttcgatttgtttattgcaccctttgccaattggagggataccatcctcactcaaatagctccaacttcttttggcccacaagaattgcctcaagtttgcaggtaatctctactctctccactctctgtctctgttcttttgcctaattacgatagctaacatgccccccaagacttttctcgtcggtaaagtgggtgttggtgtttggagctgttgtttgcaattcatatggttaagctatgcttgttacgtataatcatttctgggatccatgaatgtagttgaggatattattatattaaacgatgtttctcagctcttggagtgggtattacctagtggaaacttactctattagtggttgggaattctaactctgcgattgaggttcaggattagcttgatacatacttagacaagtccatttaagtggttctattaggaaattcttttggattaatcgcaatcgtaacgtctttgcacttgatttagcgttccatgctcagacattttggacactcgttcgtgtgtcttgaagatcatcagattctgccattttatttttactgacgtgttgttcttaaagcttctttttgttttcagcttgttatatatgtatatatattaaattactaacagttgtctatctaaatagataacaaattattgtaattggtgttatttattgtgattgatgataaagatttttgttgcagagacattctacttgattattcaaaacatatggagaatgttggagaattgatatttggactactttcagaggcccttggtctgaaatcaactcatctcgtggacataggttgcaatgagggacatgccttgctctgccacttttatccatcgtgccctcagccggaactcaccattggcatcaccgagcacgctgatggtaccttcatcaccgtactgctgcaagaccatatcggaggtcttcaagtgctacatgacaacaaatgggtagaaattccacccattcctggagctttagtcgtcaatgtcggcaacttactgcaggcaatgttttgttatgcattcattaagtaacaagatacacatatatagaaaatataatgatgggtttgggttcctttatggatgcagttgatctcgaatgacaaatttgtgagctcgatccatagagtgttggctactcgaaatggtccaagggtatcggtggcaactttcttctccactggctatgcagaaaccttcaaactttatggaccaatcgaagaattgttgtccgaacaaaatcctcccaaatataaacaaactactgtgagagattaccgtctctacttttctaaaaaagggttggatggaatggatcctctcacacattttaggatttgattttactgaactgtttttttggttcataattctaaggtttgttaaatgtttaagttcgactttgttagagtgtgtgatttttaaacactataacattgattttattgaaatgtcttttcctaccctgttgtaaggtttgttatgtctttagaaattatttgttaaacctttgattgaattaggtttgagtatgtcaactaagtttaatttagtatctacaatttcaaagattggaatttaaacttcagtaattaaaaaaagagaaagaattagacaaaatgttttaaaagttggaatttagtaaaagaaattttggatatgaatctcatatctttaattacttaggttaatatgttttttaaacactttgaacactttgaacactgggatatttttgaatcatgactcgtacgaaaaaccaagaccagagagaaaggcagcatgagggggtctgagttatgaactatcttctcatgttttctgtctgtatttgccgacagtggattagcaagcattaacgtccttctcacagcaaaaataactatctccattcagataaactgattaaattaaaagaccatacacagtacactcctcaaacaaaaatggcaaacctcacacctctctctaaacttgatgaaaccttcgacagagtttccgaactcaaagccttcgaccaaactaaggccggcgtcaaaggccttgtggactccggcgttgccgaaattccaagaatattctactacccgcacaaagaacgttcaaattccgacaagacttctgttaccgacgaaccccacttgggtgttccagtggtggatttggtagacatcgataaagacccattcaaacgcagaaaagtggtggacaaaatccgtgaagcttccgaaagttgggggtttttccaagtgcttaaccatggggttccggcgagtgttcaggatgagatcataaatgggacacgtcaatttttcgaacaggatattgaaatgaagaaacaatattatacaagagacaacactaagcctttcgtttacaattccaatttcgatttgtttagcacatccactgccaattggagagacaccgtcttcattcaaatggccccaaatcctcccaacccgcaagactttccactagtttgcaggtaaactctctgcctctccattctctatctttttgttgcatgggtgactttgagggtcagattaatcgtaaaagcgtttcgtacgtgaattgagtgaactcctatcagggcttagtatactatttttacgtctataatattgtgtatcggtaatggatgtatatatattattgaaaggtgtggaattgtttatgagtaacaaaggtagattagtgatgcatgagaatgagaatagaagataattgtataaaaatgtctagatgtgtgcagaatatattccttttaaatgttatggatttcaaattttgtgacaaattcaaagttaagttcaagaataatttaatttaacataaatgactgatttagtgtagttttgaaaataaataaataaatttaatcatgcaaattttcaatgaaaaagaattgggttcatcttagaatgtaccaataaaagtatcaaatcttttttaaaatattttcattaatggttcttttaaaattaatgttaattaattttctctaccaataagcattggattaacttaaagctaagttaaaaattatttgaaagtattttgccgtcgttcgacactaaagtgtctattttgaaactttatggatcaaatagagatgaacttcaaacccaaaaaaggaaaaaaaaacgttttcgtactgtgaagagagatatgagatcatcttgttatggtgcattttggatttgagtcattggtaaagaaaacaaacgatctttctgttaatgtgcaattactgtactaaaggtttatatattttttattttacttttaagattctaaaagttaactatttttctccataaataaaaaaaagtcaagattgtctaataaaatatcaaggtgttattaagatgtttattttgttagaatacttgtggcctcctagcataccttttgaatatgttatttgttaggaactgattgaatatcacggagtaaagaattttggtgttactttctactttgtgaattttgttgcagagacatcctagttgagtattcaaaacaaatggaaaaggttggagaaatgatatttgggctactttcagaggcccttggtttgcaatcaacccatctcctggaattggattgcagtgaaggacatgcctttatgtgccactattatccatcctgtcctcagccagaactcaccattggcaccacccagcactccgatagtagcttcatcactgtgctgcttcaaaaccatatcggaggtcttcaagtgcttcatcacaacaaatgggtggatgttccgcctgttcctggagcctttgccgtcaatgtcggcagcttattgcaggcaaggttttcattattaactattatcatgcattcattaagctaagctatataatatattgttgtgttttcatggtttgattaattgagttccttttatgagagcatgcagttgatttcaaatgacaaatttgtaagctcggtgcatagagtggtggctaatcgtgagggtccaagaatatcagtggcaagtgccttcagcaccggcactataccaacctccaaactctatggacccatcaagcaattgttgtcccaacaaaatcctccaaagtatagacaaatcactgttaaagagtatcgtctctactttgctaaaaaagggttggatggaacggatgctctcacacattttagactttgatttactcttctcttttttggtttccctattccaagctttactaaaatgtttgaactatgttaaggttgaatttctttgttagaggtgtaaacttttaatttgtaaaacatgttagtgtatagttatcgttagtgttagaaattgtgtgaggtcgtgaatattttatttgttcactattttcttctttttggttgacgtggcaaaaagtgttttgaaagtttgtgaagaatatttggatgagacaaagattttatttgggtcctattgtgaacatattttttttttcttaaaagaagaagatttgtctttgtagtagatatttgtgtgtacgaatttttattcttgttatgagaaaagttgttgcaatgcacgatgatagaaaagtgaagaacaacttctcgattgaaccacccgttaaaaaaatagaacctaaagcgtgtaagttttctttcattcgtgtggtttgttgttgttgcttttatttagtgacgattagtgccgaggttataagatatgaagttcaagtttctgttgtatgatataaggaacttttgtttacgccttgatgaaggatgtgattcaggggagttcctctagtttactacttaatctgttgattggtgagaaacttgattaagagggagattttggtaaccaaaccaaatctatatttgaaacagtttagtgatagtgttactttaatattgtattactgatagagtactgagtgaagaaaattagtgttaaaccttctactgagaatcataatagtttgattatgatttatatatcaaaaaaataaaatctctgatcttagagtaaaggaccctctagacgtaaatattgtgtcattgaactagattaacaatgtattgtgtatttctactctctttacttattgttttgtatgttgtatcctctgtagttaactgtgagttcgtcccaaacaatgttgtttctttacctgttttaacaattaaggtcaaatgacaatggtaacattaactataacaataaatatgacatatttgtatttttaagtagaagtgatgaaagacaattaaataggtttgggattcaagctgatcaggagtgatccatcaataaaatttgattaggattacataaattttagaatgaatgggtaaaaatgtcatcaatctctataatttttgaagttagaatttggttgaaatgtcaatgttattgactcatactaataatctataagtgtatcactaattaaatatgaaactttgctatttttttataaatattttaatgcatttcttatatttgaaaata

SEQ ID NO: 74 - DNA, 1000 bp, Cucumis sativus
  source 1..1000: mol_type = genomic DNA; note = Promoter of 2OGD1 gene
tagaaattccacccattcctggagctttagtcgtcaatgtcggcaacttactgcaggcaatgttttgttatgcattcattaagtaacaagatacacatatatagaaaatataatgatgggtttgggttcctttatggatgcagttgatctcgaatgacaaatttgtgagctcgatccatagagtgttggctactcgaaatggtccaagggtatcggtggcaactttcttctccactggctatgcagaaaccttcaaactttatggaccaatcgaagaattgttgtccgaacaaaatcctcccaaatataaacaaactactgtgagagattaccgtctctacttttctaaaaaagggttggatggaatggatcctctcacacattttaggatttgattttactgaactgtttttttggttcataattctaaggtttgttaaatgtttaagttcgactttgttagagtgtgtgatttttaaacactataacattgattttattgaaatgtcttttcctaccctgttgtaaggtttgttatgtctttagaaattatttgttaaacctttgattgaattaggtttgagtatgtcaactaagtttaatttagtatctacaatttcaaagattggaatttaaacttcagtaattaaaaaaagagaaagaattagacaaaatgttttaaaagttggaatttagtaaaagaaattttggatatgaatctcatatctttaattacttaggttaatatgttttttaaacactttgaacactttgaacactgggatatttttgaatcatgactcgtacgaaaaaccaagaccagagagaaaggcagcatgagggggtctgagttatgaactatcttctcatgttttctgtctgtatttgccgacagtggattagcaagcattaacgtccttctcacagcaaaaataactatctccattcagataaactgattaaattaaaagaccatacacagtacactcctcaaacaaaa

SEQ ID NO: 75 - DNA, 1000 bp, Cucumis sativus
  source 1..1000: mol_type = genomic DNA; note = promoter of 2OGD2 gene
tgttggttgtgagtgctctatggtcttgacctcaaactcacattccttttgtttgatctcgatcttgttcaaactcacctctatttaagggatttttgagcctacagtgattgaatgcgtgactaactcaactaaacaataaagaactaaggttcttggtcttttaactctcatttgaagatacttattattctcgttttttggctgtggagtgaaactatttataaattggtcctctaatacacatgttcatatttgtgtgtgatgaaaggagggttggggacgtttttggcacttgaggtccacacttggaattcgccattgacaccactaaacatgctaacaataccttcatatcgcaatgctttattaaatatttgattaaaatgtgttaaaactgagtctgttgagtctggagatgttcttttgtatcaactatcttttgttgtgtcaagatcattaatatatagaaacaataataattatcaaagtaaaggcataggccattttaatgtgacaaaaaccaaggacccatataaggccattcacgtcctcaccccctcaaacccaatcatttcttaactatcctctacttttggaccaacaatttgtgattagttgctggttttgtttgacgttggagggtaaaacttcattagcagacaagattacataaacactggtttgaatgaggtttttcaaggggagactcttccaccttaatctaataaggagtgatttgcatgatcaaggagaggattttgtttagcaaaatttaactttgaaattgtagtgacaaagtggctttactaaactgtatcagtgtagagtgtgaatctaagttgattaaaaatggtatgttagtttaagtttgtttactactgactatacagttattgttttctcaagagggataccatacccgatcactcgtcccaatcaaaacaaagaaacaaaaaaataaaagcggaaatatttgttggtggaaaattttgtgtgg

SEQ ID NO: 76 - DNA, 1000 bp, Cucumis sativus
  source 1..1000: mol_type = genomic DNA; note = promoter of 2OGD3 gene
ataaaaacggaagaagaaatacaaaagaggatcacaaaaaaagaaaagaaaaaagaatggttataaaagaagaagaagaaaggaagataaaaaaaacaaaagaagtacaaaacgaagaaaagaggaaagaaacatttaagactaaagttgatgattttacatatgatgacataaacaaaaacaccaattttaaaaagtacatatcttttatttgaagctttccttcttattttgaggacactcccacaaaatttccatttttggtaagtgttactgccgtgggctatgaattaagttggtcttttatttattgttttggttatttatttttttaattatctccaaaattttagcaagcactatttcatttactctttttttttcttctttttattataaatattagtccattaaattgaataaatgcaataagtttcatttattttttttctctcttgacaatatatcttagagagtatttatgtttgttttcttcgagaagagattttatggatcatttgttttaaaatcaataactgttagttctttgagatctttagatatatatatatatatatcaaattaattagatatatagcacaattttaaaaaaattacaaatataacaaaatttgtcaaattctatcaatgatataagtctatcactgaccgactatgttgtaaatattaatctatcacttatataccatatgaaatctatcagcaatacaagtctatcgatgataattttgtaatatttacaattttttaaaatgttgaaatatccttaattttgtaataatttacaatttttttaaaatgttgaaatatcctttattattattgttaaaatagtcgtaaattccaattttcatatatttttatttgagcccacaatttgatcggagactattgaagtctccgtcaccatcaaatttctctcccctcaagatgaatcgaacatcgcctcctcaaaattggagtttcgaaatacacaaaa

SEQ ID NO: 77 - DNA, 1000 bp, Cucumis sativus
  source 1..1000: mol_type = genomic DNA; note = promoter of 2OGD4 gene
atcaagcacacatttaaattgcaattatattgacgtaccatcccccataataattcgtggaattgtttagatccatctgtcttatattttctttcttcatctttccatcccacaaaaatattttttttttgtggatgagcgatgtgatgaagaacgtaaatgatagatatgcctcaagtttgttttcataaaatacggtctttatctatctttcttctaactattttcttccactactattgtaaaagttgaagttgaaattaaacaaaacaatatggcttctagataattagtggtcaatataatttggaaacactatataacaaaaaaagaagaaaaatagaggcagtagataagattcaaatttaaaatgttaaagttgtgaagggaatagaatttagtaataataaagtattatttaatatatattataatataacattttaaaattaatattaaaatataaaaaaatgtaaatgacaggtgttgctagctaaatgatgccacgattattttgaatcaaaattaggaactaagtcaaacccaaagtttaatattaagggcaacacttaggatgcacccatccctatgaatttccttttcatgactgagataaataattaagaatatttaataaaaattagggatatagaacaaaaatagataaaacataaataataggtcgggatgcaaaaatttcgtaaatgcgcatttattggcattcataattgatgacatgcatttgtatttgtgaataataaaaaaagatgcaaatatgtaatttgtcgttgtaatgcacccaagaggtacgatgtaggaggggacatgaaaccatgagctaacaaaataaaataaaagaatttgttaaaatatttcgagtgttagtcgcgttagatttaactaaatctttatatcaatgggagtctagaaacttgaaactgaaattgagattctcaaacgaaaatctaatcagagaagattagattagttgcaaattaca

SEQ ID NO: 78 - DNA, 1000 bp, Cucumis sativus
  source 1..1000: mol_type = genomic DNA; note = promoter of 2OGD5 gene
aataccataaaattgagagagattgagtctcaagtccaccgtaaattcacgtgtttgaattgtcatattgaagaaaaaaatgtacgaaaattgaaatcgtggaacactctatcaataattcgttggaccaccttggctaaaagcaatttatttttgaaaggtctaaaagcaactcttcttgcacctcatgaaacaaaagtgtcatattcatatatgcttatttacaaggctaattcttctcttcttccaccttctctttctttcattttttttttttaaattcaacatattaaaggttagtcaaactatataaaagataaatgcaaccaaataaaaatgtgccacgtggtactctttttggaaaatattagaaaattgatgtttattttgagtctctcccttattataaatcaacttttttaattctctaccttcattttttcttcttctttttatattttcaaccaaacttatttatattttaaaaatagtaaaataaattaaaatatttataagatatagcataattttggatttaaagaatagaaactaataaaaaattattcttattttggagaaacttatttataactagtctgtataaaaaaaccaagctcaccttaacacaatttcccttgagataattaaacacaaaaataaatatacccctaattccttaaaatattaataaataaattctaaaatataaacttttcaacatgactgagtttaaaattcaaaggaaaacctatatgaattatgaacatctatctatttatccaatcagtcaaaatttgaaaattcctttctagtggacaacttgaacttgaattacaattagagcccaccaccaacttacaaaccctggctatcctactacttcaccatttatatcccaccccttcccaccattcaccaaaccacttttacaattttgaaatcacactcttcattccataaattcaatctcttccatcaacccaacagatccatatccata

SEQ ID NO: 79 - DNA, 1000 bp, Cucumis sativus
  source 1..1000: mol_type = genomic DNA; note = promoter of 2OGD6 gene
gatacggattgtttttaagatcaaacaactggatgaaaaaaaataagtttttttaactctgaaacactccacaagaaaaattaatcaatcctcttaaatgttctgcatgtaattcaacacatgatttaaaaatttaaaaattcttagactaatcttgaaaacaatcacaaagcctaaatctcaaatcatccaaatcttcttaaaacaatcatagcaaaactaagtctctccaataaaaatcctaatgcaacactaaaatgaaaaaaatatccgtgcttaacttaaattaagaaatactaaaattacatcaaatttggtttgaatcctatattggtgttgttctattttgatttataaacttctaaaaaacaactttttaattttattttttttaaaaaaagtaatctatggtcctttatttatccctaactaaattttttaaagtttattttatgtggtcatggatacaacttctattttattaattcattcattctactcaatgttcttaacttcaagattccaatttgaaaataaacttaaaagattccaatttgaaaataaaaagaatgggtgggagccgaagaggaaatgtaagaaaattgagttttagagggaaaaataataaaattagtttttgagttcgtttgattcacctttattgattatgtttttgtgctatataaaatatttatagaataaattagtactatcgtttttggttatatatcaattcaattaattgattaatctaatgttcaatcttgaaaatttatggactaaaatatgatttaattcaataaaattttattaataacgtgatgccttagagagttcatatcacactctactaatccacatgtcaaacgtaaatatgttggatatttatttcaacgaattcttgtacttagcaaacctcacaaatttgataagaaatgtttcattgatatatgagtatactcaatatttctaactcaagttttaactttattatgtgttg

SEQ ID NO: 80 - DNA, 1000 bp, Cucumis sativus
  source 1..1000: mol_type = genomic DNA; note = promoter of 2OGD7 gene
taatacaaaacgggcatttaaggatttttccaatatgtctgattgacttgatgtattgactttagaggattgatacattttaatttacttaatggttaagttctctttgtaagaatagttaaatcggaaaaaagtatggctagctatacatcccctaggtaagaagataaccatcatcttataaagaaaatagctcatggagatataaagtgttcaagtaacggattgatcaccacttgtttgattttcaaaccaacttatatatattgtttatatacacaacctattatttattcaaaagaacccacgtgcttaattaccctattcatattccaatctaaaataattatttattgaggagttgggttaaacatgacaacttctctgctgtctagaaaaaaatattttttatcaagttgaaccagtcaatcaactattggttattggcatgtgcaactattaatcttgtttaatattttttccttttcttcttctttatatttgtttgttaaagtgtttattatttcacatcaaataaatataatttaaactctaaacataaactattattaaaaatactaaagtattttataccataaactattaattattataatctaataacgataacttggtgtcggtccacaaatttattctataggtagtcatacatatagtaaaattattgctagttggatgtttacatggttgaactcagtatctagatttattagcgacttaataaatgaatatctatcatatgcggtgaatatatagactatacgatcttagtgtgtggttaagagttgtataacttttaatatgcaaaccattccaaagtcttaacacaattatttatttaaagtgagcagggacaaaccgataaatgcccccatcatttctttattaaatatgtctacgattacaatggacaacccctaaccacttacaatttttcaaaacacgttctttaggggcaaaggcaactgttccatccaca

SEQ ID NO: 81 - DNA, 1000 bp, Cucumis sativus
  source 1..1000: mol_type = genomic DNA; note = promoter of 2 OGD8 gene
ctatttcaaacataaatcatcccaacatcaagatgacattacaatttatgagagaactttggcaagattgacatttttaacgaagaaaaaattgaagctagaattcatcttttatttccattaagaaattagtgcattccatatttagaattaatcttaaattgaacttaattttatgtagaaatgatttaatgtaattcaaattttgctgggaacaacaaaaactagcaaattctaggaaaacattagttaattaagattaataggtttcacttaaattaaagataaatcagcaaactaaataacttaactttaattttcaaacaaaaactcaatgtcttggaacacctcacatattcttacacgaaagaaacaatgattaattttgcaaacaaatttacatgatcaaagtcacgctttcttggagaaaatgaaagaactaaagcaccctacgtttgataacaattgaaattttctaaaaaagaagcaaacctcattaagaagttgatcttaagtacccaagtctcgaccatatcgagaatacaagaagaaggcgttctagatggggtcgtgacaacatcaatcattcaacaatgcacattaattgtatcaaataatatcatttacatcaagtgtatcataatctaaaagatatcaagaaatatcaaatgtatcgagtgcatcaaggagtatcacatgtatacgaacatgaattagagaatagcatgcctatgaagtgtataaagtgttatgagtatataagtttattgaatgtatatgaatagcaaggactttgttacgaatgtgttacatgtgtggaactggacaaagcataatagaaatgaaagagcatacacatacttattttgcactatacatatatataaccatttctaaaaataaaaagaattaagtttcctttccatttaatacaaaagctataagccattcattcatatagaataatacacagtccaataaatcatttccataacagaaa

SEQ ID NO: 82 - DNA, 1000 bp, Cucumis sativus
  source 1..1000: mol_type = genomic DNA; note = promoter of HR-like gene
aaggcactaacgatcaaaactcctctatttatgtaaatggttttgcctcaaacttatttttgtgattcgtttggccattcacattattttagaaaaaaattccccaattaattgagaatcaagaatggatcttagtcaaatcaccacaagaaaaaaaaaaactaagttgatttctaaggatgacttgattagcaagaatggtaagcattattctttttcattttagatttgaagtaagaattaaatgaaattttagtttgcaaaattaatcaccctatttagtccataacaactccttttacattcttgcacataatttttaacgatttctttagacttgtagcatgtataaattttaaataactatttcttttaaacaaaaactaaactaaacttgctcctaactcaactctctaacaatttgtttcgtttgcttgaaaaagataaacttaaaacataagtttttataatgagtaacaatttagtactaatagaataggataaaagaaatgtcacattagctggtgctagtctggcctcaatgttttgtgttatataacccttgtttttggaagttttaagtacaactgggaagtttaattttaatttgaacttttattgacactaaaattaaaaaacttacacttaaatataaaatttaaataatatattaaaaatggaatggttataaaatttcaaactttttaaaattgaaaatttagggattcactttaaagataaatttattaaacaaatcctttataaatgccaaatcaaaactgaataaattagtcagccgactaaaaagatatagactcgaggatttagagatttttttagaaaaataaatttgatttgatggtttgaaatttgaaattttgggaagtggtgtgacactccaaattcctttttgtaggtcctcatttataactgtgttttgccatcaattttttttgcctctaagagttgcgagccagattacctttgtccattttcaaat

SEQ ID NO: 83 - DNA, 100 bp, Cucumis sativus
  source 1..100: mol_type = unassigned DNA
gacgtagtagatatagaaattttatacctttggtttcttcaaaatataaacaaacgctatagaaattaaaataggaaagtcttcgattcgtcccatattc

SEQ ID NO: 84 - DNA, 100 bp, Cucumis sativus
  source 1..100: mol_type = unassigned DNA
ttcaacatggaggtaaacgccattctagaagagaaactcaaaaagattcgatctgtggtcggccttgactttccggattcctttattcatcggacgttgt

SEQ ID NO: 85 - DNA, 1546 bp, Cucumis sativus
  source 1..1546: mol_type = unassigned DNA; note = SNP_36 at position 887
atgggattcttctccgcccttggacgcctcctctttgcctccttcttcatcctctctgcttgtcagatgtaagttcacttgtattcctcactcaattctcatatttattggatctagactattgttgctattttggcttgtattccactccttttcatttcccaaatagctattttggctatttgggttttgcttccatttccaaagaaacgaaaactcaaatgaaaatggctatgctagttgttcttatgtgataatttttacttttaatgggccagatttattatgttcccatttattggtcttttatttggttattttcattgagagtggagggctcaaagcattttcactgccctaattttattatctataggccttcatttgtatatgtatctatattttacacagatatttatttattgtttttttaatatctgtgtgtttttcttaggttcagctcttttttaggtatgggtgttatatcaaggggatgaaattgcattgaaaatttccaattttcaaatttccattagctaatcctgggataacaaaaccaaggactcaatcaccagctgccttacctcattgaactaataaatgaaagtttaattatgagtgatctgtttttcaccaatttggtttcataaataaatagaagaaaagtaatctttaatctatctctgttggatgcacaatcttcctgaaaatcatttttttttggctaaaaaccaatttttgaaagttttatttagtttgaacacaggccaatctcacctgcactatgtttacatttaccttgttaggtttaacaataatggtgggccagcagctgtggaattgtcttcaaaggtagctgttctgaggagaaatttatcatccaagttggggttgctatacctgatattgatgtaaggccatgttaggccttattcaccacttcaatattgacaatattgaccatacctgtagactgaaattttgataattttctcaggtcagacatgtggttggtctttcattatctctcaaagggattggagctcttctatttgttttgggctcaagagttggagcttatcttttggtaaggattcaccatgttcaaaatttagcattgtacacatacatttgctacccacatttattcctaactaatgttactctaaatttatgcatctgcaaattgccctctctttcagcttctttacttggctattatcactccggttcttttcgatttcttcaaccttagtctactgaaacctacttttggtatcatgttaagtgatttcttagaggtttgtccatgtgctttctaactttttccatcttttcattttaagcatctccaatttaatcataagaatcctattctcttggcaatttaatcataagaaatttaatcataagaatcctattctcttggcagcacattgcactttttggtgctctccttttcttcatagggatgaagggcacaagtccaaagaagcactcaaaaaagaagacccaaaaacaaaaagcaaaagcaaaagcacattag

(19) United States
(12) Patent Application Publication
(10) Pub. No.: US 2025/0089639 A1
(43) Pub. Date: Mar. 20, 2025

(54) DIDYMELLA BRYONIAE INTERNAL FRUIT ROT RESISTANCE IN CUCUMIS SATIVUS PLANTS

(71) Applicant: NUNHEMS B.V., Nunhem (NL)

(72) Inventors: Daniele LIBERTI, El Ejido (ES); Hans Peter KOELEWIJN, Nunhem (NL); Frank BEENDERS, Nunhem (NL); Laura REINIERS, Nunhem (NL); Wim VRIEZEN, Nunhem (NL); Lieke MERTENS, Nunhem (NL)

(21) Appl. No.: 18/965,676

(22) Filed: Dec. 2, 2024

Related U.S. Application Data
(62) Division of application No. 17/912,364, filed on Sep. 16, 2022.

(30) Foreign Application Priority Data
Mar. 18, 2020 (EP) 20163899.6
Sep. 8, 2020 (EP) 20194997.1

(51) Int. Cl. A01H 1/00; A01H 5/08; A01H 6/34

(52) U.S. Cl. CPC A01H 1/1255; A01H 5/08; A01H 6/346

(57) ABSTRACT

The present invention relates to cultivated cucumber plants comprising modifications in their genome, whereby the modifications confer reduced susceptibility to internal fruit rot symptoms caused by Didymella bryoniae, and to methods for generating such plants, and their use.

This application is a Divisional of U.S. patent application Ser. No. 17/912,364, filed Sep. 16, 2022, which is a National Stage Entry of PCT/EP2021/056558, filed Mar. 15, 2021, which claims priority to EP Application Serial No. 20163899.6, filed Mar. 18, 2020, and EP Application Serial No. 20194997.1, filed Sep. 8, 2020, the disclosures of each of which are hereby incorporated by reference in their entireties.

The sequence listing of the present application is submitted electronically as an XML file named “039621.01320_Sequence_Listing.xml”, created on Nov. 26, 2024, and having a size of 165,704 bytes. This sequence listing submitted electronically is an integral part of the specification and is incorporated herein by reference in its entirety.

The present invention relates to the field of cucumber breeding and modification of the cucumber genome. In one aspect an introgression of a Quantitative Trait Locus (QTL) located on chromosome 5 of the cultivated cucumber genome and/or an introgression of a QTL on chromosome 3 of the cultivated cucumber genome is provided, which can be used to increase resistance against internal fruit rot caused by the ascomycete Didymella bryoniae (abbreviated herein as DB) in cultivated cucumbers (Cucumis sativus var. sativus), such as long cucumbers, pickling cucumbers (e.g. American pickling, European pickling types), slicing cucumbers (e.g. American slicing), short cucumbers, European greenhouse cucumbers, Beit-Alpha type cucumbers, oriental trellis type cucumbers (also marketed as ‘burpless’), Asian cucumbers (which can be further subdivided into different types, such as Indian Mottled cucumber, Chinese Long cucumber, Korean cucumber and Japanese cucumber types, whereby the first belongs to the Indian cucumber group and the last three are part of the East Asian cucumber group).

Further, the causal genes underlying QTL3 were identified, whereby plants comprising modifications of those genes can be generated, e.g. through targeted genome editing, mutagenesis or transformation. Plants and plant parts comprising such modifications are one aspect of the invention.

The QTL on chromosome 5 is referred herein as QTL5.1 and the QTL on chromosome 3 is referred to as QTL3.1. In one aspect, both are introgressions from the same wild cucumber, referred to as MYCR3 (a proprietary accession). A plant of the accession was used to map and to introgress the QTLs into the European long cucumber type, into different genetic backgrounds (using different recurrent parents). A representative sample of seeds comprising both QTLs has been deposited and from the deposit, or from descendants of this deposit, one or both QTLs can be easily transferred into any other cucumber type, such as short cucumber types, or into any other long cucumber breeding line or variety. Alternatively, other donors can be identified which comprise the same QTLs, e.g. comprising the same SNP haplotypes for QTL3.1 and/or for QTL5.1 or the same SNP haplotype for at least several, e.g. at least 10 or more SNP markers, especially for at least 10 or more consecutive SNP markers, or which comprise a duplication comprising QTL3.1, such as a duplication of SEQ ID NO: 72 (or a genomic region comprising at least 95% sequence identity to SEQ ID NO: 72) or a duplication of the genomic region starting at (and including) SEQ ID NO: 83 and ending at (and including) SEQ ID NO: 84 of chromosome 3. Seeds comprising both introgression fragments in homozygous form, i.e. QTL3.1 and QTL5.1 in homozygous form and comprising donor SNP markers/sequences indicative of the introgression fragments in homozygous form, were deposited under accession number NCIMB 43530. Seeds of the genetic control, lacking both introgression fragments were deposited under accession number NCIMB 43531.

QTL5.1 was initially mapped to be linked to SNP markers SNP_01 to SNP_18 and was later fine-mapped to the region linked to SNP_12 to SNP_15, with additional markers added in the region. See FIG. 6. Thus QTL5.1 lies in-between SNP_12 and SNP_15 on chromosome 5.

QTL3.1 was initially mapped to the region linked to SNP_19 to SNP_42 and was fine-mapped to SNP_35 and 659 nucleotides downstream of SNP_36. This meant that QTL3.1 is closely linked to SNP_35 and SNP_36 on chromosome 3. The entire region was sequenced, which showed that QTL3.1 (provided herein as SEQ ID NO: 72) was duplicated on chromosome 3. The duplication of QTL3.1 was part of a large duplication of around 150 kb (actually 147292 bases) on chromosome 3, see grey bars in FIG. 4, whereby the beginning and the end of the large duplicated region are provided herein in SEQ ID NO: 83 and SEQ ID NO: 84, respectively. The resistant plant thus contained QTL3.1 twice on chromosome 3, as shown in FIG. 4. As the resistant plant, of which seeds were deposited, is homozygous for QTL3.1, it in fact contained QTL3.1 four times, two copies on each chromosome 3. In contrast, the susceptible plant contained only one copy of QTL3.1 on chromosome 3 (and in the homozygous seeds deposited therefore two copies of QTL3.1).

This structural variation for QTL3.1 revealed by sequencing means that the nomenclature used herein has, in one aspect, a revised meaning. When referring to a resistant plant “comprising QTL3.1” in homozygous or heterozygous form compared to a susceptible plant “lacking QTL3.1”, this in fact in one aspect refers to a resistant plant “comprising a duplication of QTL3.1” in homozygous or heterozygous form, compared to a susceptible plant “lacking a duplication of QTL3.1”.

Sequencing of the QTL3.1 region showed that there are 9 genes in the fine-mapped QTL3.1 region. Expression analysis further showed that in the resistant plant (homozygous for the duplication of QTL3.1) these genes were all expressed to a higher extent than in the susceptible plant (homozygous for chromosome 3 lacking a duplication of QTL3.1). The gene expression of each gene was about 1.3 to 2.6 times that of the susceptible plant, as shown in Table 7. The enhanced expression of the 9 genes in the resistant plant therefore causes the increase in resistance against DB/reduced susceptibility to internal fruit rot caused by DB. Further, 8 of the 9 genes encode the same type of protein, namely 2-oxoglutarate and Fe(II) dependent oxygenase enzymes. Although the in-vivo function of these enzymes is not clear, they have been shown to be upregulated following fungal infection (Schenk et al. Plant Physiology 2003, Vol. 132, p 999-1010), indicating their involvement in disease resistance. It is, therefore, logical to assume that the & upregulated 2-oxoglutarate and Fe(II) dependent oxygenase (abbreviated as 2ODG1 to 200D8, or simply OGD1 to OGD8, herein) all have the same function in the resistance of the fruits to infection and that it is the overall gene expression level and thus the overall 2-oxoglutarate and Fe(II) dependent oxygenase enzyme level being produced in the plant confers the enhanced resistance. The ninth gene encodes a HR-like protein (HR meaning Hypersensitive Response, which is a plant defense response against pathogens).

This means that, apart from introgression of the duplication of QTL3.1 from a wild donor into cultivated cucumber, one can also enhance the gene expression of one or more or all of the 8 OGD genes and/or the HR-like gene in cultivated cucumber to enhance resistance to DB internal fruit infection. How this can be done is known to the skilled person, but will be described herein. For example the whole cluster of these 9 genes may be duplicated (e.g. QTL3.1, or SEQ ID NO: 72, may be duplicated) for example with the aid of targeted genome editing techniques, or one or more genes may be duplicated individually. Alternatively the cis-regulatory elements (especially the promoters) may be modified to enhance gene expression or one or more of the 9 genes. A further possibility is to generate transformants (GMOs), comprising extra copies of one or more or all of the 9 genes.

Different cucumbers have different base-line susceptibilities to DB internal fruit infection and these differences are seen between cucumber types, but they can also be seen to some extent between lines or genotypes within a cucumber type. In long cucumber types lacking QTL3.1 (i.e. lacking the duplication of QTL3.1) and QTL5.1, generally about 25% to 45% (sometimes also more, but generally less than 70%) of the fruits which develop from spore-inoculated flowers become infected. In some slicer cucumber types or some pickling cucumber types lacking QTL3.1 (i.e. lacking the duplication of QTL3.1) and QTL5.1, generally more than 70% of the inoculated flowers result in fruits which are infected, often 80% to 95% become infected. This means that the effect of the DB fruit resistance conferring QTLs of the invention is most prominent in cucumber lines and cucumber types which have a low base-line susceptibility to DB fruit infection. In some genetic backgrounds the QTLs result in such a low percentage of internal fruit infection, that chemical control by fungicides may not be required anymore or may only need to be applied rarely.

In different long cucumber lines, the presence of QTL5.1 and QTL3.1 (i.e. the duplication of QTL3.1), both in homozygous form, was found to lead to full, or almost full, resistance against fruit infection (less than 2%, or less than 1.5% percent of spore-inoculated flowers result in fruits having internal symptoms of fruit infection, preferably less than 1% or less than 0.5% percent of fruits with internal symptoms of infection). Base-line susceptibility of the lines was reduced by more than 25% compared to the genetic control cucumber plant lacking both QTLs (meaning with regard to QTL3.1: lacking the duplication of QTL3.1), leading to full resistance or almost full resistance of the lines, see also Examples. In cucumber lines which have a higher base-line susceptibility to fruit infection by DB, the presence of QTL5.1 and QTL3.1 (i.e. the duplication of QTL3.1), both in homozygous form, may not lead to full resistance, but to a partial resistance against fruit infection, as the reduction in base-line susceptibility may not be sufficient to reach full resistance levels. Optionally, the combination of QTL5.1 with a higher copy number of QTL3.1 or an increased expression of one or more of the 9 genes may lead to full resistance against infection in these backgrounds also, or at least to a higher resistance level than the combination of QTL5.1 and the duplication of QTL3.1.

Thus, the combination of both QTL3.1 (i.e. the duplication of QTL3.1) and QTL5.1 reduces the base-line susceptibility against internal fruit infection (i.e. increases resistance) by at least 10%, 15%, 20%, 25%, 30%, 35%, 40%, 45%, 50%, 55%, 60%, 65%, 70%, 75% or more. To determine the effect of the QTLs, both QTLs can be crossed into a recurrent parent lacking the QTLs, and then the internal fruit rot resistance of the recurrent parent can be compared to the internal fruit rot resistance of the line comprising both QTLs in homozygous form, in a fruit rot disease assay described herein, e.g. in the Examples.

The base-line susceptibility is the percentage of infected fruits of the control cucumber plant (e.g. the recurrent parent or the genetic control) lacking both QTLs (i.e. lacking QTL5.1 and lacking the duplication of QTL3.1), e.g. 25% of inoculated flowers may result in the developing fruits showing internal fruit rot symptoms, while in the line comprising both QTLs (i.e. QTL5.1 and the duplication of QTL3.1) in homozygous form less than e.g. 1.5% of inoculated flowers may lead to the developing fruits showing internal fruit rot symptoms, i.e. the QTLs reduced the base-line susceptibility by 23.5%. Similarly, if in the control cucumber plant (e.g. in the recurrent parent or the genetic control) 80% of inoculated flowers result in fruits showing internal fruit rot symptoms, while in the line comprising both QTLs only 35% of inoculated flowers result in fruits showing internal fruit rot symptoms, the QTLs reduced the base-line susceptibility by 45%.

It was found that the highest effect on DB fruit resistance is seen when both QTL3.1 (i.e. the duplication of QTL3.1) and QTL5.1 are present together in a plant and when both are in homozygous form. Such a plant can be described as having two copies of QTL3.1 (i.e. two copies of the duplication of QTL3.1) and two copies of QTL5.1. As it is now known that QTL3.1 is in fact a duplication of QTL3.1., the homozygous plant is preferably described as having four copies of QTL3.1 (two on each chromosome) and two copies of QTL5.1 (one on each chromosome). When both QTLs are in heterozygous form (i.e. the plant comprises 3 copies of QTL3.1 and one copy of QTL5.1) or when only one of the QTLs is present the effect is smaller. The smallest effect is seen for individual QTLs in heterozygous form. As can be seen in Table 5, the more copies of QTL3.1 (i.e. the duplication of QTL3.1) and QTL5.1 are present, the higher the effect on internal fruit rot resistance, with both QTLs in homozygous form showing the highest effect. The effect that QTL3.1 and QTL5.1 together (homozygous or heterozygous) result in a higher resistance than when only one of the QTLs is present is herein referred to as ‘additive effect’. In one aspect it is, therefore, preferred that a cucumber plant comprises at least one copy of the duplication of QTL3.1 and at least one copy of QTL5.1. In one aspect it is, therefore, preferred that a cucumber plant comprises at least one copy of the duplication of QTL3.1 (or a higher gene expression of one or more or all of the 8 2OGD genes and/or of the gene encoding the HR-like protein) and at least one copy of QTL5.1.

The individual QTLs, i.e. either QTL3.1 (i.e. the duplication of QTL3.1) or QTL5.1 alone (in homozygous or heterozygous form), may only have a relatively small effect on conferring internal fruit rot resistance, i.e. individually they reduce the base-line susceptibility of the recurrent parent by only 2%, 3%, 4%, 5%, 6%, 7%, 8%, 9%, 10%, 15%, 20%, 25%, 30%, or more.

This additive effect of QTL3.1 (i.e. the duplication of QTL3.1) and QTL5.1 means that the QTLs are preferably used in combination (e.g. both in homozygous form or both in heterozygous form), although they can also be used individually. The embodiments describing herein for each QTL separately therefore also apply to the combination of the QTLs. The effect of the duplication of QTL3.1 can be achieved by different means, e.g. duplication of the entire region from SNP_35 to 659 nt downstream of SNP_36 (i.e. duplication of SEQ ID NO: 72 and 73) or increasing gene expression of one or more or all of the 8 2OGD genes and/or of the gene encoding the HR-like protein, for example by gene copy number increases or modifying the promoters to enhance expression. The genomic sequences of the 9 genes are present on SEQ ID NO: 72 and 73, but the putative promoter sequences (i.e. the 1000 bp upstream of the ATG codon) are also provided herein in SEQ ID NO: 74 to 82.

As it is now known that there is a dosage effect of QTL3.1, and a dosage effect (copy number increase) of the expression of genes encoding 2OGD proteins and/or a HR-like protein, any increase in copy number of QTL3.1 above the wild type copy number found in susceptible plants (one copy of QTL3.1 on each chromosome) and/or any increase in gene expression of genes encoding 2OGD enzymes and/or HR-like protein above the wild type gene expression (i.e. the wild type comprises 16 expressed genes encoding 2OGD enzymes and 2 expressed genes encoding HR-like protein) is encompassed herein. The mRNA transcript level and the protein level of a 2OGD enzyme and/or HR-like protein is thus increased in the resistant plant compared to the susceptible plant lacking the duplication of QTL3.1, i.e. comprising only one copy of QTL3.1 on each chromosome. This will be described elsewhere herein.

In one aspect a cultivated cucumber plant comprising an introgression fragment on chromosomes 5 is provided, comprising QTL5.1, whereby the introgression fragment significantly increases the fruit resistance against DB (significantly lowers the percentage of fruits showing internal symptoms of infection after inoculation of the flower with DB spores) of the cultivated cucumber comprising the introgression compared to the control cucumber (e.g. genetic control line) lacking the introgression. Also one or more molecular markers (especially Single Nucleotide Polymorphisms or SNPs and/or the sequences comprising the SNPs) which are present on the introgression fragment and which are indicative of the presence of the introgression fragment and methods of using such markers are provided herein for QTL5.1. Likewise seeds, plant parts, cells and/or tissues comprising QTL5.1 on chromosome 5 are provided.

In one aspect the plants, seeds, plant parts, cells and/or tissues comprise the introgression fragment from a wild cucumber, whereby the introgression fragment comprising QTL5.1, which QTL is located physically in the region starting at 3.7 Mb and ending at 4.03 Mb of chromosome 5, or starting at the physical position of SNP_01 at nucleotide 51 in SEQ ID NO: 1 (i.e. on chromosome 5 at nucleotide 3701817 (of the cucumber genome Chinese Long v2 found at cucurbitgenomics.org)) and ending at SNP_18 at nucleotide 51 in SEQ ID NO: 18 i.e. on chromosome 5 at nucleotide 4028826 (of the cucumber genome Chinese Long v2 found at cucurbitgenomics.org). In one aspect the other regions of chromosome 5, i.e. from 0 Mb to 3.7 Mb and/or from 4.03 Mb to the end of chromosome 5 comprise or consist of cultivated cucumber chromosome regions.

In a further aspect the plants, seeds, plant parts, cells and/or tissues comprise the introgression fragment from a wild cucumber, whereby the introgression fragment comprising QTL5.1, which QTL is located physically in the region starting at 3.81 Mb and ending at 3.97 Mb of chromosome 5, or starting at the physical position of SNP_12 at nucleotide 51 in SEQ ID NO: 12 (i.e. on chromosome 5 at nucleotide 3823864 (of the cucumber genome Chinese Long v2 found at cucurbitgenomics.org)) and ending at SNP_15 at nucleotide 51 in SEQ ID NO: 15 i.e. on chromosome 5 at nucleotide 3967955 (of the cucumber genome Chinese Long v2 found at cucurbitgenomics.org). See e.g. FIG. 6. In one aspect the other regions of chromosome 5, i.e. from 0 Mb to 3.81 Mb and/or from 3.97 Mb to the end of chromosome 5 comprise or consist of cultivated cucumber chromosome regions.

In one aspect QTL5.1 (i.e. the introgression fragment comprising the QTL) is present in heterozygous form in a cultivated cucumber plant, cell or tissue, especially in long cucumber. In another aspect QTL5.1 (i.e. the introgression fragment comprising the QTL) is present in homozygous form in a cultivated cucumber plant, cell or tissue, especially in long cucumber. In a specific aspect the cultivated cucumber plant is an F1 hybrid, especially an F1 hybrid generated by crossing two inbred parent lines, whereby at least one of the parent lines comprises the QTL5.1 (i.e. the introgression fragment comprising the QTL) in homozygous form. In a specific aspect the cultivated cucumber plant does not comprise any other introgression fragments on chromosome 5 of the cucumber genome which affect DB fruit resistance.

In one aspect a cultivated cucumber plant comprising an introgression fragment on chromosomes 3 is provided, comprising QTL3.1 (i.e. a duplication of QTL3.1), whereby the introgression fragment significantly increases the fruit resistance against DB (significantly lowers the percentage of fruits showing internal symptoms of infection after inoculation of the flower with DB spores) of the cultivated cucumber comprising the introgression compared to the control cucumber (e.g. genetic control line) lacking the introgression. Also, one or more molecular markers (especially Single Nucleotide Polymorphisms or SNPs and/or the sequences comprising the SNPs) which are present on the introgression fragment and which are indicative of the presence of the introgression fragment and methods of using such markers are provided herein for QTL3.1. Likewise seeds, plant parts, cells and/or tissues comprising QTL3.1 (i.e. a duplication of QTL3.1) on chromosome 3 are provided.

In one aspect the plants, seeds, plant parts, cells and/or tissues comprise the introgression fragment from a wild cucumber, whereby the introgression fragment comprising QTL3.1 (i.e. a duplication of QTL3.1), which QTL is located physically in the region starting at 9.0 Mb and ending at 9.4 Mb of chromosome 3, or starting at the physical position of SNP_19 at nucleotide 51 in SEQ ID NO: 19 (i.e. on chromosome 3 at nucleotide 9047770 (of the cucumber genome Chinese Long v2 found at cucurbitgenomics.org)) and ending at SNP_42 at nucleotide 51 in SEQ ID NO: 42 i.e. on chromosome 3 at nucleotide 9357469 (of the cucumber genome Chinese Long v2 found at cucurbitgenomics.org). In one aspect the other regions of chromosome 3, i.e. from 0 Mb to 9.0 Mb and/or from 9.4 Mb to the end of chromosome 3 comprise or consist of cultivated cucumber chromosome regions.

In a further aspect the plants, seeds, plant parts, cells and/or tissues comprise the introgression fragment from a wild cucumber, whereby the introgression fragment comprising QTL3.1 (i.e. a duplication of QTL3.1), which QTL is located physically in the region starting at 9.22 Mb and ending at 9.27 Mb of chromosome 3, or starting at the physical position of SNP_35 at nucleotide 51 in SEQ ID NO: 35 (i.e. on chromosome 3 at nucleotide 9237416 of the cucumber genome Chinese Long v2 found at cucurbitgenomics.org) and ending 659 nucleotides downstream of SNP_36 at nucleotide 51 in SEQ ID NO: 36 i.e. on chromosome 3 at nucleotide 9265595 (SNP_36 is at nucleotide 9264936 of the cucumber genome Chinese Long v2 found at cucurbitgenomics.org). The sequence of QTL3.1 starting at SNP_35 and ending 659 nucleotides downstream of SNP_36 is provided in SEQ ID NO: 72. Thus, in one aspect the plants, seeds, plant parts, cells and/or tissues comprise the introgression fragment from a wild cucumber comprising a duplication of the QTL3.1 region, i.e. a duplication of the genomic sequence starting at SNP_35 and ending 659 nucleotides downstream of SNP_36, or a duplication of SEQ ID NO: 72 or a duplication of a chromosome 3 region comprising at least 95%, 96%, 97%, 98% or 99% sequence identity to SEQ ID NO: 72 when aligned pairwise. As DNA is double stranded, it is understood that when referring to a duplication of the plus strand, e.g. SEQ ID NO: 72 or a sequence comprising at least 95% sequence identity to SEQ ID NO: 72, this also refers to the duplication of the minus strand, e.g. SEQ ID NO: 73 or the minus strand of a sequence comprising at least 95% sequence identity to SEQ ID NO: 72. The duplication of QTL3.1, i.e. of a region comprising SEQ ID NO: 72 or a region comprising at least 95% sequence identity to SEQ ID NO: 72 (and comprising the 8 OGD genes and the gene encoding the HR-like protein), is present in the seeds deposited herein under accession number NCIMB 43530 but is also obtainable from other cucumber lines or accessions or wild donors or can be generated by inducing a duplication of the region. It is noted that SEQ ID NO: 72/73 is the consensus sequence, and not the sequence of the MYCR3 donor. The encoded proteins were however found to be the same in the donor and the consensus. Encompassed herein are therefore variants of SEQ ID NO: 72/73, comprising at least 95%, 96%, 97%, 98% and 99% sequence identity to SEQ ID NO: 72 or the complement strand SEQ ID No: 73, which encode the 9 proteins described herein.

In a further aspect plants, seeds, plant parts, cells and/or tissues comprise the introgression fragment from a wild cucumber comprising a duplication of the larger region, which comprises the QTL3.1 region, i.e. a duplication of the genomic sequence starting at (and preferably including) SEQ ID NO: 83 and ending at (and preferably including) SEQ ID NO: 84. Thus in one aspect the cucumber plant or plant part comprises a duplication of the region flanked by SEQ ID NO: 83 and SEQ ID NO: 84 or a duplication of the region starting at nucleotide 1 of SEQ ID NO: 83 and ending at nucleotide 100 of SEQ ID NO: 84. The region in between comprises QTL3.1, i.e. SEQ ID NO: 72 or a sequence comprising at least 95% sequence identity to SEQ ID NO: 72 (and comprising the 8 OGD genes and the gene encoding the HR-like protein). This larger duplication is present in the seeds deposited herein under accession number NCIMB 43530 but is also obtainable from other cucumber lines or accessions or wild donors or can be generated by inducing a duplication of the region.

In yet another aspect the plants, seeds, plant parts, cells and/or tissues comprise in their genome a duplication (i.e. at least two copies in the genome, e.g. at least 2 copies on one chromosome) of the genomic sequence starting at SNP_35 and ending 659 nucleotides downstream of SNP_36, i.e. of SEQ ID NO: 72 or a sequence comprising at least 95% sequence identity to SEQ ID NO: 72. In one aspect the duplication is a duplication of the genomic sequence of chromosome 3 starting at (and preferably including) SEQ ID NO: 83 and ending at (and preferably including) SEQ ID NO: 84. In a further aspect the plants, seeds, plant parts, cells and/or tissues comprise in their genome a duplication (i.e. at least two copies in the haploid genome, e.g. at least 2 copies on one chromosome) of one or more or all of the genes encoding the proteins OGD1 to OGD8 of SEQ ID NO: 52 to SEQ ID NO: 59 (or a protein comprising at least 95%, 96%, 97%, 98% or 99% amino acid sequence identity to any one of these) and/or the HR-like protein of SEQ ID NO: 60 (or a protein comprising at least 95%, 96%, 97%, 98% or 99% amino acid sequence identity to the HR-like protein of SEQ ID NO: 60).

In one aspect the plant comprising such a duplication in its genome is not obtained/obtainable exclusively by an essentially biological process, such as defined by Rule 28 (2) EPC. In one aspect the duplication is human induced. In one aspect the duplication is induced by a targeted genome editing technique, such as a Crispr based technique or by transgenic methods. The duplication and/or the two copies may both be present on chromosome 3. The duplication and/or the two copies may also be present on different chromosomes, e.g. one on chromosome 3 and one on a different chromosome. Different ways of duplicating the region or translocating the region to another chromosome exist, such as methods making use of targeted genome editing techniques, such as Crispr based techniques. For example Peter G. Lynagh et al. (doi: https://doi.org/10.1101/400507) have published an article entitled ‘Translocation and duplication from CRISPR-Cas9 editing in Arabidopsis thaliana’, wherein cuts in different chromosomes can result in translocations, or two cuts within a chromosome result in the duplication of the intervening segment.

In one aspect QTL3.1 (e.g. the introgression fragment comprising the duplication of QTL3.1, or the duplication of one or more of the 8 OGD genes and/or the gene encoding the HR-like protein) is present in heterozygous form in a cultivated cucumber plant, cell or tissue, especially in long cucumber. In another aspect QTL3.1 (e.g. the introgression fragment comprising the duplication of QTL3.1, or the duplication of one or more of the 8 OGD genes and/or the gene encoding the HR-like protein) is present in homozygous form in a cultivated cucumber plant, cell or tissue, especially in long cucumber. In a specific aspect the cultivated cucumber plant is an F1 hybrid, especially an F1 hybrid generated by crossing two inbred parent lines, whereby at least one of the parent lines comprises the QTL3.1 (e.g. the introgression fragment comprising the duplication of QTL3.1, or the duplication of one or more of the 8 OGD genes and/or the gene encoding the HR-like protein) in homozygous form.

The present QTLs, QTL3.1 (e.g. the duplication of QTL3.1) and QTL5.1, conferring cucumber fruit resistance are in one aspect not from a Cucumis sativus var. hardiwickii accession, e.g. not from accession PI183967. Accession PI183967 has been sequenced and the sequence is available on cucurbitgenomics.org. Comparing the OGD proteins and the HR-like protein described herein with those of PI183967 shows that for example OGD4 of SEQ ID NO: 55 only has 68.54% sequence identity to the protein in PI183967. The QTLs may, optionally, be combined with one or more of the QTLs from PI183967 conferring cucumber plant seedling resistance against Gummy Stem Blight described by Liu et al (Plant Disease 2017, 101:1145-1152), named gsb3.2, gsb3.3, gsb4.1, gsb5.1 or gsb6.1; or with cucumber plant stem resistance QTLs from PI183967 described in Zhang et al. 2017 (Mol. Breeding 37:49), named gsb-s1.1, gsb-s2.1, gsb-s6.1, gsb-s6.2 and gsb-s6.3.

In one aspect a cultivated cucumber plant is provided comprising both QTL5.1 and QTL3.1 (i.e. the duplication of QTL3.1 or the duplication of one or more of the 8 OGD genes and/or the gene encoding the HR-like protein) described herein (e.g. the introgression fragments comprising the QTLs), either both in homozygous form, e.g. in an inbred parent line (or in an F1 hybrid generated by crossing two inbred parent lines, both comprising QTL5.1 and QTL3.1 in homozygous form), or both in heterozygous form, e.g. in an F1 hybrid generated by crossing an inbred parent line comprising both QTL5.1 and QTL3.1 (i.e. the duplication of QTL3.1) in homozygous form with an inbred parent line lacking both QTL5.1 and QTL3.1 (i.e. lacking the duplication of QTL3.1). The presence of both QTL5.1 in heterozygous form and (the duplication of) QTL3.1 in heterozygous form in a single plant also leads to a significant increase of internal fruit rot resistance compared to a plant lacking both QTLs, albeit not as much as the presence of both QTL5.1 and (the duplication of) QTL3.1 in homozygous form in a single plant. As mentioned, in the Cucumis sativus seeds deposited by the applicant under accession number NCIMB 43530, QTL5.1 and QTL3.1 (i.e. the duplication of QTL3.1 on chromosome 3) are both present in homozygous form and these seeds, or progeny thereof, can be used as a source of one or both QTLs. However, QTL5.1 and QTL3.1 (i.e. the duplication of QTL3.1 on chromosome 3) can also be used independently to generate cucumber plants, breeding lines and varieties with increased resistance against DB fruit infection (a lower percentage of fruit infection than in the control lacking the QTLs/lacking QTL5.1 and lacking the duplication of QTL3.1).

As mentioned above, QTL5.1 alone or QTL3.1 (i.e. the duplication of QTL3.1 on chromosome 3) alone also have a positive effect on internal fruit rot resistance by reducing base-line susceptibility by e.g. 2%, 3%, 4%, 5%, 6%, 7%, 8%, 10%, 15%, 20% or more, especially when the QTLs are in homozygous form. In heterozygous form QTL5.1 alone or QTL3.1 (i.e. the duplication of QTL3.1 on chromosome 3) alone can also result in a positive effect on internal fruit rot resistance, but the effect may be difficult to measure and may only be seen in certain genetic backgrounds and not in others. This can be tested in an internal fruit rot resistance assay as e.g. described herein in the Examples.

In yet another aspect of the invention a cultivated cucumber plant is provided comprising both QTL5.1 and QTL3.1 (i.e. the duplication of QTL3.1 on chromosome 3) of the invention, whereby one of the QTLs is in homozygous form and the other QTL is in heterozygous form. See e.g. Table 5 showing a copy number effect. Thus, the different aspects include herein cucumber plants (and plant parts) with various numbers of copies of the DB resistance QTLs, especially in long cucumber, such as European Greenhouse cucumber types. As QTL3.1 is actually a duplication of QTL3.1 on the chromosome, this means that plants comprising two copies of QTL3.1 on chromosome 3 (heterozygous for the duplication of QTL3.1) or four copies on chromosome 3 (homozygous for the duplication of QTL3.1) and/or plants comprising one copy of QTL5.1 (heterozygous) or two copies of QTL5.1 (homozygous) are provided. In one aspect the plant is homozygous for the duplication of QTL3.1 or is homozygous for QTL5.1. In one aspect the plant is homozygous for the duplication of QTL3.1 and is homozygous for QTL5.1.

In still another aspect, the cultivated cucumber plant comprises QTL5.1 and/or QTL3.1 (i.e. the duplication of QTL3.1) from a donor (e.g. from NCIMB 43530 or progeny thereof), while apart from the introgression fragment the remaining regions of chromosome 5 and/or 3 is a genome of cultivated cucumber, in one aspect of European greenhouse cucumber. Thus, in one aspect the chromosome 5 and/or the chromosome 3 of the cultivated cucumber plant comprise no other introgression fragments. Optionally, however, the chromosome 5 and/or chromosome 3 may comprise other introgression fragments, e.g. from other donors in different regions of the chromosomes.

In one embodiment the other chromosomes are all cultivated cucumber genome, e.g. European greenhouse cucumber genome. That is to say that in one aspect of the invention the cultivated cucumber comprises only one introgression fragment from a wild cucumber in its genome (comprising either QTL5.1 or QTL3.1 in homozygous or heterozygous form) or comprises only two introgression fragments from a wild cucumber in its genome, one comprising QTL5.1 and one comprising QTL3.1, while the remaining genome is a genome of cultivated cucumber. In one aspect, the two introgression fragments are from the same wild cucumber, e.g. from the same species, preferably from the same accession number, optionally even from the same plant of that accession number.

In one aspect the source of the QTL3.1 (or of the duplication of QTL3.1) and/or QTL5.1 is NCIMB 43530, or progeny thereof, or commercial varieties developed by the applicant comprising the QTLs. In another aspect, the source of the QTL3.1 (or of the duplication of QTL3.1) and/or QTL5.1 is a wild cucumber accession comprising the donor haplotype of the SNP markers linked to the QTL, e.g. the haplotype of the donor for at least 3, 4, 5, 6, 7, 8, 9, 10, 11, 12 or more (or all) of the SNP markers SNP_01 to SNP_18 linked to QTL5.1 or of the SNP markers SNP_19 to SNP_42 linked to QTL3.1.

Also any wild or cultivated cucumber plant comprising a duplication of one or more or all of the 9 genes (2OGD1 to 2OGD8 and HR-like) on chromosome 3, or comprising a duplication of the regions comprising these genes, e.g. comprising a duplication of SEQ ID NO: 72 or a sequence/region comprising at least 95%, 96%, 97%, 98%, 99% sequence identity to SEQ ID NO: 72, may be used as source. The sequence of SEQ ID NO: 72 is from the reference genome Chinese Long V2, and donor sequences, such as MYCR3 and other donors may have some minor nucleotide variation in the sequence, especially in the non-coding sequence. It is for example estimated that the MYCR3 donor has 97%, 98% or even 99% sequence identity to SEQ ID NO: 72, while the encoded proteins are identical. In one aspect a donor comprises a duplication of the region of chromosome 3, which comprises the region starting at SEQ ID NO: 83 (and preferably including SEQ ID NO: 83) and ending at SEQ ID NO: 84 (and preferably including SEQ ID NO: 84). This region comprises SNP_29 to SNP_36, as shown in FIG. 4. In one aspect, therefore, a duplication of the region comprising SNP_29 to SNP_36 is present in the genome of e.g. the donor, or a duplication of the region comprising SNP_35 and SNP_36 is present in the genome, or a duplication of one or more or all of the genes encoding the proteins OGD1 to OGD8 and/or the HR-like protein is present. The presence of such duplications can be analyzed by sequencing or by quantitative PCR or by more recently developed methods, such as droplet digital PCR or ddPCR (see Jouanin et al. 2020, Journal of Cereal Science 92, p 1-9, 102903, ISSN 0733-5210), which can determine copy number of mutations or sequences.

In another aspect, the source of the QTL3.1 (or of the duplication of QTL3.1) and/or QTL5.1 is a wild cucumber accession comprising the donor haplotype of the SNP markers linked to the QTL, e.g. the haplotype of the donor for at least 3, 4, 5, 6, 7, 8, 9, 10 or 11 of the SNP markers SNP_12, SNP_45, SNP_13, SNP_14, SNP_46, SNP_47, SNP_48, SNP_49, SNP_50, SNP_51 and SNP_15 linked to QTL5.1 (see FIG. 6), or the donor SNP haplotype for SNP_29 to SNP_36, or for SNP_30 to SNP_36 or for SNP_31 to SNP_36, or for SNP_32 to SNP_36 or for SNP_33 to SNP_36, or for SNP_34 to SNP_36, or for SNP_35 to SNP_36, or comprising the region starting at SNP_35 and ending 659 nucleotides downstream of SNP_36 comprising QTL3.1 (i.e. comprising SEQ ID NO: 72 or a sequence comprising at least 95% sequence identity to SEQ ID NO: 72), or comprising a duplication of the region starting at SNP_35 and ending 659 nucleotides downstream of SNP_36 comprising QTL3.1, or comprising a duplication a sequence comprising SEQ ID NO: 72 or a sequence comprising at least 95% sequence identity to SEQ ID NO: 72, or comprising a duplication of the region starting at (and preferably comprising) SEQ ID NO: 83 and ending at (and preferably comprising) SEQ ID NO: 84, or comprising the genes encoding the 2OGD proteins of SEQ ID NO: 52 to SEQ ID NO: 59 (or proteins comprising at least 95%, 96%, 97%, 98% or 99% sequence identity to any of these) and/or comprising a gene encoding a HR-like protein of SEQ ID NO: 60 (or a protein comprising at least 95%, 96%, 97%, 98% or 99% sequence identity to SEQ ID NO: 60), or comprising a duplication of one or more of the genes encoding the 2OGD proteins of SEQ ID NO: 52 to SEQ ID NO: 59 (or proteins comprising at least 95%, 96%, 97%, 98% or 99% sequence identity to any of these) and/or comprising a duplication of a gene encoding a HR-like protein of SEQ ID NO: 60 (or a protein comprising at least 95%, 96%, 97%, 98% or 99% sequence identity to SEQ ID NO: 60).

In a different embodiment, the cultivated cucumber plant of the invention may, in addition to QTL5.1 and/or QTL3.1, comprise one or more other introgression fragments from wild cucumber or wild relatives of cucumber in its genome.

Although seeds deposited under NCIMB 43530 (and progeny thereof, or commercial varieties developed therefrom) are one optional source of QTL3.1 (or the duplication of QTL3.1 or a duplication of one or more of the 8 OGD genes and/or the gene encoding the HR-like protein) and/or QTL5.1 (and of the introgression fragments comprising these QTLs), the SNP markers and/or the sequences comprising the SNP markers or the sequences of the QTL region or genes provided can be used to identify other donors comprising QTL3.1 (or the duplication of QTL3.1) and/or QTL5.1 e.g. by identifying a donor comprising the donor haplotype of the SNP markers linked to the QTL, e.g. the haplotype of the donor for at least 3, 4, 5, 6, 7, 8, 9, 10, 11, 12 or more (or all) of the SNP markers SNP_01 to SNP_18 linked to QTL5.1, preferably for SNP_12, SNP_45, SNP_13, SNP_14, SNP_46, SNP_47, SNP_48, SNP_49, SNP_50, SNP_51 and/or SNP_15 linked to QTL5.1 (see FIG. 6) or of the SNP markers SNP_19 to SNP_42 linked to QTL3.1, preferably for the region or comprising the region starting at SNP_35 and ending 659 nucleotides downstream of SNP_36 comprising QTL3.1 (i.e. comprising SEQ ID NO: 72 or a sequence comprising at least 95% sequence identity to SEQ ID NO: 72), or comprising a duplication of the region starting at SNP_35 and ending 659 nucleotides downstream of SNP_36 comprising QTL3.1, or comprising the genes encoding the 2OGD proteins of SEQ ID NO: 52 to SEQ ID NO: 59 (or proteins comprising at least 95%, 96%, 97%, 98% or 99% sequence identity to any of these) and/or comprising a gene encoding a HR-like protein of SEQ ID NO: 60 (or a protein comprising at least 95%, 96%, 97%, 98% or 99% sequence identity to SEQ ID NO: 60), or comprising a duplication of one or more of the genes encoding the 2OGD proteins of SEQ ID NO: 52 to SEQ ID NO: 59 (or proteins comprising at least 95%, 96%, 97%, 98% or 99% sequence identity to any of these) and/or comprising a duplication of a gene encoding a HR-like protein of SEQ ID NO: 60 (or a protein comprising at least 95%, 96%, 97%, 98% or 99% sequence identity to SEQ ID NO: 60).

The left and right flanking sequences of the SNP markers provided herein are the genomic DNA sequences of the introgression fragment, i.e. of the MYCR3 donor, as present in the deposited seeds in homozygous form.

It is also noted that the deposited seeds are not seeds of a cucumber variety, but the seeds can be used to develop many different distinct, uniform and stable cucumber varieties (as defined by UPOV) comprising QTL3.1 (i.e. a duplication of QTL3.1) and/or QTL5.1, preferably in homozygous form.

### BACKGROUND

Cultivated cucumber (Cucumis sativus var. sativus L.) is an important vegetable crop worldwide. It belongs to the family Cucurbitaceae. It is thought to originate from South East Asia from wild ancestors with small, bitter fruits, such as Cucumis sativus var. hardwickii.

The cultivated cucumber genome has seven pairs of chromosomes (n=7) and a haploid genome size of about 367 Mb (Megabases) with an estimated total of about 26,682 genes. The cucumber genome was the first vegetable genome to be sequenced (Huang et al. 2009, Nature Genetics, Volume 41, Number 12, p 1275-1283 and world wide web at cucurbitgenomics.org/organism/2).

Didymella bryoniae is an ascomycete fungal plant pathogen which causes ‘Gummy Stem Blight’ of species in the family Cucurbitaceae, such as melon, watermelon, gourd and cucumber. Ascospores (produced by pseudothecia) or conidiospores (produced by pycnidia) infect plant tissue, especially leaves via wounds. Lesions appear, which grow rapidly and on which pycnidia or pseudothecia can form, producing new spores. Similar symptoms can develop on the stem, which can become ‘gummy’, giving the fungus its name.

In cucumber resistance against this fungus has been studied for seedling stage (leaf) resistance or stem resistance, and QTLs have been identified in a Cucumis sativus var. hardiwickii accession, PI183967, which reduce either leaf symptoms following spraying of spores onto the seedlings (Liu et al, Plant Disease 2017, 101:1145-1152) or which reduce stem symptoms following soaking of trimmed stems in spore suspensions for 30 minutes (Zhang et al. 2017, Mol. Breeding 37:49). The donor accession PI183967 shows partial leaf resistance following inoculation and also the stem resistance was partial. For leaf resistance six QTLs were mapped in different seasons, five of which came from PI183967 (gsb3.2, gsb3.3, gsb4.1, gsb5.1 or gsb6.1) and one came from the susceptible parent (gsb3.1). Only the QTL gbs5.1 was detected in all seasons and accounted for the highest phenotypic variation to leaf resistance. QTL gsb5.1 was located between SSR15321 and SSR07711 on chromosome 5, i.e. in the region around base 7026115 of chromosome 5 (Chinese long genome v2). In contrast, the QTL5.1 of the instant invention, conferring internal fruit resistance, is in the region starting at base 3701817 and ending at 4028826 of chromosome 5 (Chinese long genome v2, world wide web at cucurbitgenomics.org/organism/2).

For stem resistance five QTLs were mapped in different seasons, gsb-s1.1, gsb-s2.1, gsb-s6.1, gsb-s6.2 and gsb-s6.3.

The inventors developed a disease assay to study the infection of cucumber flowers following inoculation with spores of DB. Once this assay was developed, they used it to find a plant accession which had a high ability to prevent fruit infection via the flower. This is the first time that it has been tried to develop a resistance assay for fruit infection by the fungus via the flower and that, using this new approach, QTLs have been identified which confer internal fruit rot resistance, i.e. QTLs which reduce the percentage of fruits infected by DB following flower inoculation with spores of Didymella bryoniae. In different long cucumber lines the presence of both QTL3.1 (i.e. the duplication of QTL3.1) and QTL5.1 in homozygous form reduced the percentage of fruits showing symptoms to less than 1.5%, and in some genetic backgrounds even to 0%, see Examples. This means that plants which have these two QTLs can be grown under conditions where the fungus may be present, and where the fungus may even cause symptoms on stems and leaves of the plants, but whereby at least 98.5% of the cucumber fruits remain free of fungal infection.

### FIGURES

FIG. 1 shows a cucumber fruit showing no symptoms following inoculation of the flower with DB spores and three cucumber fruits showing symptoms of internal fruit infection following inoculation of the flower with DB spores. In the fruit disease assay the fruit on the left, without any symptoms, is scored as ‘resistant’ or as ‘showing no symptoms of infection’ and the three fruits on the right, showing symptoms of infection, are scored as ‘susceptible’ or as ‘showing symptoms of infection’ (despite the varying degree of internal browning at the flower end). The scoring is, thus, a scoring to differentiate between fruits which are not infected/no symptoms at 10 days following flower inoculation and fruits which are infected/show symptoms at 10 days following flower inoculation. Of all inoculated flowers of a plant line (e.g. 10 plants of a plant genotype, for example of a line comprising QTL3.1 (i.e. the duplication of QTL3.1) and QTL5.1 in homozygous form), the percentage of fruits ‘showing symptoms of infection’ is determined and compared to the percentage of fruits ‘showing symptoms of infection’ of the control plant line lacking the QTLs, e.g. the genetic control. See also Examples.

FIGS. 2 and 3 shows intact fruits and sliced open fruits, respectively, whereby no symptoms of fungal infection are seen externally, but when sliced open the internal fruit infection (browning at the flower end) can be seen. The bottom fruit comprises QTL3.1 (i.e. the duplication of QTL3.1) and QTL5.1 in homozygous form, while the top fruit does not.

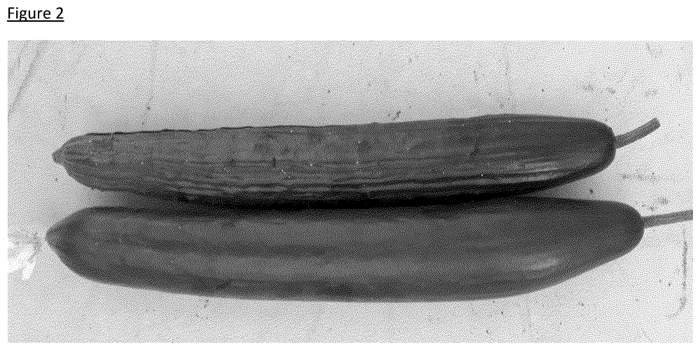

FIG. 4 shows a schematic diagram of the structural variation found to underly QTL3.1. After fine mapping QTL3.1 was found to be located between SNP_35 and 659 nt downstream of SNP_36. Sequencing of the region revealed that QTL3.1 was duplicated on chromosome 3 in the resistant plant (as part of a duplication of about 150 kb region, shown as grey bar, of which the first and last 100 nucleotides are provided in SEQ ID NO: 83 and 84), while the susceptible plant did not contain this duplication and therefore contained only one QTL3.1 on chromosome 3. Nine genes were found to be located in the QTL3.1 region (provided in SEQ ID NO: 72 or a sequence comprising at least 95% identity to SEQ ID NO: 72) and the higher expression of these genes in the resistant plant versus the susceptible plant causes the reduced susceptibility to internal fruit rot caused by DB. The gene order is given, with the gene encoding OGD1 being on the left and the gene encoding HR-like protein being on the right.

FIG. 5 shows a multiple sequence alignment of the 2OGD proteins (OGD1 to OGD8) found in the QTL3.1 region and the most similar Arabidopsis proteins, encoded by the genes At1g06650 and At1g06620. The conserved Pfam domains ‘DIOX-N’ (Pfam 14226) and ‘20G-FEII-OXY’ (Pfam 03171) are indicated.

FIG. 6 shows a schematic diagram of the region on chromosome 5 comprising QTL5.1 and the SNP markers linked to QTL5.1. On the left the SNP markers linked to QTL5.1 in the original mapping are shown (SNP_01 to SNP_18) and on the right the fine-mapped location of QTL5.1 and the SNP markers linked to QTL5.1 after fine mapping are shown (SNP_12 to SNP_15, with additional markers added in the region).

FIG. 7 shows a schematic representation of the QTL3.1 region and the genes lying on the plus strand (SEQ ID NO: 72) or the minus strand (SEQ ID NO: 73). SEQ ID NO: 72 starts at SNP_35 and ends 659 nucleotides downstream of SNP_36. SNP_36 was found to be located in an intron of the HR-like gene, which is on the plus strand of the DNA (see FIG. 8). The 8 OGD genes are on the minus strand of the genomic DNA. The plus and minus strands are provided herein in SEQ ID NO: 72 and 73, respectively. Pairwise alignment of the promoter sequences or the cDNA sequences with SEQ ID NO: 72 or 73 can be used to retrieve the genomic sequences of the gene, including the intron sequences. The genomic sequences (as well as mRNA and protein sequences) are also retrievable from the world wide web at cucurbitgenomics.org from the Cucumis sativus var. sativus cv 9930 (Chinese Long) V2 database.

FIG. 8 shows the location of SNP_36 in the gene encoding the HR-like protein (exons are shaded grey). The sequence of FIG. 8 is also provided in SEQ ID NO: 85. The sequence corresponds to SEQ ID NO: 72 from nucleotide 26635 to the end of the sequence, nucleotide 28180. SNP_36 is in the intron of the gene and the QTL3.1 region comprises the region starting at SNP_35 (nucleotide 1 of SEQ ID NO: 72) and ending 659 nucleotides downstream of SNP_36, at nucleotide 28180 of SEQ ID NO: 72.

### GENERAL DEFINITIONS

The indefinite article “a” or “an” does not exclude the possibility that more than one of the element is present, unless the context clearly requires that there be one and only one of the elements. The indefinite article “a” or “an” thus usually means “at least one”.

As used herein, the term “plant” includes the whole plant or any parts or derivatives thereof, such as plant organs (e.g., harvested or non-harvested storage organs, tubers, fruits, leaves, seeds, etc.), plant cells, plant protoplasts, plant cell or tissue cultures from which whole plants can be regenerated, plant calli, plant cell clumps, and plant cells that are intact in plants, or parts of plants, such as embryos, pollen, ovules, ovaries, fruits (e.g., harvested tissues or organs, such as harvested cucumber fruits or parts thereof), flowers, leaves, seeds, tubers, bulbs, clonally propagated plants, roots, root-stocks, stems, root tips and the like. Also any developmental stage is included, such as seedlings, immature and mature, etc. When “seeds of a plant” are referred to, these either refer to seeds from which the plant can be grown or to seeds produced on the plant, after self-fertilization or cross-fertilization.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of one or two loci or genes (or phenotypic characteristics due to these specific loci or genes), but which can otherwise differ from one another enormously as regards the other loci or genes. Thus, e.g. a plant defined only by the presence of (a duplication of) QTL3.1 and/or QTL5.1 is not a plant variety, as thousands of other genes which define a plant variety are undefined and a plant defined only by the presence of (a duplication of) QTL3.1 and/or QTL5.1 is not uniform and stable for these thousands of genes and the characteristics conferred by these genes. QTL3.1 and/or QTL5.1 can be used to develop many different plant varieties, e.g. a long cucumber variety which is uniform and stable for all its physiological and morphological characteristics such as leaf size or shape, leaf margins and color, fruit size and color, warts, bitterness, plant height, etc. and which also comprises QTL3.1 and/or QTL5.1.

“F1, F2, F3, etc.” refers to the consecutive related generations following a cross between two parent plants or parent lines. The plants grown from the seeds produced by crossing two plants or lines is called the F1 generation. Selfing the F1 plants results in the F2 generation, etc.

“F1 hybrid” plant (or F1 hybrid seed) is the generation obtained from crossing two inbred parent lines. Thus, F1 hybrid seeds are seeds from which F1 hybrid plants grow. F1 hybrids are more vigorous and higher yielding, due to heterosis. Inbred lines are essentially homozygous at most loci in the genome.

A “plant line” or “breeding line” refers to a plant and its progeny. As used herein, the term “inbred line” refers to a plant line which has been repeatedly selfed and is nearly homozygous. Thus, an “inbred line” or “parent line” refers to a plant which has undergone several generations (e.g. at least 4, 5, 6, 7 or more) of inbreeding, resulting in a plant line with a high uniformity.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The term “gene” means a DNA sequence comprising a region (transcribed region), which is transcribed into an RNA molecule (e.g. an mRNA/messenger RNA or an RNAi molecule) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. A gene may be an endogenous gene (in the species of origin) or a chimeric gene (e.g. a transgene or cis-gene).

The term “locus” (loci plural) means a specific place or places or a site on a chromosome where for example a QTL, a gene or genetic marker is found. The DB internal fruit rot resistance locus is, thus, the location in the genome of cucumber, where QTL3.1 or QTL5.1 is found. In cultivated cucumber of the invention the QTLs are found on chromosome 3 and/or 5 (using the chromosome assignment of Huang et al. 2009, Nature Genetics, Volume 41, Number 12, p 1275-1283 and world wide web at//cucurbitgenomics.org/organism/2 and described therein as “Cucumber (Chinese Long) v2 Genome”) i.e. in one aspect they are introgressed into the cultivated cucumber genome (i.e. onto chromosome 3 and/or 5) from a wild cucumber (also referred to as donor herein).

A “quantitative trait locus”, or “QTL” is a chromosomal locus that encodes for one or more alleles that affect the expressivity of a continuously distributed (quantitative) phenotype. The DB fruit rot resistance conferring quantitative trait loci are named QTL3.1 and QTL5.1 herein.

“Cucumber genome” and “physical position on the cucumber genome” and “chromosome 3 and/or 5” refers to the physical genome of cultivated cucumber, world wide web at//cucurbitgenomics.org/under “Cucumber (Chinese Long) v2 Genome”/, and the physical chromosomes and the physical position on the chromosomes. So, for example SNP_01 at nucleotide 51 od SEQ ID NO: 1 is located at the nucleotide (or ‘base’) positioned physically at nucleotide 3701817 of chromosome 5.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). In cucumber, wild or primitive cucumber accessions (e.g. landraces) or wild relatives of cultivated cucumber can be used to introgress fragments of the wild genome (donor) into the genome of cultivated cucumber, Cucumis sativus var. sativus L. Such a cultivated cucumber plant thus has a “genome of cultivated Cucumis sativus var. sativus”, but comprises in the genome a fragment of a wild or primitive cucumber or of a wild relative of cucumber, e.g. an introgression fragment of a related wild Cucumis sativus genome, such as Cucumis sativus var. hardwickii, C. sativus var. sikkimensis Cucumis sativus var. xishuangbannesis, or another wild cucumber or wild relative of cucumber. So, for example, a cultivated cucumber is provided herein comprising a genome of cultivated cucumber, and in that genome one introgression fragment on chromosome 3 and/or 5 from a wild cucumber donor accession which confer internal fruit rot resistance caused by DB compared to the cultivated cucumber genome lacking the introgression fragments (and having a chromosomes 3 and 5 of cultivated cucumber, without the introgression fragments). It is understood that the term “introgression fragment” never includes a whole chromosome, but only a part of a chromosome, and that the introgression fragment is characterized by the donor SNP markers or donor SNP haplotype of 1, 2, 3, 4, 5, 6, 7, 8, 9, 10 or more SNP markers, especially consecutive SNP markers. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 350,000 bp, 200,000 bp (equals 200 kilo base pairs) or less, about 100,000 bp (100 kb) or less, about 50,000 bp (50 kb) or less, about 25,000 bp (25 kb) or less.

“Cultivated cucumber” or “domesticated cucumber” refers to plants of Cucumis sativus var. sativus i.e. varieties, breeding lines or cultivars, cultivated by humans and having good agronomic characteristics, especially producing edible and marketable fruits of good size and quality and uniformity; such plants are not “wild cucumber” or “primitive cucumber” plants, i.e. plants which generally have much poorer yields and poorer agronomic characteristics than cultivated plants and are less uniform genetically and in their physiological and/or morphological characteristics. “Wild plants” of “wild cucumber” include for example ecotypes, landraces or wild accessions or wild relatives of a species. Cultivated cucumber plants (lines or varieties) can also be distinguished from wild or primitive cucumber accessions by the significantly lower amount of SNPs (less than 2,000,000 SNPs) and INDELs (insertions/deletions of shorter than 5 bp; less than 150,000 INDELs) in the genome and their significantly lower nucleotide diversity (equal to or less than 2.3×10⁻³ π), as described in Table 1 of Qi et al, Nature Genetics December 2013, Vol 45, No. 12, pages 1510-1518. SNP numbers, INDEL numbers and nucleotide diversity can be determined as described herein, especially in the section ‘Online Methods’.

“Indian cucumber group” refers to wild or wild relatives of cucumbers from India, having a high amount of SNPs (more than 3,000,000 SNPs) and INDELs (insertions/deletions of shorter than 5 bp; more than 200,000 INDELs) in the genome and high nucleotide diversity (more than 3.0×10⁻³ π or even more than 4.0×10⁻³ π).

“Eurasian cucumber group” refers to cultivated cucumbers from central or western Asia, Europe and the United States, having a low amount of SNPs (less than 2,000,000 SNPs, or less than 1,500,000 SNPs) and INDELs (insertions/deletions of shorter than 5 bp; less than 150,000 INDELs) in the genome and a low nucleotide diversity (equal to or less than 2.3×10⁻³ π, preferably less than 2.0×10⁻³ π).

“East Asian cucumber group” refers to cultivated cucumbers from East Asia, such as China, Korea and Japan, having a low amount of SNPs (less than 2,000,000 SNPs, or less than 1,500,000 SNPs) and INDELs (insertions/deletions of shorter than 5 bp; less than 150,000 INDELs, preferably less than 100,000) in the genome and a low nucleotide diversity (equal to or less than 2.3×10⁻³ π, preferably less than 2.0×10⁻³ π or even less than 1.5×10⁻³ π).

“Xishuangbanna cucumber group” refers to cucumbers from the Xishuangbanna region of China, having a low amount of SNPs (less than 2,000,000 SNPs, or less than 1,500,000 SNPs or even less than 100,000 SNPs) and INDELs (insertions/deletions of shorter than 5 bp; less than 150,000 INDELs, preferably less than 100,000) in the genome and a low nucleotide diversity (equal to or less than 2.3×10⁻³ π, preferably less than 2.0×10⁻³ π or even less than 1.5×10⁻³ π).

“Wild cucumber” or “primitive cucumber” refers to C. sativus var. sativus which generally have much poorer yields and poorer agronomic characteristics than cultivated plants and are less uniform genetically and in their physiological and/or morphological characteristics. Wild plants include for example ecotypes, landraces or wild accessions or wild relatives of a species.

“Wild relatives of cucumber” refer to Cucumis sativus var. hardwickii, C. sativus var. sikkimensis, Cucumis sativus var. xishuangbannesis.

“Landrace(s)” refers to primitive cultivars of Cucumis sativus var. sativus developed in local geographic regions, which often show a high degree of genetic variation in their genome and exhibit a high degree of morphological and/or physiological variation within the landrace (e.g. large variation in fruit size, etc.), i.e. are significantly less uniform than cultivated cucumber. Landraces are, therefore, herein included in the group “wild cucumber”, which is distinct from “cultivated cucumber”.

“Uniformity” or “uniform” relates to the genetic and phenotypic characteristics of a plant line or variety. Inbred lines are genetically highly uniform as they are produced by several generations of inbreeding. Likewise, and the F1 hybrids which are produced from such inbred lines are highly uniform in their genotypic and phenotypic characteristics and performance.

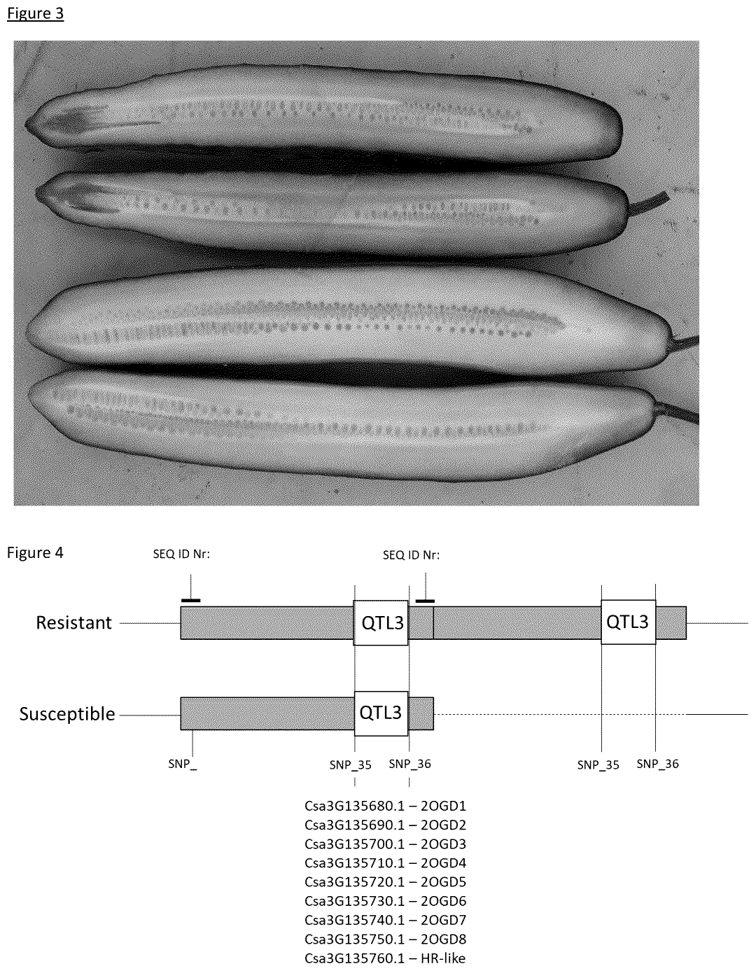

The term “internal fruit rot resistance allele” or “fruit rot resistance allele” or “DB fruit rot resistance allele” or “DB resistance allele” refers to an allele found at the locus QTL3.1 (or the duplicated QTL3.1) and/or QTL5.1 introgressed, in one aspect, into cultivated cucumber (onto cultivated C. sativus var. sativus chromosome 3 or 5 respectively) from a wild donor. The term thus, also encompasses alleles obtainable from other Cucumis donor accessions, which e.g. comprise the same donor haplotype for at least 3, 4, 5, 6, 7, 8, 9, 10, 11, 12 or more (or all, or all but 1, 2, or 3) of the SNP markers linked to the QTL. When the allele is present in homozygous form at the (duplicated) QTL3.1 and/or QTL5.1 locus in the genome, the plant, plant line or variety comprises a significantly reduced susceptibility (increased resistance) to internal fruit rot than the control lacking the QTLs, preferably the genetic control. In cultivated cucumber plants lacking the introgression fragment, the C. sativus var. sativus allele found at the same locus on chromosome 3 or chromosome 5 is herein referred to as “wild type” allele (wt). As resistance conferred by QTL3.1 turned out to be due to a duplication of QTL3.1 on the chromosome (see FIG. 4), the “wild type” may herein refer to the absence of the duplication of QTL3.1, i.e. a plant “lacking QTL3.1” refers to a plant ‘lacking the duplication of QTL3.1’. The genotype or haplotype of the SNP markers provided herein, which are physically linked to the allele, is also indicative of the wild type allele or of the QTL being in homozygous or heterozygous form. E.g. the genotype of SNP_01 indicative of QTL5.1 is ‘AG’ (QTL5.1/wt) or ‘AA’ (QTL5.1/QTL5.1), while the genotype indicative of the wild type is ‘GG’ (wt/wt), the genotype of SNP_02 indicative of indicative of QTL5.1 is ‘GA’ (QTL5.1/wt) or ‘GG’ (QTL5.1/QTL5.1), while the genotype indicative of the wild type is ‘AA’ (wt/wt), etc. Likewise the haplotype for SNP_01 and SNP_02 indicative of QTL5.1 being in homozygous form is AA-GG.

QTL3.1 was found to lie in between SNP_35 and 659 nt downstream of SNP_36 (SEQ ID NO: 72 and 73), and the resistant plant was found to be resistant due to a duplication of QTL3.1 being present in the plant. Therefore, a plant comprising “QTL3.1” refers herein in one aspect to a plant comprising a duplication of QTL3.1 on chromosome 3, i.e. a duplication of all or part of the region between SNP_35 and (659nt downstream of) SNP_36 or a duplication of one or more or all of the 8 OGD genes and/or the gene encoding the HR-like protein, while a plant “lacking” QTL3.1 refers in one aspect to a plant lacking a duplication of QTL3.1 on chromosome 3 e.g. comprising only one copy of the region between SNP_35 and SNP_36 and comprising only one copy of the 8 OGD genes and the gene encoding the HR-like protein on chromosome 3.

Regarding QTL3.1 the term “internal fruit rot resistance alleles” in one aspect refers to one or more or all of the nine genes which are located in the QTL3.1 region, which encode proteins referred to as OGD1 to OGD8 (2OGD proteins) and HR-like protein (Hypersensitive Response-like protein).

“OGD proteins” or “2OGD proteins” refers to 2-oxoglutarate and Fe(II) dependent oxygenase proteins.

“SNP marker” refer herein to single nucleotide polymorphisms of a genomic sequence linked to QTL5.1 or to QTL3.1, whereby a specific nucleotide (e.g. for SNP_01 an Adenine at nucleotide 51 of SEQ ID NO:1, or an Adenine at nucleotide 51 of a sequence comprising at least 95%, 96% or 97% sequence identity to SEQ ID NO: 1), or sequence comprising the specific nucleotide, is linked to the QTL. This nucleotide, or sequence comprising the nucleotide, is also referred to as the ‘SNP genotype’ or ‘SNP nucleotide’ of the plant or plant part, and SNP_01 may be ‘A’ (haploid, on one chromosome) or ‘AA’ (diploid, on both chromosomes). Markers SNP_01 to SNP_18 are linked to QTL5.1 and are present on the introgression fragment which comprises QTL5.1. Markers SNP_19 to SNP_42 are linked to QTL3.1 and the markers SNP_29 to SNP_36 are present on the duplication of the region comprises QTL3.1.

“INDEL marker” is herein a marker whereby one nucleotide is inserted/deleted when comparing two sequences. For example, the markers referred herein to as SNP_23 and SNP_36 are in fact INDEL markers, and not SNP markers. However, purely for ease of describing these markers, they are referred to as SNP markers throughout the specification. SNP_23 comprises the nucleotide Cytosine at nucleotide 51 of SEQ ID NO: 23 (donor sequence) or comprises Cytosine-Adenine at the same position (recurrent parent sequence), as shown in SEQ ID NO: 43. Similarly, SNP_36 comprises the nucleotide Thymine at nucleotide 51 of SEQ ID NO: 36 (donor sequence) or comprises Thymine-Guanine at the same position (recurrent parent sequence), as shown in SEQ ID NO: 44.

The ‘haplotype’ or “haploid genotype” refers to the haploid genotype of several genetic loci in a plant, especially of several SNP markers or several sequences comprising the SNP markers. For QTL5.1 the SNP haplotype may thus be the haploid genotype of at least 2, 3, 4, 5, 6 or more (e.g. all 18) SNP markers of SNP_01 to SNP_18 (or of the sequences comprising the SNP markers). For example, the plant comprising QTL5.1 may comprise a ‘A’ for SNP_01 at nucleotide 51 in SEQ ID NO: 1 (or at nucleotide 51 of a sequence which is at least 95%, 96% or 97% identical to SEQ ID NO: 1), a ‘G’ for SNP_02 at nucleotide 51 in SEQ ID NO: 2 (or at nucleotide 51 of a sequence which is at least 95%, 96% or 97% identical to SEQ ID NO: 2), a ‘C’ for SNP_03 in SEQ ID NO: 3, it thus has the SNP haplotype A-G-C for SNP_01 to SNP_03, which is the SNP haplotype of SNP_01 to SNP_03 of the wild cucumber donor (also referred to as donor SNP haplotype).

A genetic element, an introgression fragment, or a gene or allele conferring a trait (such as internal fruit rot resistance) is said to be “obtainable from” or can be “obtained from” or “derivable from” or can be “derived from” or “as present in” or “as found in” a plant or seed or tissue or cell if it can be transferred from the plant or seed in which it is present into another plant or seed in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only seeds deposited and comprising the genetic element, locus, introgression fragment, gene or allele can be used, but also progeny/descendants from such seeds which have been selected to retain the genetic element, locus, introgression fragment, gene or allele, can be used and are encompassed herein, such as commercial varieties developed from the deposited seeds or from descendants thereof. Whether a plant (or genomic DNA, cell or tissue of a plant) comprises the same genetic element, locus, introgression fragment, gene or allele as obtainable from the deposited seeds can be determined by the skilled person using one or more techniques known in the art, such as phenotypic assays, whole genome sequencing, molecular marker analysis, trait mapping, chromosome painting, allelism tests and the like, or combinations of techniques.

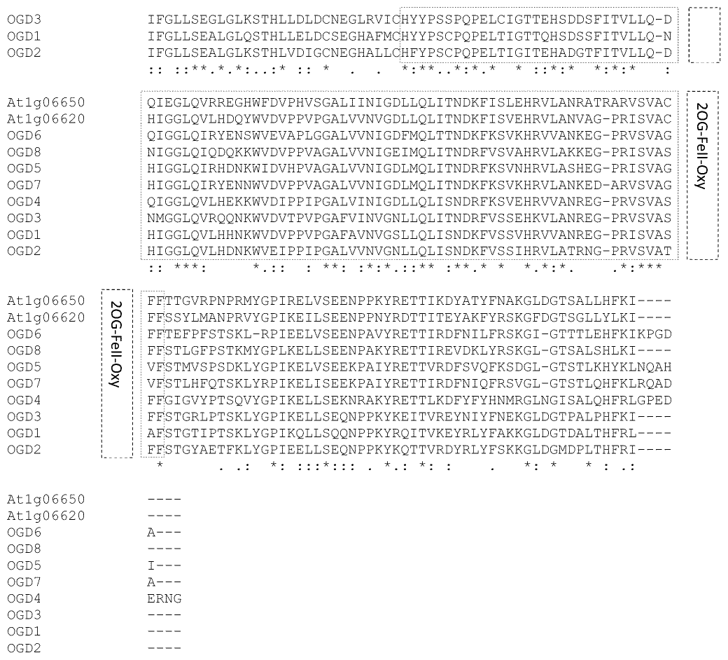

A “Variant” or “orthologous” sequence or a “variant QTL3.1” or a “variant of QTL5.1” refers to a DB fruit rot resistance conferring QTL (QTL3.1 or QTL5.1), or an introgression fragment comprising the QTL, which is derived from a different wild cucumber donor plant than the QTL3.1 (e.g. the duplication of QTL3.1) and QTL5.1 present in NCIMB 43530. Such a variant QTL can e.g. be identified as having the same SNP haplotype as the QTLs present in NCIMB43530 for at least 3, 4, 5, 6, 7, 8, 9, 10, 11, 12, 13, 14, 15, 16, 17, 18 or more markers (preferably consecutive markers) selected from SNP_01 to SNP_18 for a variant of QTL5.1, preferably one or more markers selected from SNP_12 to SNP_15 as shown in FIG. 6, and selected from SNP_19 to SNP_42 for a variant of QTL3.1, more preferably SNP_29 to SNP_36, or preferably as comprising a duplication of the QTL3.1 region (between SNP_35 and 659 nt downstream of SNP_36) or a duplication of one or more of the genes encoding an OGD protein and/or a HR-like protein. So for example a plant comprising a variant QTL5.1 may comprise a SNP haplotype A-G-C-G for SNP_01 to SNP_04, i.e. an ‘A’ for SNP_01 at nucleotide 51 in SEQ ID NO: 1 (or at nucleotide 51 of a sequence which is at least 95%, 96% or 97% identical to SEQ ID NO: 1), a ‘G’ for SNP_02 at nucleotide 51 in SEQ ID NO: 2 (or at nucleotide 51 of a sequence which is at least 95%, 96% or 97% identical to SEQ ID NO: 2), a ‘C’ for SNP_03 at nucleotide 51 in SEQ ID NO: 3 (or at nucleotide 51 of a sequence which is at least 95%, 96% or 97% identical to SEQ ID NO: 3) and a ‘G’ for SNP_04 at nucleotide 51 in SEQ ID NO: 4 (or at nucleotide 51 of a sequence which is at least 95%, 96% or 97% identical to SEQ ID NO: 4). In addition the variant QTL confers in homozygous form reduced susceptibility to internal fruit infection following flower inoculation with DB spores, as described herein.

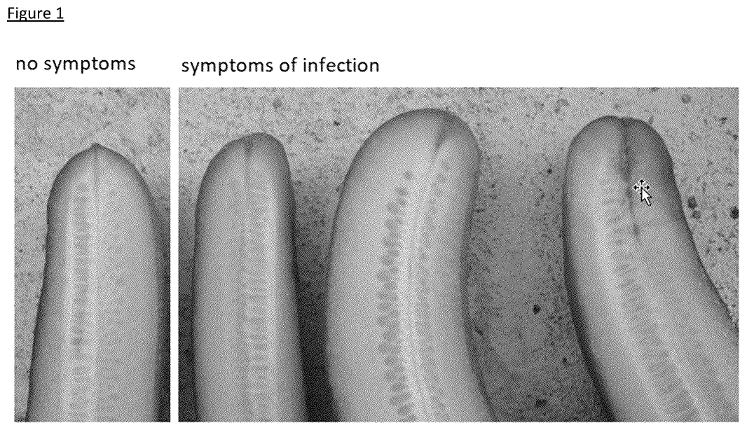

“DB fruit infection” or “internal fruit rot infection” or “internal fruit symptoms” refers to the ability of Didymella bryoniae spores (conidia or ascospores) to infect the fruits and develop internal symptoms inside the cucumber fruit following inoculation of the flower with spores, as described e.g. in the fruit infection assay (or DB fruit rot resistance assay). The symptoms are seen at the flower end, as browning, and are at first very slight and then spread deeper into the fruit, see FIG. 1 (three fruits on the right side with increasing spread of symptoms).

An “increased resistance against internal fruit rot infection” or a “significantly increased DB fruit resistance” or a “reduced susceptibility to internal fruit infection” refers to a cultivated cucumber plant, plant line, hybrid or variety comprising e.g. an introgression fragment on chromosome 3 and/or 5, comprising QTL3.1 (e.g. the duplication of QTL3.1) and/or QTL5.1, having (due to the QTLs) a lower percentage of fruits (that develop from flowers inoculated with Didymella bryoniae spores) which show internal fruit infection symptoms compared to the control plant lacking both QTLs (i.e. lacking QTL5.1 and lacking the duplication of QTL3.1 on chromosome 3), preferably the genetic control plant.

“Base line susceptibility” refers to the percentage of fruits (that develop from spore-inoculated flowers) which show internal fruit symptoms of a control plant lacking QTL3.1 (e.g. lacking the duplication of QTL3.1 on chromosome 3) and QTL5.1. As different cucumber lines or types have different base-line susceptibility to Didymella bryoniae fruit infection, the effect of QTL3.1 (e.g. the duplication of QTL3.1, or the enhanced expression of one or more genes encoding 2OGD proteins and/or a HR-like protein) and/or QTL5.1 can be measured as the reduction of base-line susceptibility. If the susceptible control (preferably the genetic control) has a base-line susceptibility of 80% (i.e. 80% of the spore-inoculated flowers develop fruits with internal fruit rot symptoms), then the presence of QTL3.1 (e.g. the duplication of QTL3.1, or the enhanced expression of one or more genes encoding 2OGD proteins and/or a HR-like protein) and/or QTL5.1 (in homozygous form) can reduce the base-line susceptibility by at least 3%, 5%, 10%, 15%, 20%, 25%, 30%, 40%, 50%, or more.

“Fruit infection assay” or “internal fruit rot assay” or “DB fruit rot resistance assay” refers to an assay as e.g. described in the examples, whereby e.g. at least 8, 9 or 10 plants of a plant comprising QTL5.1 and/or QTL3.1 (e.g. the duplication of QTL3.1, or the enhanced expression of one or more genes encoding 2OGD proteins and/or a HR-like protein) and at least 8, 9, or 10 plants of a susceptible control lacking both QTLs (preferably the genetic control) are grown and over a period of several weeks the newly opened flowers are inoculated with a Didymella bryoniae spore suspension (e.g. 3×10⁴ spores/ml, 10 μl per flower). About 10 days post inoculation of the flower, the fruit is sliced open and is either assessed as being free of internal symptoms (FIG. 1, left hand fruit) or as having internal fruit rot symptoms (FIG. 1, three right hand fruits). The percentage of fruits having internal symptoms is calculated for the plant comprising one or both QTLs and for the control plant lacking both QTLs.

“Control plant” is a cultivated cucumber genotype, breeding line, hybrid or variety lacking the introgression fragments or lacking a modification of gene expression and/or copy number of one or more genes encoding 2OGD proteins and/or a HR-like protein. The control plant is preferably of the same type as the plant comprising the introgression fragment(s) or the modified copy number or gene expression, e.g. long cucumber type, pickling type, short cucumber type, slicer type, etc. For example, the original DB susceptible parent line into which the QTL5.1 and/or QTL3.1 (e.g. the duplication of QTL3.1) was introgressed (also referred to as the recurrent parent) is a suitable control.

“Genetic control” is a cultivated cucumber genotype, breeding line, variety or hybrid which has the same or very similar cultivated genome as the cucumber plant comprising the introgression on chromosome 3 and/or 5 except that it lacks the introgressions on chromosome 3 and 5, i.e. chromosome 3 and 5 are “wild type”, i.e. cultivated cucumber genome. This is for example a backcross line in the backcrossing program which does not contain the introgression fragments.

The term “marker assay” refers to a molecular marker assay which can be used to test whether on cultivated C. sativus var. sativus chromosome 3 and/or 5 an introgression from a wild cucumber is present which introgression fragment comprises the DB fruit rot resistance QTL3.1 (e.g. the duplication of QTL3.1) and/or QTL5.1 (or whether a wild cucumber accession comprises the QTL3.1 (e.g. a duplication of QTL3.1) and/or QTL5.1, or a variant thereof, in its genome), by determining the genotype or haplotype of any one or more markers linked to the QTL3.1 and/or to QTL5.1, e.g. the genotype or haplotype of one or more SNP markers selected from SNP_01 to SNP_18 for QTL5.1 or the genotype or haplotype of one or more SNP markers selected from SNP_19 to SNP_42 for QTL3.1.

“Flanking markers” are markers which are on either side of the QTL, i.e. the QTL is located on the chromosomal region in-between the flanking markers, e.g. the QTL5.1 (or a variant QTL5.1) is in between SNP_01 at nucleotide 51 of SEQ ID NO: 1 and SNP_18 at nucleotide 51 of SEQ ID NO: 18, preferably in between SNP_12 and SNP_15 as e.g. shown in FIG. 6. QTL3.1 (or a variant QTL3.1) is in one aspect in between SNP_19 at nucleotide 51 of SEQ ID NO: 19 and SNP_42 at nucleotide 51 of SEQ ID NO: 42. In another aspect QTL3.1 is in between SNP_35 and SNP_37, or in between SNP_35 and the nucleotide 659 nucleotides downstream of SNP_36.

The SNP markers provided herein, i.e. SNP_01 to SNP_18, preferably SNP_12, SNP_45, SNP_13, SNP_14, SNP_46 to SNP_51 and SNP_15 (see FIG. 6) for chromosome 5 and SNP_19 to SNP_42 for chromosome 3, are located in the given order on the introgression fragment. “Consecutive” markers refers to markers in the same consecutive order, so e.g. two consecutive markers may be SNP_01 and SNP_02; SNP_02 and SNP_03; SNP_03 and SNP_04, etc. and three consecutive markers may be SNP_01 and SNP_02 and SNP_03; SNP_02 and SNP_03 and SNP_04; etc.

“Duplicated” means at least two copies being present in the haploid genome, preferably on the chromosome, while in the wild type haploid genome only one copy is present. “Duplicated SNP markers” are for example SNP_29 to SNP_36 being present in at least two copies on e.g. chromosome 3. Duplicated genes are also present in at least two copies on e.g. chromosome 3.

“Average” or “mean” refers herein to the arithmetic mean and both terms are used interchangeably. The term “average” or “mean” thus refers to the arithmetic mean of several measurements. The skilled person understands that the phenotype of a plant line or variety depends to some extent on growing conditions and that, therefore, arithmetic means of at least 8, 9, 10, 15, 20, 30, 40, 50 or more plants (or plant parts) are measured, preferably in randomized experimental designs with several replicates and suitable control plants grown under the same conditions in the same experiment. “Statistically significant” or “statistically significantly” different or “significantly” different refers to a characteristic of a plant line or variety that, when compared to a suitable control (e.g. the genetic control) show a statistically significant difference in that characteristic (e.g. the p-value is less than 0.05, p<0.05, using ANOVA) from the mean of the control.

A “recombinant chromosome” refers to a chromosome having a new genetic makeup arising through crossing-over between homologous chromosomes, e.g. a “recombinant chromosome 3” or a “recombinant chromosome 5”, i.e. a chromosome 3 or 5 which is not present in either of the parent plants and arose through a rare double crossing-over event between homologous chromosomes of a chromosome 3 or 5 pair. Herein, for example, recombinant cucumber chromosome 5 is provided comprising an introgression from a wild donor cucumber, which comprises a QTL that enhances internal fruit rot resistance and recombinant cucumber chromosome 3 is provided comprising an introgression from a wild donor cucumber, which comprises a QTL that enhances internal fruit rot resistance.

The term “traditional breeding techniques” encompasses herein crossing, backcrossing, selfing, selection, double haploid production, embryo rescue, protoplast fusion, marker assisted selection, mutation breeding etc., all as known to the breeder (i.e. methods other than genetic modification/transformation/transgenic methods), by which, for example, a recombinant chromosome 3 or 5 can be obtained, identified and/or transferred.

“Backcrossing” refers to a breeding method by which a (single) trait, such as a DB resistance QTL, can be transferred from a (generally inferior) genetic background (e.g. a wild cucumber, also referred to as “donor”) into a different (generally superior) genetic background (also referred to as “recurrent parent”), e.g. cultivated cucumber. An offspring of a cross (e.g. an F1 plant obtained by crossing a wild cucumber with a cultivated cucumber; or an F2 plant or F3 plant, etc., obtained from selfing the F1) is “backcrossed” to the parent with the different (generally superior) genetic background, e.g. to the cultivated parent. After repeated backcrossing, the trait of the first (generally inferior) genetic background will have been incorporated into the different (generally superior) genetic background.

“Marker assisted selection” or “MAS” is a process of using the presence of molecular markers, which are genetically and physically linked to a particular locus or to a particular chromosome region (e.g. introgression fragment), to select plants for the presence of the specific locus or region (introgression fragment). For example, a molecular marker genetically and physically linked to a DB fruit rot resistance QTL, can be used to detect and/or select cucumber plants comprising the DB fruit rot resistance QTL on chromosome 3 and/or 5. The closer the genetic linkage of the molecular marker to the locus (e.g. about 7 cM, 6 CM, 5 CM, 4 CM, 3 cM, 2 cM, 1 cM, 0.5 cM or less), the less likely it is that the marker is dissociated from the locus through meiotic recombination. Likewise, the closer two markers are linked to each other (e.g. within 7 cM or 5 cM, 4 cM, 3 cM, 2 cM, 1 cM or less) the less likely it is that the two markers will be separated from one another (and the more likely they will co-segregate as a unit).

A marker “within 7 cM or within 5 cM, 3 CM, 2 CM, or 1 cM” of another marker refers to a marker which genetically maps to within the 7 cM or 5 cM, 3 CM, 2 cM, or 1 cM region flanking the marker (i.e. either side of the marker). Similarly, a marker within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker).

“LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favor the presence of linkage and a LOD score greater than 3.0 is considered evidence for linkage. A LOD score of +3 indicates 1000 to 1 odds that the linkage being observed did not occur by chance.

“Vegetative propagation”, “vegetative reproduction” or “clonal propagation” are used interchangeably herein and mean the method of taking part of a plant and allowing that plant part to form at least roots where plant part is, e.g., defined as or derived from (e.g. by cutting of) leaf, pollen, embryo, cotyledon, hypocotyl, cells, protoplasts, meristematic cell, root, root tip, pistil, anther, flower, shoot tip, shoot, stem, fruit, petiole, etc. When a whole plant is regenerated by vegetative propagation, it is also referred to as a vegetative propagation.

In one aspect propagation by grafting, e.g. a scion onto a rootstock, is included herein.

“Cell culture” or “tissue culture” refers to the in vitro culture of cells or tissues of a plant.

“Regeneration” refers to the development of a plant from cell culture or tissue culture or vegetative propagation.

“Non-propagating cell” refers to a cell which cannot be regenerated into a whole plant.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

The term “nucleic acid sequence” (or nucleic acid molecule) refers to a DNA or RNA molecule in single or double stranded form, particularly a DNA encoding a protein or protein fragment according to the invention.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule.

The terms “protein” or “polypeptide” are used interchangeably and refer to molecules consisting of a chain of amino acids, without reference to a specific mode of action, size, 3-dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

“Sequence identity” and “sequence similarity” can be determined by alignment of two peptide (protein, amino acid) or two nucleotide sequences using global or local alignment algorithms. Sequences may then be referred to as “substantially identical” or “substantial identity” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm for aligning two sequences, over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS, accessible at world wide web under ebi.ac.uk/Tools/emboss/. Alternatively sequence similarity or identity may be determined by searching against databases (e.g. EMBL, GenBank) by using commonly known algorithms and output formats such as FASTA, BLAST, etc., but hits should be retrieved and aligned pairwise to compare sequence identity. Two proteins or two protein domains, or two nucleic acid sequences have “substantial sequence identity” if the percentage sequence identity is at least 80%, 90%, 91%, 92%, 93%, 94%, 95%, 96%, 97%, 98%, 99% or more (as determined by Emboss “needle” using default parameters, i.e. gap creation penalty=10, gap extension penalty=0.5, using scoring matrix DNAFULL for nucleic acids an Blosum62 for proteins).

When referring herein to a SNP nucleotide or SNP genotype at a specific nucleotide position, e.g. at nucleotide 51 of SEQ ID NO: 1, “or at nucleotide 51 of a sequence comprising at least 95%, 96%, 97%, 98% or 99% sequence identity to the SEQ ID NO” or “or at nucleotide 51 of a sequence which is at least 95%, 96% or 97% identical to the SEQ ID NO”, this means that the SNP nucleotide or SNP genotype is present in a variant sequence at a nucleotide corresponding to (or equivalent to) the same nucleotide (e.g. corresponding to/equivalent to nucleotide 51 of SEQ ID NO: 1) in the variant sequence, i.e. in a sequence comprising at least 95%, 96% or 97%, 98% or 99% sequence identity to the mentioned SEQ ID NO. Pairwise alignment if the two sequences can be used to identify the nucleotide corresponding to (or equivalent to) the indicated SNP.

When reference is made to a nucleic acid sequence (e.g. DNA or genomic DNA) having “substantial sequence identity to” a reference sequence or having a sequence identity of at least 80%, e.g. at least 85%, 90%, 95%, 96%, 97%, 98% or 99% nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100nt) are for example those which include at least one wash in 0.2×SSC at 63° C. for 20 min, or equivalent conditions. Stringent conditions for DNA-DNA hybridisation (Southern blots using a probe of e.g. 100nt) are for example those which include at least one wash (usually 2) in 0.2×SSC at a temperature of at least 50° C., usually about 55° C., for 20 min, or equivalent conditions.

“Fine-mapping” refers to methods by which the position of a QTL can be determined more accurately (narrowed down) and by which the size of the introgression fragment comprising the QTL is reduced. For example Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL. In this way sub-fragments of the introgression fragments for QTL3.1 or QTL5.1 can be identified which comprises the QTL, but which are shorter than the fragment found in the deposited seeds (NCIMB 43530), and which consequently lack one or more of the SNP markers of the introgression fragment, especially on either side of the fragment. Thus, for example a sub-fragment comprising QTL5.1 may comprise SNP_12 to SNP_15 (as shown in FIG. 6) and the QTL5.1 (conferring reduced susceptibility to internal fruit rot when in homozygous form). The same applies for QTL3.1, where a sub-fragment comprising QTL3.1 may comprise at least 3, 4, 5 or 6 consecutive markers selected from SNP_19 to SNP_42. In one aspect QTL3.1 has been fine mapped to be located between SNP_35 and 659 nt downstream of SNP_36. In one aspect QTL5.1 was fine mapped to be located in between SNP_12 and SNP_15.

“Targeted gene/genome modification” or “targeted gene/genome editing” refers to methods whereby a target gene, such as the causal gene (or candidate gene) underlying QTL5.1 or QTL3.1, is modified by e.g. CRISPR based methods (Crispr-Cas9 or Crispr-Cpf1, etc.), TALENs or other methods known in the art. Genes can be knocked-in (inserted) or edited by single nucleotide insertions or deletions or gene parts, such as promoters, can be replaced. See for example Chen et al. 2019, Ann Rev of Plant Biology 70:667-97.

“Candidate gene” or “causal gene” is the gene which is assumed to underly and to be causal of the trait conferred by the QTL5.1 or QTL3.1. The candidate gene can be found by fine-mapping and looking at the genes present in the narrowed-down region. The candidate gene underlying QTL5.1 or QTL3.1 can, once known, be modified by e.g. mutagenesis techniques (using e.g. chemical or radiation to induce mutations) or by targeted gene editing techniques and the plant comprising a mutant allele of the target gene (such as an allele having enhanced expression or reduced expression or no expression of the gene, or an allele encoding a reduced function or loss of function protein) can be analysed for its resistance to internal fruit rot. The causal genes underlying QTL3.1 have been identified as being eight genes encoding 2OGD proteins, referred herein to as OGD1 to OGD8, and/or a gene encoding a HR-like protein.

“TILLING” (Targeting Induced Local Lesions IN Genomes) refers to a method of selecting a plant comprising mutations in an endogenous gene, such as the causal gene underlying QTL5.1 or QTL3.1, as described by McCallum et al. (2000, Plant Physiology 123, 439-442). A ‘TILLING mutant’ refers to a plant which comprises a mutant allele of the target gene, leading to a change in gene expression or a change in function of the encoded protein.

“Expression of a gene” refers to the process wherein a DNA region, which is operably linked to appropriate regulatory regions, particularly a promoter, is transcribed into an RNA.

An “active protein” or “functional protein” is a protein which has protein activity as measurable in vivo, e.g. by the phenotype conferred by the protein. A “wild type” protein is a fully functional protein, as present in the wild type plant. A “mutant protein” is herein a protein comprising one or more mutations in the nucleic acid sequence encoding the protein, whereby the mutation(s) results in (the mutant allele encoding) a “reduced-function” or “loss-of-function” protein, as e.g. measurable in vivo, e.g. by the phenotype conferred by the mutant allele (e.g. in homozygous form).

A “mutation” in a nucleic acid molecule coding for a protein is a change of one or more nucleotides compared to the wild type sequence, e.g. by replacement, deletion or insertion of one or more nucleotides. Examples of such a mutation are point mutation, nonsense mutation, missense mutation, splice-site mutation, frame shift mutation or a mutation in a regulatory sequence, such as a promoter sequence.

A “point mutation” is the replacement of a single nucleotide, or the insertion or deletion of a single nucleotide.

A “nonsense” mutation is a (point) mutation in a nucleic acid sequence encoding a protein, whereby a codon is changed into a stop codon. This results in a premature stop codon being present in the mRNA and in a truncated protein. A truncated protein may have reduced function or loss of function.

A “missense” or non-synonymous mutation is a (point) mutation in a nucleic acid sequence encoding a protein, whereby a codon is changed to code for a different amino acid. The resulting protein may have reduced function or loss of function.

A “splice-site” mutation is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have reduced function or loss of function.

A “frame-shift” mutation is a mutation in a nucleic acid sequence encoding a protein by which the reading frame of the mRNA is changed, resulting in a different amino acid sequence. The resulting protein may have reduced function or loss of function.

A mutation in a regulatory sequence, e.g. in a promoter of a gene or in an enhancer sequence of a gene (referred to as cis-regulatory sequences), is a change of one or more nucleotides compared to the wild type sequence, e.g. by replacement, deletion or insertion of one or more nucleotides, leading for example to reduced or no mRNA transcript of the gene being made (reduced gene expression) or to more mRNA transcript being made (increased gene expression). In one aspect a mutation in a regulatory sequence of a protein includes a higher level of wild type protein (e.g. due to a higher expression of the allele) being made. Rodriguez-Leal et al., 2017, Cell 171, 470-480 describe for example mutating cis-regulatory elements to create a continuum of mutant alleles with different expression.

“cis regulatory elements” are elements that are part of the gene and regulate gene expression, such as the promoter or enhancer elements. In contrast “trans-regulatory elements” are elements that interact with the gene, such as a transcription factor, noncoding RNA or signaling molecules. Mutations in cis regulatory elements and in trans-regulatory elements can enhance gene expression or reduce gene expression.

A “mutation” in a protein is a change of one or more amino acid residues compared to the wild type sequence, e.g. by replacement, deletion or insertion of one or more amino acid residues.

“Silencing” refers to a down-regulation or complete inhibition of gene expression of the target gene or gene family.

“Enhanced expression” refers herein to an increase of expression of a gene in a plant or plant part which is higher than the normal expression of the gene, which can be achieved by various means, such as expression from a different promoter (such as a constitutive promoter), increased expression due to gene copy number increase (dosage), increased expression from a modified cis-regulatory element such as a modified promoter or modified enhancer element, increased expression due to a modified trans-regulatory element.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence.

“Wild type OGD1 allele” (WT) refers herein to a version of a gene encoding a fully functional cucumber OGD1 protein (wild type OGD1 protein). Such a sequence encoding a fully functional OGD1 protein of SEQ ID NO: 52 is for example the wild type OGD1 cDNA (mRNA) sequence depicted in SEQ ID NO: 61. The protein sequence encoded by this wild type OGD1 mRNA is depicted in SEQ ID NO: 52. It consists of 371 amino acids. Other fully functional OGD1 protein-encoding alleles (i.e. variant alleles, or allelic variants) may exist in other cucumber plants or wild cucumber or wild relatives of cucumber and may comprise substantial sequence identity with SEQ ID NO: 52, i.e. at least about 90%, 91%, 93%, 94%, 95%, 96%, 97%, 98% or 99% sequence identity with SEQ ID NO: 52. Such fully functional wild type OGD1 proteins are herein referred to as “variants” of SEQ ID NO: 52.

“Wild type OGD2 allele” (WT) refers herein to a version of a gene encoding a fully functional cucumber OGD2 protein (wild type OGD2 protein). Such a sequence encoding a fully functional OGD2 protein of SEQ ID NO: 53 is for example the wild type OGD2 cDNA (mRNA) sequence depicted in SEQ ID NO: 62. The protein sequence encoded by this wild type OGD2 mRNA is depicted in SEQ ID NO: 53. It consists of 412 amino acids. Other fully functional OGD2 protein-encoding alleles (i.e. variant alleles, or allelic variants) may exist in other cucumber plants or wild cucumber or wild relatives of cucumber and may comprise substantial sequence identity with SEQ ID NO: 53, i.e. at least about 90%, 91%, 93%, 94%, 95%, 96%, 97%, 98% or 99% sequence identity with SEQ ID NO: 53. Such fully functional wild type OGD2 proteins are herein referred to as “variants” of SEQ ID NO: 53.

“Wild type OGD3 allele” (WT) refers herein to a version of a gene encoding a fully functional cucumber OGD3 protein (wild type OGD3 protein). Such a sequence encoding a fully functional OGD3 protein of SEQ ID NO: 54 is for example the wild type OGD3 cDNA (mRNA) sequence depicted in SEQ ID NO: 63. The protein sequence encoded by this wild type OGD3 mRNA is depicted in SEQ ID NO: 54. It consists of 368 amino acids. Other fully functional OGD3 protein-encoding alleles (i.e. variant alleles, or allelic variants) may exist in other cucumber plants or wild cucumber or wild relatives of cucumber and may comprise substantial sequence identity with SEQ ID NO: 54, i.e. at least about 90%, 91%, 93%, 94%, 95%, 96%, 97%, 98% or 99% sequence identity with SEQ ID NO: 54. Such fully functional wild type OGD3 proteins are herein referred to as “variants” of SEQ ID NO: 54.

“Wild type OGD4 allele” (WT) refers herein to a version of a gene encoding a fully functional cucumber OGD4 protein (wild type OGD4 protein). Such a sequence encoding a fully functional OGD4 protein of SEQ ID NO: 55 is for example the wild type OGD4 cDNA (mRNA) sequence depicted in SEQ ID NO: 64. The protein sequence encoded by this wild type OGD4 mRNA is depicted in SEQ ID NO: 55. It consists of 386 amino acids. Other fully functional OGD4 protein-encoding alleles (i.e. variant alleles, or allelic variants) may exist in other cucumber plants or wild cucumber or wild relatives of cucumber and may comprise substantial sequence identity with SEQ ID NO: 55, i.e. at least about 90%, 91%, 93%, 94%, 95%, 96%, 97%, 98% or 99% sequence identity with SEQ ID NO: 55. Such fully functional wild type OGD4 proteins are herein referred to as “variants” of SEQ ID NO: 55.

“Wild type OGD5 allele” (WT) refers herein to a version of a gene encoding a fully functional cucumber OGD5 protein (wild type OGD5 protein). Such a sequence encoding a fully functional OGD5 protein of SEQ ID NO: 56 is for example the wild type OGD5 cDNA (mRNA) sequence depicted in SEQ ID NO: 65. The protein sequence encoded by this wild type OGD5 mRNA is depicted in SEQ ID NO: 56. It consists of 374 amino acids. Other fully functional OGD5 protein-encoding alleles (i.e. variant alleles, or allelic variants) may exist in other cucumber plants or wild cucumber or wild relatives of cucumber and may comprise substantial sequence identity with SEQ ID NO: 56, i.e. at least about 90%, 91%, 93%, 94%, 95%, 96%, 97%, 98% or 99% sequence identity with SEQ ID NO: 56. Such fully functional wild type OGD5 proteins are herein referred to as “variants” of SEQ ID NO: 56.

“Wild type OGD6 allele” (WT) refers herein to a version of a gene encoding a fully functional cucumber OGD6 protein (wild type OGD6 protein). Such a sequence encoding a fully functional OGD6 protein of SEQ ID NO: 57 is for example the wild type OGD6 cDNA (mRNA) sequence depicted in SEQ ID NO: 66. The protein sequence encoded by this wild type OGD6 mRNA is depicted in SEQ ID NO: 57. It consists of 393 amino acids. Other fully functional OGD6 protein-encoding alleles (i.e. variant alleles, or allelic variants) may exist in other cucumber plants or wild cucumber or wild relatives of cucumber and may comprise substantial sequence identity with SEQ ID NO: 57, i.e. at least about 90%, 91%, 93%, 94%, 95%, 96%, 97%, 98% or 99% sequence identity with SEQ ID NO: 57. Such fully functional wild type OGD6 proteins are herein referred to as “variants” of SEQ ID NO: 57.

“Wild type OGD7 allele” (WT) refers herein to a version of a gene encoding a fully functional cucumber OGD7 protein (wild type OGD7 protein). Such a sequence encoding a fully functional OGD7 protein of SEQ ID NO: 58 is for example the wild type OGD7 cDNA (mRNA) sequence depicted in SEQ ID NO: 67. The protein sequence encoded by this wild type OGD7 mRNA is depicted in SEQ ID NO: 58. It consists of 381 amino acids. Other fully functional OGD7 protein-encoding alleles (i.e. variant alleles, or allelic variants) may exist in other cucumber plants or wild cucumber or wild relatives of cucumber and may comprise substantial sequence identity with SEQ ID NO: 58, i.e. at least about 90%, 91%, 93%, 94%, 95%, 96%, 97%, 98% or 99% sequence identity with SEQ ID NO: 58. Such fully functional wild type OGD7 proteins are herein referred to as “variants” of SEQ ID NO: 58.

“Wild type OGD8 allele” (WT) refers herein to a version of a gene encoding a fully functional cucumber OGD8 protein (wild type OGD8 protein). Such a sequence encoding a fully functional OGD8 protein of SEQ ID NO: 59 is for example the wild type OGD8 cDNA (mRNA) sequence depicted in SEQ ID NO: 68. The protein sequence encoded by this wild type OGD8 mRNA is depicted in SEQ ID NO: 59. It consists of 374 amino acids. Other fully functional OGD8 protein-encoding alleles (i.e. variant alleles, or allelic variants) may exist in other cucumber plants or wild cucumber or wild relatives of cucumber and may comprise substantial sequence identity with SEQ ID NO: 59, i.e. at least about 90%, 91%, 93%, 94%, 95%, 96%, 97%, 98% or 99% sequence identity with SEQ ID NO: 59. Such fully functional wild type OGD8 proteins are herein referred to as “variants” of SEQ ID NO: 59.

“Wild type HR-like allele” (WT) refers herein to a version of a gene encoding a fully functional cucumber HR-like protein (wild type HR-like protein). Such a sequence encoding a fully functional HR-like protein of SEQ ID NO: 60 is for example the wild type HR-like cDNA (mRNA) sequence depicted in SEQ ID NO: 69. The protein sequence encoded by this wild type HR-like mRNA is depicted in SEQ ID NO: 60. It consists of 138 amino acids. Other fully functional HR-like protein-encoding alleles (i.e. variant alleles, or allelic variants) may exist in other cucumber plants or wild cucumber or wild relatives of cucumber and may comprise substantial sequence identity with SEQ ID NO: 60, i.e. at least about 90%, 91%, 93%, 94%, 95%, 96%, 97%, 98% or 99% sequence identity with SEQ ID NO: 60. Such fully functional wild type HR-like proteins are herein referred to as “variants” of SEQ ID NO: 60.

### DETAILED DESCRIPTION

Using a special disease assay to identify QTLs affecting fruit infection rather than leaf or stem infection, two QTLs (referred to as QTL3.1 and QTL5.1), which originated from a wild donor, were found to confer an increased resistance/reduced susceptibility against Didymella bryoniae fruit infection compared to a (control) cucumber plant lacking the QTLs (e.g. the recurrent parent or genetic control). QTL5.1 was initially mapped to be linked to the donor SNP markers SNP_01 to SNP_18 and was later fine-mapped to be located in-between SNP_12 and SNP_15, with additional SNP markers mapped to the region, as shown in FIG. 6. QTL3.1 was initially mapped to be linked to the donor SNP markers for SNP_19 to SNP_42 and was later fine mapped to be located in the region between SNP_35 and 659 nucleotides downstream of SNP_36. Sequencing of this region revealed that there was actually a duplication in the resistant plant, and that the fine-mapped QTL3.1 was duplicated as part of that duplication on chromosome 3. In the fine-mapped QTL3.1 region (between SNP_35 and 659 nt downstream of SNP_36) a cluster of nine genes were found, eight of which encode 2OGD proteins (2-oxoglutarate and Fe(II) dependent oxygenase proteins) and one encodes a HR-like protein.

Expression analysis showed that all nine genes had a significantly increased expression in the resistant plant (homozygous for the duplication of QTL3.1 and therefore comprising four copies of the gene cluster) compared to the susceptible plant (lacking the duplication and thus only containing two copies of QTL3.1, one on each chromosome and therefore containing two copies of the gene cluster). Gene expression of the cluster was significantly increased in the ovary tissue of the resistant plant, which is the cucumber fruit tissue, and the higher enzymatic activity is therefore thought to prevent DB infection of the fruit. Especially the eight 2OGD genes are thought to have a significant effect on preventing DB infection of the fruit, as their average expression was twice as high in the ovary tissue of the resistant cucumbers.

As there were no other genes in the QTL3.1 region and as the genes are implicated in plant defense, it was concluded that this higher expression (due to higher copy number) was indeed causal of the resistance (or reduced susceptibility) against fruit infection via the flowers. In tomato gene dosage (i.e. copy number) has been shown to be implicated in dose-dependent phenotypes such as fruit size and Crispr-Cas9 genome editing was used to modify dosage number and to generate plants with different gene dosages (Alonge et al., 2020, Cell 182, 1-17).

Having identified the causal genes underlying QTL3.1 enables modification of their expression in cucumber. Increasing the expression of one or more or all of the nine genes enables generating plants having reduced susceptibility against Didymella bryoniae fruit infection. Ways to increase expression of one or more or all of the eight 2OGD genes and/or the gene encoding the HR-like protein are for example increasing gene dosage in the plant (for example through gene or gene cluster duplication), overexpressing the genes (e.g. generating transgenic plants), modifying the cis-regulatory elements or the trans-regulatory elements of the genes. Ways to insert or replace genes or modify genes or parts thereof, such as promoter replacement are described in e.g. Chen et al. 2019, Ann Rev of Plant Biology 70:667-97.

As the highest resistance against Didymella bryoniae fruit infection was seen in plants comprising the duplication of QTL3.1 and QTL5.1 together, one aspect herein is to combine the increased expression of one or more of the nine genes of QTL3.1 with QTL5.1.

### Didymella Fruit Rot Resistance Through Modification of Gene Expression

In one aspect a Cucumis sativus var. sativus plant is provided of which the genome is modified in such a way that the gene expression of one or more genes encoding a 2-oxoglutarate and Fe(II) dependent oxygenase protein (2OGD protein) and/or a HR-like protein is higher than in the plant comprising an unmodified genome, such as a wild type plant, wherein the cucumber genes encoding a cucumber 2-oxoglutarate and Fe(II) dependent oxygenase protein and a HR-like protein are the genes encoding
an OGD1 protein comprises the amino acid sequence of SEQ ID NO: 52 or a sequence comprising at least 95% sequence identity to SEQ ID NO: 52,
an OGD2 protein comprises the amino acid sequence of SEQ ID NO: 53 or a sequence comprising at least 95% sequence identity to SEQ ID NO: 53,
an OGD3 protein comprises the amino acid sequence of SEQ ID NO: 54 or a sequence comprising at least 95% sequence identity to SEQ ID NO: 54,
an OGD4 protein comprises the amino acid sequence of SEQ ID NO:55 or a sequence comprising at least 95% sequence identity to SEQ ID NO: 55,
an OGD5 protein comprises the amino acid sequence of SEQ ID NO: 56 or a sequence comprising at least 95% sequence identity to SEQ ID NO: 56,
an OGD6 protein comprises the amino acid sequence of SEQ ID NO: 57 or a sequence comprising at least 95% sequence identity to SEQ ID NO: 57,
an OGD7 protein comprises the amino acid sequence of SEQ ID NO: 58 or a sequence comprising at least 95% sequence identity to SEQ ID NO: 58,
an OGD8 protein comprises the amino acid sequence of SEQ ID NO: 59 or a sequence comprising at least 95% sequence identity to SEQ ID NO: 59,
a HR-like protein comprises the amino acid sequence of SEQ ID NO: 60 or a sequence comprising at least 95% sequence identity to SEQ ID NO: 60.

In one aspect a Cucumis sativus var. sativus plant is provided comprising an increased resistance against internal fruit rot symptoms caused by Didymella bryoniae, wherein the plant genome is modified to increase the gene expression of one or more or all genes encoding a 2-oxoglutarate Fe(II)-dependent oxygenase (OGD) protein selected from
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and/or wherein the plant genome is modified to increase the gene expression of a gene encoding a HR-like protein comprising the amino acid sequence of SEQ ID NO: 60 or a sequence comprising at least 95% sequence identity to SEQ ID NO: 60.

As the 2OGD proteins are enzymes which have the same function in vivo, an increase in expression of any of the eight 2OGD proteins in the plant increases the resistance against DB fruit infection. The wild type susceptible plant for example lacks the duplication of QTL3.1 which was found in the resistant plant. The susceptible plant thus contained 2 copies of each of the 9 genes, meaning it contained 16 copies of 2OGD genes being expressed. The resistant plant comprising a duplication of QTL3.1 on chromosome 3 contained either 3 copies of the 9 genes (heterozygous) or 4 copies of the 9 genes (homozygous), meaning it contained 24 copies or 32 copies of 2OGD genes (and 3 or 4 copies of the gene encoding the HR-like protein). Therefore, any expression of a 2OGD gene which results in more 2OGD protein being made will increase the resistance level.

In one aspect a Cucumis sativus var. sativus plant is provided comprising an increased resistance against internal fruit rot symptoms caused by Didymella bryoniae, wherein the plant genome is modified to increase the average gene expression of eight OGD genes, OGD1 to OGD8, encoding a 2-oxoglutarate Fe(II)-dependent oxygenase (OGD) proteins, to be at least 1.2 times, 1.3 times, preferably at least 1.4 times, 1.5 times, 1.6 times, 1.7 times, 1.8 times, 1.9 times, 2.0 times, 2.1 times, 2.2 times, 2.3 times, 2.4 times, 2.5 times that of the average gene expression of the eight OGD genes in a wild type, susceptible plant comprising two copies of each OGD gene, wherein the eight OGD genes are the genes encoding an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and/or wherein the plant genome is modified to increase the average gene expression of a gene encoding a HR-like protein to be at least 1.2 times that of the average gene expression of said gene in a wild type, susceptible plant comprising two copies of the gene, wherein said gene is a gene encoding a protein comprising the amino acid sequence of SEQ ID NO: 60 or a sequence comprising at least 95% sequence identity to SEQ ID NO: 60.

Thus the plant genome may be modified to increase the gene expression of a gene encoding a HR-like protein comprising the amino acid sequence of SEQ ID NO: 60 or a sequence comprising at least 95% sequence identity to SEQ ID NO: 60. The average expression of this gene may also be increased to be at least 1.2 times, preferably at least 1.3 times, 1.4 times, 1.5 times, 1.6 times, 1.7 times, 1.8 times, 1.9 times, 2.0 times, 2.1 times, 2.2 times, 2.3 times, 2.4 times, 2.5 times that of the average gene expression of the HR-like gene in a wild type, susceptible plant comprising two copies of the HR-like gene.

In one aspect the plant can be modified to increase the gene expression by a modification of a cis-regulatory element of one or more or all of the 2OGD genes and/or the gene encoding the HR-like protein. Thus, for example the promoter can be modified to that the gene expression is higher than for the unmodified promoter. The promoter sequences are found upstream (5′) of the coding sequence and are present on the genomic QTL3.1 sequences SEQ ID NO: 72 (for the promoter of the HR-like gene) and SEQ ID NO: 73 (for the promoters of the 2OGD genes), and also the 1000 bases upstream of the translation start codon (ATG codon) are provided herein in SEQ ID NO: 74 to 82, which comprise the promoter sequence. The promoter sequences can thus be modified to enhance transcription of the 2OGD and/or HR-like gene. Modifications can be introduced by e.g. CRISPR based methods or mutagenesis, see e.g. Rodriguez-Leal et al., 2017 (supra) and Chen et al. (supra).

Or enhancer elements can be modified or introduced to enhance gene expression. Mutations in a cis-regulatory element can enhance gene expression, leading to increased mRNA levels (or corresponding cDNA levels) and increased protein levels. Mutations can be generated by known methods, such as treatment with a mutagenic chemical or radiation, or by targeted genome modification. Preferably the gene expression of one or more of the 2OGD genes and/or the gene encoding the HR-like protein is enhanced compared to the level of the wild type (unmodified) genes and/or compared to the level found in the susceptible plant lacking the duplication of QTL3.1.

In another aspect the plant can be modified to increase the gene expression by a modification of a trans-regulatory element of one or more or all of the 2OGD genes and/or the gene encoding the HR-like protein.

In another aspect the plant can be modified to increase the gene expression by an increased dosage (copy number) of one or more or all of the 2OGD genes and/or the gene encoding the HR-like protein in the plant, or by the introduction of a transgene expressing one or more one or more or all of the 2OGD genes and/or the gene encoding the HR-like protein. An increased copy number is thus a copy number which is higher than in the wild type plant, which comprises 1 copy on each chromosome 3 (i.e. 1 copy in a haploid genome, two copies in the diploid genome). The copy number of one or more or all of the 2OGD genes and/or the HR-like gene may thus be increased to 2, 3, 4 or more copies in the haploid genome, or 4, 5, 6, 7 or 8 in the diploid genome. As mentioned, the duplication of QTL3.1 (Seq ID NO: 72) on chromosome 3 is just one example whereby the copy number of the haploid genome is increased from 1 copy to 2 copies of OGD1 to OGD8 and the HR-like gene (and 4 copies in the homozygous diploid genome). One can also generate other duplications, e.g. whereby only the 2OGD genes are duplicated in the haploid genome, e.g. on chromosome 3.

So, for example targeted genome editing methods, such as Crispr-Cas9 or Cpf1 systems may be used to duplicate (or triplicate or quadruple, etc.) one or more or all of the 2OGD genes and/or the gene encoding the HR-like protein in the genome, e.g. insertion of extra copies on chromosome 3 of the genome, see e.g. Chen et al. (supra). In one aspect even the entire cluster of genes encoding OGD1 to OGD8 (optionally also the HR-like protein) may be e.g. duplicated. A duplication of one or more of the genes or the gene cluster may involve a translocation or a (uneven) recombination event leading to a duplication. Also CRISPR based methods or GMO methods can be used to increase gene dosage, i.e. increase copy number of one or more or all of the genes. A cucumber plant comprising a duplication of one or more or all of the genes on e.g. chromosome 3 can be identified by for example sequencing or identifying a plant comprising increased transcript levels or protein levels for one or more or all of the genes. Also digital PCR can be used to determine whether a plant or plant part comprises a higher copy number than the wild type plant.

Another way of increasing the expression is by transforming the plant to generate a genetically modified plant expressing one or more of the genes, e.g. under the control of a constitutive promoter (e.g. the 35S promoter) or an inducible promoter, or its own promoter. This can be done by making a vector comprising the coding region operably linked to a promoter region and transforming the plant, e.g. by Agrobacterium mediated transformation using methods known in the art. The chimeric gene is integrated into the plant genome and a transgenic plant expressing the transgenes can be obtained. Such a plant can then be tested for it's resistance to DB fruit infection as described herein. The percentage of infected fruits should be significantly lower than in the control plant (e.g. the non-transgenic, wild type plant).

The increase in gene expression of the genes encoding one or more or all of the 2OGD proteins and/or the HR-like protein is an increase relative to the gene expression of the wild type, non-modified gene, found in the susceptible plant. So in one aspect the gene expression of any one of a gene encoding OGD1, OGD2, OGD3, OGD4, OGD5, OGD6, OGD7, OGD8 of SEQ ID NO: 52 to 59 (or proteins comprising at least 95% sequence identity to any of these), or the average gene expression of genes encoding OGD1 to OGD8 of SEQ ID NO: 52 to 59 (or proteins comprising at least 95% sequence identity to any of these), and/or the gene expression of the gene encoding the HR-like protein (or a protein comprising at least 95% sequence identity to any of these) is higher than the wild type gene expression of e.g. the gene (or genes) found in a cucumber plant lacking a duplication of QTL3.1, preferably at least 1.2 times or 1.3 times the expression level, more preferably at least 1.4 times, 1.5 times, 1.6 times, 1.7 times, 1.8 times, 1.9 times, 2.0 times, 2.1 times, 2.2 times, 2.3 times, 2.4 times, 2.5 times, 2.6 times, 2.7 times, 2.8 times, 2.9 times, 3.0 times, 3.5 times, 4.0 times or more.

The wild type gene expression or the endogenous expression in the wild type, susceptible cucumber plant can be measured as e.g. described in the examples. The susceptible control plant is a plant comprising one copy of QTL3.1 on each chromosome 3, i.e. it comprises two copies of each of the OGD genes and two copies of the gene encoding the HR like protein.

In one aspect the plant comprises an average increased expression of genes encoding 2OGD proteins (i.e. encoding OGD1 to OGD8 of SEQ ID NO: 52 to 59 or proteins comprising at least 95% identity to any of these) which is at least 1.2 times or 1.3 times the expression level of the average level of genes encoding 2OGD proteins found in the wild type plant/susceptible control plant, more preferably at least 1.4 times, 1.5 times, 1.6 times, 1.7 times, 1.8 times, 1.9 times, 2.0 times, 2.1 times, 2.2 times, 2.3 times, 2.4 times, 2.5 times, 2.6 times, 2.7 times, 2.8 times, 2.9 times, 3.0 times, 3.5 times, 4.0 times or more.

In another aspect the plant comprises an increased expression of one or more genes selected from OGD1 to OGD8 (or genes encoding proteins comprising at least 95% identity to any of these) which is at least 1.2 times or 1.3 times the expression level of the gene found in the wild type plant/susceptible control plant, more preferably at least 1.4 times, 1.5 times, 1.6 times, 1.7 times, 1.8 times, 1.9 times, 2.0 times, 2.1 times, 2.2 times, 2.3 times, 2.4 times, 2.5 times, 2.6 times, 2.7 times, 2.8 times, 2.9 times, 3.0 times, 3.5 times, 4.0 times or more.

In one aspect the plant comprises an increased expression of the gene encoding the HR-like protein of SEQ ID NO: 60, or encoding a HR-like protein comprising at least 95% sequence identity to SEQ ID NO: 60, which is at least 1.2 times or 1.3 times the expression level of the gene found in the wild type plant/susceptible control plant, more preferably at least 1.4 times, 1.5 times, 1.6 times, 1.7 times, 1.8 times, 1.9 times, 2.0 times, 2.1 times, 2.2 times, 2.3 times, 2.4 times, 2.5 times, 2.6 times, 2.7 times, 2.8 times, 2.9 times, 3.0 times, 3.5 times, 4.0 times or more.

The increase in the expression can, for example, be determined by measuring the quantity of RNA transcripts (or cDNA) encoding one or more or all of the 2OGD proteins or the HR-like protein, e.g. using Northern blot analysis or RT-PCR. See also the Examples. Alternatively, the amount of protein can be measured.

So in one aspect the amount of protein of one or more of OGD1, OGD2, OGD3, OGD4, OGD5, OGD6, OGD7, OGD8 or HR-like protein (or a protein comprising at least 95% sequence identity to any of these) is higher than the amount of protein in the plant comprising the wild type gene expression of e.g. the gene (or genes) found in a cucumber plant lacking a duplication of QTL3.1, preferably at least 1.2 times or 1.3 times the protein level, more preferably at least 1.4 times, 1.5 times, 1.6 times, 1.7 times, 1.8 times, 1.9 times, 2.0 times, 2.1 times, 2.2 times, 2.3 times, 2.4 times, 2.5 times, 2.6 times, 2.7 times, 2.8 times, 2.9 times, 3.0 times, 3.5 times, 4.0 times or more.

In one aspect the plant comprises an average increased in 2OGD proteins (OGD1 to OGD8 or proteins comprising at least 95% identity to any of these) which is at least 1.2 times or 1.3 times the amount of 2OGD proteins found in the wild type plant/susceptible control plant, more preferably at least 1.4 times, 1.5 times, 1.6 times, 1.7 times, 1.8 times, 1.9 times, 2.0 times, 2.1 times, 2.2 times, 2.3 times, 2.4 times, 2.5 times, 2.6 times, 2.7 times, 2.8 times, 2.9 times, 3.0 times, 3.5 times, 4.0 times or more.

The increase in the amount of a 2OGD protein and/or HR-like protein can, for example, be determined by immunological methods such as Western blot analysis, ELISA (Enzyme Linked Immuno Sorbent Assay) or RIA (Radio Immune Assay).

The increase in gene expression or protein amounts is in one aspect in the ovary tissue of the fruits and/or in the leaf tissue of the plant, but may also be in other tissues.

In one aspect a cucumber plant is provided which comprises in its diploid genome at least at least three expressed copies of the gene encoding the HR-like protein, optionally at least 4 expressed copies, and/or at least 24 expressed copies of a gene encoding a 2-oxoglutarate Fe(II) dependent oxygenase protein (optionally at least 32 expressed copies), wherein the gene encoding a HR-like protein is a gene encoding the protein of SEQ ID NO: 60 or a sequence comprising at least 95% sequence identity to SEQ ID NO: 60 and wherein a gene encoding a 2-oxoglutarate Fe(II) dependent oxygenase protein is selected from the group of genes encoding
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59.

In one aspect the (at least) 24 genes encoding a 2-oxoglutarate Fe(II) dependent oxygenase protein are three copies of each gene of the group of genes encoding (or the, at least, 36 genes are four copies of each gene):
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59.

In one aspect a cucumber plant is provided which comprises in its diploid genome at least at least four expressed copies of the gene encoding the HR-like protein and/or at least 36 expressed copies of a gene encoding a 2-oxoglutarate Fe(II) dependent oxygenase protein, wherein the gene encoding a HR-like protein is a gene encoding the protein of SEQ ID NO: 60 or a sequence comprising at least 95% sequence identity to SEQ ID NO: 60 and wherein a gene encoding a 2-oxoglutarate Fe(II) dependent oxygenase protein is selected from the group of genes encoding:
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59.

In one aspect the (at least) 36 genes encoding a 2-oxoglutarate Fe(II) dependent oxygenase protein are four copies of each gene of the group of genes encoding:
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59.

In one embodiment the duplicated copies of the HR-like gene and/or the one or more genes selected from the genes encoding the proteins OGD1 to OGD8 of SEQ ID NO: 52 to 59 (or proteins comprising at least 95% sequence identity to any of these) are located on chromosome 3. In another aspect they are located on another chromosome, selected from chromosome 1, 2, 4, 5, 6 and 7. So, chromosome 3 may for example be a wild type chromosome 3, while e.g. chromosome 5 has been modified to comprise of one or more of the QTL3.1 genes, preferably in homozygous form. For example, the entire QTL3.1 region with all nine genes could be duplicated on chromosome 5.

In one aspect the plant comprises a gene expression of one or more or all of the genes encoding a 2-oxoglutarate Fe(II) dependent oxygenase protein selected from OGD1 to OGD8 (or an average expression of OGD1 to OGD8) and/or of the gene encoding the HR-like protein whereby the expression is at least 1.2 times or at least 1.3 times the expression in the wild type plant.

In one aspect the plant comprises a duplication in its haploid genome of one or more or all genes encoding a 2-oxoglutarate Fe(II) dependent oxygenase (OGD) protein selected from
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59, and/or comprises a duplication in its genome of a gene encoding a HR-like protein comprising the amino acid sequence of SEQ ID NO: 60 or a sequence comprising at least 95% sequence identity to SEQ ID NO: 60.

In one aspect the duplication is on chromosome 3 of the cucumber genome.

In one aspect the plant or plant part comprises a duplication in its haploid genome of SEQ ID NO: 72 (and the complementary strand SEQ ID NO: 73) or of a sequence comprising at least 95%, 96%, 97%, 98% or 99% sequence identity to SEQ ID NO: 72 (or its complement). In one aspect the duplication is on chromosome 3.

In a further aspect the plant or plant part comprises a duplication in its haploid genome of the region of chromosome 3 starting at (and preferably including) SEQ ID NO: 83 and ending at (and preferably including) SEQ ID NO: 84. In one aspect the duplication is on chromosome 3. This region comprises SEQ ID NO: 72 (and the complementary strand SEQ ID NO: 73) or a sequence comprising at least 95%, 96%, 97%, 98% or 99% sequence identity to SEQ ID NO: 72 (or its complement). The sequence of the region between SEQ ID NO: 83 and 84 is e.g. the chromosome 3 region as found in the reference cucumber genome found on cucurbitgenomics.org such as the Chinese Long V2 genome or a genomic sequence which is at least 96%, 97%, 98%, 99% identical to that sequence (when aligned pairwise using e.g. Needle).

As mentioned, the wild type plant is for example a cucumber plant whose genome has not been modified, such as a Cucumis sativus var. sativus line or variety, comprising on chromosome 3 a single copy of the wild type genes encoding the proteins OGD1 to OGD8 and HR-like protein, i.e. the haploid genome comprises a single copy of OGD1 to OGD8 and the gene encoding the HR like protein.

The plant comprising a modified genome as described above will have an increased resistance (reduced susceptibility) against internal fruit rot infection caused by Didymella bryoniae. This can be measured in a disease assay as described.

In one aspect the plant comprises at least four expressed copies of any one of the gene in its diploid genome, preferably at least four expressed copies of the gene encoding the HR-like protein and/or at least four expressed copies of a gene encoding an OGD protein selected from OGD1 to OGD8 and/or at least four expressed copies of two, three, four, five, six, seven or all eight of the genes encoding an OGD protein selected from OGD1 to OGD8, or at least four expressed copies of all nine genes. Four expressed copies of all nine genes may for example be due to a duplication of the QTL3.1 region starting at SNP_35 and ending at (659nt downstream of) SNP_36 on chromosome 3, and wherein the plant is homozygous for the chromosome 3 comprising the duplication. However, also genome editing can be used to duplicate on or more of the genes.

### QTLs

In one aspect the present invention relates to a cultivated Cucumis sativus var. sativus plant comprising one or two QTLs, on chromosome 3 and/or chromosome 5, introgressed from a wild cucumber, which confer an increased resistance/reduced susceptibility against Didymella bryoniae fruit infection compared to a (control) cucumber plant lacking the QTLs (e.g. the recurrent parent or genetic control). Thus, the increased DB fruit rot resistance is conferred by an introgression fragment on cultivated cucumber chromosome 5 (comprising QTL5.1 or a variant thereof) and/or on chromosome 3 (comprising a duplication of QTL3.1 or a variant thereof), wherein said introgression fragment is from a wild cucumber. In one aspect the introgression fragment is from a donor called MYCR3, but it can also be from a different donor (e.g. a newly identified donor comprising the same SNP haplotype as MYCR3 for at least 3, 4, 5, 6, 7, 8, 9, 10 or more of the SNP markers linked to the QTL), as described elsewhere herein, especially for one or more of SNP_29, SNP_30, SNP_31, SNP_32, SNP_33, SNP_34, SNP_35 and/or SNP_36 (present on the duplicated region), or SNP_35 and/or SNP_36 regarding QTL3.1 and one or more of the markers SNP_12 to SNP_15 (as shown in FIG. 6) for QTL5.1. Seeds comprising an introgression fragment from MYCR3 comprising QTL5.1 and comprising markers SNP_01 to SNP_18, especially comprising the SNP markers of FIG. 6 to identify the introgression fragment comprising the QTL5.1 have been deposited. The seeds also contain an introgression fragment from MYCR3 comprising QTL3.1, which is now known to be a duplication of QTL3.1 and comprising markers e.g. SNP_19 to SNP_42 to identify the introgression fragment comprising the QTL3.1 which is now known to lie in between SNP_35 and 659 nt down stream of SNP_36 and the causal genes in this region have been identified. The region of SEQ ID NO: 72 (i.e. the region comprising the donor SNPs for SNP_29 to SNP_36) is duplicated on chromosome 3 of the deposited seeds, and as the seeds are homozygous they contain 4 copies of the region, two on each chromosome 3.

It is noted that QTL5.1 and (the duplication of) QTL3.1 enhance internal fruit rot resistance on their own, but they are preferably combined in a single plant, as there is an additive effect of the QTLs. Stacking both QTLs is thus an advantage, as together they generally result in a much higher DB fruit rot resistance level than individually. Depending on the genetic background, (the duplication of) QTL3.1 on its own can have a small effect, reducing base-line susceptibility by only a few percent (e.g. 3%, 4% or 5%) or it can have a much larger effect, reducing base-line susceptibility by at least 10%, 15%, 20% or more. Similarly, QTL5.1 on its own may reduce base-line susceptibility by e.g. 5%, 10%, 15%, 20% or more. The combined effect on the other hand is generally much larger, reducing base-line susceptibility by at least 15%, 20%, 25%, 30%, 40% or more. In some cucumber plants, as shown in the Examples for long cucumber lines, the percentage of fruits showing internal fruit rot infection symptoms can be reduced to 1.5% or less, such as equal to or less than 1% or even 0%, when both QTLs are present in homozygous form. Such high resistance to internal fruit infections can mean that no fungicide treatments, or a significantly reduced treatment, might be needed when cultivating such plants. This is advantageous to the grower and also to the consumer.

### QTL5.1

When reference is made herein to an introgression fragment on chromosome 5 comprising an internal fruit rot resistance QTL this encompasses various sizes of introgression fragments, e.g. the fragment as found in NCIMB 43530, or derived from another wild donor, comprising the SNP genotype and haplotype of the donor for all SNP markers (SNP_01 to SNP_18), but also smaller introgression fragments (sub-fragments) which comprise the QTL5.1 but which comprise the SNP haplotype indicative of the QTL with fewer SNP markers, e.g. only 3, 4, 5, 6, 7, 8, 9 or 10 SNP markers of the group SNP_01 to SNP_18, optionally only 3, 4, 5, 6, 7, 8, 9 or 10 SNP consecutive markers selected from SNP_01 to SNP_18. Such smaller fragments are thus smaller introgression fragments, which comprise QTL5.1 (or a variant thereof) and which lack the SNP donor genotype of 1, 2, 3, 4, 5, 6, 7, 8, 9, 10, 11, 12 or more of markers SNP_01 to SNP_18, where however the fragment retains QTL5.1 or a variant thereof, i.e. it still confers internal fruit rot resistance (compared to the control, e.g. the genetic control) e.g. when the introgression fragment is in homozygous form in the cultivated cucumber genome. As QTL5.1 has been fine mapped to lie in-between SNP_12 and SNP_15 (see FIG. 6), such a smaller fragment preferably comprises the donor SNP haplotype for one or more or all of SNP_12, SNP_45, SNP_13, SNP_14, SNP_46, SNP_47, SNP_48, SNP_49, SNP_50, SNP_51 and SNP_15.

A plant may thus comprise an introgression fragment from a wild cucumber on chromosome 5, comprising Quantitative Trait Locus QTL5.1, wherein the introgression fragment comprising QTL5.1 comprises a haplotype of at least 3, 5, 6, 7, 8, 9, 10, or 11 markers selected from:
a Guanine for SNP_12 at nucleotide 51 of SEQ ID NO: 12 or at nucleotide 51 of a sequence comprising at least 97% identity to SEQ ID NO: 12,
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 or at nucleotide 51 of a sequence comprising at least 97% identity to SEQ ID NO: 13,
a Guanine for SNP_14 at nucleotide 51 of SEQ ID NO: 14 or at nucleotide 51 of a sequence comprising at least 97% identity to SEQ ID NO: 14,
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 or at nucleotide 51 of a sequence comprising at least 97% identity to SEQ ID NO: 15.

Thus, in one aspect a cultivated cucumber plant is provided comprising an introgression fragment from a wild donor of cucumber, wherein the introgression fragment comprises QTL5.1, or a variant thereof, and wherein the introgression fragment comprises all or part of the region starting at nucleotide (or base) 3701817 of chromosome 5 and ending at nucleotide (or base) 4028826 of chromosome 5. In other words, all or part of the region starting at nucleotide 3701817 of chromosome 5 and ending at nucleotide 4028826 of chromosome 5 is, in one aspect, from a wild donor of cucumber and comprises QTL5.1 or a variant thereof.

In another aspect a cultivated cucumber plant is provided comprising an introgression fragment from a wild donor of cucumber, wherein the introgression fragment comprises QTL5.1, or a variant thereof, and wherein the introgression fragment comprises all or part of the region starting at nucleotide (or base) 3823864 of chromosome 5 and ending at nucleotide (or base) 3967955 of chromosome 5. In other words, all or part of the region starting at nucleotide 3823864 of chromosome 5 and ending at nucleotide 3967955 of chromosome 5 is, in one aspect, from a wild donor of cucumber and comprises QTL5.1 or a variant thereof.

In one aspect QTL5.1 (or a variant thereof) is located in-between marker SNP_01 at nucleotide 51 of SEQ ID NO: 1 (or at nucleotide 51 of a sequence comprising at least 97% identity to SEQ ID NO: 1) and marker SNP_18 at nucleotide 51 of SEQ ID NO: 18 (or at nucleotide 51 of a sequence comprising at least 97% identity to SEQ ID NO: 18). This larger region is the region to which the QTL was initially mapped. Later fine mapping showed that QTL5.1 is located in the sub-region between SNP_12 and SNP_15.

In one aspect QTL5.1 (or a variant thereof) is located in-between marker SNP_12 at nucleotide 51 of SEQ ID NO: 12 (or at nucleotide 51 of a sequence comprising at least 97% identity to SEQ ID NO: 12) and marker SNP_15 at nucleotide 51 of SEQ ID NO: 15 (or at nucleotide 51 of a sequence comprising at least 97% identity to SEQ ID NO: 15).

In one aspect all (or less, e.g. 17, 16, 15, 14, 13, 12, 11, 10 or less, e.g. 9, 8, 7, 6, 5, 4 or 3 SNP markers, especially consecutive SNP markers) of SNP_01 to SNP_18 markers indicative of QTL5.1, especially of SNP_12 to SNP_15 (as shown in FIG. 6), have the SNP haplotype of the donor introgression, i.e. SNP_01 comprises an Adenine at nucleotide 51 of SEQ ID NO: 1 (or at nucleotide 51 of a sequence comprising at least 97% identity to SEQ ID NO: 1), SNP_02 comprises a Guanine at nucleotide 51 of SEQ ID NO: 2 (or at nucleotide 51 of a sequence comprising at least 97% identity to SEQ ID NO: 2), SNP_03 comprises a Cytosine at nucleotide 51 of SEQ ID NO: 3 (or at nucleotide 51 of a sequence comprising at least 97% identity to SEQ ID NO: 3), SNP_04 comprises a Guanine at nucleotide 51 of SEQ ID NO: 4 (or at nucleotide 51 of a sequence comprising at least 97% identity to SEQ ID NO: 4), SNP_05 comprises a Adenine at nucleotide 51 of SEQ ID NO: 5 (or at nucleotide 51 of a sequence comprising at least 97% identity to SEQ ID NO: 5), SNP_06 comprises a Adenine at nucleotide 51 of SEQ ID NO: 6 (or at nucleotide 51 of a sequence comprising at least 97% identity to SEQ ID NO: 6), SNP_07 comprises a Cytosine at nucleotide 51 of SEQ ID NO: 7 (or at nucleotide 51 of a sequence comprising at least 97% identity to SEQ ID NO: 7), SNP_08 comprises a Cytosine at nucleotide 51 of SEQ ID NO: 8 (or at nucleotide 51 of a sequence comprising at least 97% identity to SEQ ID NO: 8), SNP_09 comprises a Adenine at nucleotide 51 of SEQ ID NO: 9 (or at nucleotide 51 of a sequence comprising at least 97% identity to SEQ ID NO: 9), SNP_10 comprises a Adenine at nucleotide 51 of SEQ ID NO: 10 (or at nucleotide 51 of a sequence comprising at least 97% identity to SEQ ID NO: 10), SNP_11 comprises a Cytosine at nucleotide 51 of SEQ ID NO: 11 (or at nucleotide 51 of a sequence comprising at least 97% identity to SEQ ID NO: 11), SNP_12 comprises a Guanine at nucleotide 51 of SEQ ID NO: 12 (or at nucleotide 51 of a sequence comprising at least 97% identity to SEQ ID NO: 12), SNP_13 comprises a Adenine at nucleotide 51 of SEQ ID NO: 13 (or at nucleotide 51 of a sequence comprising at least 97% identity to SEQ ID NO: 13), SNP_14 comprises a Guanine at nucleotide 51 of SEQ ID NO: 14 (or at nucleotide 51 of a sequence comprising at least 97% identity to SEQ ID NO: 14), SNP_15 comprises a Guanine at nucleotide 51 of SEQ ID NO: 15 (or at nucleotide 51 of a sequence comprising at least 97% identity to SEQ ID NO: 15), SNP_16 comprises a Cytosine at nucleotide 51 of SEQ ID NO: 16 (or at nucleotide 51 of a sequence comprising at least 97% identity to SEQ ID NO: 16), SNP_17 comprises a Adenine at nucleotide 51 of SEQ ID NO: 17 (or at nucleotide 51 of a sequence comprising at least 97% identity to SEQ ID NO: 17) and SNP_18 comprises a Guanine at nucleotide 51 of SEQ ID NO: 18 (or at nucleotide 51 of a sequence comprising at least 97% identity to SEQ ID NO: 18).

As QTL5.1 has been fine mapped to lie in between SNP_12 and SNP_15, as shown in FIG. 6, the other markers outside this region, i.e. SNP_1 to SNP_11 and SNP_16 to SNP_18 do not need to have the donor SNP haplotype although they may optionally have it.

In one aspect all (or less, e.g. 10 or less, e.g. 9, 8, 7, 6, 5, 4 or 3 SNP markers, especially consecutive SNP markers) of SNP_12 to SNP_15 markers indicative of QTL5.1 have the SNP haplotype of the donor introgression, i.e. SNP_12 comprises a Guanine at nucleotide 51 of SEQ ID NO: 12 (or at nucleotide 51 of a sequence comprising at least 97% identity to SEQ ID NO: 12), SNP_45 comprises an Adenine at nucleotide 51 of SEQ ID NO: 45 or at nucleotide 51 of a sequence comprising at least 97% identity to SEQ ID NO: 45, SNP_13 comprises a Adenine at nucleotide 51 of SEQ ID NO: 13 (or at nucleotide 51 of a sequence comprising at least 97% identity to SEQ ID NO: 13), SNP_14 comprises a Guanine at nucleotide 51 of SEQ ID NO: 14 (or at nucleotide 51 of a sequence comprising at least 97% identity to SEQ ID NO: 14), SNP_46 comprises a Thymine at nucleotide 51 of SEQ ID NO: 46 or at nucleotide 51 of a sequence comprising at least 97% identity to SEQ ID NO: 46, SNP_47 comprises a Thymine at nucleotide 51 of SEQ ID NO: 47 or at nucleotide 51 of a sequence comprising at least 97% identity to SEQ ID NO: 47, SNP_48 comprises a Cytosine for SNP_48 at nucleotide 51 of SEQ ID NO: 48 or at nucleotide 51 of a sequence comprising at least 97% identity to SEQ ID NO: 48, SNP_49 comprises a Guanine at nucleotide 51 of SEQ ID NO: 49 or at nucleotide 51 of a sequence comprising at least 97% identity to SEQ ID NO: 49, SNP_50 comprises a Thymine at nucleotide 51 of SEQ ID NO: 50 or at nucleotide 51 of a sequence comprising at least 97% identity to SEQ ID NO: 50, SNP_51 comprises an Adenine at nucleotide 51 of SEQ ID NO: 51 or at nucleotide 51 of a sequence comprising at least 97% identity to SEQ ID NO: 51, SNP_15 comprises a Guanine at nucleotide 51 of SEQ ID NO: 15 (or at nucleotide 51 of a sequence comprising at least 97% identity to SEQ ID NO: 15).

In one aspect QTL5.1 is located in between SNP_12 and SNP_15, or in between SNP_12 and SNP_47, or in between SNP_47 and SNP_15 and the introgression fragment may comprise the donor haplotype for all SNP markers, or only for 3, 4, 5, 6, 7, 8, 9 or 10 of the SNP markers selected from SNP_12 to SNP_15 (as shown in FIG. 6), especially at least 3, 4, 5, 6, 7, 8, 9 or 10 consecutive SNP markers selected from SNP_12 to SNP_15. The donor haplotype is as described herein above. Further fine-mapping or sequencing can be used to identify in which sub-region of the region starting at SNP_12 and ending at SNP_15 the QTL is found. It may lie between any two markers, e.g. between SNP_12 and SNP_45, or between SNP_45 and SNP_13, etc.

In another aspect the introgression fragment of the invention (comprising QTL5.1 or a variant thereof) is a fragment comprising a smaller sub-fragment (part) of the region starting at 3701817 bp and ending at 4028826 bp of chromosome 5, e.g. comprising one or more or all of SNP_12 to SNP_15 and having a size of e.g. having a size of e.g. 350 kb, 325 kb, 200 kb, 145 kb, 100 kb, 50 kb, 35 kb, 30 kb, 20 kb, or less and comprising the QTL or a variant thereof. In one aspect the part is at least 5 kb, 10 kb, 20 kb in size, or more. The smaller fragment retains QTL5.1, i.e. the smaller fragment confers an increase internal fruit rot resistance (reduced susceptibility to internal fruit rot), e.g. as described for the whole introgression fragment.

In one aspect the cultivated cucumber plant of the invention comprises an introgression fragment from a wild cucumber donor, which introgression fragment comprises QTL5.1 or a variant thereof, wherein the introgression fragment comprises all of part of the region starting at 3.7 Mb and ending at 4.1 Mb of the physical chromosome 5; in another aspect starting at 3.701817 Mb and ending at 4.028826 Mb.

In one aspect the cultivated cucumber plant of the invention comprises an introgression fragment from a wild cucumber donor, which introgression fragment comprises QTL5.1 or a variant thereof, wherein the introgression fragment comprises all of part of the region starting at 3.83 Mb and ending at 3.97 Mb of the physical chromosome 5; in another aspect starting at 3.823864 Mb and ending at 3.967955 Mb.

In one aspect the introgression fragment on chromosome 5 comprising QTL5.1 is obtainable by crossing a plant grown from NCIMB 43530 (or ancestors thereof or descendent/progeny therefrom) with another cucumber plant, especially a cultivated cucumber plant, in one aspect a long cucumber type.

### QTL3.1

When reference is made herein to an introgression fragment on chromosome 3 comprising an internal fruit rot resistance QTL this encompasses various sizes of introgression fragments, e.g. the fragment as found in NCIMB 43530, or derived from another wild donor, comprising the SNP genotype and haplotype of the donor for all SNP markers (SNP_19 to SNP_42), but also smaller introgression fragments (sub-fragments) which comprise the QTL3.1 but which comprise the SNP haplotype indicative of the QTL with fewer SNP markers, e.g. only 3, 4, 5, 6, 7, 8, 9 or 10 SNP markers of the group SNP_19 to SNP_42, optionally only 3, 4, 5, 6, 7, 8, 9 or 10 SNP consecutive markers selected from SNP_19 to SNP_42. Such smaller fragments are thus smaller introgression fragments, which comprise QTL3.1 (or a variant thereof) and which lack the SNP donor genotype of 1, 2, 3, 4, 5, 6, 7, 8, 9, 10, 11, 12 or more of markers SNP_19 to SNP_42, where however the fragment retains QTL3.1 or a variant thereof, i.e. it still confers internal fruit rot resistance (compared to the control, e.g. the genetic control) e.g. when the introgression fragment is in homozygous form in the cultivated cucumber genome.

As QTL3.1 is now known to be located between SNP_35 and 659 nt downstream of SNP_36 (i.e. in the region of SEQ ID NO: 72 and 73), which region comprises 9 genes, and that this region is part of a larger duplication of a region starting at SEQ ID NO: 83 and ending at SEQ ID NO: 84, the introgression fragment on chromosome 3 comprising an internal fruit rot resistance QTL encompasses introgression fragments which a) comprise a duplication on chromosome 3 of SEQ ID NO: 72 or a sequence comprising at least 95% sequence identity to SEQ ID NO: 72, or b) comprise a duplication on chromosome 3 of the region flanked by SEQ ID NO: 83 and SEQ ID NO: 84, or starting at nucleotide 1 of SEQ ID NO: 83 and ending at nucleotide 100 of SEQ ID NO: 84, or c) comprising the donor SNP haplotype for at least 2, 3, 4, 5, 6, 7 or all 8 SNP markers selected from SNP_29, SNP_30, SNP_31, SNP_32, SNP_33, SNP_34, SNP_35, SNP_36, all of which are present on the introgression fragment in the duplicated region or d) comprising a duplication or one or more or all of the 8 OGD genes and/or of the HR-like protein on chromosome 3 or e) comprising alleles of one or more or all of the 8 OGD genes and/or of the HR-like protein which have a higher expression than the wild type alleles and therefore lead to higher amounts of protein.

The above aspects a) to d) can be achieved via introgression fragments or by other methods, such as genome editing, transgenic methods, etc. and when referring to plants ‘comprising introgression fragments’ herein, in one aspect also such alternative means of modifying the genome are encompassed herein. These are also described elsewhere.

Thus, in one aspect a cultivated cucumber plant is provided comprising an introgression fragment from a wild donor of cucumber, wherein the introgression fragment comprises QTL3.1, or a variant thereof, and wherein the introgression fragment comprises all or part of the region starting at nucleotide (or base) 9237416 of chromosome 3 and ending at nucleotide (or base) 9264936 of chromosome 3. In other words, all or part of the region starting at nucleotide 9237416 of chromosome 3 and ending at nucleotide 9264936 of chromosome 3 is, in one aspect, from a wild donor of cucumber and comprises QTL3.1 or a variant thereof. Preferably the introgression fragment comprises a duplication of all or part of this region. The region contains 9 genes, eight of which are genes encoding 2OGD enzymes and one gene encodes a HR-like protein.

The donor used herein had a duplication of 9 genes, but other donors with different structural variation may be identified and such structural variation may be introgressed. For example, donors comprising duplications or triplications on chromosome 3 of one or more of the genes encoding a protein selected from
an OGD1 protein comprises the amino acid sequence of SEQ ID NO: 52 or a sequence comprising at least 95% sequence identity to SEQ ID NO: 52,
an OGD2 protein comprises the amino acid sequence of SEQ ID NO: 53 or a sequence comprising at least 95% sequence identity to SEQ ID NO: 53,
an OGD3 protein comprises the amino acid sequence of SEQ ID NO: 54 or a sequence comprising at least 95% sequence identity to SEQ ID NO: 54,
an OGD4 protein comprises the amino acid sequence of SEQ ID NO:55 or a sequence comprising at
an OGD5 protein comprises the amino acid sequence of SEQ ID NO: 56 or a sequence comprising at least 95% sequence identity to SEQ ID NO: 56,
an OGD6 protein comprises the amino acid sequence of SEQ ID NO: 57 or a sequence comprising at least 95% sequence identity to SEQ ID NO: 57,
an OGD7 protein comprises the amino acid sequence of SEQ ID NO: 58 or a sequence comprising at least 95% sequence identity to SEQ ID NO: 58,
an OGD8 protein comprises the amino acid sequence of SEQ ID NO: 59 or a sequence comprising at least 95% sequence identity to SEQ ID NO: 59,
a HR-like protein comprises the amino acid sequence of SEQ ID NO: 60 or a sequence comprising at least 95% sequence identity to SEQ ID NO: 60.

Even though QTL3.1 has been fine mapped to be between SNP_35 and (659 nt downstream of) SNP_36, the SNP markers linked to QTL3.1 are also useful in identifying plants comprising QTL3.1.

The other SNP markers found on the introgression fragment, but outside the duplicated region (SNP_19 to SNP_28 and SNP_37 to SNP_42) can also be used for selection of the introgression fragment, as described before, as these are also indicative of the presence of the introgression fragment, but as they are not on the duplicated region these markers can be removed without losing the QTL3.1.

In one aspect QTL3.1 (or a variant thereof) is located in-between marker SNP_19 at nucleotide 51 of SEQ ID NO: 19 (or at nucleotide 51 of a sequence comprising at least 97% identity to SEQ ID NO: 19) and marker SNP_42 at nucleotide 51 of SEQ ID NO: 42 (or at nucleotide 51 of a sequence comprising at least 97% identity to SEQ ID NO: 42).

In one aspect all (or less, e.g. 23, 22, 21, 20, 19, 18, 17, 16, 15, 14, 13, 12, 11, 10 or less, e.g. 9, 8, 7, 6, 5, 4 or 3 SNP markers, especially consecutive SNP markers) of SNP_19 to SNP_42 markers indicative of QTL3.1 have the SNP haplotype of the donor introgression, i.e. SNP_19 comprises an Guanine at nucleotide 51 of SEQ ID NO: 19 (or at nucleotide 51 of a sequence comprising at least 97% identity to SEQ ID NO: 19), SNP_20 comprises a Guanine at nucleotide 51 of SEQ ID NO: 20 (or at nucleotide 51 of a sequence comprising at least 97% identity to SEQ ID NO: 20), SNP_21 comprises a Thymine at nucleotide 51 of SEQ ID NO: 21 (or at nucleotide 51 of a sequence comprising at least 97% identity to SEQ ID NO: 21), SNP_22 comprises a Adenine at nucleotide 51 of SEQ ID NO: 22 (or at nucleotide 51 of a sequence comprising at least 97% identity to SEQ ID NO: 22), SNP_23 comprises a single nucleotide deletion, ie. comprises only a Cytosine at nucleotide 51 of SEQ ID NO: 23 (or at nucleotide 51 of a sequence comprising at least 97% identity to SEQ ID NO: 23) instead of a Cytosine-Adenine at nucleotide 51 of SEQ ID NO: 23 (as shown in SEQ ID NO: 43), SNP_24 comprises a Adenine at nucleotide 51 of SEQ ID NO: 24 (or at nucleotide 51 of a sequence comprising at least 97% identity to SEQ ID NO: 24), SNP_25 comprises a Guanine at nucleotide 51 of SEQ ID NO: 25 (or at nucleotide 51 of a sequence comprising at least 97% identity to SEQ ID NO: 25), SNP_26 comprises a Thymine at nucleotide 51 of SEQ ID NO: 26 (or at nucleotide 51 of a sequence comprising at least 97% identity to SEQ ID NO: 26), SNP_27 comprises a Cytosine at nucleotide 51 of SEQ ID NO: 27 (or at nucleotide 51 of a sequence comprising at least 97% identity to SEQ ID NO: 27), SNP_28 comprises a Guanine at nucleotide 51 of SEQ ID NO: 28 (or at nucleotide 51 of a sequence comprising at least 97% identity to SEQ ID NO: 28), SNP_29 comprises a Cytosine at nucleotide 51 of SEQ ID NO: 29 (or at nucleotide 51 of a sequence comprising at least 97% identity to SEQ ID NO: 29), SNP_30 comprises a Adenine at nucleotide 51 of SEQ ID NO: 30 (or at nucleotide 51 of a sequence comprising at least 97% identity to SEQ ID NO: 30), SNP_31 comprises a Guanine at nucleotide 51 of SEQ ID NO: 31 (or at nucleotide 51 of a sequence comprising at least 97% identity to SEQ ID NO: 31), SNP_32 comprises a Guanine at nucleotide 51 of SEQ ID NO: 32 (or at nucleotide 51 of a sequence comprising at least 97% identity to SEQ ID NO: 32), SNP_33 comprises a Guanine at nucleotide 51 of SEQ ID NO: 33 (or at nucleotide 51 of a sequence comprising at least 97% identity to SEQ ID NO: 33), SNP_34 comprises a Guanine at nucleotide 51 of SEQ ID NO: 34 (or at nucleotide 51 of a sequence comprising at least 97% identity to SEQ ID NO: 34), SNP_35 comprises a Thymine at nucleotide 51 of SEQ ID NO: 35 (or at nucleotide 51 of a sequence comprising at least 97% identity to SEQ ID NO: 35), SNP_36 comprises a deletion, ie. comprises only a Thymine at nucleotide 51 of SEQ ID NO: 36 (or at nucleotide 51 of a sequence comprising at least 97% identity to SEQ ID NO: 36) instead of a Thymine-Guanine at nucleotide 51 of SEQ ID NO: 36 (as shown in SEQ ID NO: 44),, SNP_37 comprises a Cytosine at nucleotide 51 of SEQ ID NO: 37 (or at nucleotide 51 of a sequence comprising at least 97% identity to SEQ ID NO: 37), SNP_38 comprises a Guanine at nucleotide 51 of SEQ ID NO: 38 (or at nucleotide 51 of a sequence comprising at least 97% identity to SEQ ID NO: 38), SNP_39 comprises a Cytosine at nucleotide 51 of SEQ ID NO: 39 (or at nucleotide 51 of a sequence comprising at least 97% identity to SEQ ID NO: 39), SNP_40 comprises a Thymine at nucleotide 51 of SEQ ID NO: 40 (or at nucleotide 51 of a sequence comprising at least 97% identity to SEQ ID NO: 40), SNP_41 comprises a Adenine at nucleotide 51 of SEQ ID NO: 41 (or at nucleotide 51 of a sequence comprising at least 97% identity to SEQ ID NO: 41), SNP_42 comprises a Adenine at nucleotide 51 of SEQ ID NO: 42 (or at nucleotide 51 of a sequence comprising at least 97% identity to SEQ ID NO: 42).

In one aspect QTL3.1 the introgression fragment may comprise the donor haplotype for all SNP markers, or only for 3, 4, 5, 6, 7, 8, 9 or 10 of the SNP markers selected from SNP_19 to SNP_42, especially at least 3, 4, 5, 6, 7, 8, 9 or 10 consecutive SNP markers selected from SNP_19 to SNP_42. In another aspect QTL3.1 is located on a sub-fragment comprising the donor haplotype of one or more or all of markers SNP_29, SNP_30, SNP_31, SNP_32, SNP_33, SNP_34, SNP_35, SNP_36. The donor haplotype is as described herein above.

Although SNP haplotype selection is useful to select for QTL3.1 e.g. from the seeds deposited herein, the knowledge of the underlying genes provides much more specific ways of selecting QTL3.1 and plants comprising introgression fragments with duplications of QTL3.1 or with duplications of one or more of the genes selected from OGD1 to OGD8 and the gene encoding the HR-like protein.

Donor plants can be screened for the presence of a duplication on chromosome 3 of SEQ ID NO: 72 or a sequence comprising at least 95% sequence identity to SEQ ID NO: 72, or a duplication on chromosome 3 of the region flanked by SEQ ID NO: 83 and SEQ ID NO: 84, or starting at nucleotide 1 of SEQ ID NO: 83 and ending at nucleotide 100 of SEQ ID NO: 84, or comprising a duplication or one or more or all of the 8 OGD genes and/or of the HR-like protein on chromosome 3. Also donors can be screened for comprising genes which have a higher expression than the wild type, for one or more of the 8 OGD genes and/or for the gene encoding the HR-like protein.

When such a donor plant has been identified, it can be crossed with a susceptible plant and the duplication or the genes having a higher expression than the wild type can be introgressed into the susceptible plant, e.g. by backcrossing. The plant can then be tested for its resistance to internal fruit rot caused by DB.

As the instant duplication was found in a wild donor, it is possible that other wild donors exist comprising the same or similar structural variation or alleles having a higher gene expression. These donors can therefore equally be used as a source for QTL3.1. Alternatively, the duplications or the alleles having enhanced gene expression can be generated de novo, as already described.

In one aspect the introgression fragment on chromosome 3 comprising QTL3.1 is obtainable by crossing a plant grown from NCIMB 43530 (or ancestors thereof or descendent/progeny therefrom) with another cucumber plant, especially a cultivated cucumber plant, in one aspect a long cucumber type.

When referring to the SNP markers herein, which are indicative of the presence of the introgression fragment on chromosome 5 or 3 (and either of the QTLs present on the introgression fragment), it is understood that the donor SNP genotype (or SNP haplotype, when referring to several markers) which is indicative of the introgression fragment is referred to, i.e. the SNP genotype (and haplotype) as e.g. provided in Table 1 herein for chromosome 5 and in Table 2 for chromosome 3. It is noted that the SNP marker genotype (and haplotype) can distinguish between the introgression fragment being in homozygous or heterozygous form, as shown in the Tables. In homozygous form the nucleotide is identical, while in heterozygous form the nucleotide is not identical. The SNP genotype of the ‘wild type’ chromosome lacking the introgression fragment is the other genotype, also listed in Table 1 and 2 (under genotype of recurrent parent). So, e.g. the genotype of SNP_01 indicative of the introgression fragment comprising QTL5.1 is ‘AA’ (QTL5.1/QTL5.1) or ‘AG’ (QTL5.1/wt) while the SNP genotype indicative of the wild type/genetic control/control (lacking the introgression fragment) is ‘GG’ (wt/wt). Thus, when referring to a plant or plant part (e.g. cell) comprising the introgression fragment in homozygous or heterozygous form, it is understood that the SNP markers linked to the introgression fragment have the corresponding SNP genotype or SNP haplotype (when referring to several markers).

So in one aspect, a cultivated Cucumis sativus var. sativus plant is provided comprising an introgression fragment on chromosome 5 and/or 3 in homozygous or heterozygous form, i.e. comprising QTL5.1 and/or QTL3.1 (i.e. the duplication of QTL3.1 or the duplication of one or more of the OGD genes or the gene encoding the HR-like protein, or alleles having higher expression) from a wild donor, wherein said introgression fragment confers an increase in internal fruit rot resistance (also referred to as a reduction in base-line susceptibility) compared to the control cucumber plant lacking the introgression fragment on chromosome 5 and 3, e.g. the genetic control or control line or variety of the same cucumber type, when grown under the same conditions.

The increase in internal fruit rot resistance is phenotypically expressed as a (statistically) significantly lower average number of fruits developing from spore-inoculated flowers, of the cultivated cucumber plant line or variety comprising the introgression fragment on chromosome 5 and/or 3 (preferably in homozygous form), that develop symptoms of internal fruit rot, compared to the control line or variety lacking the introgression fragment on chromosome 5 and 3 (e.g. the genetic control) when tested in e.g. the internal fruit rot assay as described herein. As already mentioned, the increase in internal fruit rot resistance due to the QTL or QTLs can also be expressed as a reduction of base-line susceptibility to internal fruit rot of the plant line or variety comprising the introgression fragment(s) (comprising QTL5.1 and/or QTL3.1, preferably in homozygous form) compared to the control line or variety (preferably the genetic control) lacking the introgression fragments and lacking the QTLs.

In a preferred embodiment cucumber plants (and plant parts), especially long cucumber plants, are provided in which the presence of both QTL5.1 and QTL3.1 in homozygous form results in equal to or less than 2.0%, preferably equal to or less than 1.5% of the inoculated flowers, preferably equal to or less than 1.0%, or equal to or less than 0.5%, or even 0% of the inoculated flowers showing symptoms of internal fruit rot. In the internal fruit rot assay developed herein, even the slightest browning or discoloration inside the fruit (at the flower end), which develops from the spore-inoculated flower, is scored as ‘symptoms of internal fruit rot’ (see FIG. 1). Thus in such plants, equal to or less than 2.0%, or equal to or less than 1.5%, of the spore-inoculated flowers develop fruits which show any symptoms of internal fruit rot at all, while in the control significantly more of the inoculated flowers develop fruits which show symptoms of internal fruit rot.

As different cucumber lines or types have a different base-line susceptibility, the effect of the QTLs is in one aspect expressed as a reduction in base-line susceptibility.

In one aspect the presence of both QTL5.1 and QTL3.1 in homozygous form reduces the base-line susceptibility of a cucumber line or type by at least 10%, 15%, 20%, 25%, 30%, 40% or more. Thus, for example, in the long cucumber lines used in the Examples, the base-line susceptibility is reduced by at least 20% compared to the recurrent parent and even over 40% compared to another DB susceptible long cucumber control plant.

When determining the reduction in base-line susceptibility of a plant comprising one or both QTLs, it is therefore important to include the appropriate controls in the test for measuring the percentage of DB inoculated fruits that develop symptoms of internal fruit rot. The controls should be of the same cucumber type, but lacking QTL5.1 and lacking the duplication of QTL3.1, and they are ideally genetically similar to the plant line with the QTL or QTLs. For example, the control may be the recurrent parent line or a genetic control. In this way the effect of one or both QTLs on the reduction of base-line susceptibility can be measured in any cucumber line or type.

The plants of the invention, therefore, comprise a genome of cultivated cucumber, with at least one or two recombinant chromosomes 5 (i.e. heterozygous or homozygous) and/or with at least one or two recombinant chromosomes 3 (i.e. heterozygous or homozygous). The recombinant chromosomes comprise a fragment of a wild donor, which is easily distinguishable from the cultivated cucumber genome by molecular marker analysis, sequence analysis, digital PCR (to determine copy number), gene expression analysis, sequencing, chromosome painting and similar techniques. Regarding chromosome 3, the duplication of the region between SNP_35 and 659 nucleotides downstream of SNP_36 can also easily be identified by analyzing gene copy number (e.g. by digital PCR) or gene expression of the 9 genes in the region.

In one aspect the introgression fragment on chromosome 5 is from a wild donor, comprises the DB fruit rot resistance QTL, QTL5.1, or a variant thereof, and comprises all or part of the region starting at nucleotide 3701817 and ending at nucleotide 4028826 of the chromosome. Thus, the introgression fragment comprises the QTL5.1 or a variant thereof and one or more or all (e.g. 2, 3, 4, 5, 6, 7, 8, 9, 10, 11, 12, 13, 14, 15, 16, 17, 18) SNP markers of the wild donor cucumber selected from SNP_01 to SNP_18 as shown in Table 1.

As QTL5.1 was fine mapped, in another aspect the introgression fragment on chromosome 5 is from a wild donor, comprises the DB fruit rot resistance QTL, QTL5.1, or a variant thereof, and comprises all or part of the region starting at nucleotide 3823864 and ending at nucleotide 3967955 of the chromosome. Thus, the introgression fragment comprises the QTL5.1 or a variant thereof and one or more or all (e.g. 2, 3, 4, 5, 6, 7, 8, 9, 10, 11) SNP markers of the wild donor cucumber selected from SNP_12, SNP_45, SNP_13, SNP_14, SNP_46, SNP_47, SNP_48, SNP_49, SNP_50, SNP_51 and SNP_15 as shown in FIG. 6.

In a preferred aspect the introgression fragment on chromosome 5 is from a wild donor, comprises the DB fruit rot resistance QTL, QTL5.1, or a variant thereof, and comprises all or part of the region starting at nucleotide 3823864 and ending at nucleotide 3967955 of the chromosome. the introgression fragment comprises QTL5.1 or a variant thereof and one or more or all SNP markers of the wild donor selected from SNP_12, SNP_45, SNP_13, SNP_14, SNP_46, SNP_47, SNP_48, SNP_49, SNP_50, SNP_51 and SNP_15.

In one aspect the introgression fragment on chromosome 3 is from a wild donor, comprises the DB fruit rot resistance QTL, QTL3.1, or a variant thereof, and comprises a) duplication on chromosome 3 of SEQ ID NO: 72 or a sequence comprising at least 95% sequence identity to SEQ ID NO: 72, or b) a duplication on chromosome 3 of the region flanked by SEQ ID NO: 83 and SEQ ID NO: 84, or starting at nucleotide 1 of SEQ ID NO: 83 and ending at nucleotide 100 of SEQ ID NO: 84, or c) the donor SNP haplotype for at least 2, 3, 4, 5, 6, 7 or all 8 SNP markers selected from SNP_29, SNP_30, SNP_31, SNP_32, SNP_33, SNP_34, SNP_35, SNP_36, all of which are present on the introgression fragment in the duplicated region or d) a duplication or one or more or all of the 8 OGD genes and/or of the HR-like protein on chromosome 3 or e) alleles of one or more or all of the 8 OGD genes and/or of the HR-like protein which have a higher expression than the wild type alleles and therefore lead to higher amounts of protein.

The wild donor SNP haplotype for SNP_29, SNP_30, SNP_31, SNP_32, SNP_33, SNP_34, SNP_35 and SNP_36 is shown in Table 2.

As QTL3.1 was fine mapped, in another aspect the introgression fragment on chromosome 3 is from a wild donor, comprises the DB fruit rot resistance QTL, QTL3.1, or a variant thereof, and comprises all or part of the region starting at nucleotide 9237416 and ending at nucleotide 9264936 of the chromosome. Thus, the introgression fragment comprises the QTL3.1 or a variant thereof and comprises all or part of the region starting at SNP_35 and ending 659 nt downstream of SNP_36, preferably this region is duplicated on the chromosome. In one aspect this region or part of this region is duplicated one or more times on the chromosome. In one aspect one or more of the genes are duplicated on the chromosome, wherein the genes are of one or more of the genes encoding a protein selected from
an OGD1 protein comprises the amino acid sequence of SEQ ID NO: 52 or a sequence comprising at least 95% sequence identity to SEQ ID NO: 52,
an OGD2 protein comprises the amino acid sequence of SEQ ID NO: 53 or a sequence comprising at least 95% sequence identity to SEQ ID NO: 53,
an OGD3 protein comprises the amino acid sequence of SEQ ID NO: 54 or a sequence comprising at least 95% sequence identity to SEQ ID NO: 54,
an OGD4 protein comprises the amino acid sequence of SEQ ID NO:55 or a sequence comprising at
an OGD5 protein comprises the amino acid sequence of SEQ ID NO: 56 or a sequence comprising at least 95% sequence identity to SEQ ID NO: 56,
an OGD6 protein comprises the amino acid sequence of SEQ ID NO: 57 or a sequence comprising at least 95% sequence identity to SEQ ID NO: 57,
an OGD7 protein comprises the amino acid sequence of SEQ ID NO: 58 or a sequence comprising at least 95% sequence identity to SEQ ID NO: 58,
an OGD8 protein comprises the amino acid sequence of SEQ ID NO: 59 or a sequence comprising at least 95% sequence identity to SEQ ID NO: 59,
a HR-like protein comprises the amino acid sequence of SEQ ID NO: 60 or a sequence comprising at least 95% sequence identity to SEQ ID NO: 60.

In one aspect the presence of the introgression fragment on chromosomes 5 or 3 in the genome of the plant or plant cell or plant tissue (or in the DNA extracted therefrom) is detectable by a molecular marker assay which detects one or more molecular markers of the introgression fragment e.g. the donor SNP for one or more of SNP_12 to SNP_15 (as shown in FIG. 6) linked to QTL5.1 or, for QTL3.1, by molecular analysis which detects duplications of genomic sequences (e.g. the donor genotype or haplotype for one or more of SNP_29 to SNP_36, or the copy number/duplication of these SNP markers, or SEQ ID NO: 72, or the region between SEQ ID NO: 83 and 84) or duplications of genes (OGD1 to OGD8 and/or HR-like gene) in the genome by e.g. digital PCR or sequencing or RT-PCR.

When reference is made herein to one or more molecular markers being “detectable” by a molecular marker assay, this means of course that the plant or plant part comprises the one or more markers in its genome, as the marker would otherwise not be detectable.

### Cucumber Plants Comprising an Introgression Fragment on Chromosome 5 (DB Fruit Rot Resistance QTL 5.1)

QTL5.1 is located in the region between SNP_01 at nucleotide 51 of SEQ ID NO: 1 (or in a variant thereof) and SNP_18 at nucleotide 51 of SEQ ID NO: 18 (or a variant thereof) and has now been fine mapped to be located in the region between SNP_12 at nucleotide 51 of SEQ ID NO: 12 and SNP_15 at nucleotide 51 of SEQ ID NO: 15, with more markers (SNP_45 to SNP_51) added to the region, see FIG. 6.

Therefore, in one aspect a cultivated Cucumis sativus var. sativus plant is provided comprising an introgression fragment on chromosome 5 in homozygous or heterozygous form, wherein said introgression fragment confers an increase in internal fruit rot resistance caused by DB (compared to the plant lacking the introgression fragment, e.g. the genetic control) and wherein said introgression fragment is detectable by a molecular marker assay (i.e. the plant comprises one or more molecular markers) which detects at least 1, preferably at least 2 or 3, or at least 4, 5, 6, 7, 8, 9, 10, 11, 12, 13, 14, 15, 16, 17 or 18 of the markers selected from the group consisting of:
a the AA (homozygous) or AX or AG (heterozygous) genotype for the Single Nucleotide Polymorphism marker SNP_01 at nucleotide 51 of SEQ ID NO: 1 (or in a variant thereof);
b) the GG (homozygous) or AX or AG (heterozygous) genotype for the Single Nucleotide Polymorphism marker SNP_02 at nucleotide 51 of SEQ ID NO: 2 (or in a variant thereof);
c) the CC (homozygous) or CX or CT (heterozygous) genotype for the Single Nucleotide Polymorphism marker SNP_03 at nucleotide 51 of SEQ ID NO: 3 (or in a variant thereof);
d) the GG (homozygous) or GX or GA (heterozygous) genotype for the Single Nucleotide Polymorphism marker SNP_04 at nucleotide 51 of SEQ ID NO: 4 (or in a variant thereof);
e) the AA (homozygous) or AX or AC (heterozygous) genotype for the Single Nucleotide Polymorphism marker SNP_05 at nucleotide 51 of SEQ ID NO: 5 (or in a variant thereof);
f) the AA (homozygous) or AX or AG (heterozygous) genotype for the Single Nucleotide Polymorphism marker SNP_06 at nucleotide 51 of SEQ ID NO: 6 (or in a variant thereof);
g) the CC (homozygous) or CX or CT (heterozygous) genotype for the Single Nucleotide Polymorphism marker SNP_07 at nucleotide 51 of SEQ ID NO: 7 (or in a variant thereof);
h) the CC (homozygous) or CX or CA (heterozygous) genotype for the Single Nucleotide Polymorphism marker SNP_08 at nucleotide 51 of SEQ ID NO: 8 (or in a variant thereof);
i) the AA (homozygous) or AX or AG (heterozygous) genotype for the Single Nucleotide Polymorphism marker SNP_09 at nucleotide 51 of SEQ ID NO: 9 (or in a variant thereof);
j) the AA (homozygous) or AX or AG (heterozygous) genotype for the Single Nucleotide Polymorphism marker SNP_10 at nucleotide 51 of SEQ ID NO: 10 (or in a variant thereof);
k) the CC (homozygous) or CX or CT (heterozygous) genotype for the Single Nucleotide Polymorphism marker SNP_11 at nucleotide 51 of SEQ ID NO: 11 (or in a variant thereof);
l) the GG (homozygous) or GX or GA (heterozygous) genotype for the Single Nucleotide Polymorphism marker SNP_12 at nucleotide 51 of SEQ ID NO: 12 (or in a variant thereof);
m) the AA (homozygous) or AX or AG (heterozygous) genotype for the Single Nucleotide Polymorphism marker SNP_13 at nucleotide 51 of SEQ ID NO: 13 (or in a variant thereof);
n) the GG (homozygous) or GX or GA (heterozygous) genotype for the Single Nucleotide Polymorphism marker SNP_14 at nucleotide 51 of SEQ ID NO: 14 (or in a variant thereof);
o) the GG (homozygous) or GX or GT (heterozygous) genotype for the Single Nucleotide Polymorphism marker SNP_15 at nucleotide 51 of SEQ ID NO: 15 (or in a variant thereof);
p) the CC (homozygous) or CX or CA (heterozygous) genotype for the Single Nucleotide Polymorphism marker SNP_16 at nucleotide 51 of SEQ ID NO: 16 (or in a variant thereof);
q) the AA (homozygous) or AX or AG (heterozygous) genotype for the Single Nucleotide Polymorphism marker SNP_17 at nucleotide 51 of SEQ ID NO: 17 (or in a variant thereof);
r) the GG (homozygous) or GX or GA (heterozygous) genotype for the Single Nucleotide Polymorphism marker SNP_18 at nucleotide 51 of SEQ ID NO: 18 (or in a variant thereof).

As mentioned previously, when referring to a SNP in a variant sequence, that variant sequence comprises at least 97% sequence identity with the mentioned sequence.

Therefore, in one aspect a cultivated Cucumis sativus var. sativus plant is provided comprising an introgression fragment on chromosome 5 in homozygous or heterozygous form, wherein said introgression fragment confers an increase in internal fruit rot resistance caused by DB (compared to the plant lacking the introgression fragment, e.g. the genetic control) and wherein said introgression fragment is detectable by a molecular marker assay (i.e. the plant comprises one or more molecular markers) which detects at least 1, preferably at least 2 or 3, or at least 4, 5, 6, 7, 8, 9, 10 or 11 of the markers selected from the group consisting of:
i) a GG (homozygous) or GX or GA (heterozygous) genotype for SNP_12 at nucleotide 51 of SEQ ID NO: 12 or at nucleotide 51 of a sequence comprising at least 97% identity to SEQ ID NO: 12,
ii) a AA (homozygous) or AX or AG (heterozygous) genotype for SNP_45 at nucleotide 51 of SEQ ID NO: 45 or at nucleotide 51 of a sequence comprising at least 97% identity to SEQ ID NO: 45,
iii) a AA (homozygous) or AX or AG (heterozygous) genotype for SNP_13 at nucleotide 51 of SEQ ID NO: 13 or at nucleotide 51 of a sequence comprising at least 97% identity to SEQ ID NO: 13,
iv) a GG (homozygous) or GX or GA (heterozygous) genotype for SNP_14 at nucleotide 51 of SEQ ID NO: 14 or at nucleotide 51 of a sequence comprising at least 97% identity to SEQ ID NO: 14,
v) a TT (homozygous) or TX or TC (heterozygous) genotype for SNP_46 at nucleotide 51 of SEQ ID NO: 46 or at nucleotide 51 of a sequence comprising at least 97% identity to SEQ ID NO: 46,
vi) a TT (homozygous) or TX or TC (heterozygous) genotype for SNP_47 at nucleotide 51 of SEQ ID NO: 47 or at nucleotide 51 of a sequence comprising at least 97% identity to SEQ ID NO: 47,
vii) a CC (homozygous) or CX or CA (heterozygous) genotype for SNP_48 at nucleotide 51 of SEQ ID NO: 48 or at nucleotide 51 of a sequence comprising at least 97% identity to SEQ ID NO: 48,
viii) a GG (homozygous) or GX or GT (heterozygous) genotype for SNP_49 at nucleotide 51 of SEQ ID NO: 49 or at nucleotide 51 of a sequence comprising at least 97% identity to SEQ ID NO: 49,
ix) a TT (homozygous) or TX or TA (heterozygous) genotype for SNP_50 at nucleotide 51 of SEQ ID NO: 50 or at nucleotide 51 of a sequence comprising at least 97% identity to SEQ ID NO: 50,
x) a AA (homozygous) or AX or AG (heterozygous) genotype for SNP_51 at nucleotide 51 of SEQ ID NO: 51 or at nucleotide 51 of a sequence comprising at least 97% identity to SEQ ID NO: 51,
xi) a GG (homozygous) or GX or GT (heterozygous) genotype for SNP_15 at nucleotide 51 of SEQ ID NO: 15 or at nucleotide 51 of a sequence comprising at least 97% identity to SEQ ID NO: 15.

In one aspect the introgression fragment comprises the haplotype of at least 3, 4, 5 or 6 of the markers listed under a) to r) above, or listed under i) to xi) above, and comprises the QTL5.1 or a variant thereof. In one aspect the introgression fragment is obtainable from (has as source) seeds deposited under NCIMB43530 or progeny thereof, i.e. material derived from the deposit by selfing and/or crossing.

In one aspect the introgression fragment comprises the haplotype of at least 6, 7, 8, 9 or 10 of the markers listed under a) to r) above, or listed under i) to xi) above, and comprises the QTL5.1 or a variant thereof. In one aspect the introgression fragment is obtainable from (has as source) seeds deposited under NCIMB43530 or progeny thereof.

In one aspect the introgression fragment comprises the haplotype of at least 3, 4, 5 or 6 consecutive markers of the markers listed under a) to r) above, or listed under i) to xi) above, and comprises the QTL5.1 or a variant thereof. In one aspect the introgression fragment is obtainable from (has as source) seeds deposited under NCIMB43530 or progeny thereof.

In one aspect the introgression fragment comprises the haplotype of at least 6, 7, 8, 9 or 10 consecutive markers of the markers listed under a) to r) above, or listed under i) to xi) above, and comprises the QTL5.1 or a variant thereof. In one aspect the introgression fragment is obtainable from (has as source) seeds deposited under NCIMB43530 or progeny thereof.

The combination of such unique haplotypes of the introgression fragment in combination with the phenotype conferred by the QTL5.1 (especially in homozygous form and/or in combination with QTL3.1) make it easy for the person skilled in the art to generate and/or identify a plant or plant part which comprises QTL5.1. This does not only apply to plants which have as a source the MYCR3 donor (as present in NCIMB43530, or progeny thereof), but also plants which have as a source other wild donors, having the haplotypes described above and the QTL5.1, or a variant thereof, i.e. conferring the same phenotype as QTL5.1 from MYCR3.

The skilled person can also develop other molecular markers, e.g. markers in-between marker SNP_01 and SNP_18 and/or within 7 cM or within 5 CM of any one of SNP_01 to SNP_18, and/or within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb or less of any one of SNP_01 to SNP_18. Such markers may also be a stretch of nucleotide, CAPS markers, INDELs, etc. The skilled person can, for example, sequence the introgression fragment found in seeds deposited under accession number NCIMB43530 and use the sequence information to develop new markers and marker assays.

Fine mapping can be carried out to identify which sub-region of the introgression fragment on which QTL5.1 (or a variant thereof) is located on, e.g. in the region comprising SNP_12 to SNP_15, e.g. between SNP_12 and SNP_14, or between SNP_14 and SNP_48, or between SNP_48 and SNP_15.

In one aspect QTL5.1 (or a variant thereof) is located in the region comprising SNP_12 and SNP_14, or comprising SNP_14 and SNP_48, or comprising SNP_48 and SNP_15.

Therefore, in one aspect a cultivated Cucumis sativus var. sativus plant is provided comprising an introgression fragment on chromosome 5 in homozygous or heterozygous form, wherein said introgression fragment confers an increase in internal fruit rot resistance caused by DB (compared to the plant lacking the introgression fragment, e.g. the genetic control) and wherein said introgression fragment comprises the following markers:
a GG (homozygous) or GX or GA (heterozygous) genotype for SNP_12 at nucleotide 51 of SEQ ID NO: 12 or at nucleotide 51 of a sequence comprising at least 97% identity to SEQ ID NO: 12,
a AA (homozygous) or AX or AG (heterozygous) genotype for SNP_45 at nucleotide 51 of SEQ ID NO: 45 or at nucleotide 51 of a sequence comprising at least 97% identity to SEQ ID NO: 45,
a AA (homozygous) or AX or AG (heterozygous) genotype for SNP_13 at nucleotide 51 of SEQ ID NO: 13 or at nucleotide 51 of a sequence comprising at least 97% identity to SEQ ID NO: 13,
a GG (homozygous) or GX or GA (heterozygous) genotype for SNP_14 at nucleotide 51 of SEQ ID NO: 14 or at nucleotide 51 of a sequence comprising at least 97% identity to SEQ ID NO: 14, or the following markers:
a GG (homozygous) or GX or GA (heterozygous) genotype for SNP_14 at nucleotide 51 of SEQ ID NO: 14 or at nucleotide 51 of a sequence comprising at least 97% identity to SEQ ID NO: 14,
a TT (homozygous) or TX or TC (heterozygous) genotype for SNP_46 at nucleotide 51 of SEQ ID NO: 46 or at nucleotide 51 of a sequence comprising at least 97% identity to SEQ ID NO: 46,
a TT (homozygous) or TX or TC (heterozygous) genotype for SNP_47 at nucleotide 51 of SEQ ID NO: 47 or at nucleotide 51 of a sequence comprising at least 97% identity to SEQ ID NO: 47,
a CC (homozygous) or CX or CA (heterozygous) genotype for SNP_48 at nucleotide 51 of SEQ ID NO: 48 or at nucleotide 51 of a sequence comprising at least 97% identity to SEQ ID NO: 48, or the following markers:
a CC (homozygous) or CX or CA (heterozygous) genotype for SNP_48 at nucleotide 51 of SEQ ID NO: 48 or at nucleotide 51 of a sequence comprising at least 97% identity to SEQ ID NO: 48,
a GG (homozygous) or GX or GT (heterozygous) genotype for SNP_49 at nucleotide 51 of SEQ ID NO: 49 or at nucleotide 51 of a sequence comprising at least 97% identity to SEQ ID NO: 49,
a TT (homozygous) or TX or TA (heterozygous) genotype for SNP_50 at nucleotide 51 of SEQ ID NO: 50 or at nucleotide 51 of a sequence comprising at least 97% identity to SEQ ID NO: 50,
a AA (homozygous) or AX or AG (heterozygous) genotype for SNP_51 at nucleotide 51 of SEQ ID NO: 51 or at nucleotide 51 of a sequence comprising at least 97% identity to SEQ ID NO: 51,
a GG (homozygous) or GX or GT (heterozygous) genotype for SNP_15 at nucleotide 51 of SEQ ID NO: 15 or at nucleotide 51 of a sequence comprising at least 97% identity to SEQ ID NO: 15.

The introgression fragment comprising the QTL may, thus, be large (comprising SNP_01 to SNP_18), or may be smaller and lack markers, it may e.g. comprise SNP_12 to SNP_15, or SNP_12 to SNP_14, or SNP_14 to SNP_48 or SNP_48 to SNP_15, but it may still confer internal fruit rot resistance on the cultivated cucumber plant, i.e. it can still comprise the QTL (QTL5.1 or a variant). Such smaller introgression fragments are an embodiment of the invention. Plants having smaller introgression fragments (e.g. sub-fragments) which still confer the enhanced internal fruit rot resistance (i.e. contain the QTL5.1 or a variant) can be generated using known techniques, such as fine-mapping or similar techniques. For example by starting with a plant comprising the introgression fragment as found in seeds deposited under accession number NCIMB 43530, or another wild donor comprising QTL5.1, and crossing such a plant with another cultivated cucumber plant and selfing the progeny of said cross, and/or backcrossing the progeny, to generate a population of plants which will contain recombinants having a smaller introgression fragment on chromosome 5, which fragment still confers internal fruit rot resistance in relation to a plant lacking the introgression fragment (such as the genetic control, e.g. plants grown from seeds deposited under NCIMB43531), e.g. a fragment comprising the wild donor genotype or haplotype of markers SNP_12 to SNP_15, or SNP_12 to SNP_14, or SNP_14 to SNP_48 or SNP_48 to SNP_15.

Marker assays can be used to select recombinants and to determine the size of the smaller introgression fragment. One or more of SNP markers or the donor genotype or haplotype may be missing. The cultivated cucumber genotype (or haplotype) is then detected for the SNP marker. The internal fruit rot resistance of plants comprising such a smaller introgression fragment can then be compared in an internal fruit rot assay as described herein. If the percentage of fruits (that develop from spore-inoculated flowers) having internal fruit rot symptoms is significantly reduced compared to the control, then the smaller introgression fragment (or sub-fragment) has retained the QTL5.1.

Alternatively, the same or variant QTL (QTL5.1 or variant QTL5.1) may be introgressed from a different wild donor, whereby optionally not all SNP markers disclosed herein are present, i.e. the SNP haplotype may be slightly different than for the MYCR3 donor. Such alternative wild cucumber sources can be identified using the SNP markers provided herein, by screening germplasm (i.e. accessions of) wild cucumber using a marker assay to detect the genotype or haplotype of markers SNP_01 to SNP_18, or of subgroups thereof, such as 6, 7, 8, 9, 10 consecutive markers, e.g. SNP_12 to SNP_15, or SNP_12 to SNP_14, or SNP_14 to SNP_48 or SNP_48 to SNP_15. Plants comprising the same or variant QTL5.1 from other sources are also an embodiment of the invention. As long as the donor has a SNP haplotype which is identical to the MYCR3 donor described herein for at least 3, 4, 5, 6, 7, 8, 9, 10 or more (or all) of the SNPs of SNP_01 to SNP_18 (especially for at least 3, 4, 5, 6, 7, 8, 9, 10 or more consecutive SNP markers of SNP_01 to SNP_18), more preferably for at least 2, 3, 4, 5, 6, 7, 8, 9, 10 or all 11 markers selected from SNP_12, SNP_45, SNP_13, SNP_14, SNP_46, SNP_47, SNP_48, SNP_49, SNP_50, SNP_51 and SNP_15, the donor may comprise QTL5.1. The skilled person can then introgress the QTL5.1 (or a variant thereof) into cultivated cucumber in order to enhance internal fruit rot resistance as described herein and in order to confirm that the QTL reduces susceptibility to internal fruit rot when present in cultivated cucumber.

As described above, in one embodiment the cultivated cucumber plant of the invention comprises an introgression fragment comprising QTL5.1 and at least a subset of SNP markers with the genotype or haplotype of the wild donor, i.e. for at least 3, 4, 5, 6, 7, 8, 9 or 10 (or more) SNP markers of SNP_01 to SNP_18, especially for at least 3, 4, 5, 6, 7, 8, 9. 10 or more consecutive SNP markers of SNP_01 to SNP_18, more preferably for at least t 2, 3, 4, 5, 6, 7, 8, 9, 10 or all 11 markers selected from SNP_12, SNP_45, SNP_13, SNP_14, SNP_46, SNP_47, SNP_48, SNP_49, SNP_50, SNP_51 and SNP_15. Examples of sub-fragments are fragments which have the same SNP haplotype for one of the following groups of SNP markers as the MYCR3 donor described herein (see also Table 1): SNP_12 to SNP_15, SNP_12 to SNP_14, SNP_14 to SNP_48, SNP_48 to SNP_15. In one aspect the cultivated cucumber plant comprises the same SNP haplotype for all, or all except 1 or 2 markers of SNP_12 to SNP_15, SNP_12 to SNP_14, SNP_14 to SNP_48, SNP_48 to SNP_15.

Thus, the introgression fragment (and a cultivated cucumber plant or plant part, e.g., a cell, comprising the introgression fragment) can be detected in a marker assay by detecting the SNP genotype or haplotype of the introgression fragment (i.e. of the wild relative of cucumber germplasm) of one or more or all of the markers above.

Thus, in one aspect, a Quantitative Trait Locus (QTL5.1) was found to be present on chromosome 5 of a wild donor cucumber which, when transferred (introgressed) into a cultivated cucumber plant, variety or breeding line, and, when present in homozygous form or heterozygous form, confers significantly enhanced internal fruit rot resistance (reduced susceptibility to internal fruit rot) onto the cultivated cucumber plant.

The QTLs (or variant QTLs) can be identified in wild donors and can then be introgressed into cultivated cucumber, e.g. using MAS, i.e. using one or more (or all) of the SNP markers provided herein to detect and/or select progeny plants (e.g. backcross plants) comprising a recombinant chromosome 5. The selected plants, i.e. the cultivated cucumber plants comprising an introgression fragment on chromosome 5, wherein the introgression fragment on chromosome 5 is detectable by the SNP haplotype or genotype of one or more of the SNP markers SNP_01 to SNP_18, one or more of the SNP markers SNP_12 to SNP_15, one or more of the SNP markers SNP_12 to SNP_14, one or more of the SNP markers SNP_14 to SNP_48, one or more of the SNP markers SNP_48 to SNP_15 (as described elsewhere herein) can then be phenotyped in internal fruit rot assays together with the suitable control plants in order to determine whether the introgression fragment indeed causes a reduced susceptibility to internal fruit rot.

Accessions of wild cucumbers are obtainable from the USDA National Plant Germplasm System collection or other seed collections, and can thus be screened for the presence of QTL5.1 using e.g. a marker assay as described herein, and accessions comprising one or more of the SNP haplotype or genotype of the donor (e.g. at least 1, 2, 3, 4, 5, 6, 7, 8, 9, 10, 11 or more SNP markers indicative of QTL5.1, preferably at least 1, 2, 3, 4, 5, 6, 7, 8, 9, 10 or 11 of the SNP markers SNP_12 to SNP_15 as shown in FIG. 6) can be crossed with a cultivated cucumber plant having normal wild-type, non-recombinant chromosomes 5. The F1 or F2 generation (or further generation, such as the F3 or a backcross generation) can then be screened for recombinant plants having the introgression fragment using molecular marker assays described herein, for detecting one or more of the SNP markers.

In a specific embodiment, the introgression fragment comprising the QTL5.1 is derivable from (or derived from) or obtainable from (or obtained from; or as present in) seeds, a representative sample of which has been deposited under accession number NCIMB 43530, or from progeny thereof. The progeny may be any progeny which retain the one or more (or all) SNP markers indicative of (and linked to) the QTL, as described. Thus, progeny are not limited to F1 or F2 progeny of the deposit, but can be any progeny, whether obtained by selfing and/or crossing with another cucumber plant.

In one embodiment the introgression fragment is identifiable by one or more of the markers described elsewhere herein, especially markers SNP_01 to SNP_18 for the introgression fragment on chromosome 5, or a subset of markers, such as one or more of the markers selected from SNP markers SNP_12 to SNP_15, SNP_12 to SNP_14, SNP_14 to SNP_48, SNP_48 to SNP_15 (see FIG. 6). In one aspect the invention provides a cultivated cucumber plant, having a genome of cultivated (domesticated) cucumber which comprises increased resistance to internal fruit rot, wherein the increased resistance is conferred by an introgression fragment on the cultivated cucumber chromosome 5, wherein said introgression fragment is obtained by (or obtainable by) crossing a cultivated plant grown from seeds deposited under NCIMB 43530 or progeny of this plant (which comprises one or more the markers disclosed herein linked to the QTL) with a cultivated cucumber plant. Thus in one aspect the cultivated cucumber plant of the invention comprises the same introgression fragment and the same recombinant chromosome 5 as present in NCIMB 43530 (comprising the wild donor genotype or haplotype for SNP_01 to SNP_18), or it comprises a shorter fragment of that introgression fragment (a sub-fragment), whereby the shorter fragment retains the genetic element conferring enhanced internal fruit rot resistance (QTL5.1), e.g. the fragment comprising the donor haplotype or genotype for SNP_12 to SNP_15, SNP_12 to SNP_14, SNP_14 to SNP_48, SNP_48 to SNP_15.

Thus in one aspect the invention relates to a plant of the invention i.e. a cultivated Cucumis sativus var. sativus plant comprising an introgression fragment from a wild cucumber on chromosome 5 in homozygous or heterozygous form and wherein said introgression fragment is the introgression fragment “as in”/is “identical to”/is “the same as in” the seeds deposited under number NCIMB 43530, or is a shorter fragment (sub fragment) thereof, but still confers internal fruit rot resistance due to the presence of QTL5.1 on the sub-fragment.

In yet another embodiment the invention relates to a plant of the invention i.e. a cultivated Cucumis sativus var. sativus plant comprising an introgression fragment from a wild donor on chromosome 5, in homozygous or heterozygous form, and wherein said introgression fragment is a variant of the introgression fragment found in seeds deposited under number NCIMB 43530, i.e. it comprises the QTL 5.1, but the genomic sequence may be different. As wild accessions will be genetically divergent, the genomic sequence of an introgression fragment comprising QTL5.1 from other wild donors will most likely not be identical to the genomic sequence as introgressed into NCIMB43530, and even the resistance conferring gene (comprising a promoter, introns and exons) may be divergent in nucleotide sequence, but the function will be the same, i.e. conferring enhanced internal fruit rot resistance. The divergence can be seen in that the SNP donor haplotype of certain SNP markers linked to QTL5.1 may be not 100% identical to the donor haplotype found in NCIMB43530. So for example not all of SNP_01 to SNP_18, or not all of SNP_12 to SNP_15 may have the same donor haplotype in other wild donors. As described elsewhere herein, other donors may comprise the same donor SNP haplotype as in NCIMB43530 for e.g. at least 6, 7, 8, 9, 10, 11 or more SNP markers selected from SNP_01 to SNP_18 or especially from SNP_12 to SNP_15 (as shown in FIG. 6), or e.g. at least 6, 7, 8, 9, 10, 11 or more consecutive SNP markers selected from SNP_01 to SNP_18, or e.g. for at least a group of SNP markers selected from SNP_01 to SNP_06, or SNP_06 to SNP_12, SNP_12 to SNP_18 or especially from SNP_12 to SNP_15 (as shown in FIG. 6). However, the internal fruit rot resistance QTL, QTL5.1 (comprising e.g. a variant or ortholog of the fruit rot resistance allele) may still be present in such wild accessions. The skilled person is capable of identifying and introgressing the QTL5.1 comprising region found in other wild cucumbers into cultivated cucumber, e.g. identifying wild cucumber accessions comprising the SNP markers or a subset thereof and transferring these SNP markers (or subset) into a cultivated cucumber plant line or variety and assessing the internal fruit rot resistance of the cultivated plant compared to the control plant lacking the SNP markers (or subset), i.e. lacking the introgression fragment.

In one embodiment a plant or plant part comprising QTL5.1 or a variant thereof is provided, wherein the introgression fragment in the plant or plant part comprising QTL5.1, comprises and/or is detectable by a molecular marker assay which detects the following genotypes or haplotypes for at least 1, preferably at least 2, 3, 4, 5, 6, 7, 8, 9, 10, 11, 12, 13, 14, 15, 16, 17 or 18 Single Nucleotide Polymorphism (SNP) markers selected from the group consisting of:
a) the AA (homozygous) or AX (heterozygous) genotype for SNP_01 at nucleotide 51 of SEQ ID NO: 1 (or at nucleotide 51 of a sequence comprising at least 97% identity to SEQ ID NO: 1),
b) the GG (homozygous) or GX (heterozygous) genotype for SNP_02 at nucleotide 51 of SEQ ID NO: 2 (or at nucleotide 51 of a sequence comprising at least 97% identity to SEQ ID NO: 2),
c) the CC (homozygous) or CX (heterozygous) genotype for SNP_03 at nucleotide 51 of SEQ ID NO: 3 (or at nucleotide 51 of a sequence comprising at least 97% identity to SEQ ID NO: 3),
d) the GG (homozygous) or GX (heterozygous) genotype for SNP_04 at nucleotide 51 of SEQ ID NO: 4 (or at nucleotide 51 of a sequence comprising at least 97% identity to SEQ ID NO: 4),
e) the AA (homozygous) or AX (heterozygous) genotype for SNP_05 at nucleotide 51 of SEQ ID NO: 5 (or at nucleotide 51 of a sequence comprising at least 97% identity to SEQ ID NO: 5),
f) the AA (homozygous) or AX (heterozygous) genotype for SNP_06 at nucleotide 51 of SEQ ID NO: 6 (or at nucleotide 51 of a sequence comprising at least 97% identity to SEQ ID NO: 6),
g) the CC (homozygous) or CX (heterozygous) genotype for SNP_07 at nucleotide 51 of SEQ ID NO: 7 (or at nucleotide 51 of a sequence comprising at least 97% identity to SEQ ID NO: 7),
h) the CC (homozygous) or CX (heterozygous) genotype SNP_08 at nucleotide 51 of SEQ ID NO: 8 (or at nucleotide 51 of a sequence comprising at least 97% identity to SEQ ID NO: 8),
i) the AA (homozygous) or AX (heterozygous) genotype for SNP_09 at nucleotide 51 of SEQ ID NO: 9 (or at nucleotide 51 of a sequence comprising at least 97% identity to SEQ ID NO: 9),
j) the AA (homozygous) or AX (heterozygous) genotype for SNP_10 at nucleotide 51 of SEQ ID NO: 10 (or at nucleotide 51 of a sequence comprising at least 97% identity to SEQ ID NO: 10),
k) the CC (homozygous) or CX (heterozygous) genotype for SNP_11 at nucleotide 51 of SEQ ID NO: 11 (or at nucleotide 51 of a sequence comprising at least 97% identity to SEQ ID NO: 11),
l) the GG (homozygous) or GX (heterozygous) genotype for SNP_12 at nucleotide 51 of SEQ ID NO: 12 (or at nucleotide 51 of a sequence comprising at least 97% identity to SEQ ID NO: 12),
m) the AA (homozygous) or AX (heterozygous) genotype for SNP_45 at nucleotide 51 of SEQ ID NO: 45 (or at nucleotide 51 of a sequence comprising at least 97% identity to SEQ ID NO: 45),
n) the AA (homozygous) or AX (heterozygous) genotype for SNP_13 at nucleotide 51 of SEQ ID NO: 13 (or at nucleotide 51 of a sequence comprising at least 97% identity to SEQ ID NO: 13),
o) the GG (homozygous) or GX (heterozygous) genotype for SNP_14 at nucleotide 51 of SEQ ID NO: 14 (or at nucleotide 51 of a sequence comprising at least 97% identity to SEQ ID NO: 14),
p) the TT (homozygous) or TX (heterozygous) genotype for SNP_46 at nucleotide 51 of SEQ ID NO: 46 (or at nucleotide 51 of a sequence comprising at least 97% identity to SEQ ID NO: 46),
q) the TT (homozygous) or TX (heterozygous) genotype for SNP_47 at nucleotide 51 of SEQ ID NO: 47 or at nucleotide 51 of a sequence comprising at least 97% identity to SEQ ID NO: 47,
r) the CC (homozygous) or CX (heterozygous) genotype for SNP_48 at nucleotide 51 of SEQ ID NO: 48 or at nucleotide 51 of a sequence comprising at least 97% identity to SEQ ID NO: 48,
s) the GG (homozygous) or GX (heterozygous) genotype for SNP_49 at nucleotide 51 of SEQ ID NO: 49 or at nucleotide 51 of a sequence comprising at least 97% identity to SEQ ID NO: 49,
t) the TT (homozygous) or TX (heterozygous) genotype for SNP_50 at nucleotide 51 of SEQ ID NO: 50 or at nucleotide 51 of a sequence comprising at least 97% identity to SEQ ID NO: 50,
u) the AA (homozygous) or AX (heterozygous) genotype for SNP_51 at nucleotide 51 of SEQ ID NO: 51 or at nucleotide 51 of a sequence comprising at least 97% identity to SEQ ID NO: 51,
v) the GG (homozygous) or GX (heterozygous) genotype for SNP_15 at nucleotide 51 of SEQ ID NO: 15 (or at nucleotide 51 of a sequence comprising at least 97% identity to SEQ ID NO: 15),
w) the CC (homozygous) or CX (heterozygous) genotype for SNP_16 at nucleotide 51 of SEQ ID NO: 16 (or at nucleotide 51 of a sequence comprising at least 97% identity to SEQ ID NO: 16),
x) the AA (homozygous) or AX (heterozygous) genotype for SNP_17 at nucleotide 51 of SEQ ID NO: 17 (or at nucleotide 51 of a sequence comprising at least 97% identity to SEQ ID NO: 17) and
y) the GG (homozygous) or GX (heterozygous) genotype for SNP_18 at nucleotide 51 of SEQ ID NO: 18 (or at nucleotide 51 of a sequence comprising at least 97% identity to SEQ ID NO: 18),
wherein preferably at least the SNP genotype or haplotype of SNP_12 to SNP_15 above, i.e. of 1) to v) are detected and comprised in the plant or plant part.

It is clear that when the introgression fragment is heterozygous and only found on one chromosome 5 of the pair, the first nucleotide mentioned above is the nucleotide of the donor (of the introgression fragment, which is part of the recombinant chromosome 5), while the second nucleotide is the nucleotide of the chromosome 5 lacking the introgression fragment, e.g. the non-recombinant chromosome 5 of the recurrent parent. The second nucleotide is herein indicated as X, meaning any nucleotide (A, G, T or C), as this second nucleotide can vary, depending on the genetic background of the cucumber line.

In one aspect nucleotide X is the nucleotide of the recurrent parent as present in the deposited seeds and as indicated in Table 1 and herein below.

Thus, in one embodiment a plant or plant part comprising QTL5.1 or a variant thereof is provided, wherein the introgression fragment in the plant or plant part comprising QTL5.1, comprises and/or is detectable by a molecular marker assay which detects the following genotypes or haplotypes for at least 3, preferably at least 4, 5, 6, 7, 8, 9, 10, 11, 12, 13, 14, 15, 16, 17 or 18 Single Nucleotide Polymorphism (SNP) markers selected from the group consisting of:
a) the AA (homozygous) or AG (heterozygous) genotype for SNP_01 at nucleotide 51 of SEQ ID NO: 1 (or at nucleotide 51 of a sequence comprising at least 97% identity to SEQ ID NO: 1),
b) the GG (homozygous) or GA (heterozygous) genotype for SNP_02 at nucleotide 51 of SEQ ID NO: 2 (or at nucleotide 51 of a sequence comprising at least 97% identity to SEQ ID NO: 2),
c) the CC (homozygous) or CT (heterozygous) genotype for SNP_03 at nucleotide 51 of SEQ ID NO: 3 (or at nucleotide 51 of a sequence comprising at least 97% identity to SEQ ID NO: 3),
d) the GG (homozygous) or GA (heterozygous) genotype for SNP_04 at nucleotide 51 of SEQ ID NO: 4 (or at nucleotide 51 of a sequence comprising at least 97% identity to SEQ ID NO: 4),
e) the AA (homozygous) or AC (heterozygous) genotype for SNP_05 at nucleotide 51 of SEQ ID NO: 5 (or at nucleotide 51 of a sequence comprising at least 97% identity to SEQ ID NO: 5),
f) the AA (homozygous) or AG (heterozygous) genotype for SNP_06 at nucleotide 51 of SEQ ID NO: 6 (or at nucleotide 51 of a sequence comprising at least 97% identity to SEQ ID NO: 6),
g) the CC (homozygous) or CT (heterozygous) genotype for SNP_07 at nucleotide 51 of SEQ ID NO: 7 (or at nucleotide 51 of a sequence comprising at least 97% identity to SEQ ID NO: 7),
h) the CC (homozygous) or CA (heterozygous) genotype SNP_08 at nucleotide 51 of SEQ ID NO: 8 (or at nucleotide 51 of a sequence comprising at least 97% identity to SEQ ID NO: 8),
i) the AA (homozygous) or AG (heterozygous) genotype for SNP_09 at nucleotide 51 of SEQ ID NO: 9 (or at nucleotide 51 of a sequence comprising at least 97% identity to SEQ ID NO: 9),
j) the AA (homozygous) or AG (heterozygous) genotype for SNP_10 at nucleotide 51 of SEQ ID NO: 10 (or at nucleotide 51 of a sequence comprising at least 97% identity to SEQ ID NO: 10),
k) the CC (homozygous) or CT (heterozygous) genotype for SNP_11 at nucleotide 51 of SEQ ID NO: 11 (or at nucleotide 51 of a sequence comprising at least 97% identity to SEQ ID NO: 11),
l) the GG (homozygous) or GA (heterozygous) genotype for SNP_12 at nucleotide 51 of SEQ ID NO: 12 (or at nucleotide 51 of a sequence comprising at least 97% identity to SEQ ID NO: 12),
m) the AA (homozygous) or AG (heterozygous) genotype for SNP_45 at nucleotide 51 of SEQ ID NO: 45 or at nucleotide 51 of a sequence comprising at least 97% identity to SEQ ID NO: 45,
n) the AA (homozygous) or AG (heterozygous) genotype for SNP_13 at nucleotide 51 of SEQ ID NO: 13 (or at nucleotide 51 of a sequence comprising at least 97% identity to SEQ ID NO: 13),
o) the GG (homozygous) or GA (heterozygous) genotype for SNP_14 at nucleotide 51 of SEQ ID NO: 14 (or at nucleotide 51 of a sequence comprising at least 97% identity to SEQ ID NO: 14),
p) the TT (homozygous) or TC (heterozygous) genotype for SNP_46 at nucleotide 51 of SEQ ID NO: 46 or at nucleotide 51 of a sequence comprising at least 97% identity to SEQ ID NO: 46,
q) the TT (homozygous) or TC (heterozygous) genotype for SNP_47 at nucleotide 51 of SEQ ID NO: 47 or at nucleotide 51 of a sequence comprising at least 97% identity to SEQ ID NO: 47,
r) the CC (homozygous) or CA (heterozygous) genotype for SNP_48 at nucleotide 51 of SEQ ID NO: 48 or at nucleotide 51 of a sequence comprising at least 97% identity to SEQ ID NO: 48,
s) the GG (homozygous) or GT (heterozygous) genotype for SNP_49 at nucleotide 51 of SEQ ID NO: 49 or at nucleotide 51 of a sequence comprising at least 97% identity to SEQ ID NO: 49,
t) the TT (homozygous) or TA (heterozygous) genotype for SNP_50 at nucleotide 51 of SEQ ID NO: 50 or at nucleotide 51 of a sequence comprising at least 97% identity to SEQ ID NO: 50,
u) the AA (homozygous) or AG (heterozygous) genotype for SNP_51 at nucleotide 51 of SEQ ID NO: 51 or at nucleotide 51 of a sequence comprising at least 97% identity to SEQ ID NO: 51,
v) the GG (homozygous) or GT (heterozygous) genotype for SNP_15 at nucleotide 51 of SEQ ID NO: 15 (or at nucleotide 51 of a sequence comprising at least 97% identity to SEQ ID NO: 15),
w) the CC (homozygous) or CA (heterozygous) genotype for SNP_16 at nucleotide 51 of SEQ ID NO: 16 (or at nucleotide 51 of a sequence comprising at least 97% identity to SEQ ID NO: 16),
x) the AA (homozygous) or AG (heterozygous) genotype for SNP_17 at nucleotide 51 of SEQ ID NO: 17 (or at nucleotide 51 of a sequence comprising at least 97% identity to SEQ ID NO: 17) and
y) the GG (homozygous) or GA (heterozygous) genotype for SNP_18 at nucleotide 51 of SEQ ID NO: 18 (or at nucleotide 51 of a sequence comprising at least 97% identity to SEQ ID NO: 18),
wherein preferably at least the SNP genotype or haplotype of SNP_12 to SNP_15 above, i.e. of 1) to v) are detected and comprised in the plant or plant part.

In one aspect the genotype or haplotype of at least 3, or at least 4, 5, 6, 7, 8, 9, 10, 11, 12, 13, 14, 15, 16, 17, 18 markers which are detected (and which are present on the introgression fragment) are consecutive markers. That means the introgression fragment comprises the genotype (homozygous introgression fragment) or haplotype (heterozygous introgression fragment) for at least 3, 4, 5, 6, 7, 8, 9, 10 or more markers, whereby these are consecutive markers, e.g. three consecutive markers are for example SNP_12, SNP_45, SNP_13, SNP_14. The genotype of the plant or plant part comprising the introgression fragment in homozygous form would then be GG-AA-AA-GG.

Without being bound in any way, the marker haplotype of any 3 or more consecutive markers out of the 18 markers is a unique identifier of the introgression fragment of the donor and/or the marker haplotype of any 6 or more of the 18 markers is a unique identifier of the introgression fragment of the donor. As the QTL5.1 was mapped to be located between SNP_12 and SNP_15, the genotype or haplotype of at least 6, 7, 8 or more of these 11 markers is a unique identifier of the introgression fragment comprising the QTL.

Herein the haplotype is indicted to be the haplotype of a specific SNP nucleotide in a specific sequence (SEQ ID Numbers), or in a sequence comprising at least 97% sequence identity (or 98% or 99%) to the specific sequence (SEQ ID Number). For example the AA (homozygous) or AG (heterozygous) genotype for SNP_01 at nucleotide 51 of SEQ ID NO: 1 “or at nucleotide 51 of a sequence comprising at least 97% identity to SEQ ID NO: 1”. The sequence of SEQ ID Number flanking the SNP (i.e. the 50 nucleotides preceding the SNP and the 50 nucleotides following the SNP) are herein the sequence of the wild donor MYCR3. However, there may be very slight sequence variation or sequencing errors in the flanking sequence surrounding the SNPs. Therefore, such flanking sequences, which comprise e.g. one, two or three nucleotides which are different from the flanking sequences provided herein in the specific SEQ ID Numbers are encompassed herein.

As mentioned, the SNP haplotype of one or more or all SNP markers linked to QTL5.1 (or a variant thereof) is useful for the genetic identification of the donor fragment and/or introgression fragment in combination with the phenotype conferred by the QTL, and thus for the identification of wild donors comprising the QTL5.1 (or a variant thereof), for generating cucumber plants or plant parts comprising the QTL5.1 (or a variant thereof) and/or detecting cucumber plants or plant parts comprising the QTL5.1 (or a variant thereof).

Therefore, in one aspect a method of using the SNP haplotype of one or more or all SNP markers linked to QTL5.1 (or a variant thereof) for breeding cucumber plants comprising the QTL5.1 (or a variant thereof) and/or for screening donor accessions or cultivated cucumber lines or varieties for the presence of QTL5.1 is one embodiment herein.

Thus, in one embodiment a method for detecting, selecting and/or breeding a plant or plant part comprising QTL5.1 or a variant thereof is provided, comprising carrying out a molecular marker assay and optionally selecting a plant or plant part which comprises the following genotypes or haplotypes for at least 3, preferably at least 4, 5, 6, 7, 8, 9, 10, 11, 12, 13, 14, 15, 16, 17 or 18 Single Nucleotide Polymorphism (SNP) markers selected from the group consisting of:
a) the AA (homozygous) or AX or AG (heterozygous) genotype for SNP_01 at nucleotide 51 of SEQ ID NO: 1 (or at nucleotide 51 of a sequence comprising at least 97% identity to SEQ ID NO: 1),
b) the GG (homozygous) or GX or GA (heterozygous) genotype for SNP_02 at nucleotide 51 of SEQ ID NO: 2 (or at nucleotide 51 of a sequence comprising at least 97% identity to SEQ ID NO: 2),
c) the CC (homozygous) or CX or CT (heterozygous) genotype for SNP_03 at nucleotide 51 of SEQ ID NO: 3 (or at nucleotide 51 of a sequence comprising at least 97% identity to SEQ ID NO: 3),
d) the GG (homozygous) or GX or GA (heterozygous) genotype for SNP_04 at nucleotide 51 of SEQ ID NO: 4 (or at nucleotide 51 of a sequence comprising at least 97% identity to SEQ ID NO: 4),
e) the AA (homozygous) or AX or AC (heterozygous) genotype for SNP_05 at nucleotide 51 of SEQ ID NO: 5 (or at nucleotide 51 of a sequence comprising at least 97% identity to SEQ ID NO: 5),
f) the AA (homozygous) or AX or AG (heterozygous) genotype for SNP_06 at nucleotide 51 of SEQ ID NO: 6 (or at nucleotide 51 of a sequence comprising at least 97% identity to SEQ ID NO: 6),
g) the CC (homozygous) or CX or CT (heterozygous) genotype for SNP_07 at nucleotide 51 of SEQ ID NO: 7 (or at nucleotide 51 of a sequence comprising at least 97% identity to SEQ ID NO: 7),
h) the CC (homozygous) or CX or CA (heterozygous) genotype SNP_08 at nucleotide 51 of SEQ ID NO: 8 (or at nucleotide 51 of a sequence comprising at least 97% identity to SEQ ID NO: 8),
i) the AA (homozygous) or AX or AG (heterozygous) genotype for SNP_09 at nucleotide 51 of SEQ ID NO: 9 (or at nucleotide 51 of a sequence comprising at least 97% identity to SEQ ID NO: 9),
j) the AA (homozygous) or AX or AG (heterozygous) genotype for SNP_10 at nucleotide 51 of SEQ ID NO: 10 (or at nucleotide 51 of a sequence comprising at least 97% identity to SEQ ID NO: 10),
k) the CC (homozygous) or CX or CT (heterozygous) genotype for SNP_11 at nucleotide 51 of SEQ ID NO: 11 (or at nucleotide 51 of a sequence comprising at least 97% identity to SEQ ID NO: 11),
l) the GG (homozygous) or GX or GA (heterozygous) genotype for SNP_12 at nucleotide 51 of SEQ ID NO: 12 (or at nucleotide 51 of a sequence comprising at least 97% identity to SEQ ID NO: 12),
m) the AA (homozygous) or AX or AG (heterozygous) genotype for SNP_45 at nucleotide 51 of SEQ ID NO: 45 or at nucleotide 51 of a sequence comprising at least 97% identity to SEQ ID NO: 45,
n) the AA (homozygous) or AX or AG (heterozygous) genotype for SNP_13 at nucleotide 51 of SEQ ID NO: 13 (or at nucleotide 51 of a sequence comprising at least 97% identity to SEQ ID NO: 13),
o) the GG (homozygous) or GX or GA (heterozygous) genotype for SNP_14 at nucleotide 51 of SEQ ID NO: 14 (or at nucleotide 51 of a sequence comprising at least 97% identity to SEQ ID NO: 14),
p) the TT (homozygous) or TX or TC (heterozygous) genotype for SNP_46 at nucleotide 51 of SEQ ID NO: 46 or at nucleotide 51 of a sequence comprising at least 97% identity to SEQ ID NO: 46,
q) the TT (homozygous) or TX or TC (heterozygous) genotype for SNP_47 at nucleotide 51 of SEQ ID NO: 47 or at nucleotide 51 of a sequence comprising at least 97% identity to SEQ ID NO: 47,
r) the CC (homozygous) or CX or CA (heterozygous) genotype for SNP_48 at nucleotide 51 of SEQ ID NO: 48 or at nucleotide 51 of a sequence comprising at least 97% identity to SEQ ID NO: 48,
s) the GG (homozygous) or GX or GT (heterozygous) genotype for SNP_49 at nucleotide 51 of SEQ ID NO: 49 or at nucleotide 51 of a sequence comprising at least 97% identity to SEQ ID NO: 49,
t) the TT (homozygous) or TX or TA (heterozygous) genotype for SNP_50 at nucleotide 51 of SEQ ID NO: 50 or at nucleotide 51 of a sequence comprising at least 97% identity to SEQ ID NO: 50,
u) the AA (homozygous) or AX or AG (heterozygous) genotype for SNP_51 at nucleotide 51 of SEQ ID NO: 51 or at nucleotide 51 of a sequence comprising at least 97% identity to SEQ ID NO: 51,
v) the GG (homozygous) or GX or GT (heterozygous) genotype for SNP_15 at nucleotide 51 of SEQ ID NO: 15 (or at nucleotide 51 of a sequence comprising at least 97% identity to SEQ ID NO: 15),
w) the CC (homozygous) or CX or CA (heterozygous) genotype for SNP_16 at nucleotide 51 of SEQ ID NO: 16 (or at nucleotide 51 of a sequence comprising at least 97% identity to SEQ ID NO: 16),
x) the AA (homozygous) or AX or AG (heterozygous) genotype for SNP_17 at nucleotide 51 of SEQ ID NO: 17 (or at nucleotide 51 of a sequence comprising at least 97% identity to SEQ ID NO: 17) and
y) the GG (homozygous) or GX or GA (heterozygous) genotype for SNP_18 at nucleotide 51 of SEQ ID NO: 18 (or at nucleotide 51 of a sequence comprising at least 97% identity to SEQ ID NO: 18),
wherein preferably at least the SNP genotype or haplotype of one or more or all of markers of SNP_12 to SNP_15 above, i.e. of 1) to v) is detected and used to select a plant or plant part.

The plant or plant parts may be of one or more wild donor accessions, or of one or more breeding lines or varieties. X may be any nucleotide.

Optionally the selected plant or plants may be phenotyped for internal fruit rot resistance.

In one aspect the location of the QTL5.1 is further fine mapped, so that it is known in which region in between SNP_12 and SNP_15 the QTL5.1 is located. Fine mapping can be done by generating recombination events in the chromosome 5 region, so that different plant lines comprise different size introgression fragments. The QTL5.1 can then be found to be present in one or more of the following subregions selected from the regions from SNP_12 to SNP_14, from SNP_14 to SNP_48, from SNP_48 to SNP_15. The line comprising the QTL5.1 will show the increase in DB fruit rot resistance conferred by the QTL.

In one embodiment the presence of the introgression fragment, or the chromosome 5 region (or variant or orthologous chromosome 5 region), comprising QTL5.1 (or a variant thereof), is detectable by a molecular marker assay which detects the wild donor SNP haplotype or genotype of at least 3, preferably at least 4 or more Single Nucleotide Polymorphism (SNP) markers of the sub-groups consisting of: SNP_12 to SNP_15, SNP_12 to SNP_14, from SNP_14 to SNP_48, from SNP_48 to SNP_15.

The SNP genotype refers to two nucleotides, and genomic sequences comprising one of these two nucleotides, one on each chromosome 5. So a plant having a AA genotype for SNP_01 has an identical nucleotide (A) on both chromosomes (i.e. is homozygous for the introgression fragment), while a plant having e.g. an AG genotype for SNP_01 has one chromosome with an A at nucleotide 51 of SEQ ID NO: 1 (or at the equivalent nucleotide of a genomic sequence comprising at least 97% sequence identity to SEQ ID NO:1) and one chromosome with a G at nucleotide 51 of SEQ ID NO: 1 and is heterozygous for the introgression fragment. The nucleotide for the chromosome lacking the introgression fragment may also be indicated as X, wherein X is selected from any nucleotide (A, T, G, C). The genotype for SNP_01, wherein the introgression fragment is in heterozygous form, may thus be indicated as AX. The same applies to the other SNP markers. As the genomic sequences around the SNP markers provided herein may vary slightly, e.g. in introgression fragments from other wild donors (i.e. variants or orthologous chromosome 5 regions) it is clear that the nucleotide sequences before and after the SNP may not be 100% identical to the sequences provided herein. Therefore sequences having at least 97% sequence identity to the sequences provided herein (when aligned over the entire length as defined), but which comprise the same SNP genotype, are encompassed herein.

In one aspect, the introgression fragment, or the chromosome 5 region (or variant or orthologous chromosome 5 region) comprising the QTL (QTL5.1 or variant), which is detectable by the above one or more markers is from a wild cucumber. In one aspect it is the same introgression fragment as found on chromosome 5 in seeds deposited under accession number NCIMB43530, or a smaller fragment (sub-fragment) retaining the QTL.

SNP markers SNP_01 (at nucleotide 3701817 of chromosome 5) to SNP_18 (at nucleotide 4028826 of chromosome 5) span a region of about 0.33 Mb on chromosome 5 and on this donor region the QTL is found. It has been fine mapped to lie in between SNP_12 at nucleotide 3823864 of chromosome 5 and SNP_15 at nucleotide 3967955 of chromosome 5, i.e. a region of 144091 bases. In one aspect the introgression fragment on chromosome 5 is equal to or less than 1 Mb in size, preferably equal to or less than 0.5 Mb in size, more preferably equal to or less than 0.33 Mb. In one aspect the introgression fragment is at least 0.1 Mb, 0.2 Mb, 1.0 Mb, 0.45 Mb, 0.3 Mb in size, or it may even only be 145 000 bases in size, or even less after further fine mapping. Thus, various ranges of introgression fragment sizes are encompassed herein, such as fragments less than 1 Mb but preferably more than 0.1 Mb which retain the QTL5.1 and the donor genotype or donor haplotype for one or more of the SNP markers of SNP_01 to SNP_18, especially SNP_12 to SNP_15 (as shown in FIG. 6), or for one or more of the SNP markers of a subgroup of SNP markers comprising QTL5.1 selected from: SNP_12 to SNP_15, SNP_12 to SNP_14, SNP_14 to SNP_48 and SNP_48 to SNP_15. As mentioned before, the location of the QTL5.1 in the region spanning SNP_12 to SNP_15 can be determined by further fine-mapping and recombinants comprising QTL5.1 and a smaller introgression fragment (sub-fragment) can be generated. The size of an introgression fragment can be easily determined by e.g. whole genome sequencing or Next Generation Sequencing, e.g. as described in Qi et al. 2013 (supra) or in Huang et al. 2009 (supra). Especially introgression regions can be easily distinguished from cultivated genomic regions due to the larger amount of genetic variation (SNPs, INDELs, etc.) in the introgression region.

To obtain the introgression fragment present on chromosome 5 from the deposited seeds (NCIMB 43530), i.e. to transfer the introgression fragment comprising the QTL to another cultivated cucumber plant, a plant is grown from the seed and the plant is crossed with a cultivated cucumber plant to obtain F1 seeds. As NCIMB 43530 contains two recombinant chromosomes 5 (comprising the introgression fragment comprising QTL5.1 in homozygous form) all of the F1 seed and plants grown therefrom will contain one recombinant chromosome 5 from the NCIMB 43530 parent and one non-recombinant chromosome 5 from the other cultivated parent. By further selfing and/or crossing and/or backcrossing, QTL5.1 can be transferred into any cucumber breeding line or variety. Thus, by traditional breeding one can transfer the recombinant chromosome 5 from NCIMB 43530 into other cultivated cucumber lines or varieties. Progeny plants which comprise the QTL5.1 can be screened for, and selected for, by the presence of the donor genotype or haplotype for one or more of the above SNP markers.

To generate shorter introgression fragments, e.g. sub-fragments of the fragment present in NCIMB 43530, meiosis needs to take place and plants comprising the recombinant chromosomes 5, and especially new meiotic recombination events within the introgression fragment, need to be identified. For example, seeds of NCIMB43530 can be selfed one or more times to produce F1, F2 or F3 plants (or further selfing generations), and/or F1, F2 or F3 plants (etc.) comprising the recombinant chromosome 5 can be backcrossed to a cultivated parent. Plants which comprise the recombinant chromosome 5 can be screened for, and selected for, by the presence of the donor genotype or haplotype of one or more of the above SNP markers in order to identify plants comprising a smaller introgression fragment. Such new recombinants can then be tested for the presence of the QTL5.1 on the smaller introgression fragment by determining the internal fruit rot resistance compared to the (genetic) control lacking the introgression fragment.

Similarly, cultivated cucumber plants comprising QTL5.1 (or a variant thereof) can be generated and/or identified using different methods. For example, to obtain a cultivated cucumber plant comprising a introgression fragment from a wild donor of cucumber, first a wild donor is identified which comprises the donor SNP genotype or haplotype one or more of the SNP markers linked to QTL5.1 disclosed herein, e.g. any one, or more, or all of the markers described herein above. The identified donor plant is crossed with a cultivated cucumber plant to obtain F1 seeds. The F1 can be selfed to produce F2, F3, etc. plants, and/or F2 plants or F3 plants, etc., can be backcrossed to the cultivated cucumber parent. Plants which are comprising QTL5.1 (or a variant thereof) can be screened for, and/or selected for, by the presence of the donor genotype or haplotype of one or more of the above SNP markers and/or screened for, and/or selected for, an increased internal fruit rot resistance phenotype compared to e.g. the initial cultivated parent (lacking the introgression). Alternatively or in addition, QTL mapping or sequencing can be carried out in order to identify further molecular markers linked to the QTL5.1 (or a variant thereof) and/or to generate cultivated cucumber plants comprising an introgression fragment on chromosome 5 which confers internal fruit rot resistance.

In one embodiment the introgression fragment in a cultivated cucumber plant or plant part, or a donor fragment in a wild cucumber plant or plant part, comprising QTL5.1 (or a variant thereof), comprises:
a) the donor SNP haplotype of at least 3, 4, 5, 6 or more SNP markers of SNP_01 to SNP_18, preferably of at least 3, 4, 5 or more of SNP_12 to SNP_15;
b) the donor SNP haplotype of at least 3, 4, 5, 6 or more consecutive SNP markers of SNP_01 to SNP_18 preferably of at least 3, 4, 5 or more of SNP_12 to SNP_15;
c) the donor SNP haplotype of SNP_12 to SNP_15, and/or of SNP_12 to SNP_14, and/or of SNP_14 to SNP_48 and/or of SNP_48 to SNP_15;
   wherein the donor SNP haplotype is:
an Adenine for SNP_01 at nucleotide 51 of SEQ ID NO: 1 (or at nucleotide 51 of a sequence comprising at least 97% identity to SEQ ID NO: 1),
a Guanine for SNP_02 at nucleotide 51 of SEQ ID NO: 2 (or at nucleotide 51 of a sequence comprising at least 97% identity to SEQ ID NO: 2),
a Cytosine for SNP_03 at nucleotide 51 of SEQ ID NO: 3 (or at nucleotide 51 of a sequence comprising at least 97% identity to SEQ ID NO: 3),
a Guanine for SNP_04 at nucleotide 51 of SEQ ID NO: 4 (or at nucleotide 51 of a sequence comprising at least 97% identity to SEQ ID NO: 4),
an Adenine for SNP_05 at nucleotide 51 of SEQ ID NO: 5 (or at nucleotide 51 of a sequence comprising at least 97% identity to SEQ ID NO: 5),
an Adenine for SNP_06 at nucleotide 51 of SEQ ID NO: 6 (or at nucleotide 51 of a sequence comprising at least 97% identity to SEQ ID NO: 6),
a Cytosine for SNP_07 at nucleotide 51 of SEQ ID NO: 7 (or at nucleotide 51 of a sequence comprising at least 97% identity to SEQ ID NO: 7),
a Cytosine for SNP_08 at nucleotide 51 of SEQ ID NO: 8 (or at nucleotide 51 of a sequence comprising at least 97% identity to SEQ ID NO: 8),
an Adenine for SNP_09 at nucleotide 51 of SEQ ID NO: 9 (or at nucleotide 51 of a sequence comprising at least 97% identity to SEQ ID NO: 9),
an Adenine for SNP_10 at nucleotide 51 of SEQ ID NO: 10 (or at nucleotide 51 of a sequence comprising at least 97% identity to SEQ ID NO: 10),
a Cytosine for SNP_11 at nucleotide 51 of SEQ ID NO: 11 (or at nucleotide 51 of a sequence comprising at least 97% identity to SEQ ID NO: 11),
a Guanine for SNP_12 at nucleotide 51 of SEQ ID NO: 12 (or at nucleotide 51 of a sequence comprising at least 97% identity to SEQ ID NO: 12),
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 (or at nucleotide 51 of a sequence comprising at least 97% identity to SEQ ID NO: 13),
a Guanine for SNP_14 at nucleotide 51 of SEQ ID NO: 14 (or at nucleotide 51 of a sequence comprising at least 97% identity to SEQ ID NO: 14),
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 (or at nucleotide 51 of a sequence comprising at least 97% identity to SEQ ID NO: 15),
a Cytosine for SNP_16 at nucleotide 51 of SEQ ID NO: 16 (or at nucleotide 51 of a sequence comprising at least 97% identity to SEQ ID NO: 16),
an Adenine for SNP_17 at nucleotide 51 of SEQ ID NO: 17 (or at nucleotide 51 of a sequence comprising at least 97% identity to SEQ ID NO: 17) and
a Guanine for SNP_18 at nucleotide 51 of SEQ ID NO: 18 (or at nucleotide 51 of a sequence comprising at least 97% identity to SEQ ID NO: 18).

Thus, different wild cucumber accessions (or selections or selfings thereof) can be screened for the SNP haplotype of a), b) or c) above to identify other donor accessions than MYCR3 which comprise QTL5.1. Optionally the donor accessions can be screened also for the presence of QTL3.1 and/or phenotypically for the susceptibility or resistance to internal fruit rot caused by DB. A wild cucumber accession identified or selected for having the donor SNP haplotype of a), b) or c) can then be used to backcross QTL5.1 into cultivated cucumber.

Thereby a cultivated cucumber can be generated comprising an introgression fragment comprising QTL5.1 (or a variant thereof) and comprising the donor SNP haplotype of a), b) or c) above. The cultivated cucumber is preferably tested phenotypically for the susceptibility or resistance to internal fruit rot caused by DB. The base-line susceptibility should be significantly reduced in the cultivated cucumber plant comprising QTL5.1. As QTL5.1 and QTL3.1 are additive, the effect of QTL5.1 is optionally, in one aspect, determined in combination with QTL3.1, present together in the cultivated cucumber.

Also provided are seeds from which a plant of the invention can be grown, as are cucumber fruits harvested from a plant of the invention and comprising the recombinant chromosome 5 in their genome. Likewise a plant cell, tissue or plant part of a plant or of a seed is provided comprising at least one recombinant chromosome 5, wherein said recombinant chromosome 5 comprises an introgression fragment from a wild cucumber and wherein said introgression fragment comprises QTL5.1 (or a variant thereof) conferring significantly resistance to internal fruit rot.

In one embodiment also a method for identifying and/or selecting plant or plant part comprising an introgression fragment in a cultivated cucumber plant or plant part, or a donor fragment in a wild cucumber plant or plant part, comprising QTL5.1 (or a variant thereof), is provided comprising DNA analysis to identify and/or select a plant or plant part, wherein the fragment comprises:
a) the donor SNP haplotype of at least 3, 4, 5, 6 or more SNP markers of SNP_01 to SNP_18, preferably of at least 3, 4, 5 or more of SNP_12 to SNP_15;
b) the donor SNP haplotype of at least 3, 4, 5, 6 or more consecutive SNP markers of SNP_01 to SNP_18 preferably of at least 3, 4, 5 or more of SNP_12 to SNP_15;
c) the donor SNP haplotype of SNP_12 to SNP_15, and/or of SNP_12 to SNP_14, and/or of SNP_14 to SNP_48 and/or of SNP_48 to SNP_15;
   wherein the donor SNP haplotype is:
an Adenine for SNP_01 at nucleotide 51 of SEQ ID NO: 1 (or at nucleotide 51 of a sequence comprising at least 97% identity to SEQ ID NO: 1),
a Guanine for SNP_02 at nucleotide 51 of SEQ ID NO: 2 (or at nucleotide 51 of a sequence comprising at least 97% identity to SEQ ID NO: 2),
a Cytosine for SNP_03 at nucleotide 51 of SEQ ID NO: 3 (or at nucleotide 51 of a sequence comprising at least 97% identity to SEQ ID NO: 3),
a Guanine for SNP_04 at nucleotide 51 of SEQ ID NO: 4 (or at nucleotide 51 of a sequence comprising at least 97% identity to SEQ ID NO: 4),
an Adenine for SNP_05 at nucleotide 51 of SEQ ID NO: 5 (or at nucleotide 51 of a sequence comprising at least 97% identity to SEQ ID NO: 5),
an Adenine for SNP_06 at nucleotide 51 of SEQ ID NO: 6 (or at nucleotide 51 of a sequence comprising at least 97% identity to SEQ ID NO: 6),
a Cytosine for SNP_07 at nucleotide 51 of SEQ ID NO: 7 (or at nucleotide 51 of a sequence comprising at least 97% identity to SEQ ID NO: 7),
a Cytosine for SNP_08 at nucleotide 51 of SEQ ID NO: 8 (or at nucleotide 51 of a sequence comprising at least 97% identity to SEQ ID NO: 8),
an Adenine for SNP_09 at nucleotide 51 of SEQ ID NO: 9 (or at nucleotide 51 of a sequence comprising at least 97% identity to SEQ ID NO: 9),
an Adenine for SNP_10 at nucleotide 51 of SEQ ID NO: 10 (or at nucleotide 51 of a sequence comprising at least 97% identity to SEQ ID NO: 10),
a Cytosine for SNP_11 at nucleotide 51 of SEQ ID NO: 11 (or at nucleotide 51 of a sequence comprising at least 97% identity to SEQ ID NO: 11),
a Guanine for SNP_12 at nucleotide 51 of SEQ ID NO: 12 (or at nucleotide 51 of a sequence comprising at least 97% identity to SEQ ID NO: 12),
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 (or at nucleotide 51 of a sequence comprising at least 97% identity to SEQ ID NO: 13),
a Guanine for SNP_14 at nucleotide 51 of SEQ ID NO: 14 (or at nucleotide 51 of a sequence comprising at least 97% identity to SEQ ID NO: 14),
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49, a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 (or at nucleotide 51 of a sequence comprising at least 97% identity to SEQ ID NO: 15),
a Cytosine for SNP_16 at nucleotide 51 of SEQ ID NO: 16 (or at nucleotide 51 of a sequence comprising at least 97% identity to SEQ ID NO: 16),
an Adenine for SNP_17 at nucleotide 51 of SEQ ID NO: 17 (or at nucleotide 51 of a sequence comprising at least 97% identity to SEQ ID NO: 17) and
a Guanine for SNP_18 at nucleotide 51 of SEQ ID NO: 18 (or at nucleotide 51 of a sequence comprising at least 97% identity to SEQ ID NO: 18),
The DNA analysis in the above method can involve e.g. SNP genotyping, DNA sequencing, PCR analysis or other methods to determine the SNP haplotype of the plant or plant part.

### Cucumber Plants Comprising an Introgression Fragment on Chromosome 3 (DB Fruit Rot Resistance QTL 3.1)

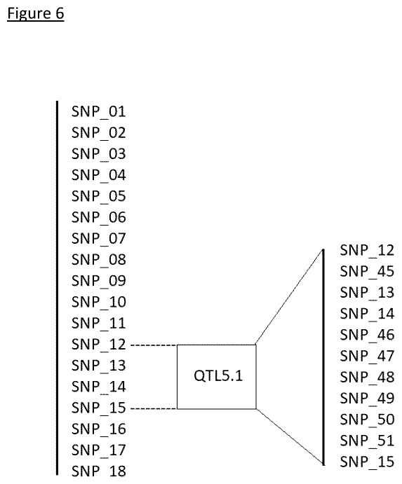

QTL3.1 was initially mapped to the region between SNP_19 at nucleotide 51 of SEQ ID NO: 19 (or in a variant thereof) and SNP_42 at nucleotide 51 of SEQ ID NO: 42 (or a variant thereof). Later it was fine-mapped to the region between SNP_35 and 659 nt downstream of SNP_36, and it was found that the plant comprising the QTL actually contained a duplication of the region between SNP_35 and 659 nt downstream of SNP_36 on chromosome 3, depicted in SEQ ID NO: 72 and 73 (as part of a larger duplication on chromosome 3 which comprises SNP_29 to SNP_36 and starts at SEQ ID NO: 83 and ends at SEQ ID NO: 84). The region between SNP_35 and 659 nt downstream of SNP_36 (i.e. SEQ ID NO: 72 and 73, see FIG. 7) contains 8 OGD genes and one gene encoding a HR-like protein. The (homozygous) resistant plant therefore contained 32 OGD genes and four genes encoding the HR like protein, while the susceptible plant contained 16 OGD genes and two genes encoding the HR-like protein. The introgression fragment from the donor therefore turned out to be structural variation, also referred to as copy number variation (CNV) by comprising a duplication of the region containing 9 genes.

The embodiments below therefore apply to the introgression of the larger region, as present in the deposited seeds, i.e. the region defined by SNP_19 to SNP_42, whereby SNP_19 to SNP_29 and SNP_37 to SNP_42 flank the duplicated region, which comprises SNP_29 to SNP_36 (including the QTL3.1) or smaller regions comprising the duplicated region. As all SNP markers (SNP_19 to SNP_42) are polymorphic between the resistant and susceptible plant, any of the markers or combination of markers (SNP haplotype) can be used to select plants comprising the duplication of the 9 genes. In one aspect the donor SNP genotype or haplotype of one or more of the SNP markers selected from SNP_29 to SNP_36 are used to select a plant comprising the duplication of QTL3.1, or the copy number of one or more of SNP_29 to SNP_36 or of one or more of the 9 genes are used to select a plant comprising the duplication. Especially the donor SNP haplotype of SNP_35 and/or SNP_36 is used to select a plant or plant part comprising the duplication of QTL3.1.

Although the SNP markers can be used to select QTL3.1, especially from the donor used herein, there are now also other ways to select for (and/or to generate) plants comprising a duplication of one or more of the 8 OGD genes and/or the gene encoding the HR-like protein. These other ways will be described in a separate section further below. For example plants comprising a higher copy number or higher expression of one or more of the 8 OGD genes and/or the gene encoding the HR like protein can be selected or generated.

In the methods/plants below comprising the donor SNP genotype or haplotype for SNP markers linked to QTL3.1, in one aspect at least SNP_35 and/or SNP_36 has the donor SNP haplotype or genotype and/or the plant comprises the region of SEQ ID NO: 72 or 73 (or a region comprising at least 95% sequence identity to SEQ ID NO: 72 or 73) and/or a duplication of the region of SEQ ID NO: 72 or 73 (or a region comprising at least 95% sequence identity to SEQ ID NO: 72 or 73). In another aspect one or more of the SNP markers selected from SNP_29 to SNP_36 has the donor SNP genotype or haplotype.

Therefore, in one aspect a cultivated Cucumis sativus var. sativus plant is provided comprising an introgression fragment on chromosome 3 in homozygous or heterozygous form (or a modification in its genome), wherein said introgression fragment (or the genome modification) confers an increase in internal fruit rot resistance caused by DB (compared to the plant lacking the introgression fragment, or genome modification, e.g. the genetic control),

wherein said introgression fragment is detectable by a molecular marker assay (i.e. the plant comprises one or more molecular markers) which detects at least
a) the TT (homozygous) or TC (heterozygous) genotype for the Single Nucleotide Polymorphism marker SNP_35 at nucleotide 51 of SEQ ID NO: 35 (or in a variant thereof); and/or
b) the TT (homozygous) or TTG (heterozygous) genotype for the Single Nucleotide Polymorphism marker SNP_36 at nucleotide 51 of SEQ ID NO: 36 (or in a variant thereof);
   and/or wherein the plant comprises
c) a duplication of SEQ ID NO: 72 or a region comprising at least 95% sequence identity to SEQ ID NO: 72 on e.g. chromosome 3; or
d) a duplication of the chromosome 3 region starting at nucleotide 1 of SEQ ID NO: 83 and ending at nucleotide 100 of SEQ ID NO: 84 on e.g. chromosome 3;
e) a duplication of one or more or all of the following genes, e.g. on chromosome 3:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60.

In another aspect a cultivated Cucumis sativus var. sativus plant is provided comprising an introgression fragment on chromosome 3 in homozygous or heterozygous form, wherein said introgression fragment confers an increase in internal fruit rot resistance caused by DB (compared to the plant lacking the introgression fragment, e.g. the genetic control) and wherein said introgression fragment is detectable by a molecular marker assay (i.e. the plant comprises one or more molecular markers) which detects at least 1, preferably at least 2 or 3, or at least 4, 5, 6, 7, 8, 9, 10, 11, 12, 13, 14, 15, 16, 17, 18. 19, 20, 21, 22, 23 or 24 of the markers selected from the group consisting of:
a the GG (homozygous) or GA (heterozygous) genotype for the Single Nucleotide Polymorphism marker SNP_19 at nucleotide 51 of SEQ ID NO: 19 (or in a variant thereof);
b) the GG (homozygous) or GT (heterozygous) genotype for the Single Nucleotide Polymorphism marker SNP_20 at nucleotide 51 of SEQ ID NO: 20 (or in a variant thereof);
c) the TT (homozygous) or TC (heterozygous) genotype for the Single Nucleotide Polymorphism marker SNP_21 at nucleotide 51 of SEQ ID NO: 21 (or in a variant thereof);
d) the AA (homozygous) or AG (heterozygous) genotype for the Single Nucleotide Polymorphism marker SNP_22 at nucleotide 51 of SEQ ID NO: 22 (or in a variant thereof);
e) the CC (homozygous) or CCA (heterozygous) genotype for the Single Nucleotide Polymorphism marker SNP_23 at nucleotide 51 of SEQ ID NO: 23 (or in a variant thereof);
f) the AA (homozygous) or AC (heterozygous) genotype for the Single Nucleotide Polymorphism marker SNP_24 at nucleotide 51 of SEQ ID NO: 24 (or in a variant thereof);
g) the GG (homozygous) or GA (heterozygous) genotype for the Single Nucleotide Polymorphism marker SNP_25 at nucleotide 51 of SEQ ID NO: 25 (or in a variant thereof);
h) the TT (homozygous) or TC (heterozygous) genotype for the Single Nucleotide Polymorphism marker SNP_26 at nucleotide 51 of SEQ ID NO: 26 (or in a variant thereof);
i) the CC (homozygous) or CT (heterozygous) genotype for the Single Nucleotide Polymorphism marker SNP_27 at nucleotide 51 of SEQ ID NO: 27 (or in a variant thereof);
j) the GG (homozygous) or GA (heterozygous) genotype for the Single Nucleotide Polymorphism marker SNP_28 at nucleotide 51 of SEQ ID NO: 28 (or in a variant thereof);
k) the CC (homozygous) or CA (heterozygous) genotype for the Single Nucleotide Polymorphism marker SNP_29 at nucleotide 51 of SEQ ID NO: 29 (or in a variant thereof);
l) the AA (homozygous) or AG (heterozygous) genotype for the Single Nucleotide Polymorphism marker SNP_30 at nucleotide 51 of SEQ ID NO: 30 (or in a variant thereof);
m) the GG (homozygous) or GT (heterozygous) genotype for the Single Nucleotide Polymorphism marker SNP_31 at nucleotide 51 of SEQ ID NO: 31 (or in a variant thereof);
n) the GG (homozygous) or GA (heterozygous) genotype for the Single Nucleotide Polymorphism marker SNP_32 at nucleotide 51 of SEQ ID NO: 32 (or in a variant thereof);
o) the GG (homozygous) or GC (heterozygous) genotype for the Single Nucleotide Polymorphism marker SNP_33 at nucleotide 51 of SEQ ID NO: 33 (or in a variant thereof);
p) the GG (homozygous) or GA (heterozygous) genotype for the Single Nucleotide Polymorphism marker SNP_34 at nucleotide 51 of SEQ ID NO: 34 (or in a variant thereof);
q) the TT (homozygous) or TC (heterozygous) genotype for the Single Nucleotide Polymorphism marker SNP_35 at nucleotide 51 of SEQ ID NO: 35 (or in a variant thereof);
r) the TT (homozygous) or TTG (heterozygous) genotype for the Single Nucleotide Polymorphism marker SNP_36 at nucleotide 51 of SEQ ID NO: 36 (or in a variant thereof);
s) the CC (homozygous) or CT (heterozygous) genotype for the Single Nucleotide Polymorphism marker SNP_37 at nucleotide 51 of SEQ ID NO: 37 (or in a variant thereof);
t) the GG (homozygous) or GA (heterozygous) genotype for the Single Nucleotide Polymorphism marker SNP_38 at nucleotide 51 of SEQ ID NO: 38 (or in a variant thereof);
u) the CC (homozygous) or CA (heterozygous) genotype for the Single Nucleotide Polymorphism marker SNP_39 at nucleotide 51 of SEQ ID NO: 39 (or in a variant thereof);
v) the TT (homozygous) or TC (heterozygous) genotype for the Single Nucleotide Polymorphism marker SNP_40 at nucleotide 51 of SEQ ID NO: 40 (or in a variant thereof);
w) the AA (homozygous) or AG (heterozygous) genotype for the Single Nucleotide Polymorphism marker SNP_41 at nucleotide 51 of SEQ ID NO: 41 (or in a variant thereof);
x) the AA (homozygous) or AG (heterozygous) genotype for the Single Nucleotide Polymorphism marker SNP_42 at nucleotide 51 of SEQ ID NO: 42 (or in a variant thereof).

As mentioned previously, when referring to a SNP in a variant sequence, that variant sequence comprises at least 95%, 96% or 97% sequence identity with the mentioned sequence.

In one aspect the introgression fragment comprises the haplotype of at least 3, 4, 5, 6, 7, 8, 9, 10, 11, 12, 13, 14, 15, 16, 17 or 18 of the markers listed under a) to x) above and comprises the QTL3.1 or a variant thereof.

In one aspect the introgression fragment is obtainable from (has as source) seeds deposited under NCIMB 43530 or progeny thereof, i.e. material derived from the deposit by selfing and/or crossing.

In one aspect the introgression fragment comprises the haplotype of at least 18, 19, 20, 21 or 22 of the markers listed under a) to x) above and comprises the QTL3.1 or a variant thereof. In one aspect the introgression fragment is obtainable from (has as source) seeds deposited under NCIMB43530 or progeny thereof.

In one aspect the introgression fragment comprises the haplotype of at least 3, 4, 5, 6, 7, 8, 9, 10, 11, 12, 13, 14, 15, 16, 17 or 18 consecutive markers of the markers listed under a) to x) above and comprises the QTL3.1 or a variant thereof. In one aspect the introgression fragment is obtainable from (has as source) seeds deposited under NCIMB43530 or progeny thereof.

In one aspect the introgression fragment comprises the haplotype of at least 18, 19, 20, 21 or 22 consecutive markers of the markers listed under a) to x) above and comprises the QTL3.1 or a variant thereof. In one aspect the introgression fragment is obtainable from (has as source) seeds deposited under NCIMB43530 or progeny thereof.

The combination of such unique haplotypes of the introgression fragment in combination with the phenotype conferred by the (duplication of) QTL3.1 (especially in homozygous form and/or in combination with QTL5.1) make it easy for the person skilled in the art to generate and/or identify a plant or plant part which comprises QTL3.1 (e.g. the duplication of QTL3.1). This does not only apply to plants which have as a source the MYCR3 donor (as present in NCIMB43530, or progeny thereof), but also plants which have as a source other wild donors, having the haplotypes described above and the QTL3.1 or a variant thereof, i.e. conferring the same phenotype as QTL3.1 from MYCR3.

As it is now known that resistance is in one aspect due to a duplication of QTL3.1 and that a higher expression of one or more OGD genes and/or of the HR-like gene confers resistance, other donors can be easily selected which comprise the same or similar structural variation as the present donor, e.g. donors having higher copy numbers (e.g. duplications) of one or more OGD genes and/or the HR-like gene or donors having a higher expression of one or more OGD genes and/or the HR-like gene, e.g. through a more active promoter. Plants or plant parts can be screened for such structural variation or CNV and donors can be selected comprising e.g. a duplication of the chromosome 3 region of SEQ ID NO: 72 or of the region between SEQ ID NO: 83 and 84, or a duplication of one or more of the OGD1 to OGD8 genes and/or of the gene encoding the HR-like protein. Many methods for detecting CNV are known, e.g. digital PCR, microarray and Polymerase Chain Reaction (PCR) based technologies (such as quantitative PCR), the paralogue ratio test (PRT), sequencing methods, hybridization-based methods, and SNP array and array-based comparative genomic hybridization (CGH). See also Lye and Purugganan 2019, Trends in Plant Science, April 2019, Vol. 24, No. 4 and Zmieriko et al. 2014, Theor Appl Geneti 127:1-18.

The skilled person can also develop other molecular markers, e.g. markers in-between marker SNP_19 and SNP_42 and/or within 7 cM or within 5 CM of any one of SNP_19 to SNP_42, and/or within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb or less of any one of SNP_19 to SNP_42. Such markers may also be a stretch of nucleotide, CAPS markers, INDELs, etc. The skilled person can, for example, sequence the introgression fragment found in seeds deposited under accession number NCIMB43530 and use the sequence information to develop new markers and marker assays.

In one aspect QTL3.1 (or a variant thereof) is located in the region flanked by SEQ ID NO: 83 and SEQ ID NO: 84, and in another aspect QTL3.1 (or a variant thereof) is located in the region of SEQ ID NO: 72 or 73 or a sequence comprising at least 95%, 96%, 97% or more sequence identity to either of these.

Therefore, in one aspect a cultivated Cucumis sativus var. sativus plant is provided comprising an introgression fragment on chromosome 3 in homozygous or heterozygous form, wherein said introgression fragment confers an increase in internal fruit rot resistance caused by DB (compared to the plant lacking the introgression fragment, e.g. the genetic control) and wherein said introgression fragment comprises the following markers:
the TT (homozygous) or TC (heterozygous) genotype for the Single Nucleotide Polymorphism marker SNP_35 at nucleotide 51 of SEQ ID NO: 35 (or in a variant thereof); and/or
the TT (homozygous) or TTG (heterozygous) genotype for the Single Nucleotide Polymorphism marker SNP_36 at nucleotide 51 of SEQ ID NO: 36 (or in a variant thereof),
   and wherein the region of SEQ ID NO: 72 or a region comprising at least 95% sequence identity to SEQ ID NO: 72 is present at least twice in the haploid genome, preferably on chromosome 3.

In a further aspect a cultivated Cucumis sativus var. sativus plant is provided comprising an introgression fragment on chromosome 3 in homozygous or heterozygous form, wherein said introgression fragment confers an increase in internal fruit rot resistance caused by DB (compared to the plant lacking the introgression fragment, e.g. the genetic control) and wherein said introgression fragment comprises
a) a duplication of the chromosome 3 region flanked by SEQ ID NO: 83 and 84 or starting at nucleotide 1 of SEQ ID NO: 83 and ending at nucleotide 100 of SEQ ID NO: 84,
b) a duplication of the chromosome 3 region of SEQ ID NO: 72 or a region comprising at least 95% sequence identity to SEQ ID NO: 72, and/or
c) a duplication of one or more or all genes encoding a 2-oxoglutarate Fe(II) dependent oxygenase (OGD) protein selected from
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and/or a duplication of a gene encoding a HR-like protein comprising the amino acid sequence of SEQ ID NO: 60 or a sequence comprising at least 95% sequence identity to SEQ ID NO: 60.

In a further aspect a cultivated Cucumis sativus var. sativus plant is provided comprising an introgression fragment on chromosome 3 in homozygous or heterozygous form, wherein said introgression fragment confers an increase in internal fruit rot resistance caused by DB (compared to the plant lacking the introgression fragment, e.g. the genetic control) and wherein said introgression fragment confers an increased average gene expression of eight OGD genes, OGD1 to OGD8, encoding a 2-oxoglutarate Fe(II)-dependent oxygenase (OGD) proteins, to be at least 1.3 times that of the average gene expression of the eight OGD genes in a wild type, susceptible plant lacking the introgression fragment (comprising two copies of each OGD gene), wherein the eight OGD genes are the genes encoding
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
   and/or wherein introgression fragment confers an average gene expression of a gene encoding a HR-like protein to be at least 1.3, 1.4, 1.5, 1.6, 1.7, 1.8, 1.9, 2.0, 2.5, 3.0, 3.5, 4.0 times that of the average gene expression of said gene in a wild type, susceptible plant lacking the introgression fragment (comprising two copies of the gene), wherein said gene is a gene encoding a protein comprising the amino acid sequence of SEQ ID NO: 60 or a sequence comprising at least 95% sequence identity to SEQ ID NO: 60.

The introgression fragment can therefore either increase the copy number of one or more of the OGD genes and/or the gene encoding the HR-like protein, or it can introgress alleles which are higher expressed, e.g. where the cis-regulatory elements (such as the promoter or enhancers) or trans-regulatory elements result in a higher gene expression and higher protein levels. The introgression fragment leads to an effective higher dosage of one or more of the OGD genes and/or the gene encoding the HR-like protein, whereby ‘effective higher dosage’ (or ‘increased effective dosage’) herein means that more transcript and more protein is produced in the plant, especially in the ovary tissue, compared to the plant lacking the introgression fragment. By e.g. duplicating a gene, the effective dosage will be about 2 times.

In one aspect the introgression fragment comprising the QTL3.1 may be large (e.g. comprising one or more or all of the donor SNP nucleotides for SNP_19 to SNP_42), or may be smaller (e.g. comprise the donor nucleotide for one or more of SNP_29 to SNP_36 and/or comprising a duplication of the region starting at SEQ ID NO: 83 and ending at SEQ ID NO: 84, or comprising SEQ ID NO: 72, especially a duplication of all or part of SEQ ID NO: 72, or comprising an increased copy number and/or increased expression of one or more of the 8 OGD genes and/or the gene encoding the HR-like protein) and optionally lack one or more or all of the donor SNP markers, but it may still confer internal fruit rot resistance on the cultivated cucumber plant, i.e. it can still comprise the QTL (QTL3.1 or a variant). Such smaller and alternative introgression fragments are an embodiment of the invention.

In one aspect plants having smaller introgression fragments (e.g. sub-fragments) which still confer the enhanced internal fruit rot resistance (i.e. contain the duplication of QTL3.1 or a variant) can be generated using known techniques, such as fine-mapping or similar techniques. For example only the donor SNPs for SNP_35 and/or SNP_36 may be present, or only the donor SNPs for SNP_29 to SNP_36 may be present. For example by starting with a plant comprising the introgression fragment as found in seeds deposited under accession number NCIMB 43530 and crossing such a plant with another cultivated cucumber plant and selfing the progeny of said cross, and/or backcrossing the progeny, to generate a population of plants which will contain recombinants having a smaller introgression fragment on chromosome 3, which fragment still confers internal fruit rot resistance in relation to a plant lacking the introgression fragment (such as the genetic control, e.g. plants grown from seeds deposited under NCIMB43531), e.g. a fragment comprising the wild donor genotype or haplotype of one or more of SNP_29 to SNP_36 and/or comprising a duplication of the region starting at SEQ ID NO: 83 and ending at SEQ ID NO: 84 and/or comprising a duplication of SEQ ID NO: 72 or a sequence comprising at least 95% identity to SEQ ID NO: 72, and/or comprise a duplication of one or more or all of the 8 OGD genes and/or of the gene encoding the HR-like protein.

For example marker assays can be used to select recombinants and to determine the size of the smaller introgression fragment. One or more of SNP markers or the donor genotype or haplotype may be missing. The cultivated cucumber genotype (or haplotype) is then detected for the SNP marker. The internal fruit rot resistance of plants comprising such a smaller introgression fragment can then be compared in an internal fruit rot assay as described herein. If the percentage of fruits (that develop from spore-inoculated flowers) having internal fruit rot symptoms is significantly reduced compared to the control, then the smaller introgression fragment (or sub-fragment) has retained the (duplication of) QTL3.1.

Alternatively, the same or variant QTL (QTL3.1 or variant QTL3.1) may be introgressed from a different wild donor, whereby optionally not all SNP markers disclosed herein are present, i.e. the SNP haplotype may be slightly different than for the MYCR3 donor. Such alternative wild cucumber sources can be identified using the SNP markers provided herein, by screening germplasm (i.e. accessions of) wild cucumber using a marker assay to detect the genotype or haplotype of markers SNP_19 to SNP_42, or of subgroups thereof, such as 6, 7, 8, 9, 10, 11, 12, 13, 14, 15, 16 or more consecutive markers, especially comprising the donor SNP haplotype for SNP_35 and/or SNP_36, or comprising the donor SNP haplotype for one or more or all markers of SNP_29 to SNP_36. Plants comprising the same or variant QTL3.1 from other sources are also an embodiment of the invention. As long as the donor has a SNP haplotype which is identical to the MYCR3 donor described herein for at least 3, 4, 5, 6, 7, 8, 9, 10, 11, 12, 13, 14, 15, 16, 17, 18, 19, 20, 21 or more (or all) of the SNPs of SNP_19 to SNP_42 (especially for at least 3, 4, 5, 6, 7, 8, 9, 10, 11, 12, 13 or more consecutive SNP markers of SNP_19 to SNP_42), in particular for one or more or all markers of SNP_29 to SNP_36, especially for SNP_35 and/or SNP_36, the donor may comprise a duplication of QTL3.1.

However, as the genes are known, the skilled person can easily screen other donors for CNV of all or part of the region of SEQ ID NO: 72 or 73 (or a region comprising at least 95% identity to either of these), or for an increased copy number or increased expression of one or more of the genes encoding the 8 OGD proteins and/or the HR-like protein. As long as the donor comprises structural variation which increases the effective higher dosage of one or more of the genes encoding the 8 OGD proteins and/or the HR-like protein when introgressed into cultivated cucumber, the donor is a suitable donor for QTL3.1. The skilled person can then introgress the QTL3.1 (or a variant thereof) into cultivated cucumber in order to enhance internal fruit rot resistance as described herein and in order to confirm that the QTL reduces susceptibility to internal fruit rot when present in cultivated cucumber.

Obviously, increasing copy number and/or expression of one or more of the genes can also be identified or generated de novo in cultivated cucumber.

As described above, in one embodiment the cultivated cucumber plant of the invention comprises an introgression fragment comprising QTL3.1 and at least a subset of SNP markers with the genotype or haplotype of the wild donor, i.e. for at least 3, 4, 5, 6, 7, 8, 9, 10, 11, 12, 13, 14, 15, 16, 17, 18, 19, 20, 21 or 22 (or more) SNP markers of SNP_19 to SNP_42, especially for at least 3, 4, 5, 6, 7, 8, 9, 10, 11, 12, 13 or more consecutive SNP markers of SNP_19 to SNP_42, especially one or more SNP markers selected from SNP_29 to SNP_36, and in one aspect at least SNP_35 and/or SNP_36.

Thus, the introgression fragment (and a cultivated cucumber plant or plant part, e.g., a cell, comprising the introgression fragment) can be detected in a marker assay by detecting the SNP genotype or haplotype of the introgression fragment (i.e. of the wild relative of cucumber germplasm) of one or more or all of the markers above. However, the introgression fragment (and a cultivated cucumber plant or plant part, e.g., a cell, comprising the introgression fragment) can also be detected by the increased copy number or increased gene expression of one or more of the 8 OGD genes and/or the gene encoding the HR-like protein, as the introgression fragment for example comprises a duplication of all 9 genes or a subset thereof, such as all 8 OGD genes or the introgression fragment comprises an enhanced expression of one or more or all of the 8 genes encoding the OGD proteins and/or of the gene encoding the HR-like protein. The introgression fragment can thus be identified by having a gene dosage of one or more of the 8 OGD genes and/or the gene encoding the HR-like protein which is higher than the wild type chromosome 3 dosage (lacking the introgression fragment) or an introgression fragment comprising alleles of one or more of the 8 OGD genes and/or of the gene encoding the HR like protein having a higher gene expression than the wild type genes.

Thus, in one aspect, a Quantitative Trait Locus (QTL3.1) was found to be present on chromosome 3 of a wild donor cucumber which, when transferred (introgressed) into a cultivated cucumber plant, variety or breeding line, and confers significantly enhanced internal fruit rot resistance (reduced susceptibility to internal fruit rot) onto the cultivated cucumber plant, due to an enhancement of the effective higher dosage of 8 OGD genes and/or one gene encoding a HR-like protein.

The QTLs (or variant QTLs) can be identified in e.g. wild donors and can then be introgressed into cultivated cucumber, e.g. using MAS, i.e. using one or more (or all) of the SNP markers provided herein to detect and/or select progeny plants (e.g. backcross plants) comprising a recombinant chromosome 3. The selected plants, i.e. the cultivated cucumber plants comprising an introgression fragment on chromosome 3 can then be phenotyped in internal fruit rot assays together with the suitable control plants in order to determine whether the introgression fragment indeed causes a reduced susceptibility to internal fruit rot.

Accessions of wild cucumbers are obtainable from the USDA National Plant Germplasm System collection or other seed collections, and can thus be screened for the presence of QTL3.1 using e.g. a marker assay as described herein or sequencing the region, or analyzing the copy number and/or gene expression of the 8 OGD genes and/or the gene encoding the HR-like protein, and accessions comprising an enhanced copy number (identified by e.g. digital PCR) and/or enhanced expression of one or more of the 8 OGD genes and/or the gene encoding the HR-like protein can be crossed with a cultivated cucumber plant having normal wild-type, non-recombinant chromosomes 3. The F1 or F2 generation (or further generation, such as the F3 or a backcross generation) can then be screened for recombinant plants having the introgression fragment using e.g. molecular marker assays described herein, for detecting one or more of the SNP markers or other methods for detecting the gene copy number or gene expression.

In a specific embodiment, the introgression fragment comprising the QTL3.1 is derivable from (or derived from) or obtainable from (or obtained from; or as present in) seeds, a representative sample of which has been deposited under accession number NCIMB 43530, or from progeny thereof. The progeny may be any progeny which retain the one or more (or all) SNP markers indicative of (and linked to) the QTL, especially one or more of SNP_29 to SNP_36, or at least SNP_35 and/or SNP_36, as described, and which retain the duplication of all or part of SEQ ID NO: 72 (or a sequence comprising at least 95% sequence identity thereto) comprising the 9 genes. The progeny in one aspect retain the duplication of all 9 genes, but also progeny which retain the duplication of 1, 2, 3, 4, 5, 6, 7 or 8 of the 9 genes are encompassed herein. Thus, progeny are not limited to F1 or F2 progeny of the deposit, but can be any progeny, whether obtained by selfing and/or crossing with another cucumber plant. In one aspect the progeny retain the large duplication, starting at nucleotide 1 of SEQ ID NO: 83 and ending at nucleotide 100 of SEQ ID NO: 84.

In one embodiment the introgression fragment is identifiable by one or more of the markers described elsewhere herein, especially markers SNP_19 to SNP_42 for the introgression fragment on chromosome 3, or a subset of markers, such as one or more of the markers selected from SNP markers SNP_19 to SNP_27, or SNP_27 to SNP_35, SNP_35 to SNP_42, or SNP_21 to SNP_30, or SNP_30 to SNP_40, or SNP_29 to SNP_36, or SNP_35 to SNP_36. In another embodiment the introgression fragment is identifiable by the duplication of one or more or all genes selected from OGD1 to OGD8 and HR-like protein or by enhanced expression of one or more or all genes selected from OGD1 to OGD8 and HR-like protein or by the presence of a duplication of the region starting at nucleotide 1 of SEQ ID NO: 83 and ending at nucleotide 100 of SEQ ID NO: 84, or by the presence of a duplication of SEQ ID NO: 72 or a sequence comprising at least 95% identity to SEQ ID NO: 72. In one aspect the invention provides a cultivated cucumber plant, having a genome of cultivated (domesticated) cucumber which comprises increased resistance to internal fruit rot, wherein the increased resistance is conferred by an introgression fragment on the cultivated cucumber chromosome 3, wherein said introgression fragment is obtained by (or obtainable by) crossing a cultivated plant grown from seeds deposited under NCIMB 43530 or progeny of this plant (which optionally comprises one or more the markers disclosed herein linked to the QTL) with a cultivated cucumber plant. Thus in one aspect the cultivated cucumber plant of the invention comprises the same introgression fragment and the same recombinant chromosome 3 as present in NCIMB 43530 (comprising the wild donor genotype or haplotype for SNP_19 to SNP_42), or it comprises a shorter fragment of that introgression fragment (a sub-fragment), whereby the shorter fragment retains the genetic element conferring enhanced internal fruit rot resistance (e.g. retains the duplication of at least 1, 2, 3, 4, 5, 6, 7, 8 or 9 genes selected from OGD1 to OGD8 and HR-like protein).

Thus, in one aspect the invention relates to a plant of the invention i.e. a cultivated Cucumis sativus var. sativus plant comprising an introgression fragment from a wild cucumber on chromosome 3 in homozygous or heterozygous form and wherein said introgression fragment is the introgression fragment “as in”/is “identical to”/is “the same as in” the seeds deposited under number NCIMB 43530, or is a shorter fragment (sub fragment) thereof, but still confers internal fruit rot resistance due to the presence of QTL3.1 on the sub-fragment. In one aspect the short sub-fragment retains the duplication of at least 1, 2, 3, 4, 5, 6, 7, 8 or 9 genes selected from OGD1 to OGD8 and HR-like protein. Thus in one aspect the subfragment retains the duplication of all or part of the region of SEQ ID NO: 72 or a sequence comprising at least 95% sequence identity to SEQ ID NO: 72.

In yet another embodiment the invention relates to a plant of the invention i.e. a cultivated Cucumis sativus var. sativus plant comprising an introgression fragment from a wild donor on chromosome 3, in homozygous or heterozygous form, and wherein said introgression fragment is a variant of the introgression fragment found in seeds deposited under number NCIMB 43530, i.e. it comprises the QTL 3.1, but the genomic sequence may be different. As wild accessions will be genetically divergent, the genomic sequence of an introgression fragment comprising QTL3.1 from other wild donors will most likely not be identical to the genomic sequence as introgressed into NCIMB43530, and even the resistance conferring genes (comprising a promoter, introns and exons) may be divergent in nucleotide sequence, but the function will be the same, i.e. conferring enhanced internal fruit rot resistance. The divergence can be seen in that the donor haplotype of certain SNP markers linked to QTL3.1 may be not 100% identical to the donor haplotype found in NCIMB43530. So for example not all of SNP_19 to SNP_42 may have the same donor haplotype in other wild donors. As it is now known that the resistance is due to an effective higher dosage of one or more of OGD1 to OGD8 and/or HR-like protein, the SNP haplotype, which is in non-coding parts of the genome, may be different from the MYCR3 donor used herein. Thus other donors may have a different SNP haplotype for one or more or all of SNP_19 to SNP_42, and the genes encoding OGD1 to OGD8 and the HR-like protein may also encode proteins with 95%, 96%, 97%, 98% or 99% amino acid sequence identity to any one of these proteins.

Other donors may comprise the same donor SNP haplotype as in NCIMB43530 for e.g. at least 6, 7, 8, 9, 10, 11 or more SNP markers selected from SNP_19 to SNP_42, or e.g. at least 6, 7, 8, 9, 10, 11 or more consecutive SNP markers selected from SNP_19 to SNP_42, or e.g. for at least a group of SNP markers selected from SNP_19 to SNP_27, or SNP_27 to SNP_35, SNP_35 to SNP_42, or SNP_21 to SNP_30, or SNP_30 to SNP_40, or SNP_29 to SNP_36, or SNP_35 to SNP_36, or they may have a different SNP donor haplotype but still comprise an effective higher dosage of OGD1 to OGD8 and/or HR-like protein, e.g. a duplication of one or more of the OGD1 to OGD8 genes and/or of the gene encoding the HR-like protein or an enhanced expression of one or more or all of the genes. Thereby the internal fruit rot resistance QTL, QTL3.1 (comprising e.g. a variant or ortholog of the fruit rot resistance allele) may still be present in such wild accessions. The skilled person is capable of identifying and introgressing the QTL3.1 comprising region found in other wild cucumbers into cultivated cucumber, e.g. identifying wild cucumber accessions comprising such a QTL3.1 into a cultivated cucumber plant line or variety and assessing the internal fruit rot resistance of the cultivated plant compared to the control plant lacking the introgression fragment.

In one embodiment a plant or plant part comprising (a duplication of) QTL3.1 or a variant thereof is provided, wherein the introgression fragment in the plant or plant part comprising QTL3.1, comprises and/or is detectable by a molecular marker assay which detects the following genotypes or haplotypes for at least 1, preferably at least 2, 3, 4, 5, 6, 7, 8, 9, 10, 11, 12, 13, 14, 15, 16, 17, 18, 19, 20, 21, 22, 23 or 24 Single Nucleotide Polymorphism (SNP) markers selected from the group consisting of:
a) the GG (homozygous) or GX (heterozygous) genotype for the Single Nucleotide Polymorphism marker SNP_19 at nucleotide 51 of SEQ ID NO: 19 (or in a variant thereof);
b) the GG (homozygous) or GX (heterozygous) genotype for the Single Nucleotide Polymorphism marker SNP_20 at nucleotide 51 of SEQ ID NO: 20 (or in a variant thereof);
c) the TT (homozygous) or TX (heterozygous) genotype for the Single Nucleotide Polymorphism marker SNP_21 at nucleotide 51 of SEQ ID NO: 21 (or in a variant thereof);
d) the AA (homozygous) or AX (heterozygous) genotype for the Single Nucleotide Polymorphism marker SNP_22 at nucleotide 51 of SEQ ID NO: 22 (or in a variant thereof);
e) the CC (homozygous) or CX (heterozygous) genotype for the Single Nucleotide Polymorphism marker SNP_23 at nucleotide 51 of SEQ ID NO: 23 (or in a variant thereof);
f) the AA (homozygous) or AX (heterozygous) genotype for the Single Nucleotide Polymorphism marker SNP_24 at nucleotide 51 of SEQ ID NO: 24 (or in a variant thereof);
g) the GG (homozygous) or GX (heterozygous) genotype for the Single Nucleotide Polymorphism marker SNP_25 at nucleotide 51 of SEQ ID NO: 25 (or in a variant thereof);
h) the TT (homozygous) or TX (heterozygous) genotype for the Single Nucleotide Polymorphism marker SNP_26 at nucleotide 51 of SEQ ID NO: 26 (or in a variant thereof);
i) the CC (homozygous) or CX (heterozygous) genotype for the Single Nucleotide Polymorphism marker SNP_27 at nucleotide 51 of SEQ ID NO: 27 (or in a variant thereof);
j) the GG (homozygous) or GX (heterozygous) genotype for the Single Nucleotide Polymorphism marker SNP_28 at nucleotide 51 of SEQ ID NO: 28 (or in a variant thereof);
k) the CC (homozygous) or CX (heterozygous) genotype for the Single Nucleotide Polymorphism marker SNP_29 at nucleotide 51 of SEQ ID NO: 29 (or in a variant thereof);
l) the AA (homozygous) or AX (heterozygous) genotype for the Single Nucleotide Polymorphism marker SNP_30 at nucleotide 51 of SEQ ID NO: 30 (or in a variant thereof);
m) the GG (homozygous) or GX (heterozygous) genotype for the Single Nucleotide Polymorphism marker SNP_31 at nucleotide 51 of SEQ ID NO: 31 (or in a variant thereof);
n) the GG (homozygous) or GX (heterozygous) genotype for the Single Nucleotide Polymorphism marker SNP_32 at nucleotide 51 of SEQ ID NO: 32 (or in a variant thereof);
o) the GG (homozygous) or GX (heterozygous) genotype for the Single Nucleotide Polymorphism marker SNP_33 at nucleotide 51 of SEQ ID NO: 33 (or in a variant thereof);
p) the GG (homozygous) or GX (heterozygous) genotype for the Single Nucleotide Polymorphism marker SNP_34 at nucleotide 51 of SEQ ID NO: 34 (or in a variant thereof);
q) the TT (homozygous) or TX (heterozygous) genotype for the Single Nucleotide Polymorphism marker SNP_35 at nucleotide 51 of SEQ ID NO: 35 (or in a variant thereof);
r) the TT (homozygous) or TX (heterozygous) genotype for the Single Nucleotide Polymorphism marker SNP_36 at nucleotide 51 of SEQ ID NO: 36 (or in a variant thereof);
s) the CC (homozygous) or CX (heterozygous) genotype for the Single Nucleotide Polymorphism marker SNP_37 at nucleotide 51 of SEQ ID NO: 37 (or in a variant thereof);
t) the GG (homozygous) or GX (heterozygous) genotype for the Single Nucleotide Polymorphism marker SNP_38 at nucleotide 51 of SEQ ID NO: 38 (or in a variant thereof);
u) the CC (homozygous) or CX (heterozygous) genotype for the Single Nucleotide Polymorphism marker SNP_39 at nucleotide 51 of SEQ ID NO: 39 (or in a variant thereof);
V the TT (homozygous) or TX (heterozygous) genotype for the Single Nucleotide Polymorphism marker SNP_40 at nucleotide 51 of SEQ ID NO: 40 (or in a variant thereof);
w) the AA (homozygous) or AX (heterozygous) genotype for the Single Nucleotide Polymorphism marker SNP_41 at nucleotide 51 of SEQ ID NO: 41 (or in a variant thereof);
x) the AA (homozygous) or AX (heterozygous) genotype for the Single Nucleotide Polymorphism marker SNP_42 at nucleotide 51 of SEQ ID NO: 42 (or in a variant thereof).

In one embodiment a plant or plant part comprising (a duplication of) QTL3.1 or a variant thereof is provided, wherein the introgression fragment in the plant or plant part comprising QTL3.1, comprises and/or is detectable by a molecular marker assay which detects the following genotypes or haplotypes for at least 1, preferably at least 2, 3, 4, 5, 6 or 7 Single Nucleotide Polymorphism (SNP) markers selected from the group consisting of:
a) the CC (homozygous) or CX (heterozygous) genotype for the Single Nucleotide Polymorphism marker SNP_29 at nucleotide 51 of SEQ ID NO: 29 (or in a variant thereof);
b) the AA (homozygous) or AX (heterozygous) genotype for the Single Nucleotide Polymorphism marker SNP_30 at nucleotide 51 of SEQ ID NO: 30 (or in a variant thereof);
c) the GG (homozygous) or GX (heterozygous) genotype for the Single Nucleotide Polymorphism marker SNP_31 at nucleotide 51 of SEQ ID NO: 31 (or in a variant thereof);
d) the GG (homozygous) or GX (heterozygous) genotype for the Single Nucleotide Polymorphism marker SNP_32 at nucleotide 51 of SEQ ID NO: 32 (or in a variant thereof);
e) the GG (homozygous) or GX (heterozygous) genotype for the Single Nucleotide Polymorphism marker SNP_33 at nucleotide 51 of SEQ ID NO: 33 (or in a variant thereof);
f) the GG (homozygous) or GX (heterozygous) genotype for the Single Nucleotide Polymorphism marker SNP_34 at nucleotide 51 of SEQ ID NO: 34 (or in a variant thereof);
g) the TT (homozygous) or TX (heterozygous) genotype for the Single Nucleotide Polymorphism marker SNP_35 at nucleotide 51 of SEQ ID NO: 35 (or in a variant thereof);
h) the TT (homozygous) or TX (heterozygous) genotype for the Single Nucleotide Polymorphism marker SNP_36 at nucleotide 51 of SEQ ID NO: 36 (or in a variant thereof).

It is clear that when the introgression fragment is heterozygous and only found on one chromosome 3 of the pair, the first nucleotide mentioned above is the nucleotide of the donor (of the introgression fragment, which is part of the recombinant chromosome 3), while the second nucleotide is the nucleotide of the chromosome 3 lacking the introgression fragment, e.g. the non-recombinant chromosome 3 of the recurrent parent. The second nucleotide is herein indicated as X, meaning any nucleotide (A, G, T or C), as this second nucleotide can vary, depending on the genetic background of the cucumber line.

In one aspect nucleotide X is the nucleotide of the recurrent parent as present in the deposited seeds and as indicated in Table 2 and herein below.

Thus, in one embodiment a plant or plant part comprising QTL3.1 or a variant thereof is provided, wherein the introgression fragment in the plant or plant part comprising QTL3.1, comprises and/or is detectable by a molecular marker assay which detects the following genotypes or haplotypes for at least 3, preferably at least 4, 5, 6, 7, 8, 9, 10, 11, 12, 13, 14, 15, 16, 17, 18, 19, 20, 21, 22, 23 or 24 Single Nucleotide Polymorphism (SNP) markers selected from the group consisting of:
a) the GG (homozygous) or GA (heterozygous) genotype for the Single Nucleotide Polymorphism marker SNP_19 at nucleotide 51 of SEQ ID NO: 19 (or in a variant thereof);
b) the GG (homozygous) or GT (heterozygous) genotype for the Single Nucleotide Polymorphism marker SNP_20 at nucleotide 51 of SEQ ID NO: 20 (or in a variant thereof);
c) the TT (homozygous) or TC (heterozygous) genotype for the Single Nucleotide Polymorphism marker SNP_21 at nucleotide 51 of SEQ ID NO: 21 (or in a variant thereof);
d) the AA (homozygous) or AG (heterozygous) genotype for the Single Nucleotide Polymorphism marker SNP_22 at nucleotide 51 of SEQ ID NO: 22 (or in a variant thereof);
e) the CC (homozygous) or CCA (heterozygous) genotype for the Single Nucleotide Polymorphism marker SNP_23 at nucleotide 51 of SEQ ID NO: 23 (or in a variant thereof);
f) the AA (homozygous) or AC (heterozygous) genotype for the Single Nucleotide Polymorphism marker SNP_24 at nucleotide 51 of SEQ ID NO: 24 (or in a variant thereof);
g) the GG (homozygous) or GA (heterozygous) genotype for the Single Nucleotide Polymorphism marker SNP_25 at nucleotide 51 of SEQ ID NO: 25 (or in a variant thereof);
h) the TT (homozygous) or TC (heterozygous) genotype for the Single Nucleotide Polymorphism marker SNP_26 at nucleotide 51 of SEQ ID NO: 26 (or in a variant thereof);
i) the CC (homozygous) or CT (heterozygous) genotype for the Single Nucleotide Polymorphism marker SNP_27 at nucleotide 51 of SEQ ID NO: 27 (or in a variant thereof);
j) the GG (homozygous) or GA (heterozygous) genotype for the Single Nucleotide Polymorphism marker SNP_28 at nucleotide 51 of SEQ ID NO: 28 (or in a variant thereof);
k) the CC (homozygous) or CA (heterozygous) genotype for the Single Nucleotide Polymorphism marker SNP_29 at nucleotide 51 of SEQ ID NO: 29 (or in a variant thereof);
l) the AA (homozygous) or AG (heterozygous) genotype for the Single Nucleotide Polymorphism marker SNP_30 at nucleotide 51 of SEQ ID NO: 30 (or in a variant thereof);
m) the GG (homozygous) or GT (heterozygous) genotype for the Single Nucleotide Polymorphism marker SNP_31 at nucleotide 51 of SEQ ID NO: 31 (or in a variant thereof);
n) the GG (homozygous) or GA (heterozygous) genotype for the Single Nucleotide Polymorphism marker SNP_32 at nucleotide 51 of SEQ ID NO: 32 (or in a variant thereof);
o) the GG (homozygous) or GC (heterozygous) genotype for the Single Nucleotide Polymorphism marker SNP_33 at nucleotide 51 of SEQ ID NO: 33 (or in a variant thereof);
p) the GG (homozygous) or GA (heterozygous) genotype for the Single Nucleotide Polymorphism marker SNP_34 at nucleotide 51 of SEQ ID NO: 34 (or in a variant thereof);
q) the TT (homozygous) or TC (heterozygous) genotype for the Single Nucleotide Polymorphism marker SNP_35 at nucleotide 51 of SEQ ID NO: 35 (or in a variant thereof);
r) the TT (homozygous) or TTG (heterozygous) genotype for the Single Nucleotide Polymorphism marker SNP_36 at nucleotide 51 of SEQ ID NO: 36 (or in a variant thereof);
s) the CC (homozygous) or CT (heterozygous) genotype for the Single Nucleotide Polymorphism marker SNP_37 at nucleotide 51 of SEQ ID NO: 37 (or in a variant thereof);
t) the GG (homozygous) or GA (heterozygous) genotype for the Single Nucleotide Polymorphism marker SNP_38 at nucleotide 51 of SEQ ID NO: 38 (or in a variant thereof);
u) the CC (homozygous) or CA (heterozygous) genotype for the Single Nucleotide Polymorphism marker SNP_39 at nucleotide 51 of SEQ ID NO: 39 (or in a variant thereof);
v) the TT (homozygous) or TC (heterozygous) genotype for the Single Nucleotide Polymorphism marker SNP_40 at nucleotide 51 of SEQ ID NO: 40 (or in a variant thereof);
w) the AA (homozygous) or AG (heterozygous) genotype for the Single Nucleotide Polymorphism marker SNP_41 at nucleotide 51 of SEQ ID NO: 41 (or in a variant thereof);
x) the AA (homozygous) or AG (heterozygous) genotype for the Single Nucleotide Polymorphism marker SNP_42 at nucleotide 51 of SEQ ID NO: 42 (or in a variant thereof).

In one embodiment a plant or plant part comprising QTL3.1 or a variant thereof is provided, wherein the introgression fragment in the plant or plant part comprising QTL3.1, comprises and/or is detectable by a molecular marker assay which detects the following genotypes or haplotypes for at least 3, preferably at least 4, 5, 6 or 7 Single Nucleotide Polymorphism (SNP) markers selected from the group consisting of:
a) the CC (homozygous) or CX (heterozygous) genotype for the Single Nucleotide Polymorphism marker SNP_29 at nucleotide 51 of SEQ ID NO: 29 (or in a variant thereof);
b) the AA (homozygous) or AG (heterozygous) genotype for the Single Nucleotide Polymorphism marker SNP_30 at nucleotide 51 of SEQ ID NO: 30 (or in a variant thereof);
c) the GG (homozygous) or GT (heterozygous) genotype for the Single Nucleotide Polymorphism marker SNP_31 at nucleotide 51 of SEQ ID NO: 31 (or in a variant thereof);
d) the GG (homozygous) or GA (heterozygous) genotype for the Single Nucleotide Polymorphism marker SNP_32 at nucleotide 51 of SEQ ID NO: 32 (or in a variant thereof);
e) the GG (homozygous) or GC (heterozygous) genotype for the Single Nucleotide Polymorphism marker SNP_33 at nucleotide 51 of SEQ ID NO: 33 (or in a variant thereof);
f) the GG (homozygous) or GA (heterozygous) genotype for the Single Nucleotide Polymorphism marker SNP_34 at nucleotide 51 of SEQ ID NO: 34 (or in a variant thereof);
g) the TT (homozygous) or TC (heterozygous) genotype for the Single Nucleotide Polymorphism marker SNP_35 at nucleotide 51 of SEQ ID NO: 35 (or in a variant thereof);
h) the TT (homozygous) or TTG (heterozygous) genotype for the Single Nucleotide Polymorphism marker SNP_36 at nucleotide 51 of SEQ ID NO: 36 (or in a variant thereof).

In one aspect the genotype or haplotype of at least 3, or at least 4, 5, 6, 7, 8, 9, 10, 11, 12, 13, 14, 15, 16, 17, 18, 19, 20, 21, 22, 23 or 24 markers which are detected (and which are present on the introgression fragment) are consecutive markers. That means the introgression fragment comprises the genotype (homozygous introgression fragment) or haplotype (heterozygous introgression fragment) for at least 3, 4, 5, 6, 7, 8, 9, 10 or more markers, whereby these are consecutive markers, e.g. three consecutive markers are for example SNP_34, SNP_35 and SNP_36. The genotype of the plant or plant part comprising the introgression fragment in homozygous form would then be GG-TT-TT.

Without being bound in any way, the marker haplotype of any 10 or more consecutive markers out of the 24 markers is a unique identifier of the introgression fragment of the donor and/or the marker haplotype of any 21 or more of the 24 markers is a unique identifier of the introgression fragment of the donor.

Herein the haplotype is indicted to be the haplotype of a specific SNP nucleotide in a specific sequence (SEQ ID Numbers), or in a sequence comprising at least 95%, 96% or 97% sequence identity (or 98% or 99%) to the specific sequence (SEQ ID Number). For example the GG (homozygous) or GA (heterozygous) genotype for SNP_19 at nucleotide 51 of SEQ ID NO: 19 “or at nucleotide 51 of a sequence comprising at least 97% identity to SEQ ID NO: 19”. The sequence of SEQ ID Number flanking the SNP (i.e. the 50 nucleotides preceding the SNP and the 50 nucleotides following the SNP) are herein the sequence of the wild donor MYCR3. However, there may be very slight sequence variation or sequencing errors in the flanking sequence surrounding the SNPs. Therefore, such flanking sequences, which comprise e.g. one, two or three nucleotides which are different from the flanking sequences provided herein in the specific SEQ ID Numbers are encompassed herein.

As mentioned, the SNP haplotype of one or more or all SNP markers linked to QTL3.1 (or a variant thereof) and/or the presence of a duplication of all or part of the region between SNP_35 and 659 nt downstream of SNP_36 (i.e. SEQ ID NO: 72 and 73) is useful for the genetic identification of the donor fragment and/or introgression fragment in combination with the phenotype conferred by the QTL, and thus for the identification of wild donors comprising the QTL3.1 (or a variant thereof), for generating cucumber plants or plant parts comprising the QTL3.1 (or a variant thereof) and/or detecting cucumber plants or plant parts comprising the QTL3.1 (or a variant thereof).

Therefore, in one aspect a method of using the SNP haplotype of one or more or all SNP markers linked to QTL3.1 (or a variant thereof) for breeding cucumber plants comprising the QTL3.1 (or a variant thereof) and/or for screening donor accessions or cultivated cucumber lines or varieties for the presence of QTL3.1 is one embodiment herein.

In another aspect a method of identifying a duplication of all or part of the region comprising OGD1 to OGD8 and a gene encoding a HR-like protein and/or a region comprising one or more of the genes selected from the genes encoding OGD1 to OGD8 and HR-like protein having enhanced expression compared to the wild type genes, for breeding cucumber plants comprising the QTL3.1 (or a variant thereof) and/or for screening donor accessions or cultivated cucumber lines or varieties for the presence of QTL3.1 is one embodiment herein.

Thus, in one embodiment a method for detecting, selecting and/or breeding a plant or plant part comprising QTL3.1 or a variant thereof is provided, comprising carrying out a molecular marker assay and optionally selecting a plant or plant part which comprises the following genotypes or haplotypes for at least 3, preferably at least 4, 5, 6, 7, 8, 9, 10, 11, 12, 13, 14, 15, 16, 17, 18, 19, 20, 21, 22, 23 or 24 Single Nucleotide Polymorphism (SNP) markers selected from the group consisting of:
a) the GG (homozygous) or GA or GX (heterozygous) genotype for the Single Nucleotide Polymorphism marker SNP_19 at nucleotide 51 of SEQ ID NO: 19 (or in a variant thereof);
b) the GG (homozygous) or GT or GX (heterozygous) genotype for the Single Nucleotide Polymorphism marker SNP_20 at nucleotide 51 of SEQ ID NO: 20 (or in a variant thereof);
c) the TT (homozygous) or TC or TX (heterozygous) genotype for the Single Nucleotide Polymorphism marker SNP_21 at nucleotide 51 of SEQ ID NO: 21 (or in a variant thereof);
d) the AA (homozygous) or AG or AX (heterozygous) genotype for the Single Nucleotide Polymorphism marker SNP_22 at nucleotide 51 of SEQ ID NO: 22 (or in a variant thereof);
e) the CC (homozygous) or CCA or CCX (heterozygous) genotype for the Single Nucleotide Polymorphism marker SNP_23 at nucleotide 51 of SEQ ID NO: 23 (or in a variant thereof);
f) the AA (homozygous) or AC or AX (heterozygous) genotype for the Single Nucleotide Polymorphism marker SNP_24 at nucleotide 51 of SEQ ID NO: 24 (or in a variant thereof);
g) the GG (homozygous) or GA or GX (heterozygous) genotype for the Single Nucleotide Polymorphism marker SNP_25 at nucleotide 51 of SEQ ID NO: 25 (or in a variant thereof);
h) the TT (homozygous) or TC or TX (heterozygous) genotype for the Single Nucleotide Polymorphism marker SNP_26 at nucleotide 51 of SEQ ID NO: 26 (or in a variant thereof);
i) the CC (homozygous) or CT or CX (heterozygous) genotype for the Single Nucleotide Polymorphism marker SNP_27 at nucleotide 51 of SEQ ID NO: 27 (or in a variant thereof);
j) the GG (homozygous) or GA or GX (heterozygous) genotype for the Single Nucleotide Polymorphism marker SNP_28 at nucleotide 51 of SEQ ID NO: 28 (or in a variant thereof);
k) the CC (homozygous) or CA or CX (heterozygous) genotype for the Single Nucleotide Polymorphism marker SNP_29 at nucleotide 51 of SEQ ID NO: 29 (or in a variant thereof);
l) the AA (homozygous) or AG or AX (heterozygous) genotype for the Single Nucleotide Polymorphism marker SNP_30 at nucleotide 51 of SEQ ID NO: 30 (or in a variant thereof);
m) the GG (homozygous) or GT or GX (heterozygous) genotype for the Single Nucleotide Polymorphism marker SNP_31 at nucleotide 51 of SEQ ID NO: 31 (or in a variant thereof);
n) the GG (homozygous) or GA or GX (heterozygous) genotype for the Single Nucleotide Polymorphism marker SNP_32 at nucleotide 51 of SEQ ID NO: 32 (or in a variant thereof);
the GG (homozygous) or GC or GX (heterozygous) genotype for the Single Nucleotide Polymorphism marker SNP_33 at nucleotide 51 of SEQ ID NO: 33 (or in a variant thereof);
p) the GG (homozygous) or GA or GX (heterozygous) genotype for the Single Nucleotide Polymorphism marker SNP_34 at nucleotide 51 of SEQ ID NO: 34 (or in a variant thereof);
q) the TT (homozygous) or TC or TX (heterozygous) genotype for the Single Nucleotide Polymorphism marker SNP_35 at nucleotide 51 of SEQ ID NO: 35 (or in a variant thereof);
r) the TT (homozygous) or TTG or TTX (heterozygous) genotype for the Single Nucleotide Polymorphism marker SNP_36 at nucleotide 51 of SEQ ID NO: 36 (or in a variant thereof);
s) the CC (homozygous) or CT or CX (heterozygous) genotype for the Single Nucleotide Polymorphism marker SNP_37 at nucleotide 51 of SEQ ID NO: 37 (or in a variant thereof);
t) the GG (homozygous) or GA or GX (heterozygous) genotype for the Single Nucleotide Polymorphism marker SNP_38 at nucleotide 51 of SEQ ID NO: 38 (or in a variant thereof);
u) the CC (homozygous) or CA or CX (heterozygous) genotype for the Single Nucleotide Polymorphism marker SNP_39 at nucleotide 51 of SEQ ID NO: 39 (or in a variant thereof);
v) the TT (homozygous) or TC or TX (heterozygous) genotype for the Single Nucleotide Polymorphism marker SNP_40 at nucleotide 51 of SEQ ID NO: 40 (or in a variant thereof);
w) the AA (homozygous) or AG or AX (heterozygous) genotype for the Single Nucleotide Polymorphism marker SNP_41 at nucleotide 51 of SEQ ID NO: 41 (or in a variant thereof);
x) the AA (homozygous) or AG or AX (heterozygous) genotype for the Single Nucleotide Polymorphism marker SNP_42 at nucleotide 51 of SEQ ID NO: 42 (or in a variant thereof).

In one embodiment a method for detecting, selecting and/or breeding a plant or plant part comprising QTL3.1 or a variant thereof is provided, comprising carrying out a molecular marker assay and optionally selecting a plant or plant part which comprises the following genotypes or haplotypes for at least 3, preferably at least 4, 5, 6 or 7 Single Nucleotide Polymorphism (SNP) markers selected from the group consisting of:
a) the CC (homozygous) or CX (heterozygous) genotype for the Single Nucleotide Polymorphism marker SNP_29 at nucleotide 51 of SEQ ID NO: 29 (or in a variant thereof);
b) the AA (homozygous) or AG or AX (heterozygous) genotype for the Single Nucleotide Polymorphism marker SNP_30 at nucleotide 51 of SEQ ID NO: 30 (or in a variant thereof);
c) the GG (homozygous) or GT or GX (heterozygous) genotype for the Single Nucleotide Polymorphism marker SNP_31 at nucleotide 51 of SEQ ID NO: 31 (or in a variant thereof);
d) the GG (homozygous) or GA or GX (heterozygous) genotype for the Single Nucleotide Polymorphism marker SNP_32 at nucleotide 51 of SEQ ID NO: 32 (or in a variant thereof);
e) the GG (homozygous) or GC or GX (heterozygous) genotype for the Single Nucleotide Polymorphism marker SNP_33 at nucleotide 51 of SEQ ID NO: 33 (or in a variant thereof);
f) the GG (homozygous) or GA or GX (heterozygous) genotype for the Single Nucleotide Polymorphism marker SNP_34 at nucleotide 51 of SEQ ID NO: 34 (or in a variant thereof);
g) the TT (homozygous) or TC or TX (heterozygous) genotype for the Single Nucleotide Polymorphism marker SNP_35 at nucleotide 51 of SEQ ID NO: 35 (or in a variant thereof);
h) the TT (homozygous) or TTG or TTX (heterozygous) genotype for the Single Nucleotide Polymorphism marker SNP_36 at nucleotide 51 of SEQ ID NO: 36 (or in a variant thereof);
The plant or plant parts may be of one or more wild donor accessions, or of one or more breeding lines or varieties. X may be any nucleotide.

Optionally the selected plant or plants may be phenotyped for internal fruit rot resistance.

In another embodiment a method for identifying or selecting or modifying a cucumber plant or plant part comprising
a) identifying or modifying a cucumber plant or plant part to comprise at least two copies on chromosome 3 of one or more or all of the following genes:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60, or
b) identifying or modifying a cucumber plant or plant part to comprise an increased gene expression of one or more or all of the following genes compared to the wild type plant or plant part:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60.

In one aspect the cucumber plant is a cultivated cucumber plant in which an introgression fragment comprising the above at least two copies or enhanced gene expression of one or more genes has been introduced or in which the copy number or gene expression is modified by e.g. targeted genome editing.

A wild type cultivated cucumber is any cucumber line or variety comprising a chromosome 3 on which only one copy of OGD1 to OGD8 and HR-like protein is present, and wherein these genes are wild type genes, under control of the wild type promoter, for example as in any cucumber variety such as long cucumber hybrid varieties Sheila F1, Drogba F1, Michella F1, Petrifin F1, Sensate F1, SEpalin F1, etc. Also cucumber seeds have been deposited which lack the duplication of QTL3.1 and thus comprise the wild type genes (with wild type gene expression) on chromosome 3, having been given deposit number NCIMB 43531 (DB-susceptible).

In one embodiment the presence of the introgression fragment, or the chromosome 3 region (or variant or orthologous chromosome 3 region), comprising QTL3.1 (or a variant thereof), is detectable by a molecular marker assay which detects the wild donor SNP haplotype or genotype of at least 3, preferably at least 4, 5, 6, 7, 8, 9 or more Single Nucleotide Polymorphism (SNP) markers of the sub-groups consisting of: SNP_19 to SNP_27, SNP_27 to SNP_35, SNP_35 to SNP_42, SNP_21 to SNP_30 and/or SNP_30 to SNP_40. SNP_29 to SNP_36, SNP_35 to SNP_36.

In another embodiment the presence of the introgression fragment is detectable by the duplication of the region in between SNP_35 and 659 nt downstream of SNP_36, i.e. a duplication of SEQ ID NO: 72 and 73 or a sequence comprising at least 95% sequence identity to SEQ ID NO: 72 and 73 and/or by the duplication of one or more genes encoding OGD1 to OGD8 and/or HR-like protein and/or by the presence of one or more of the genes encoding OGD1 to OGD8 and/or HR-like protein as having a higher gene expression than the wild type genes, e.g. as present in NCIMB 43531 (DB-susceptible).

The SNP genotype refers to two nucleotides, and genomic sequences comprising one of these two nucleotides, one on each chromosome 3. So a plant having a GG genotype for SNP_19 has an identical nucleotide (G) on both chromosomes (i.e. is homozygous for the introgression fragment), while a plant having e.g. an GA genotype for SNP_19 has one chromosome with an G at nucleotide 51 of SEQ ID NO: 19 (or at the equivalent nucleotide of a genomic sequence comprising at least 95%, 96% or 97% sequence identity to SEQ ID NO:19) and one chromosome with an A at nucleotide 51 of SEQ ID NO: 19 and is heterozygous for the introgression fragment. The nucleotide for the chromosome lacking the introgression fragment may also be indicated as X, wherein X is selected from any nucleotide (A, T, G, C). The genotype for SNP_19, wherein the introgression fragment is in heterozygous form, may thus be indicated as GX. The same applies to the other SNP markers. As the genomic sequences around the SNP markers provided herein may vary slightly, e.g. in introgression fragments from other wild donors (i.e. variants or orthologous chromosome 3 regions) it is clear that the nucleotide sequences before and after the SNP may not be 100% identical to the sequences provided herein. Therefore, sequences having at least 95%, 96% or 97% sequence identity to the sequences provided herein (when aligned over the entire length as defined), but which comprise the same SNP genotype, are encompassed herein.

In one aspect, the introgression fragment, or the chromosome 3 region (or variant or orthologous chromosome 3 region) comprising the QTL (QTL3.1 or variant), which is e.g. detectable by the above one or more markers or enhanced copy number or enhanced gene expression of the one or more genes, is from a wild cucumber. In one aspect it is the same introgression fragment as found on chromosome 3 in seeds deposited under accession number NCIMB43530, or a smaller fragment (sub-fragment) retaining the QTL. SNP markers SNP_19 (at nucleotide 9047770 of chromosome 3) to SNP_42 (at nucleotide 9357469 of chromosome 3) span a region of about 0.31 Mb on chromosome 3 and on this donor region the QTL is found, which is now known to lie in between SNP_35 and SNP_36 (a region of 27521 bases) and a duplication of about 150 kb flanked by (and optionally including) SEQ ID NO: 83 and 84 was found to be present in the resistant plant. In one aspect the introgression fragment on chromosome 3 is equal to or less than 1 Mb in size, preferably equal to or less than 0.5 Mb in size, more preferably equal to or less than 0.33 Mb or 0.31 Mb or 350 kb or 300 kb or 60 kb. In one aspect the introgression fragment is at least 10 kb, 15 kb, 20, 30, 40, 50 kb in size. Thus, various ranges of introgression fragment sizes are encompassed herein, such as fragments less than 1 Mb but more than 10 kb which retain the QTL3.1 The size of an introgression fragment can be easily determined by e.g. whole genome sequencing or Next Generation Sequencing, e.g. as described in Qi et al. 2013 (supra) or in Huang et al. 2009 (supra). Especially introgression regions can be easily distinguished from cultivated genomic regions due to the larger amount of genetic variation (SNPs, INDELs, etc.) in the introgression region.

To obtain the introgression fragment present on chromosome 3 from the deposited seeds (NCIMB 43530), i.e. to transfer the introgression fragment comprising the QTL to another cultivated cucumber plant, a plant is grown from the seed and the plant is crossed with a cultivated cucumber plant to obtain F1 seeds. As NCIMB 43530 contains two recombinant chromosomes 3 (comprising the introgression fragment comprising the duplication of QTL3.1 in homozygous form) all of the F1 seed and plants grown therefrom will contain one recombinant chromosome 3 from the NCIMB 43530 parent and one non-recombinant chromosome 3 from the other cultivated parent. By further selfing and/or crossing and/or backcrossing, QTL3.1 (the duplication of the region comprising at least 95% sequence identity to SEQ ID NO: 72, or a part of this duplicated region) can be transferred into any cucumber breeding line or variety. Thus, by traditional breeding one can transfer the recombinant chromosome 3 from NCIMB 43530 into other cultivated cucumber lines or varieties. Progeny plants which comprise the QTL3.1 can be screened for, and selected for, by the presence of e.g. the donor genotype or haplotype for one or more of the above SNP markers or for the copy number or gene expression of OGL1 to OGL8 and the HR-like protein.

To generate shorter introgression fragments, e.g. sub-fragments of the fragment present in NCIMB 43530, meiosis needs to take place and plants comprising the recombinant chromosomes 3, and especially new meiotic recombination events within the introgression fragment, need to be identified. For example, seeds of NCIMB43530 can be selfed one or more times to produce F1, F2 or F3 plants (or further selfing generations), and/or F1, F2 or F3 plants (etc.) comprising the recombinant chromosome 3 can be backcrossed to a cultivated parent. Plants which comprise the recombinant chromosome 3 can be screened for, and selected for, by e.g. the presence of the donor genotype or haplotype of one or more of the above SNP markers in order to identify plants comprising a smaller introgression fragment and/or for the copy number or gene expression of OGL1 to OGL8 and the HR-like protein. Such new recombinants can then be tested for the presence of the QTL3.1 on the smaller introgression fragment by determining the internal fruit rot resistance compared to the (genetic) control lacking the introgression fragment.

Similarly, cultivated cucumber plants comprising QTL3.1 (or a variant thereof) can be generated and/or identified using different methods. For example, to obtain a cultivated cucumber plant comprising a introgression fragment from a wild donor of cucumber, first a wild donor is identified which comprises e.g. the donor SNP genotype or haplotype one or more of the SNP markers linked to QTL3.1 disclosed herein, e.g. any one, or more, or all of the markers described herein above, or comprises a duplication of all or part of the region of SEQ ID NO: 72 or comprising at least 95% identity to SEQ ID NO: 72, or comprises an enhanced copy number (e.g. duplication) and/or enhanced gene expression of one or more of the genes selected from OGD1 to OGD8 and/or HR-like protein. The identified donor plant is crossed with a cultivated cucumber plant to obtain F1 seeds. The F1 can be selfed to produce F2, F3, etc. plants, and/or F2 plants or F3 plants, etc., can be backcrossed to the cultivated cucumber parent. Plants which are comprising QTL3.1 (or a variant thereof) can be screened for, and/or selected for, by e.g. the presence of the donor genotype or haplotype of one or more of the above SNP markers or the increased copy number and/or increased gene expression of one or more of the genes selected from OGD1 to OGD8 and HR-like protein and/or screened for, and/or selected for, an increased internal fruit rot resistance phenotype compared to e.g. the initial cultivated parent (lacking the introgression). Alternatively or in addition, QTL mapping or sequencing can be carried out in order to e.g. identify further molecular markers linked to the QTL3.1 (or a variant thereof) or to check the sequence information of the 9 genes in the region and/or to generate cultivated cucumber plants comprising an introgression fragment on chromosome 3 which confers internal fruit rot resistance.

In one embodiment the introgression fragment in a cultivated cucumber plant or plant part, or a donor fragment in a wild cucumber plant or plant part, comprising QTL3.1 (or a variant thereof), comprises:
a) the donor SNP haplotype of at least 3, 4, 5, 6, 7, 8, 9, 10, 11, 12 or more SNP markers of SNP_19 to SNP_42 or of at least 3, 4, 5, 6 or 7 SNP markers of SNP_29 to SNP_36 or of SNP_35 and/or SNP_36;
b) the donor SNP haplotype of at least 3, 4, 5, 6, 7, 8, 9, 10, 11, 12 or more consecutive SNP markers of SNP_19 to SNP_42, or of at least 3, 4, 5, 6 or 7 consecutive SNP markers of SNP_29 to SNP_36 or of SNP_35 and/or SNP_36;
c) the donor SNP haplotype of SNP_19 to SNP_27, and/or of SNP_27 to SNP_35, and/or of SNP_35 to SNP_42, or of SNP_21 to SNP_30 and/or of SNP_30 to SNP_40 and/or of SNP_29 to SNP_36, and/or of SNP_35 to SNP_36, and/or of SNP_35 or SNP_36;
   wherein the donor SNP haplotype is:
a Guanine for SNP_19 at nucleotide 51 of SEQ ID NO: 19 or at nucleotide 51 of a sequence comprising at least 97% identity to SEQ ID NO: 19,
a Guanine for SNP_20 at nucleotide 51 of SEQ ID NO: 20 or at nucleotide 51 of a sequence comprising at least 97% identity to SEQ ID NO: 20,
a Thymine for SNP_21 at nucleotide 51 of SEQ ID NO: 21 or at nucleotide 51 of a sequence comprising at least 97% identity to SEQ ID NO: 21,
an Adenine for SNP_22 at nucleotide 51 of SEQ ID NO: 22 or at nucleotide 51 of a sequence comprising at least 97% identity to SEQ ID NO: 22,
a Cytosine for SNP_23 at nucleotide 51 of SEQ ID NO: 23 or at nucleotide 51 of a sequence comprising at least 97% identity to SEQ ID NO: 23, instead of a Cytosine-Adenine or Cytosine-X (where X is any nucleotide) at this position,
an Adenine for SNP_24 at nucleotide 51 of SEQ ID NO: 24 or at nucleotide 51 of a sequence comprising at least 97% identity to SEQ ID NO: 24,
a Guanine for SNP_25 at nucleotide 51 of SEQ ID NO: 25 or at nucleotide 51 of a sequence comprising at least 97% identity to SEQ ID NO: 25,
a Thymine for SNP_26 at nucleotide 51 of SEQ ID NO: 26 or at nucleotide 51 of a sequence comprising at least 97% identity to SEQ ID NO: 26,
a Cytosine for SNP_27 at nucleotide 51 of SEQ ID NO: 27 or at nucleotide 51 of a sequence comprising at least 97% identity to SEQ ID NO:27,
a Guanine for SNP_28 at nucleotide 51 of SEQ ID NO: 28 or at nucleotide 51 of a sequence comprising at least 97% identity to SEQ ID NO: 28,
a Cytosine for SNP_29 at nucleotide 51 of SEQ ID NO: 29 or at nucleotide 51 of a sequence comprising at least 97% identity to SEQ ID NO: 29,
an Adenine for SNP_30 at nucleotide 51 of SEQ ID NO: 30 or at nucleotide 51 of a sequence comprising at least 97% identity to SEQ ID NO: 30,
a Guanine for SNP_31 at nucleotide 51 of SEQ ID NO: 31 or at nucleotide 51 of a sequence comprising at least 97% identity to SEQ ID NO: 31,
a Guanine for SNP_32 at nucleotide 51 of SEQ ID NO: 32 or at nucleotide 51 of a sequence comprising at least 97% identity to SEQ ID NO: 32,
a Guanine for SNP_33 at nucleotide 51 of SEQ ID NO: 33 or at nucleotide 51 of a sequence comprising at least 97% identity to SEQ ID NO: 33,
a Guanine for SNP_34 at nucleotide 51 of SEQ ID NO: 34 or at nucleotide 51 of a sequence comprising at least 97% identity to SEQ ID NO: 34,
a Thymine for SNP_35 at nucleotide 51 of SEQ ID NO: 35 or at nucleotide 51 of a sequence comprising at least 97% identity to SEQ ID NO: 35,
a Thymine for SNP_36 at nucleotide 51 of SEQ ID NO: 36 or at nucleotide 51 of a sequence comprising at least 97% identity to SEQ ID NO: 36, instead of a Thymine-Guanine or Thymine-X (where X is any nucleotide) at this position,
a Cytosine for SNP_37 at nucleotide 51 of SEQ ID NO: 37 or at nucleotide 51 of a sequence comprising at least 97% identity to SEQ ID NO: 37,
a Guanine for SNP_38 at nucleotide 51 of SEQ ID NO: 38 or at nucleotide 51 of a sequence comprising at least 97% identity to SEQ ID NO: 38,
a Cytosine for SNP_39 at nucleotide 51 of SEQ ID NO: 39 or at nucleotide 51 of a sequence comprising at least 97% identity to SEQ ID NO: 39,
a Thymine for SNP_40 at nucleotide 51 of SEQ ID NO: 40 or at nucleotide 51 of a sequence comprising at least 97% identity to SEQ ID NO: 40,
an Adenine for SNP_41 at nucleotide 51 of SEQ ID NO: 41 or at nucleotide 51 of a sequence comprising at least 97% identity to SEQ ID NO: 41,
an Adenine for SNP_42 at nucleotide 51 of SEQ ID NO: 42 or at nucleotide 51 of a sequence comprising at least 97% identity to SEQ ID NO: 42.

Thus, different wild cucumber accessions (or selections or selfings thereof) can be screened for the SNP haplotype of a), b) or c) above to identify other donor accessions than MYCR3 which comprise QTL3.1. Optionally the donor accessions can be screened also for the presence of QTL5.1 and/or phenotypically for the susceptibility or resistance to internal fruit rot caused by DB. A wild cucumber accession identified or selected for having the donor SNP haplotype of a), b) or c) can then be used to backcross QTL3.1 into cultivated cucumber.

Thereby a cultivated cucumber can be generated comprising an introgression fragment comprising QTL3.1 (or a variant thereof) and comprising the donor SNP haplotype of a), b) or c) above. The cultivated cucumber is preferably tested phenotypically for the susceptibility or resistance to internal fruit rot caused by DB. The base-line susceptibility should be significantly reduced in the cultivated cucumber plant comprising QTL3.1. As QTL3.1 and QTL5.1 are additive, the effect of QTL3.1 is optionally in one aspect determined in combination with QTL5.1, present together in the cultivated cucumber.

In a further embodiment the introgression fragment in a cultivated cucumber plant or plant part, or a donor fragment in a wild cucumber plant or plant part, comprising QTL3.1 (or a variant thereof), comprises:
a) at least two copies on chromosome 3 of one or more or all of the following genes:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60, and/or
b) genes on chromosome 3 having an increased gene expression of one or more or all of the following genes compared to the wild type genes on chromosome 3:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60

Also provided are seeds from which a plant of the invention can be grown, as are cucumber fruits harvested from a plant of the invention and comprising the recombinant chromosome 3 in their genome. Likewise a plant cell, tissue or plant part of a plant or of a seed is provided comprising at least one recombinant chromosome 3, wherein said recombinant chromosome 3 comprises an introgression fragment from a wild cucumber and wherein said introgression fragment comprises QTL3.1 (or a variant thereof) conferring significantly resistance to internal fruit rot.

In one embodiment also a method for identifying and/or selecting plant or plant part comprising an introgression fragment in a cultivated cucumber plant or plant part, or a donor fragment in a wild cucumber plant or plant part, comprising QTL3.1 (or a variant thereof), is provided comprising DNA analysis to identify and/or select a plant or plant part, wherein the fragment comprises:
a) the donor SNP haplotype of at least 3, 4, 5, 6, 7, 8, 9, 10, 11, 12 or more SNP markers of SNP_19 to SNP_42 or of at least 3, 4, 5, 6 or 7 SNP markers of SNP_29 to SNP_36 or of SNP_35 and/or SNP_36;
b) the donor SNP haplotype of at least 3, 4, 5, 6, 7, 8, 9, 10, 11, 12 or more consecutive SNP markers of SNP_19 to SNP_42, or of at least 3, 4, 5, 6 or 7 consecutive SNP markers of SNP_29 to SNP_36 or of SNP_35 and/or SNP_36;
c) the donor SNP haplotype of SNP_19 to SNP_27, and/or of SNP_27 to SNP_35, and/or of SNP_35 to SNP_42, or of SNP_21 to SNP_30 and/or of SNP_30 to SNP_40 and/or of SNP_29 to SNP_36, and/or of SNP_35 to SNP_36, and/or of SNP_35 or SNP_36;
wherein the donor SNP haplotype is:
a Guanine for SNP_19 at nucleotide 51 of SEQ ID NO: 19 or at nucleotide 51 of a sequence comprising at least 97% identity to SEQ ID NO: 19,
a Guanine for SNP_20 at nucleotide 51 of SEQ ID NO: 20 or at nucleotide 51 of a sequence comprising at least 97% identity to SEQ ID NO: 20,
a Thymine for SNP_21 at nucleotide 51 of SEQ ID NO: 21 or at nucleotide 51 of a sequence comprising at least 97% identity to SEQ ID NO: 21,
an Adenine for SNP_22 at nucleotide 51 of SEQ ID NO: 22 or at nucleotide 51 of a sequence comprising at least 97% identity to SEQ ID NO: 22,
a Cytosine for SNP_23 at nucleotide 51 of SEQ ID NO: 23 or at nucleotide 51 of a sequence comprising at least 97% identity to SEQ ID NO: 23, instead of a Cytosine-Adenine or Cytosine-X at this position,
an Adenine for SNP_24 at nucleotide 51 of SEQ ID NO: 24 or at nucleotide 51 of a sequence comprising at least 97% identity to SEQ ID NO: 24,
a Guanine for SNP_25 at nucleotide 51 of SEQ ID NO: 25 or at nucleotide 51 of a sequence comprising at least 97% identity to SEQ ID NO: 25,
a Thymine for SNP_26 at nucleotide 51 of SEQ ID NO: 26 or at nucleotide 51 of a sequence comprising at least 97% identity to SEQ ID NO: 26,
a Cytosine for SNP_27 at nucleotide 51 of SEQ ID NO: 27 or at nucleotide 51 of a sequence comprising at least 97% identity to SEQ ID NO:27,
a Guanine for SNP_28 at nucleotide 51 of SEQ ID NO: 28 or at nucleotide 51 of a sequence comprising at least 97% identity to SEQ ID NO: 28,
a Cytosine for SNP_29 at nucleotide 51 of SEQ ID NO: 29 or at nucleotide 51 of a sequence comprising at least 97% identity to SEQ ID NO: 29,
an Adenine for SNP_30 at nucleotide 51 of SEQ ID NO: 30 or at nucleotide 51 of a sequence comprising at least 97% identity to SEQ ID NO: 30,
a Guanine for SNP_31 at nucleotide 51 of SEQ ID NO: 31 or at nucleotide 51 of a sequence comprising at least 97% identity to SEQ ID NO: 31,
a Guanine for SNP_32 at nucleotide 51 of SEQ ID NO: 32 or at nucleotide 51 of a sequence comprising at least 97% identity to SEQ ID NO: 32,
a Guanine for SNP_33 at nucleotide 51 of SEQ ID NO: 33 or at nucleotide 51 of a sequence comprising at least 97% identity to SEQ ID NO: 33,
a Guanine for SNP_34 at nucleotide 51 of SEQ ID NO: 34 or at nucleotide 51 of a sequence comprising at least 97% identity to SEQ ID NO: 34, a Thymine for SNP_35 at nucleotide 51 of SEQ ID NO: 35 or at nucleotide 51 of a sequence comprising at least 97% identity to SEQ ID NO: 35,
a Thymine for SNP_36 at nucleotide 51 of SEQ ID NO: 36 or at nucleotide 51 of a sequence comprising at least 97% identity to SEQ ID NO: 36, instead of a Thymine-Guanine or Thymine-X at this position,
a Cytosine for SNP_37 at nucleotide 51 of SEQ ID NO: 37 or at nucleotide 51 of a sequence comprising at least 97% identity to SEQ ID NO: 37,
a Guanine for SNP_38 at nucleotide 51 of SEQ ID NO: 38 or at nucleotide 51 of a sequence comprising at least 97% identity to SEQ ID NO: 38,
a Cytosine for SNP_39 at nucleotide 51 of SEQ ID NO: 39 or at nucleotide 51 of a sequence comprising at least 97% identity to SEQ ID NO: 39,
a Thymine for SNP_40 at nucleotide 51 of SEQ ID NO: 40 or at nucleotide 51 of a sequence comprising at least 97% identity to SEQ ID NO: 40,
an Adenine for SNP_41 at nucleotide 51 of SEQ ID NO: 41 or at nucleotide 51 of a sequence comprising at least 97% identity to SEQ ID NO: 41,
an Adenine for SNP_42 at nucleotide 51 of SEQ ID NO: 42 or at nucleotide 51 of a sequence comprising at least 97% identity to SEQ ID NO: 42.

The DNA analysis in the above method can involve e.g. SNP genotyping, DNA sequencing, PCR analysis or other methods to determine the SNP haplotype of the plant or plant part.

In one embodiment also a method for identifying and/or selecting plant or plant part comprising an introgression fragment in a cultivated cucumber plant or plant part, or a donor fragment in a wild cucumber plant or plant part, comprising QTL3.1 (or a variant thereof), is provided comprising DNA analysis to identify and/or select a plant or plant part, wherein the fragment comprises:
a) at least two copies on chromosome 3 of one or more or all of the following genes:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60, and/or
b) genes having an increased gene expression of one or more or all of the following genes compared to the wild type genes on chromosome 3:
a gene encoding an OGD1 protein comprises the amino acid sequence of SEQ ID NO: 52 or a sequence comprising at least 95% sequence identity to SEQ ID NO: 52,
a gene encoding an OGD2 protein comprises the amino acid sequence of SEQ ID NO: 53 or a sequence comprising at least 95% sequence identity to SEQ ID NO: 53,
a gene encoding an OGD3 protein comprises the amino acid sequence of SEQ ID NO: 54 or a sequence comprising at least 95% sequence identity to SEQ ID NO: 54,
a gene encoding an OGD4 protein comprises the amino acid sequence of SEQ ID NO:55 or a sequence comprising at least 95% sequence identity to SEQ ID NO: 55,
a gene encoding an OGD5 protein comprises the amino acid sequence of SEQ ID NO: 56 or a sequence comprising at least 95% sequence identity to SEQ ID NO: 56,
a gene encoding an OGD6 protein comprises the amino acid sequence of SEQ ID NO: 57 or a sequence comprising at least 95% sequence identity to SEQ ID NO: 57,
a gene encoding an OGD7 protein comprises the amino acid sequence of SEQ ID NO: 58 or a sequence comprising at least 95% sequence identity to SEQ ID NO: 58,
a gene encoding an OGD8 protein comprises the amino acid sequence of SEQ ID NO: 59 or a sequence comprising at least 95% sequence identity to SEQ ID NO: 59,
a gene encoding a HR-like protein comprises the amino acid sequence of SEQ ID NO: 60 or a sequence comprising at least 95% sequence identity to SEQ ID NO: 60.

As mentioned previously, it is also an aspect to combine both QTL5.1 (or a variant thereof) and QTL3.1 (or a variant thereof) in a single cultivated cucumber plant and plant parts. All embodiments relating to the individual QTLs herein are, therefore, also combined in one aspect of the invention. As there is an additive effect between the QTLs and the increase in internal fruit rot resistance (reduced susceptibility to internal fruit rot) is highest when both QTLs are present together, the combination of both QTLs, preferably in homozygous form, is of great advantage for the cultivation of the cucumbers.

The molecular markers described herein may be detected according to standard method. For example SNP markers can easily be detected using a KASP-assay (see www.kpbioscience.co.uk) or other SNP genotyping assays. For developing a KASP-assay, for example 50 base pairs upstream and 50 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP assay method.

Thus, in one aspect, the SNP markers and the presence/absence of the marker associated with the QTLs is determined using a KASP assay, but equally other SNP genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

The physical size of an introgression fragment can be determined by various methods, such as physical mapping, sequencing or by visualization of the introgression using Fluorescent in situ hybridization (FISH) images (Verlaan et al. 2011, Plant Journal 68:1093-1103).

Copy number of the 9 genes or of any of the SNP markers can be determined using known methods, such as digital PCR, genomic sequence analysis, allele specific PCR, SNP arrays, hybridization, Paralogue Ratio Tests and other known methods. Gene expression can be analysed using known methods, such as qRT-PCR to quantify the mRNA/cDNA transcripts and other known methods.

Cultivated cucumber plants with smaller introgression fragments on chromosome 5 and/or 3 can be generated by generating new recombinant plants from a population of plants derived from a cross between a cultivated cucumber plant (lacking the introgressions) and a plant of the invention and selecting recombinant progeny having smaller introgression sizes. Such plants are, thus, in one aspect derived from (progeny or descendants of) the recombinant chromosome 5 and 3 present in plants of which seeds have been deposited under NCIMB43530. Such progeny or descendants which retain the QTL5.1 and/or QTL3.1, and thus the higher DB internal fruit rot resistance compared to plants lacking the introgression(s) as described herein, are encompassed herein.

In tomato, for example the large S. chilense introgression fragment on chromosome 6 (about 27 cM) which comprises the Ty-3 allele has been reduced by selecting a recombinant progeny line (LA1931-AL-F2), which comprises a much smaller S. chilense introgression fragment (about 6 cM) comprising Ty-3 (see Ji et al. 2007, Mol. Breeding 20:271-284).

The cultivated cucumber plant according to the invention may be an inbred line, an OP (open pollinated variety) or an F1 hybrid. In one aspect the F1 hybrid comprises only one recombinant chromosome 5 and/or one recombinant chromosome 3 (comprising the introgression fragment with the QTL), i.e. the F1 hybrid is heterozygous for the introgression fragment(s) and e.g. the SNP markers described herein. Such an F1 hybrid is produced by crossing two inbred parent lines, one of which possesses the introgression fragment(s) (preferably in homozygous form, although not necessarily) and collecting the F1 hybrid seeds from said cross. In another aspect the F1 hybrid may comprise the introgression fragment(s) in homozygous form, i.e. produced by crossing two inbred parent lines, each comprising the introgression fragment(s) in homozygous or heterozygous form.

The cultivated cucumber plant may be of any type. Preferably it has good agronomic and good fruit quality characteristics. The cultivated cucumber plant is in one aspect uniform, both genetically and phenotypically. Especially fruit characteristics are uniform, e.g. regarding shape, skin color, skin thickness, skin ribs, skin toughness, spines (spine color, spine density, etc.), presence/absence of warts, length and diameter at edible and marketable maturity, flavour, etc. Likewise seed characteristics (i.e. characteristics of the seeds from which the plant is grown) are uniform, e.g. seed size, seed color, etc. Thus, plants of the line or variety comprising QTL5.1 (or a variant) and/or QTL3.1 (or a variant) in homozygous or heterozygous form produce uniform fruits, meaning that there is little variation between fruits of plants grown under the same environmental conditions and when fruits are at the same developmental stage (e.g. for qualitative characteristics at least 98%, 99% or preferably 100% of all plants or plant parts, fruits or seed are identical for the characteristics; for quantitative characteristics at least 90%, 95%, 98% of all plants or plant parts, fruits or seed are identical for the characteristics).

The cultivated cucumber plant comprising QTL5.1 (or a variant thereof) and/or QTL3.1 (or a variant thereof) according to the invention may be of any type, e.g. it may be of one of the following cucumber types: pickling cucumbers (e.g. American pickling, European pickling type), slicing cucumbers (e.g. American slicing), long cucumbers, short cucumbers, European greenhouse cucumbers, Beit-Alpha type cucumbers, oriental trellis type cucumbers, Asian cucumbers (e.g. selected from Indian Mottled cucumber, Chinese Long cucumber, Korean cucumber and Japanese cucumber type). In one aspect the cultivated cucumber according to the invention is an inbred line or a F1 hybrid of a pickling cucumber type, slicing cucumber type, long cucumber type, short cucumber type, European greenhouse cucumbers, Beit-Alpha type cucumbers, oriental trellis type cucumbers, Chinese long cucumber type, Korean cucumber type or Japanese cucumber type. In a specific embodiment the cucumber is an inbred line or an F1 hybrid of a long cucumber, especially a European greenhouse cucumber, or a short cucumber.

The plant may be a single cross F1 hybrid or an inbred line, comprising QTL5.1 (or a variant) and/or QTL3.1 (or a variant) in homozygous or heterozygous form. In one aspect it is an F1 hybrid produced by crossing an (inbred) parent plant comprising QTL5.1 (or a variant) and/or QTL3.1 (or a variant) in homozygous form with an (inbred) parent plant lacking QTL5.1 and QTL3.1 (i.e. lacking introgression fragments comprising the QTLs). Thus in one aspect the F1 hybrid is heterozygous for QTL5.1 and/or QTL3.1.

In another aspect it is an F1 hybrid produced by crossing an (inbred) parent plant comprising QTL5.1 (or a variant thereof) and/or QTL3.1 (or a variant) in homozygous form with an (inbred) parent plant that also comprises QTL5.1 (or a variant thereof) and/or QTL3.1 (or a variant) in homozygous form. Thus, in one aspect the F1 hybrid is homozygous for QTL5.1 and/or QTL3.1.

In one aspect the F1 hybrid is a long cucumber type, e.g. a European greenhouse cucumber type, suitable for the traditional glasshouse cultivation or for high-wire cultivation. In the traditional glasshouse cultivation method the main stem of the plant is led up to a horizontal iron wire that is suspended at a height of about two meters above the ground. When the plant reaches this height and attaches to the wire, it is “topped” by removing its growth point in order to terminate further proliferation, whereupon lateral shoots start to develop. These lateral shoots are allowed to grow downward to a height of about 1 meter above the ground, and the growth points are then removed from them. This is followed by flowering and the development of the fruits both on the stem and on the lateral shoots or tendrils, but the fruits on the tendrils develop later than those on the stem. The fruits are harvested about 6 weeks after sowing.

In the high-wire cultivation no lateral tendrils are allowed to grow and all the harvest comes from the stem. Specific varieties have been developed by Nunhems which are highly suitable for high-wire cultivation, as they provide a gene called “compact”, see. WO2009/059777, for example varieties High-Jack, Hi-Power, Hi-Lisa. Thus, in one aspect of the invention the cultivated cucumber plant comprises additionally the compact gene described in WO2009/059777.

In another aspect the introgression fragment of the invention is present in a long cucumber type, such as variety Kasja (Nunhems), which is a long cucumber variety producing fruits of 27-38 cm. A “long cucumber type” or “long cucumber plants” are greenhouse cucumbers characterized by fruits of at least about 26 cm or 27 cm to 37 or 38 cm in length or longer (for example 40 cm, 42 cm or more), preferably with parthenocarpic fruit formation. Examples of long cucumber types are the Sabrina and Korinda varieties, or cucumber plants that are awarded a score of 7-9 for the length of the fruit according to the CPVO Protocol. Other long cucumber varieties are, for example, Bodega, Bologna, Kamaro, Flamingo, Discover, Kalunga, Kasja, Logica, Millagon. Nicola, Milika, Manuela, Frida, Activa, Alaya, Savanna, Sienna, Bella, Sheila, Bornand.

In one aspect the European greenhouse cucumber is the plant of which seeds were deposited under accession number NCIMB 43530, or progeny thereof, whereby the progeny retain the introgression fragment comprising QTL5.1 and/or QTL3.1 (as e.g. detectable by the presence of one or more markers as described elsewhere).

In another aspect the plant according to the invention is not a wild cucumber plant or a wild relative of cucumber or a landrace.

In yet another aspect the plant according to the invention is a cultivated cucumber of the Eurasian cucumber group, the East Asian cucumber group or the Xishuangbanna cucumber group or of the Indian cucumber group. In another aspect the plant according to the invention is not a cucumber of the Indian cucumber group.

In one embodiment of the invention the cultivated cucumber plant comprising QTL5.1 (or a variant) and/or QTL3.1 (or a variant) produces seedless fruits without pollination, i.e. is parthenocarpic. Such seedless fruits are also encompassed herein.

In a further embodiment of the invention the cultivated cucumber plant comprising QTL5.1 (or a variant) and/or QTL3.1 (or a variant) is primarily gynoecious or entirely gynoecious.

In a further embodiment of the invention the cultivated cucumber plant comprising QTL5.1 (or a variant) and/or QTL3.1 (or a variant) is uniform and genetically stable regarding the morphological characteristics of the fruits produced by said plant, e.g. regarding fruit shape, fruit color, skin thickness, warts, etc.

Fruit characteristics, such as average fruit length, average fruit diameter, skin thickness, presence/absence of warts, spininess, skin toughness, skin color, fruit neck shape, fruit tapering, shape of medial cross section, presence or absence of seeds (parthenocarpy), etc. depend on the cucumber type, i.e. the cultivated genetic background (gene pool) into which the QTL(s) is (are) introgressed. Thus, depending on the cucumber type, various fruit shapes, sizes and fruit types are included herein. In one aspect the fruits are seedless.

The two main types of cucumber fruit grown commercially today in the United States are fresh market (slicing) type and the processing (pickling) type. Varieties and production methods are typically adapted to the end use. Slicing cucumbers are often longer, larger and have darker and thicker skin, whereas pickling/processing cucumbers have a shorter fruit, thinner skin with interior flesh that make them more amenable to pickling. Seedless varieties are generally preferable for both fresh market and for pickling as developing and large seeds are not palatable.

In one aspect the plant of the invention is a pickling type (processing type) and produces fruits which at edible maturity and/or marketable size have an average fruit length of at least 10 cm, or at least 11 cm, or at least 12 cm, or at least 13 cm and/or a fruit length to diameter ratio of at least 2, at least 2.5, at least 3, or more.

In a different aspect the plant of the invention is a fresh market type, e.g. a long cucumber type or slicing type, and produces fruits have an average fruit length at edible maturity and/or marketable size which is longer than the pickling type, e.g. at least 15 cm, 16 cm, 17 cm, 18 cm, 19 cm, 20 cm, 25 cm, 26 cm, 27 cm, 28 cm, 29 cm, 30 cm, 32 cm, 40 cm, or more. In one aspect the fruit length/diameter ratio is at least 3.5, preferably at least 4, 5, 6, or more.

In one aspect the cucumber plant is a long cucumber type and comprises QTL5.1 and/or QTL3.1 and has an average fruit length at edible maturity and/or marketable size of at least 30 cm, preferably at least 31 cm or at least 32, 33, 34, 35, 36, 37 or 38 cm.

In a preferred aspect the plant of the invention is a long cucumber type producing fruits of marketable size, especially seedless fruits. The fruits of marketable size, and parts thereof, and food or feed products containing these, are also encompassed herein. In one embodiment the SNP markers are detectable in the fruits, fruit parts or food or feed products comprising these.

In one aspect the plant is an indeterminate cucumber. In another aspect the cucumber is determinate.

Also seeds from which a plant according to the invention can be grown is provided herein, as are cucumber fruits harvested from a plant according to the invention. These comprise the QTL(s) in their genome and can therefore be distinguished from other fruits by e.g. the presence of one or more of the SNP markers provided herein or by the copy number or gene expression of one or more of the 9 genes described herein.

In one aspect the fruits are bitter free (selected from the groups bitter and bitter free) at edible maturity and/or at marketable size of the fruits.

In a further aspect the fruit has a thin skin (selected from the groups thick and thin) at edible maturity and/or at marketable size of the fruits.

In a different embodiment the QTL(s) is (are) introgressed into a cucumber type called ‘Compact’, as described in U.S. Pat. No. 8,710,303B2. Thus, the cucumber plants according to the invention comprise the compact gene as described in U.S. Pat. No. 8,710,303B2 in homozygous or heterozygous form, e.g. as present in varieties Hi-Jack, Hi-Power, Hi-Lisa and others (Nunhems varieties).

A further embodiment of the invention is a plant cell, tissue or plant part of a plant or of a seed according to the invention comprising at least one recombinant chromosome 5 and/or at least one recombinant chromosome 3, wherein said recombinant chromosome 5 or 3 comprises an introgression fragment from a wild cucumber and wherein said introgression fragment comprises a QTL conferring increased internal fruit rot resistance compared to a plant lacking the QTLs 5.1 and a duplication of QTL3.1.

Also the use of a recombinant chromosome 5 and/or 3 comprising an introgression fragment from a wild cucumber (said introgression fragment comprising an allele conferring enhanced resistance to internal fruit rot) for breeding cucumber varieties having enhanced resistance to internal fruit rot is encompassed herein. In one aspect said recombinant chromosomes 5 and/or 3 is the recombinant chromosome 5 and/or the recombinant chromosome 3 as found in seeds deposited under accession number NCIMB 43530, or is derived from said recombinant chromosome (e.g. is a smaller fragment of the introgression fragment found in said seeds).

Likewise, the use of a chromosome 5 and/or 3 as found in seeds deposited under accession number NCIMB 43530, or in progeny thereof, for generating a cultivated cucumber plant comprising an introgression fragment on said chromosome 5 and/or 3 is encompassed herein, wherein said introgression fragment confers enhanced internal fruit rot resistance compared to the control cucumber plant lacking said introgression fragment, such as the genetic control or a control breeding line or variety. In one aspect plants grown from seeds deposited under NCIMB43531 (lacking QTL 5.1 and lacking a duplication of QTL3.1) and/or NCIMB43530 (comprising both QTL5.1 and comprising a duplication of QTL3.1 in homozygous form) may be used as control, e.g. as susceptible control and as resistant control respectively.

Similarly the use of plants grown from seeds deposited under accession number NCIMB 43530 or progeny thereof, for generating a cultivated cucumber plant comprising enhanced internal fruit rot resistance is encompassed herein, wherein said enhanced internal fruit rot resistance is conferred by an introgression fragment obtained from chromosome 5 and/or 3 of said plants or progeny thereof.

Also a method for identifying (or detecting) a cultivated C. sativus var. sativus plant or plant part comprising an introgression fragment on chromosome 5 is provided, optionally wherein said introgression fragment is as found in NCIMB 43530 or a smaller (sub) fragment derived therefrom, comprising:
a) providing a cultivated C. sativus var. sativus plant or plant parts or DNA of such plant or plant part,
b) screening said plant, plant part or DNA using a molecular marker assay which detects at least one SNP marker selected from the group consisting of:
SNP_01 to SNP_18 or SNP_12 to SNP_15 (as shown in FIG. 6) for detecting the introgression fragment on chromosome 5; and
c) identifying and/or selecting a plant comprising:
   i) at least 1, 2, 3, 4, 5, 6, 7, 8, 9, 10 or more of the SNP markers of SNP_01 to SNP_18, or SNP_12 to SNP_15 (as shown in FIG. 6), for detecting the introgression fragment on chromosome 5; or
   ii) at least 2, 3, 4 5, 6, 7, 8, 9, 10 or more consecutive markers selected from SNP_01 to SNP_18, or from SNP_12 to SNP_15 (as shown in FIG. 6), for detecting the introgression fragment on chromosome 5; or
   iii) at least 1, 2, 3, 4 5, 6, 7, 8 or more markers of a group, said group consisting of SNP_01 to SNP_06; SNP_06 to SNP_12; SNP_12 to SNP_18; SNP_03 to SNP_10; SNP_10 to SNP_16; or SNP_12 to SNP_15 (as shown in FIG. 6) or
   iv) at least 2, 3, 4 5, 6, 7, 8 or more consecutive markers of a group, said group consisting of SNP_01 to SNP_06; SNP_06 to SNP_12; SNP_12 to SNP_18; SNP_03 to SNP_10; SNP_10 to SNP_16, or SNP_12 to SNP_15 (as shown in FIG. 6).

Further a method of producing C. sativus F1 hybrid plants comprising an introgression fragment conferring enhanced internal fruit rot resistance is provided comprising:
a) providing a first inbred cucumber plant comprising a recombinant chromosome 5 in homozygous form having an introgression fragment comprising a QTL conferring enhanced internal fruit rot resistance, optionally wherein said introgression fragment is as in NCIMB 43530 or a smaller (sub-) fragment,
b) providing a second inbred cucumber plant,
c) crossing said cucumber plant of a) with said cucumber plant of b),
d) collecting F1 hybrid seeds from said cross.

The F1 hybrid seeds collected are also an embodiment of the invention.

In another aspect a method for generating progeny of NCIMB 43530 is provided, said method comprising:
a) growing a plant from seeds deposited under accession number NCIMB 43530;
b) selfing said plant one or more times and/or crossing said plant one or more times with another cucumber plant to generate progeny seeds;
c) screening said progeny seeds or plants grown from said seeds or parts of the seeds or plants using a molecular marker assay which detects at least one SNP marker selected from the group consisting of:
SNP_01 to SNP_18 or SNP_12 to SNP_15 (as shown in FIG. 6) for detecting the introgression fragment on chromosome 5;
d) identifying and/or selecting a progeny plant comprising:
   i) at least 1 of the SNP markers of SNP_01 to SNP_18 or SNP_12 to SNP_15 (as shown in FIG. 6) for detecting the introgression fragment on chromosome 5; or
   ii) at least 2, 3, or 4 consecutive markers selected from SNP_1 to SNP_18 or SNP_12 to SNP_15 (as shown in FIG. 6) for detecting the introgression fragment on chromosome 5; or
   iii) at least 1, 2, or 3 markers of a group of markers consisting of SNP_01 to SNP_06; SNP_06 to SNP_12; SNP_12 to SNP_18; SNP_03 to SNP_10, SNP_10 to SNP_16, or SNP_12 to SNP_15 (as shown in FIG. 6) for detecting the introgression fragment on chromosome 5; or
   iv) at least 2, 3 or 4 consecutive markers of a group of markers consisting of SNP_01 to SNP_06; SNP_06 to SNP_12; SNP_12 to SNP_18; SNP_03 to SNP_10, SNP_10 to SNP_16, or SNP_12 to SNP_15 (as shown in FIG. 6) for detecting the introgression fragment on chromosome 5.

The cucumber plant in step b is preferably a cultivated cucumber, such as a European greenhouse cucumber or long cucumber type.

The method optionally further comprises the step of identifying a progeny plant having enhanced resistance to internal fruit rot compared to the control.

A progeny plant generated by the above method is also an aspect of the invention. The progeny plant may comprise a shorter introgression fragment than the one found in NCIMB 43530, which retains the QTL5.1.

Also a method for identifying (or detecting) a cultivated C. sativus var. sativus plant or plant part comprising an introgression fragment on chromosome 3 is provided, optionally wherein said introgression fragment is as found in NCIMB 43530 or a smaller (sub-) fragment derived therefrom, comprising:
a) providing a cultivated C. sativus var. sativus plant or plant parts or DNA of such plant or plant part,
b) screening said plant, plant part or DNA using a molecular marker assay which detects at least one SNP marker selected from the group consisting of:
SNP_19 to SNP_42 or SNP_29 to SNP_36, or SNP_35 and/or SNP_36 for detecting the introgression fragment on chromosome 3; and
c) identifying and/or selecting a plant comprising:
   i) at least 1, 2, 3, 4, 5, 6, 7, 8, 9, 10, 11, 12, 13 or more of the SNP markers of SNP_19 to SNP_42, or of SNP_29 to SNP_36, or of SNP_35 and/or SNP_36, for detecting the introgression fragment on chromosome 3; or
   ii) at least 2, 3, 4 5, 6 7, 8, 9, 10 or more consecutive markers selected from SNP_19 to SNP_42, or from SNP_29 to SNP_36, or from SNP_35 and/or SNP_36, for detecting the introgression fragment on chromosome 3; or
   iii) at least 1, 2, 3, 4 5, 6 or more markers of a group, said group consisting of SNP_19 to SNP_27; SNP_27 to SNP_35; SNP_35 to SNP_42; SNP_21 to SNP_30, SNP_30 to SNP_40, SNP_29 to SNP_36, or SNP_35 and/or SNP_36, or
   iv) at least 2, 3, 4 5, 6, 7, 8 or more consecutive markers of a group, said group consisting of SNP_19 to SNP_27; SNP_27 to SNP_35; SNP_35 to SNP_42; SNP_21 to SNP_30, SNP_30 to SNP_40, SNP_29 to SNP_36, or SNP_35 and SNP_36.

Also a method for identifying (or detecting) a cultivated C. sativus var. sativus plant or plant part comprising an introgression fragment on chromosome 3 or comprising a modified chromosome 3 or analyzing a wild cucumber plant for structural variation on chromosome 3 is provided, comprising:
a) providing a cultivated C. sativus var. sativus or a wild cucumber plant or plant parts or DNA of such plant or plant part,
b) screening said plant, plant part or DNA for gene copy number and/or gene expression of one or more or all genes selected from genes encoding OGD1 to OGD8, encoding a 2-oxoglutarate Fe(II)-dependent oxygenase (OGD) proteins, and/or a gene encoding a HR-like protein,
   wherein the eight OGD genes are the genes encoding
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and wherein the gene encoding the HR-like protein is the gene encoding a protein comprising the amino acid sequence of SEQ ID NO: 60 or a sequence comprising at least 95% sequence identity to SEQ ID NO: 60.

The method optionally further comprises selecting a plant, e.g. a wild cucumber or a cultivated cucumber plant, which comprises a higher copy number and/or higher gene expression of one or more or all of the genes compared to a wild type cucumber comprising two copies of each gene.

In one aspect the average gene expression of the eight OGD genes is at least 1.3 times, 1.4 times, 1.5 times, 1.6 times, 1.7 times, 1.8 times, 1.9 times, 2.0 times that of the average gene expression in the wild type cucumber plant, such as a susceptible control, e.g. a plant comprising two copies of each OGD gene on chromosome 3 (i.e. comprising 16 OGD genes in its genome). Alternatively the gene expression of at least one of the OGD genes is at least 1.3 times, 1.4 times, 1.5 times, 1.6 times, 1.7 times, 1.8 times, 1.9 times, 2.0 times that of the average gene expression in the wild type cucumber plant, such as a susceptible control, e.g. a plant comprising two copies of each OGD gene on chromosome 3 (i.e. comprising 16 OGD genes in its genome). In another aspect the average gene expression of the gene encoding the HR-like protein is at least 1.3 times, 1.4 times, 1.5 times, 1.6 times, 1.7 times, 1.8 times, 1.9 times, 2.0 times that of the average gene expression in the wild type cucumber plant, such as a susceptible control, e.g. a plant comprising one copy of the gene encoding the HR-like protein on chromosome 3 (i.e. comprising 2 HR-like genes in its genome). The higher expression in the plant may be due to an increased copy number of one or more of the genes and/or an increased gene expression of one or more of the genes. In one aspect the plant comprises a duplication on e,g, chromosome 3 of one or more or all of the OGD genes and/or of the gene encoding the HR-like protein. In one aspect the plant comprises a duplication of all or part of SEQ ID NO: 72 (and its complement strand) or a sequence comprising at least 95% sequence identity to SEQ ID NO: 72. In one aspect the plant comprises a duplication of the chromosome 3 region flanked by, and optionally including, SEQ ID NO: 83 and SEQ ID NO: 84.

Further a method of producing C. sativus F1 hybrid plants comprising an introgression fragment conferring enhanced internal fruit rot resistance is provided comprising:
a) providing a first inbred cucumber plant comprising a recombinant chromosome 3 in homozygous form having an introgression fragment comprising an allele conferring enhanced internal fruit rot resistance, optionally wherein said introgression fragment is e.g. as in NCIMB 43530 or a smaller (sub-) fragment, e.g. the introgression fragment comprises a duplication of one or more or all of 8 OGD genes and/or one gene encoding a HR-like protein,
b) providing a second inbred cucumber plant,
c) crossing said cucumber plant of a) with said cucumber plant of b),
d) collecting F1 hybrid seeds from said cross,
   wherein the eight OGD genes are the genes encoding
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and wherein the gene encoding the HR-like protein is the gene encoding a protein comprising the amino acid sequence of SEQ ID NO: 60 or a sequence comprising at least 95% sequence identity to SEQ ID NO: 60.

The F1 hybrid seeds collected are also an embodiment of the invention.

In another aspect a method for generating progeny of NCIMB 43530 is provided, said method comprising:
a) growing a plant from seeds deposited under accession number NCIMB 43530;
b) selfing said plant one or more times and/or crossing said plant one or more times with another cucumber plant to generate progeny seeds;
c) optionally screening said progeny seeds or plants grown from said seeds or parts of the seeds or plants using a molecular marker assay which detects at least one SNP marker selected from the group consisting of:
SNP_19 to SNP_42, or SNP_29 to SNP_36, or SNP_35 and SNP_36 for detecting the introgression fragment on chromosome 3; and optionally
d) optionally identifying and/or selecting a progeny plant comprising:
   i) the donor SNP for at least 1 of the SNP markers of SNP_19 to SNP_42 or SNP_29 to SNP_36, or SNP_35 and SNP_36 for detecting the introgression fragment on chromosome 3; or
   ii) the donor SNP for at least 2, 3, or 4 consecutive markers selected from SNP_19 to SNP_42 or SNP_29 to SNP_36, or SNP_35 and SNP_36 for detecting the introgression fragment on chromosome 3; or
   iii) the donor SNP for at least 1, 2, or 3 markers of a group of markers consisting of SNP_19 to SNP_27; SNP_27 to SNP_35; SNP_35 to SNP_42; SNP_21 to SNP_30; SNP_30 to SNP_40; or SNP_29 to SNP_36; or SNP_35 and SNP_36 for detecting the introgression fragment on chromosome 3; or
   iv) the donor SNP for at least 2, 3 or 4 consecutive markers of a group of markers consisting of SNP_19 to SNP_27; SNP_27 to SNP_35; SNP_35 to SNP_42; SNP_21 to SNP_30, SNP_30 to SNP_40 or SNP_29 to SNP_36; or SNP_35 and SNP_36 for detecting the introgression fragment on chromosome 3.

In another aspect a method for generating progeny of NCIMB 43530 is provided, said method comprising:
a) growing a plant from seeds deposited under accession number NCIMB 43530;
b) selfing said plant one or more times and/or crossing said plant one or more times with another cucumber plant to generate progeny seeds;
c) optionally screening said progeny seeds or plants grown from said seeds or parts of the seeds or plants for copy number or gene expression of one or of the 8 OGD genes and/or the gene encoding the HR-like protein,
d) optionally identifying and/or selecting a progeny plant comprising:
   a duplication on chromosome 3 of all or part of the region of SEQ ID NO: 72 or a sequence comprising at least 95% identity to SEQ ID NO: 72, or
   a duplication on chromosome 3 of one or more or all of the 8 OGD genes, or
   a duplication on chromosome 3 of the 8 OGD genes and the gene encoding the HR-like protein,
   a duplication on chromosome 3 of the region flanked by and optionally including SEQ ID NO: 83 and 84;
      wherein the eight OGD genes are the genes encoding
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and wherein the gene encoding the HR-like protein is the gene encoding a protein comprising the amino acid sequence of SEQ ID NO: 60 or a sequence comprising at least 95% sequence identity to SEQ ID NO: 60.

The cucumber plant in step b is preferably a cultivated cucumber, such as a European greenhouse cucumber or long cucumber type.

The method optionally further comprises the step of identifying a progeny plant having enhanced internal fruit rot resistance compared to the control.

A progeny plant generated by any of the above methods is also an aspect of the invention. The progeny plant may comprise a shorter introgression fragment than the one found in NCIMB 43530, which retains the QTL3.1 and/or QTL5.1.

Also containers and packages containing or comprising seeds from which plants of the invention can be grown are provided herein. These may be labelled as containing cultivated cucumber seeds producing enhanced resistance to internal fruit rot caused by DB.

Also progeny seeds and progeny plants of plants of the invention are provided, which retain the introgression on chromosome 5 and/or 3 comprising the QTL(s), or which comprise a smaller introgression (e.g. derivable from the introgression fragment as is present in NCIMB 43530) which still confers enhanced internal fruit rot resistance. Progeny may be any generation obtained by selfing a cucumber plant according to the invention and/or crossing a cucumber plant according to the invention with another cucumber plant one or more times. Progeny are, therefore, either the generation (seeds) produced from the first cross (F1) or selfing (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another cucumber plant (and/or with a wild relative of cucumber). Progeny are preferably selected to retain the recombinant chromosome 5 and/or 3 comprising the introgression fragment from a wild cucumber (comprising the QTL(s)). Thus progeny also have the internal fruit rot resistance phenotype, preferably at least the same reduced susceptibility to internal fruit rot as the plant used in the initial cross or selfing. The presence of (or retention of) the introgression fragment comprising the QTL can be determined phenotypically and/or using the molecular marker assay(s) or other assays as described herein.

In a further aspect parts of the cucumber plants according to the invention are provided. Parts include for example cells and cell-cultures, tissue cultures, vegetative plant tissues (leaves, roots, etc.), flowers, pollen, embryos, fruits, parts of fruits, etc. The plant parts comprise the introgression fragment on chromosome 5 and/or 3, as described, and as can be detected using one or more of the markers described. Also, when whole plants are regenerated from such cucumber parts, such as cells, cell- or tissue cultures, the regenerated plants comprise the recombinant chromosome 5 and/or 3 and the fruit rot resistance phenotype.

Thus, also provided is a plant cell, tissue or plant part of a plant or of a seed according the invention comprising at least one recombinant chromosome 5 and/or 3, wherein said recombinant chromosome 5 and/or 3 comprises an introgression fragment from a wild cucumber plant and wherein said introgression fragment comprises an allele or a QTL conferring enhanced internal fruit rot resistance.

Also in vitro cell cultures and in vitro tissue cultures are encompassed herein, of cells or tissues comprising a recombinant chromosome 5 and/or 3 described. Preferably the cells or tissues can be regenerated into a whole cucumber plant, i.e. the cells are regenerable cells and the tissues comprise regenerable cells. Thus, also vegetative propagations of the plants according to the invention are an embodiment herein. Thus, a vegetatively propagated cultivated cucumber plant is provided which comprises a recombinant chromosome 5 and/or 3 as described herein. In a different aspect non-propagating cells comprising QTL5.1 and/or QTL3.1 are encompassed herein, as are tissues comprising such cells.

In a specific aspect a cucumber fruit harvested from a plant according to the invention is provided. Marketable cucumber fruits, especially for the fresh market (slicing), are generally graded according to fruit size and quality characteristics after harvest. See e.g. the United States Standards for Grades of Cucumbers, US Department of Agriculture, Effective Mar. 1, 1985 and reprinted January 1997. Herein different grades of cucumbers are distinguished. Thus, in one aspect harvested fruits are provided of U.S. Fancy grade, U.S. Extra No. 1 grade, U.S. No. 1 grade, U.S. No. 1 Small grade, U.S. No. 1 Large grade, U.S. No. 2 grade. Also containers or packages comprising or consisting of harvested cucumber fruits are provided. Again, the cells of the fruits are distinguishable from other cucumber fruits by the presence of the recombinant chromosome 5 and/or 3 (as determinable in one or more of the molecular marker assays).

In another aspect the cucumber is a long cucumber type and fruits harvested and optionally processed (e.g. sliced or diced) are provided.

In another aspect the cucumber is a pickling type and fruits harvested and optionally pickled are provided.

The invention also provides for a food or feed product comprising or consisting of a plant part described herein preferably a cucumber fruit or part thereof and/or an extract from a plant part described herein. The food or feed product may be fresh or processed, e.g., pickled, canned, steamed, boiled, fried, blanched and/or frozen, etc. For example, containers such as cans, boxes, crates, bags, cartons, Modified Atmosphere Packaging, films (e.g. biodegradable films), etc. comprising plant parts such as fruits or fruit parts (fresh and/or processed) described herein are also provided herein. 20

### Methods and Uses According to the Invention

In a further embodiment, the invention provides for a method of producing a new cultivated cucumber plant which comprises an introgression fragment on chromosome 5 and/or 3 (which confers enhanced internal fruit rot resistance) in homozygous or heterozygous form, as described. The method comprises crossing a plant of the invention, or a progeny plant thereof, either as male or as female parent, with a second cucumber plant (or a wild relative of cucumber) one or more times, and/or selfing a cucumber plant according to the invention, or a progeny plant thereof, one or more times, and selecting progeny from said crossing and/or selfing.

Thus, a method for transferring the recombinant chromosome 5 and/or 3, comprising the QTL5.1 and/or QTL3.1 respectively, from one (cultivated) cucumber plant into another (cultivated) cucumber plant is provided, especially into cucumber varieties or breeding lines for which the internal fruit rot resistance caused by DB should be increased.

The method comprises the steps of:
a) providing a first cultivate cucumber plant comprising a recombinant chromosome 5 and/or 3 having an introgression fragment comprising a QTL conferring enhanced internal fruit rot resistance, preferably in homozygous form,
b) providing a second cultivated cucumber plant, especially a plant having a wild type (non-recombinant) chromosome 5 and/or 3, e.g. a plant susceptible to internal fruit rot,
c) crossing said cucumber plant of a) with said cucumber plant of b),
d) collecting F1 hybrid seeds from said cross, and
e) optionally selfing the plant grown from said F1 hybrid seeds to produce F2 seeds or further selfing generations, and optionally selecting the F2 seeds or further selfing generation seeds having the recombinant chromosome 5 and/or 3, and
f) optionally breeding further with plants grown from said F1 or F2 or further generation selfing seeds to produce a cucumber plant having good agronomic characteristics and comprising the introgression fragment in homozygous or heterozygous form.

The presence or absence of the recombinant chromosome 5 and/or 3, and of the introgression fragment, may be determined by one or more of the molecular marker assays described herein and/or for chromosome 3 by the copy number and/or gene expression of one or more of the 8 OGD genes and/or the HR-like protein gene and/or by determining whether the internal fruit rot resistance phenotype is significantly increased compared to the plant of step b). Further breeding in step f) may comprise selfing, crossing, double haploid production, backcrossing, and combinations thereof (e.g. backcrossing and selfing), etc. Plants, plant parts and seeds obtainable by the above method are encompassed herein. In one aspect the plant of step a) may be a plant grown from seeds deposited under NCIMB43530, or progeny thereof, or a plant comprising the introgression fragment on chromosome 5 and/or 3 as present in seeds deposited under NCIMB43530, or a shorter fragment of that fragment/derived from that fragment.

Also provided is a method of producing cultivated cucumber F1 hybrid plants comprising a fruit rot resistance QTL on chromosome 5 and/or 3 comprising:
a) providing a first inbred cucumber plant comprising at least one recombinant chromosome 5 and/or 3 comprising an introgression fragment comprising a internal fruit rot resistance QTL selected from QTL5.1 or a variant thereof and/or QTL3.1 or a variant thereof,
b) providing a second inbred cucumber plant either lacking QTL5.1 and QTL3.1; or comprising at least one recombinant chromosome 5 and/or 3 comprising an introgression fragment comprising an internal fruit rot resistance QTL selected from QTL5.1 or a variant thereof and/or QTL3.1 or a variant thereof,
c) crossing said cucumber plant of a) with said cucumber plant of b),
d) collecting F1 hybrid seeds from said cross.

The inbred cucumber plant of a) and b) may be homozygous and/or heterozygous for the introgression fragment on chromosome 5 and/or 3, and they may contain introgression fragments of different sizes and/or of different origin, i.e. from different wild cucumber donors. So, for example the introgression fragment in a) may be the same or a different introgression fragment than in b). In one aspect the inbred cucumber plant of a) comprises QTL5.1 or a variant thereof and/or QTL3.1 or a variant thereof in homozygous form and/or the inbred cucumber plant of b) comprises QTL5.1 or a variant and/or QTL3.1 or a variant thereof in homozygous form. In one aspect the introgression fragment comprising QTL5.1 and/or QTL3.1 is the fragment as found in NCIMB43530 or a smaller fragment derived therefrom.

In one embodiment plants grown from line NCIMB43530, or progeny thereof, e.g. obtained by selfing and/or crossing and retaining QTL5.1 and/or QTL3.1 preferably in homozygous form, are used as a parent line for F1 hybrid seed production.

The F1 hybrid seeds preferably comprise at least one recombinant chromosome 5 and/or 3 and the F1 plants grown from the seeds do therefore produce enhanced internal fruit rot resistance compared to the control, e.g. the genetic control.

Plants and seeds obtainable by the above method are encompassed herein.

In a different aspect a method for producing a cultivated cucumber plant comprising an introgression fragment on chromosome 5 and/or 3, wherein said introgression fragment comprises a fruit rot resistance QTL, is provided, said method comprising the steps:
a) providing a first cultivated cucumber plant,
b) providing a second wild cucumber, wherein said plant comprises QTL5.1 (or a variant thereof) and/or QTL3.1 (or a variant thereof) as determinable by the presence of one or more SNP markers as described herein, i.e. the SNP haplotype or genotype indicative of the QTL or for QTL3.1 by the increased copy number and/or increased gene expression of one or more or all of the 8 OGD genes and/or the HR-like protein gene,
c) crossing said cucumber plant of a) with said cucumber plant of b),
d) collecting F1 seeds from said cross and backcrossing an F1 plant to the cucumber plant of a) to produce a backcross (BC1) population, or selfing said F1 plants one or more times to produce an F2 or F3 or higher generation selfing population,
e) optionally backcrossing a plant of d) one or more times to the cucumber plant of a) to produce a higher generation backcross population, and
f) identifying a F2, F3, or higher generation selfing, or BC1 or higher generation backcross plant which comprises an introgression on chromosome 5 and/or 3, wherein said introgression fragment comprises QTL5.1 (or a variant thereof) and/or QTL3.1 (or a variant thereof).

When referring to backcross populations in the method, the backcross populations may also be selfed, i.e. BC1S1, BC1S2, BC2S1, BC2S2, or others.

In one or more of steps b) to f) the presence of the QTL (or the introgression fragment comprising the QTL) may be tested (and plants may be selected) by carrying out a molecular marker assay as described elsewhere herein.

Using this method, one can generate and/or select new cultivated cucumber plants comprising an introgression with QTL 5.1 (or a variant) and/or QTL3.1 (or a variant thereof) from a wild source, such as a wild relative of cucumber. In one aspect both QTLs are from the same accession of wild cucumber.

In one aspect the method for producing a cultivated cucumber plant comprising an introgression fragment on chromosome 5 and/or chromosome 3, wherein said introgression fragment(s) comprise(s) a internal fruit rot resistance QTL, comprises the steps:
a) providing a first cultivated cucumber plant,
b) providing a second wild cucumber comprising one or more of the SNP markers provided herein or regarding QTL3.1 comprising an increased copy number and/or an increased gene expression of one or more of the 8 OGD genes and/or the gene encoding the HR-like protein, e.g. comprising the SNP haplotype or genotype indicative of the QTL5.1 (or a variant) and/or QTL3.1 (or a variant), or e.g. comprising a duplication of all or part of the region of SEQ ID NO: 72 or comprising at least 95% sequence identity to SEQ ID NO: 72,
c) crossing said plant of a) with said plant of b),
d) collecting F1 seeds from said cross and backcrossing an F1 plant to the cucumber plant of a) to produce a backcross (BC1) population, or selfing said F1 plants one or more times to produce an F2 or F3 population,
e) optionally selfing the backcross population to produce e.g. a BC1S1 or BC1S2 population,
f) identifying a F2, F3, BC1, BC1S1, or BC1S2 plant which comprises the (one or more) SNP markers and/or any wild-relative of cucumber genome-specific marker in between the SNP markers.

Also provided is a method for identifying a wild cucumber comprising a fruit rot resistance QTL on chromosome 5, said method comprising:
A) providing a wild cucumber accession or several accessions;
B) screening said accession(s) using a molecular marker assay which detects at least one (or at least 2, 3, 4, 5, 6, 7, 8, 9, 10 or more) SNP marker selected from the group consisting of: SNP_01 to SNP_18, especially at least 1, 2, 3, 4, 5, 6, or 7 markers selected from SNP_12, SNP_45, SNP_13, SNP_14, SNP_46 to SNP_51 and SNP_15;
C) identifying and/or selecting an accession from b) comprising a marker genotype or haplotype for at least 1, 2, 3, or more of the following markers selected from:
   a) the AA (homozygous) or AX (heterozygous) genotype for SNP_01 at nucleotide 51 of SEQ ID NO: 1 (or at nucleotide 51 of a sequence comprising at least 97% identity to SEQ ID NO: 1),
   b) the GG (homozygous) or GX (heterozygous) genotype for SNP_02 at nucleotide 51 of SEQ ID NO: 2 (or at nucleotide 51 of a sequence comprising at least 97% identity to SEQ ID NO: 2),
   c) the CC (homozygous) or CX (heterozygous) genotype for SNP_03 at nucleotide 51 of SEQ ID NO: 3 (or at nucleotide 51 of a sequence comprising at least 97% identity to SEQ ID NO: 3),
   d) the GG (homozygous) or GX (heterozygous) genotype for SNP_04 at nucleotide 51 of SEQ ID NO: 4 (or at nucleotide 51 of a sequence comprising at least 97% identity to SEQ ID NO: 4),
   e) the AA (homozygous) or AX (heterozygous) genotype for SNP_05 at nucleotide 51 of SEQ ID NO: 5 (or at nucleotide 51 of a sequence comprising at least 97% identity to SEQ ID NO: 5),
   f) the AA (homozygous) or AX (heterozygous) genotype for SNP_06 at nucleotide 51 of SEQ ID NO: 6 (or at nucleotide 51 of a sequence comprising at least 97% identity to SEQ ID NO: 6),
   g) the CC (homozygous) or CX (heterozygous) genotype for SNP_07 at nucleotide 51 of SEQ ID NO: 7 (or at nucleotide 51 of a sequence comprising at least 97% identity to SEQ ID NO: 7),
   h) the CC (homozygous) or CX (heterozygous) genotype SNP_08 at nucleotide 51 of SEQ ID NO: 8 (or at nucleotide 51 of a sequence comprising at least 97% identity to SEQ ID NO: 8),
   i) the AA (homozygous) or AX (heterozygous) genotype for SNP_09 at nucleotide 51 of SEQ ID NO: 9 (or at nucleotide 51 of a sequence comprising at least 97% identity to SEQ ID NO: 9),
   j) the AA (homozygous) or AX (heterozygous) genotype for SNP_10 at nucleotide 51 of SEQ ID NO: 10 (or at nucleotide 51 of a sequence comprising at least 97% identity to SEQ ID NO: 10),
   k) the CC (homozygous) or CX (heterozygous) genotype for SNP_11 at nucleotide 51 of SEQ ID NO: 11 (or at nucleotide 51 of a sequence comprising at least 97% identity to SEQ ID NO: 11),
   l) the GG (homozygous) or GX (heterozygous) genotype for SNP_12 at nucleotide 51 of SEQ ID NO: 12 (or at nucleotide 51 of a sequence comprising at least 97% identity to SEQ ID NO: 12),
   m) the AA (homozygous) or AX (heterozygous) genotype for SNP_13 at nucleotide 51 of SEQ ID NO: 13 (or at nucleotide 51 of a sequence comprising at least 97% identity to SEQ ID NO: 13),
   n) the GG (homozygous) or GX (heterozygous) genotype for SNP_14 at nucleotide 51 of SEQ ID NO: 14 (or at nucleotide 51 of a sequence comprising at least 97% identity to SEQ ID NO: 14),
   o) the GG (homozygous) or GX (heterozygous) genotype for SNP_15 at nucleotide 51 of SEQ ID NO: 15 (or at nucleotide 51 of a sequence comprising at least 97% identity to SEQ ID NO: 15),
   p) the CC (homozygous) or CX (heterozygous) genotype for SNP_16 at nucleotide 51 of SEQ ID NO: 16 (or at nucleotide 51 of a sequence comprising at least 97% identity to SEQ ID NO: 16),
   q) the AA (homozygous) or AX (heterozygous) genotype for SNP_17 at nucleotide 51 of SEQ ID NO: 17 (or at nucleotide 51 of a sequence comprising at least 97% identity to SEQ ID NO: 17) and
   r) the GG (homozygous) or GX (heterozygous) genotype for SNP_18 at nucleotide 51 of SEQ ID NO: 18 (or at nucleotide 51 of a sequence comprising at least 97% identity to SEQ ID NO: 18);
or identifying and/or selecting an accession from b) comprising a marker genotype or haplotype for at least 1, 2, 3, or more of the following markers selected from:
   a Guanine for SNP_12 at nucleotide 51 of SEQ ID NO: 12 or at nucleotide 51 of a sequence comprising at least 97% identity to SEQ ID NO: 12,
   an Adenine for SNP_45 at nucleotide 51 of SEQ ID NO: 45 or at nucleotide 51 of a sequence comprising at least 97% identity to SEQ ID NO: 45,
   an Adenine for SNP_13 at nucleotide 51 of SEQ ID NO: 13 or at nucleotide 51 of a sequence comprising at least 97% identity to SEQ ID NO: 13,
   a Guanine for SNP_14 at nucleotide 51 of SEQ ID NO: 14 or at nucleotide 51 of a sequence comprising at least 97% identity to SEQ ID NO: 14,
   a Thymine for SNP_46 at nucleotide 51 of SEQ ID NO: 46 or at nucleotide 51 of a sequence comprising at least 97% identity to SEQ ID NO: 46,
   a Thymine for SNP_47 at nucleotide 51 of SEQ ID NO: 47 or at nucleotide 51 of a sequence comprising at least 97% identity to SEQ ID NO: 47,
   a Cytosine for SNP_48 at nucleotide 51 of SEQ ID NO: 48 or at nucleotide 51 of a sequence comprising at least 97% identity to SEQ ID NO: 48,
   a Guanine for SNP_49 at nucleotide 51 of SEQ ID NO: 49 or at nucleotide 51 of a sequence comprising at least 97% identity to SEQ ID NO: 49,
   a Thymine for SNP_50 at nucleotide 51 of SEQ ID NO: 50 or at nucleotide 51 of a sequence comprising at least 97% identity to SEQ ID NO: 50,
   an Adenine for SNP_51 at nucleotide 51 of SEQ ID NO: 51 or at nucleotide 51 of a sequence comprising at least 97% identity to SEQ ID NO: 51,
   a Guanine for SNP_15 at nucleotide 51 of SEQ ID NO: 15 or at nucleotide 51 of a sequence comprising at least 97% identity to SEQ ID NO: 15;
   and optionally
D) introgressing said QTL from said wild accession into cultivated cucumber (e.g. by backcrossing).

In step B), C) and D) also other molecular marker tests described elsewhere herein can be used. With this method one can, thus, screen wild relatives of cucumber for the presence of one or more of the markers and, thus, the presence of QTL5.1 (or a variant thereof) and introgress the QTL into cultivated cucumber plants. Plants and seeds obtained by this method are also an embodiment of the invention.

Also provided is a method for identifying a wild relative of cucumber comprising a internal fruit rot resistance QTL on chromosome 3, said method comprising:
A) providing a wild relative of cucumber accession or several accessions;
B) screening said accession(s) using a molecular marker assay which detects at least one (or at least 2, 3, 4, 5, 6, 7, 8, 9, 10 or more) SNP marker selected from the group consisting of: SNP_19 to SNP_42, especially SNP_29 to SNP_35 or SNP_35 and/or SNP_36;
C) identifying and/or selecting an accession from b) comprising at least one or more of the following markers:
   a) the GG (homozygous) or GX (heterozygous) genotype for the Single Nucleotide Polymorphism marker SNP_19 at nucleotide 51 of SEQ ID NO: 19 (or in a variant thereof);
   b) the GG (homozygous) or GX (heterozygous) genotype for the Single Nucleotide Polymorphism marker SNP_20 at nucleotide 51 of SEQ ID NO: 20 (or in a variant thereof);
   c) the TT (homozygous) or TX (heterozygous) genotype for the Single Nucleotide Polymorphism marker SNP_21 at nucleotide 51 of SEQ ID NO: 21 (or in a variant thereof);
   d) the AA (homozygous) or AX (heterozygous) genotype for the Single Nucleotide Polymorphism marker SNP_22 at nucleotide 51 of SEQ ID NO: 22 (or in a variant thereof);
   e) the CC (homozygous) or CCX (heterozygous) genotype for the Single Nucleotide Polymorphism marker SNP_23 at nucleotide 51 of SEQ ID NO: 23 (or in a variant thereof);
   f) the AA (homozygous) or AX (heterozygous) genotype for the Single Nucleotide Polymorphism marker SNP_24 at nucleotide 51 of SEQ ID NO: 24 (or in a variant thereof);
   g) the GG (homozygous) or GX (heterozygous) genotype for the Single Nucleotide Polymorphism marker SNP_25 at nucleotide 51 of SEQ ID NO: 25 (or in a variant thereof);
   h) the TT (homozygous) or TX (heterozygous) genotype for the Single Nucleotide Polymorphism marker SNP_26 at nucleotide 51 of SEQ ID NO: 26 (or in a variant thereof);
   i) the CC (homozygous) or CX (heterozygous) genotype for the Single Nucleotide Polymorphism marker SNP_27 at nucleotide 51 of SEQ ID NO: 27 (or in a variant thereof);
   j) the GG (homozygous) or GX (heterozygous) genotype for the Single Nucleotide Polymorphism marker SNP_28 at nucleotide 51 of SEQ ID NO: 28 (or in a variant thereof);
   k) the CC (homozygous) or CX (heterozygous) genotype for the Single Nucleotide Polymorphism marker SNP_29 at nucleotide 51 of SEQ ID NO: 29 (or in a variant thereof);
   l) the AA (homozygous) or AX (heterozygous) genotype for the Single Nucleotide Polymorphism marker SNP_30 at nucleotide 51 of SEQ ID NO: 30 (or in a variant thereof);
   m) the GG (homozygous) or GX (heterozygous) genotype for the Single Nucleotide Polymorphism marker SNP_31 at nucleotide 51 of SEQ ID NO: 31 (or in a variant thereof);
   n) the GG (homozygous) or GX (heterozygous) genotype for the Single Nucleotide Polymorphism marker SNP_32 at nucleotide 51 of SEQ ID NO: 32 (or in a variant thereof);
   o) the GG (homozygous) or GX (heterozygous) genotype for the Single Nucleotide Polymorphism marker SNP_33 at nucleotide 51 of SEQ ID NO: 33 (or in a variant thereof);
   p) the GG (homozygous) or GX (heterozygous) genotype for the Single Nucleotide Polymorphism marker SNP_34 at nucleotide 51 of SEQ ID NO: 34 (or in a variant thereof);
   q) the TT (homozygous) or TX (heterozygous) genotype for the Single Nucleotide Polymorphism marker SNP_35 at nucleotide 51 of SEQ ID NO: 35 (or in a variant thereof);
   r) the TT (homozygous) or TTX (heterozygous) genotype for the Single Nucleotide Polymorphism marker SNP_36 at nucleotide 51 of SEQ ID NO: 36 (or in a variant thereof);
   s) the CC (homozygous) or CX (heterozygous) genotype for the Single Nucleotide Polymorphism marker SNP_37 at nucleotide 51 of SEQ ID NO: 37 (or in a variant thereof);
   t) the GG (homozygous) or GX (heterozygous) genotype for the Single Nucleotide Polymorphism marker SNP_38 at nucleotide 51 of SEQ ID NO: 38 (or in a variant thereof);
   u) the CC (homozygous) or CX (heterozygous) genotype for the Single Nucleotide Polymorphism marker SNP_39 at nucleotide 51 of SEQ ID NO: 39 (or in a variant thereof);
   V the TT (homozygous) or TX (heterozygous) genotype for the Single Nucleotide Polymorphism marker SNP_40 at nucleotide 51 of SEQ ID NO: 40 (or in a variant thereof);
   w) the AA (homozygous) or AX (heterozygous) genotype for the Single Nucleotide Polymorphism marker SNP_41 at nucleotide 51 of SEQ ID NO: 41 (or in a variant thereof);
   x) the AA (homozygous) or AX (heterozygous) genotype for the Single Nucleotide Polymorphism marker SNP_42 at nucleotide 51 of SEQ ID NO: 42 (or in a variant thereof).
   or
identifying and/or selecting an accession from b) comprising at least one or more of the following markers:
   a) the CC (homozygous) or CX (heterozygous) genotype for the Single Nucleotide Polymorphism marker SNP_29 at nucleotide 51 of SEQ ID NO: 29 (or in a variant thereof);
   b) the AA (homozygous) or AX (heterozygous) genotype for the Single Nucleotide Polymorphism marker SNP_30 at nucleotide 51 of SEQ ID NO: 30 (or in a variant thereof);
   c) the GG (homozygous) or GX (heterozygous) genotype for the Single Nucleotide Polymorphism marker SNP_31 at nucleotide 51 of SEQ ID NO: 31 (or in a variant thereof);
   d) the GG (homozygous) or GX (heterozygous) genotype for the Single Nucleotide Polymorphism marker SNP_32 at nucleotide 51 of SEQ ID NO: 32 (or in a variant thereof);
   e) the GG (homozygous) or GX (heterozygous) genotype for the Single Nucleotide Polymorphism marker SNP_33 at nucleotide 51 of SEQ ID NO: 33 (or in a variant thereof);
   f) the GG (homozygous) or GX (heterozygous) genotype for the Single Nucleotide Polymorphism marker SNP_34 at nucleotide 51 of SEQ ID NO: 34 (or in a variant thereof);
   g. the TT (homozygous) or TX (heterozygous) genotype for the Single Nucleotide Polymorphism marker SNP_35 at nucleotide 51 of SEQ ID NO: 35 (or in a variant thereof);
   h) the TT (homozygous) or TTX (heterozygous) genotype for the Single Nucleotide Polymorphism marker SNP_36 at nucleotide 51 of SEQ ID NO: 36 (or in a variant thereof);
   or
identifying and/or selecting an accession from b) comprising at least one or more of the following markers:
   a) the TT (homozygous) or TX (heterozygous) genotype for the Single Nucleotide Polymorphism marker SNP_35 at nucleotide 51 of SEQ ID NO: 35 (or in a variant thereof); and/or
   b) the TT (homozygous) or TTX (heterozygous) genotype for the Single Nucleotide Polymorphism marker SNP_36 at nucleotide 51 of SEQ ID NO: 36 (or in a variant thereof);
   and optionally
D) introgressing said QTL from said wild accession into cultivated cucumber (e.g. by backcrossing).

In step B), C) and D) also other molecular marker tests described elsewhere herein can be used. With this method one can, thus, screen wild relatives of cucumber for the presence of one or more of the markers and,
thus, the presence of QTL3.1 (or a variant thereof) and introgress the QTL into cultivated cucumber plants. Plants and seeds obtained by this method are also an embodiment of the invention.

Also provided is a method for identifying a cucumber plant, e.g. a wild cucumber or wild relative of cucumber comprising an internal fruit rot resistance QTL on chromosome 3, said method comprising:
A) providing a cucumber plant, e.g. a wild cucumber or wild relative of cucumber accession or several accessions;
B) screening said accession(s) for the copy number and/or gene expression of 8 OGD genes and/or a gene encoding a HR-like protein, or for the presence of a duplication on e.g. chromosome 3 of one or more of the 8 OGD genes and/or the gene encoding the HR-like protein.

In still another aspect a method for identifying a cultivated cucumber plant comprising an introgression fragment on chromosome 5 and/or 3, wherein said introgression fragment comprises a internal fruit rot resistance QTL, is provided, said method comprising: screening a cultivated cucumber plant or a population of cultivated cucumber plants or parts of such cucumber plants (e.g. fruits, cells, DNA) using a molecular marker assay which detects at least one SNP marker (preferably 2, 3, 4, 5 or more; preferably consecutive SNP markers) indicative of (linked to) QTL5.1 and/or QTL3.1 as described elsewhere herein and/or, regarding QTL3.1, screening for an increased copy number on e.g. chromosome 3 and/or increased gene expression of one or more of the 8 OGD genes and/or the gene encoding the HR-like protein.

In this method any of the molecular marker tests described elsewhere herein can be used and the gene sequences (genomic, cDNA or protein) for the OGD genes and the HR-like protein gene can be used. Thus, using this method one can detect the presence of an introgression fragment on chromosome 5 and/or 3 and comprising QTL5.1 and/or QTL3.1 in cultivated cucumber plants or plant parts.

In yet another aspect a method for detecting whether a cultivated cucumber plant comprises an introgression fragment on chromosome 5, wherein said introgression fragment comprises QTL5.1, is provided, said method comprising:
a) providing cultivated cucumber plant or a plant part,
b) screening said plant or said plant part (or DNA obtained from said plant or plant part) using a molecular marker assay which detects at least one (preferably at least 2, 3, 4, 5 or more) SNP marker selected from the group consisting of: SNP_01 to SNP_18, preferably selected from SNP_12, SNP_45, SNP_13, SNP_14, SNP_46 to SNP_51 and SNP_15.

In yet another aspect a method for detecting whether a cultivated cucumber plant comprises an introgression fragment on chromosome 3, wherein said introgression fragment comprises QTL3.1, is provided, said method comprising:
a) providing cultivated cucumber plant or a plant part,
b) screening said plant or said plant part (or DNA obtained from said plant or plant part) using a molecular marker assay which detects at least one (preferably at least 2, 3, 4, 5 or more) SNP marker selected from the group consisting of: SNP_19 to SNP_42, preferably selected from SNP_29 to SNP_36, preferably at least SNP_35 and/or SNP_36, or screening whether chromosome 3 comprises a duplication of all or part of the region of SEQ ID NO: 72 (or a sequence comprising at least 95% identity to SEQ ID NO: 72), or screening whether chromosome 3 comprises a duplication of one or more of the OGD genes and/or the gene encoding the HR-like protein.

Molecular marker screening obviously involves obtaining plant material and analyzing the genomic DNA of the material for the marker genotype or haplotype. When referring to markers herein, therefore also the marker genotype and/or the marker haplotype is referred to. So detecting one or more SNP markers, refers to determining the plant (or plant part) genotype or plant haplotype for the marker(s). Screening whether chromosome 3 comprises a duplication of the region and the genes can be done by various methods, such as PCR based methods, sequencing based methods, hybridization based methods, gene expression analysis, etc.

In this method also other molecular marker tests described elsewhere herein can be used.

Also encompassed herein is a method for producing a cultivated cucumber plant comprising an introgression fragment on chromosome 5, wherein said introgression fragment comprises QTL5.1, comprising:
a) providing a first cultivated cucumber plant lacking an introgression fragment comprising QTL5.1,
b) providing a second cultivated cucumber plant selected from plants grown from seeds deposited under accession number NCIMB43530 or progeny thereof,
c) crossing said plant of a) with said plant of b),
d) collecting F1 seeds from said cross and optionally selfing said F1 plants one or more times to produce an F2 or F3 or further selfing population,
e) optionally backcrossing the F1 plant or an F2 or F3 or further selfing plant to the plant of a) to produce a backcross population,
f) optionally selfing the backcross population one or more times,
g) identifying a F1, F2, F3, further selfing or backcross plant which comprises one or more or all of the SNP marker genotypes indicative of the introgression fragment on chromosome 5.

Also encompassed herein is a method for producing a cultivated cucumber plant comprising an introgression fragment on chromosome 3, wherein said introgression fragment comprises QTL3.1, comprising:
a) providing a first cultivated cucumber plant lacking an introgression fragment comprising QTL3.1,
b) providing a second cultivated cucumber plant selected from plants grown from seeds deposited under accession number NCIMB43530 or progeny thereof,
c) crossing said plant of a) with said plant of b),
d) collecting F1 seeds from said cross and optionally selfing said F1 plants one or more times to produce an F2 or F3 or further selfing population,
e) optionally backcrossing the F1 plant or an F2 or F3 or further selfing plant to the plant of a) to produce a backcross population,
f) optionally selfing the backcross population one or more times,
g) identifying a F1, F2, F3, further selfing or backcross plant which comprises one or more or all of the SNP marker genotypes indicative of the introgression fragment on chromosome 3 or which comprises a duplication of all or part of the region SEQ ID NO: 72 (or a region comprising at least 95% identity to this sequence), or comprises a duplication of one or more of the OGD genes and/or the gene encoding the HR-like protein.

In a further aspect a method of producing F1 hybrid plants is provided comprising:
a) providing a first inbred cucumber plant comprising at least one recombinant chromosome 5 having an introgression fragment comprising QTL5.1, wherein said introgression fragment is the fragment as found in NCIMB43530, or a shorter fragment of that introgression fragment retaining QTL5.1,
b) providing a second inbred cucumber plant with or without a recombinant chromosome 5,
c) crossing said plant of a) with said plant of b),
d) collecting F1 hybrid seeds from said cross.

In a further aspect a method of producing F1 hybrid plants is provided comprising:
a) providing a first inbred cucumber plant comprising at least one recombinant chromosome 3 having an introgression fragment comprising QTL3.1, wherein said introgression fragment is the fragment as found in NCIMB43530, or a shorter fragment of that introgression fragment,
b) providing a second inbred cucumber plant with or without a recombinant chromosome 3,
c) crossing said plant of a) with said plant of b),
d) collecting F1 hybrid seeds from said cross.

A shorter fragment of the introgression fragment is a fragment retaining the QTL, so preferably retaining all or part of the region between SNP_12 and SNP_15 (as shown in FIG. 6) and thus e.g. comprising the donor SNP haplotype or genotype for one or more of the markers selected from SNP_12 to SNP_15 (as shown in FIG. 6) comprising QTL5.1 or retaining a duplication of all or part of the region between SNP_35 and 659 nt downstream of SNP_36 comprising QTL3.1 (SEQ ID NO: 72), e.g. retaining a duplication on chromosome 3 of one or more of the 8 OGD genes and/or the HR-like protein gene.

In another aspect a method for generating progeny of NCIMB43530 retaining QTL5.1 and/or QTL3.1 is provided, said method comprising:
a) growing a plant from seeds deposited under accession number NCIMB43530;
b) selfing said plant one or more times or crossing said plant one or more times with another cultivated cucumber plant to generate progeny seeds;
c) optionally screening said progeny seeds or plants grown from said seeds or parts of the seeds or plants using a molecular marker assay which detects at least one SNP marker disclosed herein;
d) identifying and/or selecting a progeny plant retaining QTL5.1 and/or QTL3.1, e.g. retaining all or part of the region between SNP_12 and SNP_15 (as shown in FIG. 6) and thus e.g. comprising the donor SNP haplotype or genotype for one or more of the markers selected from SNP_12 to SNP_15 (as shown in FIG. 6) comprising QTL5.1 or retaining a duplication of all or part of the region between SNP_35 and 659 nt downstream of SNP_36 comprising QTL3.1 (i.e. SEQ ID NO: 72 or a sequence comprising at least 95% identity), e.g. retaining a duplication on chromosome 3 of one or more of the 8 OGD genes and/or the HR-like protein gene, and
e) optionally confirming the enhanced internal fruit rot resistance of said progeny plants.

In one aspect both QTLs are present in the progeny in d), preferably in homozygous form, and the internal fruit rot resistance in e) is preferably at least the same resistance level as for plants grown from NCIMB43530 when grown under the same conditions.

A method for generating progeny of NCIMB 43530 is provided, said method comprising:
a) growing a plant from seeds deposited under accession number NCIMB 43530;
b) selfing said plant one or more times or crossing said plant one or more times with another cucumber plant to generate progeny seeds;
c) optionally screening said progeny seeds or plants grown from said seeds or parts of the seeds or plants using a molecular marker assay which detects at least one SNP marker selected from the group consisting of:
SNP_01 to SNP_18 or SNP_12 to SNP_15 (as shown in FIG. 6, i.e. SNP_12, SNP_45, SNP_13, SNP_14, SNP_46 to SNP_51 and SNP_15) for detecting the introgression fragment on chromosome 5; and/or SNP_19 to SNP_42, preferably SNP_29 to SNP_36, preferably SNP_35 and/or SNP_36 for detecting the introgression fragment on chromosome 3 or screening the progeny plants for the presence of a duplication on chromosome 3 of all or part of the region between SNP_35 and 659 nt downstream of SNP_36 (i.e. SEQ ID NO: 72 or a sequence comprising at least 95% identity), or for a duplication on chromosome 3 of one or more or all genes encoding 8 OGD genes and/or the HR-like protein encoding gene;
d) identifying and/or selecting a progeny plant comprising:
   i) the donor SNP genotype or haplotype for at least 1, 2, 3, 4, 5, 6, 7, 8 or more of the SNP markers of SNP_01 to SNP_18, preferably at least 1, 2, 3, 4, 5, 6 or 7 of SNP_12, SNP_45, SNP_13, SNP_14, SNP_46 to SNP_51 and SNP_15 for the introgression fragment on chromosome 5 and/or at least 1, 2, 3, 4, 5, 6, 7, 8 or more of the SNP markers of SNP_19 to SNP_42, preferably at least 1, 2, 3, 4, 5, 6, 7 of SNP_29 to SNP_36 for the introgression fragment on chromosome 3; or
   ii) the donor SNP genotype or haplotype for at least 2, 3, 4, 5, 6, 7, 8, 9, 10 or more consecutive markers selected from SNP_01 to SNP_18, preferably at least 1, 2, 3, 4, 5, 6 or 7 consecutive markers of SNP_12, SNP_45, SNP_13, SNP_14, SNP_46 to SNP_51 and SNP_15 for the introgression fragment on chromosome 5 and/or at least 1, 2, 3, 4, 5, 6, 7, 8 or more consecutive markers of SNP_19 to SNP_42, preferably at least 1, 2, 3, 4, 5, 6 or 7 consecutive markers of SNP_29 to SNP_36 for the introgression fragment on chromosome 3,
   iii) or for the presence of a duplication on chromosome 3 of all or part of the region between SNP_35 and 659 nt downstream of SNP_36 (i.e. SEQ ID NO: 72 or a sequence comprising at least 95% identity), or for a duplication on chromosome 3 of one or more or all genes encoding 8 OGD genes and/or the HR-like protein encoding gene, and
e) optionally confirming the enhanced internal fruit rot resistance of said progeny plants.

A progeny plant generated by any of the above methods is also an aspect of the invention.

One can also use the methods and the markers and gene sequences described herein to reduce the size of the introgression fragment comprising the QTL5.1 and/or QTL3.1, i.e. to generate and select recombinants having a smaller introgression fragment (sub-fragments) on chromosome 5 and/or 3, but which retain the internal fruit rot resistance enhancing part of the introgression fragment (i.e. the causal gene or genes underlying the QTL are retained).

In one aspect the invention encompasses the use of a recombinant chromosome 5 and/or 3 comprising an introgression fragment from a wild cucumber donor, said introgression fragment comprising an internal fruit rot resistance QTL, for breeding cucumber varieties having enhanced internal fruit rot resistance.

Also provided is the use of a chromosome 5 and/or 3 as found in seeds deposited under accession number NCIMB43530 or progeny thereof for generating cultivated cucumber plant comprising an introgression fragment of said chromosome 5 and/or 3.

Also provided is the use of plants grown from seeds deposited under accession number NCIMB 43530, or progeny thereof, for generating a cultivated cucumber plant comprising enhanced internal fruit rot resistance, wherein said enhanced internal fruit rot resistance is conferred by an introgression fragment obtained from chromosome 5 and/or 3 of said plants or progeny, or by a sub-fragment derived therefrom retaining the QTL.

### Cucumber Plants Comprising an Increased Gene Dosage Conferring Internal DB Fruit Rot Resistance

The above embodiments relate mainly to the introgression of QTLs from donor accessions. However, as the genes underlying the resistance conferred by QTL3.1 are known, other ways to modify chromosome 3 can be used to generate the same effect as the introgression of the duplication of QTL3.1 on chromosome 3 from a wild donor.

The region between SNP_35 and 659nt downstream of SNP_36 which was duplicated in the donor MYCR3 as part of a larger duplication of the region from SEQ ID NO: 83 to SEQ ID NO: 84 (see FIGS. 4 and 7) contained 8 OGD genes, encoding 2-oxoglutarate Fe(II)-dependent oxygenase (OGD) proteins and one gene encoding a “HR-like” protein. In the resistant plant, which was homozygous for the introgression and contained 32 copies of OGD genes and 4 copies of the gene encoding the HR-like protein, the expression level of these genes was, therefore, significantly increased compared to the susceptible plant, lacking the introgression fragment and thus lacking the duplication of the region between SNP_35 and 659 nt downstream of SNP_36 and containing only 16 copies of OGD genes and 2 copies of the gene encoding the HR-like protein.

The increased expression was found in the leaves and in the ovary tissue of the fruits. As the fruit infection takes place via the flowers, it is reasonable to conclude that the increased OGD protein level reduces or even prevents infection of the fruits. The HR-like protein may also play a role, as such proteins are involved in the so-called hypersensitive response (HR), which is a defense response against infection. HR is characterized by the rapid death of cells in the local region surrounding an infection and it serves to restrict the growth and spread of pathogens to other parts of the plant. However, as this is gene encodes an uncharacterized protein only annotated as HR-like based on sequence homology to a HR-like conserved domain and as the gene is present in a much lower copy number and also as no lesions have been observed on the tissue, the main effect is believed to be caused by the OGD genes. Evolutionarily it is often seen that defense genes are multiplied, often in tandem on a chromosome, similar to what was found here for the OGD genes.

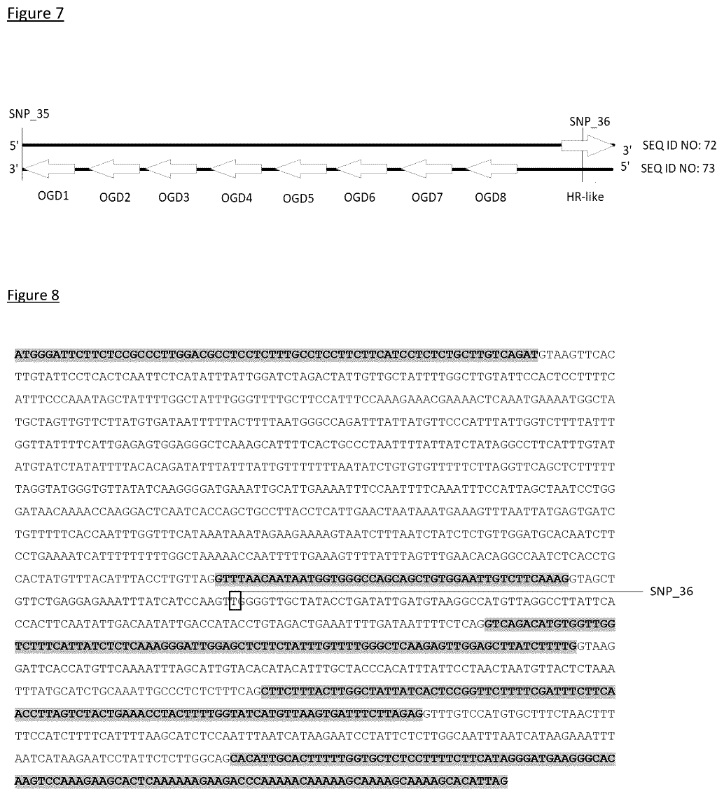

The order of the genes in the cluster is as given in FIG. 4, starting with OGD1 near SNP_35 and ending with the gene encoding the HR-like protein. SNP_36 was found to be present in an intron of the gene encoding the HR-like protein, see FIG. 8. The plus strand, with the gene encoding the HR-like protein is provided in SEQ ID NO: 72 and the complementary minus strand, with the genes encoding the 8 2OGD proteins is provided in SEQ ID NO: 73, see FIG. 7. As mentioned in the Examples, the sequence of SEQ ID NO: 72 and 73 provided herein is the sequence of the reference genome (and not of the MYCR3 donor) and may thus vary between cucumber plants. However, the encoded proteins were found to be identical in the MYCR3 donor and the reference genome.

The resistance against internal fruit rot therefore appears to be a dosage effect, especially of the OGD proteins.

Therefore, increasing the effective dosage of the OGD genes and/or the HR-like protein encoding gene in the genome of cucumber, compared to the wild type dosage in the susceptible cucumber, increases the resistance against internal fruit rot caused by DB. Besides introgressions to increase copy number or to introgress alleles having higher gene expression (e.g. due to more active promoters or the presence of enhancer elements), there are different ways to increase the effective dosage of OGD genes and/or the HR-like protein. These include targeted mutagenesis and transgenic approaches. The expression of the genes can for example be increased by increasing copy number in the plant genome (e.g. by targeted genome modification or by transgenic methods), optionally on chromosome 3, or by increasing the expression level, for example by modifying the endogenous promoter or by expressing the gene from a different promoter, such as an ovary specific promoter or a constitutive promoter, or by modifying the endogenous cis-or trans-regulatory elements of the genes.

In one aspect a cucumber plant genome is modified in such a way that the gene expression level of one or more of the 8 OGD genes, selected from OGD1 to OGD8, and/or of the gene encoding the HR-like protein is modified, especially increased, compared to the gene expression level found in a wild type, non-modified plant genome.

The gene expression level of each gene in a non-modified cucumber plant is the level of mRNA (cDNA) transcript produced by the wild type, non-modified endogenous gene in a cucumber plant having a non-modified genome with respect to the wild type genes and their copy number, such as an internal fruit rot susceptible cucumber plant comprising one copy of each of the wild type genes on each chromosome 3, such as for example the DB-susceptible plant (lacking the duplication of QTL3.1), of which a representative sample of seeds has been deposited or any other breeding line or variety.

As mentioned, the gene expression level of an OGD gene selected from OGD1 to OGD8 and/or the gene encoding the HR-like protein can be modified by:
a) increasing the copy number in the diploid genome to be more than 2 copies (which is the copy number in the wild type plant), such as 3, 4, 5, 6, 7, 8 or more; or increasing the copy number in the haploid genome to be more than 1 copy (which is the copy number of the wild type plant);
b) modifying a cis-regulatory element (e.g. the promoter or enhancer) of the gene to increase its expression;
c) modifying a trans-regulatory element of the gene to increase its expression.

The effect is thus that the diploid plant has a higher effective dosage of the 2OGD genes and/or of the gene encoding the HR-like protein, whereby more transcript and more 2OGD protein and/or HR-like protein is being made, especially in the ovary tissue.

A Cucumis sativus var. sativus plant is provided comprising an increased resistance against internal fruit rot symptoms caused by Didymella bryoniae, wherein the plant genome is modified to increase the average gene expression of eight OGD genes, OGD1 to OGD8, encoding a 2-oxoglutarate Fe(II)-dependent oxygenase (OGD) proteins, to be at least 1.3 times that of the average gene expression of the eight OGD genes in a wild type, susceptible plant comprising two copies of each OGD gene, wherein the eight OGD genes are the genes encoding
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and/or wherein the plant genome is modified to increase the average gene expression of a gene encoding a HR-like protein to be at least 1.3 times that of the average gene expression of said gene in a wild type, susceptible plant comprising two copies of the gene, wherein said gene is a gene encoding a protein comprising the amino acid sequence of SEQ ID NO: 60 or a sequence comprising at least 95% sequence identity to SEQ ID NO: 60.

In one aspect the plant is modified to increase the gene expression by a modification of a cis-regulatory element of the gene, or a modification of a trans-regulatory element of the gene, or an increased dosage of the gene in the plant, or by the introduction of a transgene expressing the gene.

The diploid plant may thus comprises at least at least three expressed copies of the gene encoding the HR-like protein and/or at least 24 expressed copies of a gene encoding a 2-oxoglutarate Fe(II) dependent oxygenase protein selected from OGD1 to OGD8. For example a plant which comprises one chromosome 3 which has a duplication of the entire cluster (i.e. OGD1 to OGD8 and HR-like) and further comprises one wild type chromosome 3 would be such a plant. However, also a plant comprising e.g. several transgenes, e.g. a wild type plant into which 8 2OGD transgenes and one transgene of the HR-like protein is introduced would be such a plant.

Apart from duplicating the cluster or generating transgenes of the cluster, one can also increase copy number of individual genes or subgroups of genes, e.g. introduce 8 copies of 2OGD1 or 4 copies of OGD1 and 4 copies of OGD2, etc.

It is also possible to increase the gene expression of one or more of the genes, by e.g. modifying the promoter or enhancer elements so that more mRNA is transcribed and more protein is made.

Preferably the plant is modified so that the gene expression of one or more or all of the genes encoding a 2-oxoglutarate Fe(II) dependent oxygenase protein selected from OGD1 to OGD8 and/or the HR-like protein is at least 1.3 times the expression in the wild type (unmodified) plant.

An easy way to increase effective dosage is to duplication one or more or all genes encoding a 2-oxoglutarate Fe(II) dependent oxygenase (OGD) protein and/or the gene encoding the HR-like protein in the genome so that the haploid genome comprises at least one additional copy of the gene. As mentioned, the wild type haploid genome has one copy of each gene (on chromosome 3) and one can increase the copy number of each of the wild type genes in the haploid genome by various methods, such as transgenesis or CRIPSR based methods whereby additional copies are introduced. Duplication does not only mean that the copy number in the haploid genome is increased from one copy to two copies, but encompasses additional copies, e.g. 3, 4, 5 or more of any one of the genes.

The genes are selected from those encoding:
an OGD1 protein comprising the amino acid sequence of SEQ ID NO: 52 or a sequence comprising at least 95% sequence identity to SEQ ID NO: 52,
an OGD2 protein comprising the amino acid sequence of SEQ ID NO: 53 or a sequence comprising at least 95% sequence identity to SEQ ID NO: 53,
an OGD3 protein comprising the amino acid sequence of SEQ ID NO: 54 or a sequence comprising at least 95% sequence identity to SEQ ID NO: 54,
an OGD4 protein comprising the amino acid sequence of SEQ ID NO:55 or a sequence comprising at least 95% sequence identity to SEQ ID NO: 55,
an OGD5 protein comprising the amino acid sequence of SEQ ID NO: 56 or a sequence comprising at least 95% sequence identity to SEQ ID NO: 56,
an OGD6 protein comprising the amino acid sequence of SEQ ID NO: 57 or a sequence comprising at least 95% sequence identity to SEQ ID NO: 57,
an OGD7 protein comprising the amino acid sequence of SEQ ID NO: 58 or a sequence comprising at least 95% sequence identity to SEQ ID NO: 58,
an OGD8 protein comprising the amino acid sequence of SEQ ID NO: 59 or a sequence comprising at least 95% sequence identity to SEQ ID NO: 59,
and/or a gene encoding a HR-like protein comprising the amino acid sequence of SEQ ID NO: 60 or a sequence comprising at least 95% sequence identity to SEQ ID NO: 60.

Also encompassed is a plant wherein the plant comprises a duplication of QTL3.1 in its haploid genome, wherein QTL3.1 is the genomic sequence of SEQ ID NO: 72 or of a sequence comprising at least 95% sequence identity to SEQ ID NO: 72. This entire region comprises the 8 OGD genes and the gene encoding the HR-like protein. A duplication of the entire region (or gene cluster) can be induced by targeted genome editing methods, such as CRISPR based methods, or be introduced by transformation, i.e. transgenic approaches. Alternatively, such a duplication can be introgressed from a cucumber donor, such as a wild donor or a cultivated donor comprising such a duplication.

In a further aspect a recombinant nucleic acid molecule, especially a recombinant DNA molecule, is provided which comprises one or more of the genes described herein. Also a DNA vector is provided comprising the recombinant DNA. The recombinant DNA molecule or DNA vector may be an isolated nucleic acid molecule.

The DNA comprising the one or more genes underlying the QTL may be present in a microorganism, such as a bacterium (e.g. Agrobacterium).

The use of such a (isolated or extracted) nucleic acid molecule and/or of such a recombinant chromosome or part thereof for generating plant cells and plants comprising an internal fruit rot resistance QTL or gene (or allele) is encompassed herein. In one aspect it may be used to generate transgenic plant cells and transgenic plants, e.g. cucumber cells, cucumber plants and parts (e.g. fruits) comprising the internal fruit rot resistance QTL or causal gene or allele and the plant comprises an enhanced internal fruit rot resistance phenotype.

Thus, transgenic plant cells, e.g. transgenic cucumber cells, comprising in their genome a recombinant a recombinant nucleic acid molecule comprising one or more of the (trans) genes or alleles are also an embodiment of the invention. In one aspect the DNA molecule comprising the gene or allele is stably integrated into the cucumber genome.

The fruit rot resistance conferring gene or allele may also be cloned and a chimeric gene may be made, e.g. operably linking a plant expressible promoter to the allele. Such a chimeric gene may be introduced into a plant cell and the plant cell may be regenerated into a whole plant to produce a transgenic plant. In one aspect the transgenic plant is a cucumber plant, or a melon plant.

Thus, transgenic plants, especially transgenic cultivated cucumber or melon plants, comprising a fruit rot resistance conferring gene or allele and having increased resistance to internal fruit rot are provided herein.

Especially cells or cell cultures comprising a recombinant chromosome 5 and/or 3 according to the invention are an embodiment, independent whether the recombinant chromosome 5 and/or 3 is introduced by transgenic methods or by breeding methods. The cells are e.g. in vitro and are regenerable into plants comprising the recombinant chromosome 5 and/or 3 of the invention.

Also the molecular marker sequences (and isolated nucleic acid molecules comprising the sequence) disclosed herein and molecular markers in between any of the mentioned molecular markers described herein, linked to the QTL5.1 and/or QTL3.1, and their use in detecting and/or generating cucumber plants comprising said

QTLs are encompassed herein.

### Fruit Rot Resistance Assay

As a novel protocol has been developed to screen plants, especially cucumbers or wild relatives of cucumber or other Cucurbitaceae (melon or watermelon), for resistance to internal fruit rot, this method is also an embodiment herein.

Wild plant accessions, e.g. wild cucumber or wild relatives of cucumber can be screened for comprising resistance genes or QTLs, but also cultivated plants (e.g. cultivated cucumber) can be screened and/or selected for comprising e.g. QTL5.1 and/or QTL3.1 or other resistance genes which confer internal fruit rot resistance. For example, plants can be mutated (e.g. by targeted mutagenesis such as CRISPR based methods or by random mutagenesis) and such mutated plants can be screened using the fruit rot resistance assay described herein.

Therefore, in one aspect a method for screening cultivated or wild cucumber plants or wild relatives of cucumber for resistance or susceptibility to internal fruit rot caused by Didymella bryoniae, said method comprising:
a) inoculating opened flowers with spores of Didymella bryoniae,
b) allowing the fruits to develop from the inoculated flowers for e.g. at least 10 days or until they have a substantial size, e.g. a marketable size,
c) cutting the fruit open (e.g. longitudinally) to score the fruit for signs of internal fruit rot, especially at the flower end of the fruit.

In one aspect of the method, the flowers which open daily are daily inoculated for a period of e.g. 1, 2 or 3 weeks with a spore suspension. So each day one checks whether flowers opened and one inoculates them. Preferably at least 15, 20, 25, 30, 40, 50 or more flowers of each plant genotype are inoculated during the period.

In one aspect the method further comprises determining the percentage of fruits that develop from spore-inoculated flowers which show internal symptoms of Didymella bryoniae in the fruit.

Scoring of the fruits can be done as described in the Examples, e.g. as having symptoms or not showing symptoms (being free of any symptoms).

The method can be used to select a plant (e.g. a wild accession, a mutated plant, a backcross line, mapping populations, etc.) wherein the percentage of fruits that develop from spore-inoculated flowers which show internal symptoms of Didymella bryoniae in the fruit is significantly lower than in a Didymella bryoniae susceptible control plant included in the screening.

The method is preferably carried out in a controlled environment, e.g. a greenhouse, and at least 5, 6, 7, 8, 9, 10, 15, 20 or more plants of a genotype are grown together with at least 5, 6, 7, 8, 9, 10, 15, 20 or more plants of a DB susceptible control. Optionally also a resistant control may be included.

Preferably at least 15, 20, 25, 30, 40, 50 or more fruits of a plant genotype is allowed to develop from spore inoculated flowers and is thus assessed for the development of internal symptoms.

### Seed Deposits

A representative sample of seeds of a Cucumis sativus var. sativus line of the long cucumber type, designated DB-resistant, comprising an introgression fragment comprising QTL5.1 and an introgression fragment comprising the duplication of QTL3.1 in homozygous form, and a genetic control (GC) lacking any introgression fragments and lacking the QTLs (i.e. lacking the duplication of QTL3.1 and lacking QTL5.1), designated DB-susceptible, were deposited by Nunhems B. V. on 5 Dec. 2019 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32 (1)). Seeds were given the following deposit numbers NCIMB 43530 (DB-resistant) and NCIMB 43531 (DB-susceptible).

The Applicant requests that samples of the biological material and any material derived therefrom be only released to a designated Expert in accordance with Rule 32 (1) EPC or related legislation of countries or treaties having similar rules and regulation, until the mention of the grant of the patent, or for 20 years from the date of filing if the application is refused, withdrawn or deemed to be withdrawn.

Access to the deposit will be available during the pendency of this application to persons determined by the Director of the U.S. Patent Office to be entitled thereto upon request. Subject to 37 C.F.R. § 1.808 (b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer and will be replaced if it ever becomes nonviable during that period. Applicant does not waive any rights granted under this patent on this application or under the Plant Variety Protection Act (7 USC 2321 et seq.).

The following non-limiting Examples describe how one can obtain plants according to the invention, comprising QTL5.1 and/or QTL3.1 and/or modify gene expression of one or more causal genes underlying QTL3.1. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard breeding methods are described in ‘Principles of Plant breeding’, Second Edition, Robert W. Allard (ISBN 0-471-02309-4).

### Examples

### Example 1-Phenotyping

### 1.1-Development of Internal Fruit Rot Resistance Assay

After extensive research the following protocol was developed to phenotype plants for internal fruit rot resistance and susceptibility.

### Internal Fruit Rot Protocol:

Ten cucumber plants per genotype are grown in greenhouse for six weeks under a temperature regime of 18° C./20° C. (day/night). 3×10⁴ spore/ml of Didymella bryoniae are resuspended in water containing 0.001% Tween20 and daily opened flowers are inoculated with the spore suspension (10 μl spore suspension per flower) over a period of 3 weeks. Ten-days post inoculation, full size cucumbers (marketable size) which had developed from the inoculated flowers are sliced in two halves to evaluate presence or absence of internal fruit rot caused by Didymella bryoniae according to FIG. 1. The number of fruits showing symptoms of internal fruit infection (FIG. 1, three fruits on the right) and the number of fruits not showing symptoms of internal fruit infection (FIG. 1, fruit on the left) is noted down, and the percentage of fruits showing symptoms of infection out of the total number of fruits developed from spore-inoculated flowers is calculated.

### Example 2-QTL Mapping

The QTLs for the internal fruit rot resistance phenotype (above) was mapped in a two mapping population developed from a wild cucumber donor, MYCR3, and two different elite long cucumber lines.

Two QTLs of the donor were found to be responsible for internal fruit rot resistance, one on chromosome 5 (QTL5.1) and one on chromosome 3 (QTL3.1, which was later in Example 6 found to be a duplication of QTL3,1).

TABLE 1
SNP markers and their genotypes and haplotypes linked to QTL5.1

| | | Genotype | | | |
|---|---|---|---|---|---|
| | Physical position | of | Genotype | | |
| | of SNP (base | introgres- | of | | |
| | number) in | sion | recurrent | Donor | |
| | genome of | fragment | parent | SNP | |
| | Chinese Long V2 | (homozygous | lacking | Haplo- | |
| SNP | (cucurbitgenomics. | for | intro- | type | Genomic sequence |
| marker | org) | QTL5.1) | gression | of QTL5.1 | comprising SNP |
| SNP_01 | 3701817 | AA | GG | A | GTGGCAATGGAGCAAG |
| | | | | | ACAACGAAGAGGTGAA |
| | | | | | GACGTTTCAGAGCTTAG |
| | | | | | G[A/G]ATCTGTGAGCAA |
| | | | | | TTGGTTGAGGCATGTGA |
| | | | | | CAGTTTAGGCTGGAAA |
| | | | | | AATCC |
| | | | | | SEQ ID NO: 1 |
| SNP_02 | 3715105 | GG | AA | G | CATCAACTTTTTAATTC |
| | | | | | TAAAACATTGGTAGTAT |
| | | | | | AGTAATGAAAAAAAGG |
| | | | | | [A/G]TATTAATTATTGCA |
| | | | | | TCACACTAATTTAATAA |
| | | | | | GAGACTAAATGGAGAA |
| | | | | | GAT |
| | | | | | SEQ ID NO: 2 |
| SNP_03 | 3725077 | CC | TT | C | GGGCATTTCATGATACC |
| | | | | | ACATGCAGTGAAAGAA |
| | | | | | GACACAACTTTGGAGAA |
| | | | | | [T/C]TATTTCATACCAA |
| | | | | | AGAATGGCATCGTGAG |
| | | | | | TTTCTTTGCAGTGGGAA |
| | | | | | TGGG |
| | | | | | SEQ ID NO: 3 |
| SNP_04 | 3735384 | GG | AA | G | AACATTCTTAATCTAAG |
| | | | | | AGCAAGTAATCACCAC |
| | | | | | AGAAAAGGAGAAGATG |
| | | | | | A[A/G]CCAATCAAGGGA |
| | | | | | ACCATACAAATTTATGA |
| | | | | | TAAAAAAAGATACGAA |
| | | | | | AAAAG |
| | | | | | SEQ ID NO: 4 |
| SNP_05 | 3740736 | AA | CC | A | TAAATTATCAAATTGGC |
| | | | | | TCATACGGTGTACAATT |
| | | | | | AGGAAAGAATGTCTAA |
| | | | | | [C/A]ACAGGAGATGAAG |
| | | | | | ATCTGCTTGGATCTTTG |
| | | | | | ACTTCAAAAGAAAAAG |
| | | | | | GGAA |
| | | | | | SEQ ID NO: 5 |
| SNP_06 | 3752787 | AA | GG | A | CTAGGACCTTGAGTCTT |
| | | | | | TCCTCATTTGTCCCACC |
| | | | | | ACTCTTGTCCACTTAC |
| | | | | | [G/A]AGAACTTGCTCTTAT |
| | | | | | CAAAGTCCGTAGAAAA |
| | | | | | GGTAGATAGAGCACGA |
| | | | | | CGA |
| | | | | | SEQ ID NO: 6 |
| SNP_07 | 3764323 | CC | TT | C | ATCCCTTTTTCCCATTTT |
| | | | | | TTTGAAAAATTAATTCT |
| | | | | | ATCGTCCTTTTACAA[C/ |
| | | | | | T]ATCTGATAACGACAA |
| | | | | | TCAAGTTGATTTTTCGG |
| | | | | | TATATCATATTCACTTC |
| | | | | | T |
| | | | | | SEQ ID NO: 7 |
| SNP_08 | 3777423 | CC | AA | C | AAAGTAAGCCTTTTTTG |
| | | | | | CTTATTTTGGTTTTTGTA |
| | | | | | AATTGGGAAAGGGAA[A/ |
| | | | | | C]ATTTTTAACGTCCAC |
| | | | | | CATTGCAGGACAGCCC |
| | | | | | ATGCTATATATAGTTCT |
| | | | | | CT |
| | | | | | SEQ ID NO: 8 |
| SNP_09 | 3780450 | AA | GG | A | TTGCTCAATCATATATT |
| | | | | | CAAGCTAATGTATTAAT |
| | | | | | ACTTTGTTGCTTTAGT[G/ |
| | | | | | A]TTGGCAAACCAAATC |
| | | | | | AGcATTTCCAaTTCACCA |
| | | | | | CTGCATATTTTGCTCTT |
| | | | | | SEQ ID NO: 9 |
| SNP_10 | 3791090 | AA | GG | A | TTTATACGAATGAACAT |
| | | | | | TTAAATAGGTAGCAAGT |
| | | | | | GCAGATTTAAAAACCA |
| | | | | | [G/A]AACATTCTTTCACT |
| | | | | | TTTCAAAACTTGGATTT |
| | | | | | TGAAAGCAAGAATCTA |
| | | | | | GCT |
| | | | | | SEQ ID NO: 10 |
| SNP_11 | 3809014 | CC | TT | C | TAAGTGTGAAATTTGTG |
| | | | | | GAAATGTGCAAAATCG |
| | | | | | TAGTTAAGAAAATAGA |
| | | | | | T[T/C]TTGAAATGAACT |
| | | | | | TTCACTACAATTTTCTT |
| | | | | | AACGCACAAATTCAGA |
| | | | | | TGTTG |
| | | | | | SEQ ID NO: 11 |
| SNP_12 | 3823864 | GG | AA | G | AAAAACTTGTACATATT |
| | | | | | AATATAGTCTTAACTCT |
| | | | | | TAACCATCACAACATC |
| | | | | | [A/G]TTCAAAAGATGCTC |
| | | | | | CTTTTAACACCATTTCT |
| | | | | | CCAAAACGAAGCTGCA |
| | | | | | GTT |
| | | | | | SEQ ID NO: 12 |
| SNP_13 | 3832094 | AA | GG | A | ATTGAATATTTGAGAAG |
| | | | | | TTCAATGAAGATATTTA |
| | | | | | AAGGAAGAAAGAAAAA |
| | | | | | [A/G]GAAACAGAGATAC |
| | | | | | ATAGGTTTGTATTGGTA |
| | | | | | ACATAAAAAATGCAAA |
| | | | | | AAGA |
| | | | | | SEQ ID NO: 13 |
| SNP_14 | 3843862 | GG | AA | G | TAGCTATGTTATTGTAT |
| | | | | | GTATTGAAAATTAATAT |
| | | | | | AGCCATTGTTTAATTT[A/ |
| | | | | | G]TTGCAATGATTATGA |
| | | | | | TATATTGCATGAGTCGT |
| | | | | | GCAAGTTATGAACAACT |
| | | | | | T |
| | | | | | SEQ ID NO: 14 |
| SNP_15 | 3967955 | GG | TT | G | TTGGATTGTTGCATTTT |
| | | | | | GAGCATTCAATTATTAA |
| | | | | | GTCATAACAGAAGAAC |
| | | | | | [T/G]ATCTCTTTAAATAA |
| | | | | | TTTTTAAAAGGTTATGT |
| | | | | | TCTCAATAATTGATCTC |
| | | | | | AA |
| | | | | | SEQ ID NO: 15 |
| SNP_16 | 3971624 | CC | AA | C | TGTAGGAATATAGAAC |
| | | | | | AAAATCAATATTGCAAT |
| | | | | | AGCAAAACTTTGAGAA |
| | | | | | A[C/A]TATAGTAAAATT |
| | | | | | ATTTCAAATTTCATCGT |
| | | | | | ATTAATATTTATAGTTA |
| | | | | | AACG |
| | | | | | SEQ ID NO: 16 |
| SNP_17 | 3985147 | AA | GG | A | TAATTTCAAGACAACAA |
| | | | | | AATTTAAANAAAAAAA |
| | | | | | AAACGAAAAAAGAGTT |
| | | | | | T[A/G]TGGGGTGAGAGA |
| | | | | | GGCTCGAACTCTCGACC |
| | | | | | TCAGGATCACTCTAAGC |
| | | | | | TATG |
| | | | | | SEQ ID NO: 17 |
| SNP_18 | 4028826 | GG | AA | G | ACACTCAAGGAAAACA |
| | | | | | ACTAATTTACTGTTTGA |
| | | | | | AGATCCAGGGGAAAAA |
| | | | | | A[A/G]AGTAACCTTGAC |
| | | | | | TGCAGCTGTAATAACTT |
| | | | | | TTTCACCAATAATTCCA |
| | | | | | ACAC |
| | | | | | SEQ ID NO: 18 |

TABLE 2
SNP markers (genotype and haplotype) linked to QTL3.1

| | | Genotype | | | |
|---|---|---|---|---|---|
| | Physical position | of | Genotype | | |
| | of SNP (base | intro- | of | | |
| | number) in | gression | recurrent | | |
| | genome of | fragment | parent | | |
| | Chinese Long V2 | (homozygous | lacking | Donor SNP | |
| SNP | (cucurbitgenomics. | for | intro- | Haplotype | Genomic sequence |
| marker | org) | QTL3.1) | gression | of QTL3.1 | comprising SNP |
| SNP_19 | 9047770 | GG | AA | G | TTCGAAATCGAAAGTTT |
| | | | | | CGAATTTCTCTGTAAAA |
| | | | | | TTACAATAAGAAAAAC |
| | | | | | [A/G]TTAAACTATTCTAC |
| | | | | | TTCCGTGGTTATAAATA |
| | | | | | AATCTTCAAGCCNTTTT |
| | | | | | TT |
| | | | | | SEQ ID NO: 19 |
| SNP_20 | 9060467 | GG | TT | G | TCACCATTTACATCAAC |
| | | | | | CTGTGTCAAAGCAATAA |
| | | | | | AAGTTTATTCAATGAT |
| | | | | | [T/G]ATGTCATTCGCCCT |
| | | | | | CAAATTAAGATTTTTTT |
| | | | | | CCTTTACTAAATCCACT |
| | | | | | TTGT |
| | | | | | SEQ ID NO: 20 |
| SNP_21 | 9071227 | TT | CC | T | GAAATTGAAATTGAAA |
| | | | | | TTCTAATTTTTCATTTCT |
| | | | | | TTTTCAGCTTTTGTTT[T/ |
| | | | | | C]AAATTAAATTAAATT |
| | | | | | AATTTGTTTATTGCTTTT |
| | | | | | GTTAACCCAAACGCTCT |
| | | | | | SEQ ID NO: 21 |
| SNP_22 | 9076137 | AA | GG | A | GTTTGAGTTTTTGGTGA |
| | | | | | AGATTTAAACTCGTTCA |
| | | | | | TCTGCTCAAATTTTGC[A/ |
| | | | | | G]AACACCCAAACAAA |
| | | | | | TCCAAATCCATAATCCA |
| | | | | | TAATTCATAATCCAATC |
| | | | | | CA |
| | | | | | SEQ ID NO: 22 |
| SNP_23 | 9086683 | DD | II | C | TTTTCAAGGAGATACAA |
| | | (deletion | (insertion | (deletion | ATTTTCATTTTTTTTTAT |
| | | of A) | of A) | of A) | ATTTCAAAATTAGCA[C/ |
| | | | | | CA]AAAGTTAAATTAAG |
| | | | | | ATAGATACGAGTATTTT |
| | | | | | TCTATTTGAATTAGCCT |
| | | | | | TA |
| | | | | | SEQ ID NO: 23 |
| SNP_24 | 9100285 | AA | CC | A | ATGTATGTTTGCACGGT |
| | | | | | CATGATTTTATTTGATA |
| | | | | | GTATTCAGTTTTGCAC[A/ |
| | | | | | C]GCCATAATTTTATGC |
| | | | | | ATGTATTTGATACTACT |
| | | | | | TATACATTTTTATATTG |
| | | | | | G |
| | | | | | SEQ ID NO: 24 |
| SNP_25 | 9104670 | GG | AA | G | TAGCAACTTCCAGCGAC |
| | | | | | ATGAAAATAAACTTCCG |
| | | | | | TGCCAAAAAAAGGTAA |
| | | | | | [G/A]GTGATATTAAGAA |
| | | | | | AAGAGATTTTAATTGAC |
| | | | | | ATCACCAAGATCATAG |
| | | | | | AATA |
| | | | | | SEQ ID NO: 25 |
| SNP_26 | 9114652 | TT | CC | T | TGTATTATTTGATGAAA |
| | | | | | AAATCATTAAAATAAA |
| | | | | | CTCTGTATTTATATTAA |
| | | | | | [T/C]TTTAGCATTTTGAA |
| | | | | | TACAAACTCTAATAAAT |
| | | | | | AATCATTTTATATTTGT |
| | | | | | TT |
| | | | | | SEQ ID NO: 26 |
| SNP_27 | 9124475 | CC | TT | C | ACAAATATTAAACACA |
| | | | | | AAAGTCAAAAAAAAAT |
| | | | | | CCAGACCTTGATGTTAT |
| | | | | | C[C/T]CTCACCTACTCTG |
| | | | | | TTTCTGAGAATGAATTT |
| | | | | | CAACGGAGTTTTGTGCG |
| | | | | | ATA |
| | | | | | SEQ ID NO: 27 |
| SNP_28 | 9136651 | GG | AA | G | TTGAAGAGAAGCTGAA |
| | | | | | GAGTGGATGATGAGCA |
| | | | | | GAAGAAGACTCGGGAA |
| | | | | | TC[G/A]GAGAGAAGATT |
| | | | | | GAAGAGATGCTATGGC |
| | | | | | TGCAGAAAAAGGCGGG |
| | | | | | ACCGGAG |
| | | | | | SEQ ID NO: 28 |
| SNP_29 | 9146287 | CC | AA | C | TATCAGAAAATTAATAT |
| | | | | | AAACTCTAGGCAAGGA |
| | | | | | TAATTATATAAAGAATA |
| | | | | | [C/A]ATTTCAGTGAATC |
| | | | | | ACAAAACCTAAGCCAG |
| | | | | | GTCCAGATTACAGTTCA |
| | | | | | AAGC |
| | | | | | SEQ ID NO: 29 |
| SNP_30 | 9156322 | AA | GG | A | TTTTACTTTTTGATAAT |
| | | | | | GAAAATTTACAACAAA |
| | | | | | TATATCCCAAGTTAACT |
| | | | | | [G/A]ATTTATATTATATA |
| | | | | | GATTTTAATTTTGAAAA |
| | | | | | AGAATATAATGTCAAA |
| | | | | | GCT |
| | | | | | SEQ ID NO: 30 |
| SNP_31 | 9164459 | GG | TT | G | TTTAACTGTTGACTGAC |
| | | | | | CTTACTCTTGGTTTTCTT |
| | | | | | CGGGAAAGTCTTCGT[G/ |
| | | | | | T]AGTTCGTTTAGTATAT |
| | | | | | GGAGAAATACTTGGGT |
| | | | | | CAAGAAGAAGAAATTA |
| | | | | | GT |
| | | | | | SEQ ID NO: 31 |
| SNP_32 | 9174546 | GG | AA | G | TGGATGCGTAAAAGTG |
| | | | | | GCTGTAGTTGGAGTTTC |
| | | | | | CGTGTGAAATCTGACGT |
| | | | | | [G/A]TGGACTTCCAAAA |
| | | | | | AGGTGTTAGGGACCCA |
| | | | | | CCTTAGAAGGAAATAA |
| | | | | | ACGAC |
| | | | | | SEQ ID NO: 32 |
| SNP_33 | 9201783 | GG | CC | G | CTTATATGAGTTTTTTTT |
| | | | | | TTTTTTTTTAAAAATAT |
| | | | | | CTTCTTCTAAGAAAT[C/ |
| | | | | | G]AAATTATTTTCCTTTT |
| | | | | | TAGAAATTTCTAAAATT |
| | | | | | TAGGAAATCAAATTATT |
| | | | | | SEQ ID NO: 33 |
| SNP_34 | 9214742 | GG | AA | G | TTGTTACAATCATCTTC |
| | | | | | AATTATTCAGCATTATC |
| | | | | | TACATGGATGGATTAT |
| | | | | | [G/A]TATAATTAGTGACA |
| | | | | | ACACATAATTTTATAAG |
| | | | | | AAATTAGAAGCAAAAT |
| | | | | | TAT |
| | | | | | SEQ ID NO: 34 |
| SNP_35 | 9237416 | TT | CC | T | CAATCAAAGTATTTGGT |
| | | | | | TGATATAAAAATGAGA |
| | | | | | AAGATTTAGATTTGGGA |
| | | | | | [T/C]ATTTTCAAATATA |
| | | | | | AGAAATGCATTAAAAT |
| | | | | | ATTTATAAAAAAATAGC |
| | | | | | AAAG |
| | | | | | SEQ ID NO: 35 |
| SNP_36 | 9264936 | DD | II | T | GGAATTGTCTTCAAAGG |
| | | (deletion | (insertion | (deletion | TAGCTGTTCTGAGGAGA |
| | | of G) | of G) | of G) | AATTTATCATCCAAGT |
| | | | | | [T/TG]GGGGTTGCTATAC |
| | | | | | CTGATATTGATGTAAGG |
| | | | | | CCATGTTAGGCCTTATT |
| | | | | | CAC |
| | | | | | SEQ ID NO: 36 |
| SNP_37 | 9294008 | CC | TT | C | TCATATTAACTGCATTC |
| | | | | | TAATTTAACAATGTAAA |
| | | | | | ATGACTTTTATTATAC[T/ |
| | | | | | C]TATTTGGAGAATTTT |
| | | | | | GTTATTTTATTATTATTG |
| | | | | | TTTTTTTTTACTACACA |
| | | | | | SEQ ID NO: 37 |
| SNP_38 | 9317494 | GG | AA | G | ACATACCTTCTAGTTCA |
| | | | | | TTGAACCTTCACACTAA |
| | | | | | TATCATGACCTAGTTG |
| | | | | | [G/A]ACAGCGGTAGACA |
| | | | | | CGTGACATTTTTTAGAT |
| | | | | | ATATTCATTCCTCTTAA |
| | | | | | CAT |
| | | | | | SEQ ID NO: 38 |
| SNP_39 | 9327900 | CC | AA | C | GAAGGAGAAGTGTTAA |
| | | | | | AAGTTGTTCATTTGACA |
| | | | | | AGGCTACGTTAAATCAC |
| | | | | | [C/A]TTTTCTCCCAATTG |
| | | | | | CCCTCAAAATCAAATTG |
| | | | | | AGATGAATTTTAGTTTT |
| | | | | | GA |
| | | | | | SEQ ID NO: 39 |
| SNP_40 | 9337703 | TT | CC | T | GATATGCATCTCAATAA |
| | | | | | AATAATTCATCCACAAT |
| | | | | | CTTTTCCATAAAAAAA |
| | | | | | [T/C]TTTTTTTTGCTCACT |
| | | | | | TTCAAAGATAGAAATAT |
| | | | | | GGGATTTGTCTTTAAAT |
| | | | | | T |
| | | | | | SEQ ID NO: 40 |
| SNP_41 | 9344313 | AA | GG | A | AGTTGGGAAATGTAAA |
| | | | | | GTAAAAGTTTGACTTAT |
| | | | | | GAACATTATGATCTACA |
| | | | | | [A/G]TTTCATTTACATTT |
| | | | | | TATATATAATAGAAAGC |
| | | | | | ATATGAAACATGAGTTT |
| | | | | | TG |
| | | | | | SEQ ID NO: 41 |
| SNP_42 | 9357469 | AA | GG | A | GGAGAGGATGAAAAAA |
| | | | | | CAAGTTAAATATAAAAT |
| | | | | | AAAAATAAAAATAAAA |
| | | | | | T[A/G]ACTTAGTTCCCAT |
| | | | | | CGTACAAAAATTTAAA |
| | | | | | ATTTCTTTTTCTTTTATC |
| | | | | | TAA |
| | | | | | SEQ ID NO: 42 |

It is noted that two of the above markers, SNP_23 and SNP_36, are INDEL markers, whereby one nucleotide is present (inserted, I) or absent (deleted, D).

The sequences provided in the sequence listing are the sequences present in the QTL donor.

A long cucumber introgression line comprising both QTL5.1 and QTL3.1 (later to be found to comprises a duplication of QTL3.1, see Example 6) in homozygous form was selected for making a seed deposit, NCIMB 43530 (DB-resistant). Also the genetic control lacking the two QTLs was deposited, NCIMB 43531 (DB-susceptible). Later it was found that the genetic control lacked the duplication of QTL3.1, see Example 6.

### Example 3-Phenotype Conferred by QTL5.1 and QTL3.1 Together (Homozygous)

Long cucumber plants comprising both QTLs from the MYCR3 donor were analyzed together with control plants in the internal fruit rot assay described above.

The results in Table 3 show that both QTLs together provide a high level of resistance to internal fruit rot in long cucumber.

As QTL3.1 from the donor MYCR3 was later found (see Example 6) to comprises a duplication of QTL3.1, the table below is understood to refer to the duplication of QTL3.1 on chromosome 3 where QTL3.1 is present and to lacking the duplication of QTL3.1 on chromosome 3 where QTL3 is mentioned to be absent.

TABLE 3
internal fruit rot results

| | | | Total | % of fruits |
|---|---|---|---|---|
| | | Number | number | with internal |
| | | of | of | fruit rot |
| Plant genotype | QTLs | Plants | Fruits | symptoms |
| NCIMB 43530 | QTL 3 + 5 present | 10 | 106 | 0% |
| (DB-resistant) | | | | |
| NCIMB 43531 | QTL 3 + 5 absent | 10 | 136 | 29% |
| (DB-susceptible, | | | | |
| genetic control) | | | | |
| Susceptible long | QTL 3 + 5 absent | 15 | 190 | 41% |
| cucumber parent | | | | |
| MYCR3 donor | QTL 3 + 5 present | 14 | 140 | 0% |

### Example 4-Phenotype Conferred by Individual QTLs (Homozygous)

To test the effect of the individual QTLs (in homozygous form), the QTLs were backcrossed from the donor into two different long cucumber elite lines, line HY128 line and line HY2719.

As QTL3.1 from the donor MYCR3 was later found (see Example 6) to comprises a duplication of QTL3.1, the table below is understood to refer to the duplication of QTL3.1 on chromosome 3 where QTL3.1 is present and to lacking the duplication of QTL3.1 on chromosome 3 where QTL3 is mentioned to be absent.

TABLE 4
internal fruit rot results in two different genetic backgrounds

| | | | | | Reduction of |
|---|---|---|---|---|---|
| | | | | % of | base-line |
| | | | | fruits with | susceptibility |
| | | Number | Total | internal | (relative to |
| | | of | number of | fruit rot | Genetic |
| Plant genotype | QTLs | plants | Fruits | symptoms | Control) |
| HY128 susceptible | QTL3 + 5 absent | 19 | 260 | 37% | |
| parent | | | | | |
| Genetic control | QTL3 + 5 absent | 8 | 109 | 27% | |
| Backcross line | QTL 3 present | 8 | 98 | 3% | 24% |
| Backcross line | QTL 3 present | 8 | 97 | 3% | 24% |
| Backcross line | QTL 5 present | 8 | 69 | 0% | 27% |
| Backcross line | QTL 5 present | 8 | 80 | 4% | 23% |
| Backcross line | QTL 3 + 5 present | 10 | 54 | 0% | 27% |
| HY2719 susceptible | QTL 3 + 5 absent | 19 | 240 | 52% | |
| parent | | | | | |
| Genetic control | QTL3 + 5 absent | 8 | 97 | 32% | |
| Backcross line | QTL 3 present | 8 | 95 | 29% | 3% |
| Backcross line | QTL 3 present | 8 | 94 | 28% | 4% |
| Backcross line | QTL 5 present | 8 | 93 | 8% | 24% |
| Backcross line | QTL 5 present | 8 | 94 | 9% | 23% |
| Backcross line | QTL 3 + 5 present | 16 | 150 | 1.3% | 30.7% |
| Backcross line | QTL 3 + 5 present | 16 | 149 | 0.7% | 31.3% |
| MYCR3 donor | QTL 3 + 5 present | 8 | 81 | 0% | 32% |

The results show that QTL3.1 (i.e. the duplication of QTL3.1) and QTL5.1 alone generally have a smaller effect on reducing the susceptibility to internal fruit rot than the combination of QTL3.1 (i.e. the duplication of QTL3.1) and QTL5.1 together and that the effect is larger in one genetic background than in another genetic background. For example in the HY128 genetic background the effect is much stronger than in the HY2719 genetic background.

### Example 5-Effect of QTL Copies being Present

As QTL3.1 from the donor MYCR3 was later found (see Example 6) to comprises a duplication of QTL3.1, the table below is understood to refer to the duplication of QTL3.1 on chromosome 3 where QTL3.1 is present and to lacking the duplication of QTL3.1 on chromosome 3 where QTL3 is mentioned to be absent.

The effect of the number of QTL copies (DB resistance alleles or donor alleles) was analyzed in two F2 populations. Plants having different combinations of QTL3.1 and QTL5.1 in homozygous or heterozygous form were analyzed for the percentage of infected fruits. The results in Table 5 show that the more QTL copies/donor alleles are present, the larger the effect on internal fruit rot resistance. The strongest effect is seen when 4 donor alleles are present, i.e. QTL3.1 (i.e. the duplication of QTL3.1 on chromosome 3) and QTL5.1 are both in homozygous form.

TABLE 5

| | Presence | Donor | Population | Population |
|---|---|---|---|---|
| Presence of QTL3.1 | of QTL5.1 | alleles | 1 | 2 |
| Absent | Absent | 0 | 61% | 54% |
| (no duplication of | | | | |
| QTL3.1) | | | | |
| Absent | heterozygous | 1 | 39% | 32% |
| (no duplication of | | | | |
| QTL3.1) | | | | |
| Absent | homozygous | 2 | 15% | 13% |
| (no duplication of | | | | |
| QTL3.1) | | | | |
| Heterozygous (one | Absent | 1 | 37% | 27% |
| chromosome with | | | | |
| duplication of QTL3.1) | | | | |
| Heterozygous | heterozygous | 2 | 19% | 16% |
| (one chromosome with | | | | |
| duplication of QTL3.1) | | | | |
| Heterozygous | homozygous | 3 | 8% | 6% |
| (one chromosome with | | | | |
| duplication of QTL3.1) | | | | |
| Homozygous | Absent | 2 | 30% | 18% |
| (two chromosomes with | | | | |
| duplication of QTL3.1) | | | | |
| Homozygous | heterozygous | 3 | 4% | 8% |
| (two chromosomes with | | | | |
| duplication of QTL3.1) | | | | |
| Homozygous | homozygous | 4 | 1% | 0% |
| (two chromosomes with | | | | |
| duplication of QTL3.1) | | | | |

### Example 6-Fine Mapping of QTL3.1

QTL3.1 was fine-mapped and was found to be linked to SNP_35 and SNP_36.

Long-read nanopore sequencing was carried out and revealed a duplication of a region of about 150.000 kb on chromosome 3 (from base 9138854 to base 9286145 of chromosome 3 in the Chinese Long V2 genome) in the resistant plant. The first and last 100 bases of the duplicated region are provided in SEQ ID NO: 83 and 84.

The region duplicated on chromosome 3 in the resistant plant contained QTL3.1, thus resulting in a duplication of QTL3.1 (starting at SNP_35 and ending 659 bases downstream of SNP_36) on chromosome 3. See FIG. 4, showing the duplication in the resistant plant. The nucleotide sequence comprising QTL3.1, i.e. starting at SNP_35 and ending 659 nucleotides downstream of SNP_36 is provided herein in SEQ ID NO: 72 (plus strand) and SEQ ID NO: 73 (minus strand). The resistant plant, which was homozygous for QTL3.1, thus contained QTL3.1 four times, while the susceptible plant, which was also homozygous, contained QTL3.1 two times.

The fine mapped QTL3.1 region (i.e. starting at SNP_35 and ending 659 nucleotides downstream of SNP_36, see SEQ ID NO: 72 and 73) contains 9 genes, 8 of these genes were annotated to encode “1-aminocyclopropane-1-carboxylate oxidase-like proteins” and one gene encodes a HR-like protein (HR means Hypersensitive Response).

The 8 genes annotated as ACO-like proteins were analyzed for their sequence identity to known ACO proteins of Arabidopsis, AtACO1 to AtACO5, as described in Houben and Van de Poel (2019, Front. Plant Sci. 10:695) and were also analyzed by BLAST against the Arabidopsis protein database using the NCBI protein-protein BLAST tool.

The sequence identity to the AtACO1 to AtACO5 proteins was found to be low (see Table 6-A), and the most similar Arabidopsis sequences were found to be two genes (At1g06620.1 and At1g06650.1), encoding proteins NP_172147.2 and NP_172150.1, respectively. These two proteins are annotated as being 2-oxoglutarate and Fe(II) dependent oxygenases, or 2-OGD.

It was, therefore, concluded that the 8 proteins found are 2-oxoglutarate and Fe(II) dependent oxygenases (rather than ACO proteins) and it was decided to name these 8 proteins 2-OGD1 to 2-OGD8, or CsOGD1 to CsOGD8 (or simply OGD1 to OGD8).

TABLE 6-A
percentage sequence identity of proteins (pairwise alignment using Emboss-Needle, default
parameters)

| | 2-OGD1 | 2OGD2 | 2-OGD3 | 2-OGD 4 | 2-OGD 5 | 2-OGD 6 | 2-OGD 7 | 2-OGD 8 |
|---|---|---|---|---|---|---|---|---|
| 2-OGD 1 | 100 | | | | | | | |
| 2-OGD 2 | 69.4 | 100 | | | | | | |
| 2-OGD 3 | 75.2 | 65.6 | 100 | | | | | |
| 2-OGD 4 | 58.2 | 53.4 | 58.8 | 100 | | | | |
| 2-OGD 5 | 54.2 | 50.4 | 57.5 | 55.4 | 100 | | | |
| 2-OGD 6 | 49.5 | 48.6 | 52.7 | 52.4 | 62.4 | 100 | | |
| 2-OGD 7 | 53.0 | 50.2 | 56.3 | 56.1 | 72.4 | 67.5 | 100 | |
| 2-OGD 8 | 54.7 | 51.8 | 57.9 | 54.8 | 68.3 | 62.2 | 71.9 | 100 |
| At1g06620.1 | 51.5 | 45.9 | 53.7 | 50.9 | 52.5 | 47.3 | 52.1 | 54.1 |
| NP_172147.2 | | | | | | | | |
| At1g06650.1 | 52.9 | 45.2 | SS.5 | 52.3 | 52.3. | 51.1 | 50.5 | 52.1 |
| NP_172150.1 | | | | | | | | |
| AtACO1 | 23.5 | 21.9 | 22.0 | 22.8 | 22.8 | 24.0 | 23.0 | 24.1 |
| AtACO2 | 24.2 | 20.1 | 22.2 | 24.1 | 22.0 | 22.6 | 23.1 | 22.4 |
| AtACO3 | 21.2 | 20.7 | 21.5 | 24.1 | 21.2 | 22.7 | 22.4 | 22.1 |
| AtACO4 | 21.9 | 19.6 | 23.6 | 24.3 | 23.8 | 23.0 | 22.4 | 22.8 |
| AtACOS | 21.3 | 20.2 | 22.8 | 25.6 | 24.6 | 21.8 | 23.0 | 24.3 |

TABLE 6-B

| Name of gene in | | | |
|---|---|---|---|
| database Chinese | Name given | | |
| Long V2 | herein to the | | |
| (cucurbitgenomics.org) | protein | Protein sequence | cDNA sequence |
| Csa3G135680.1 | 2-OGDI | SEQ ID NO: 52 | SEQ ID NO: 61 |
| Csa3G135690.1 | 2-OGD2 | SEQ ID NO: 53 | SEQ ID NO: 62 |
| Csa3G135700.1 | 2-OGD3 | SEQ ID NO: 54 | SEQ ID NO: 63 |
| Csa3G135710.1 | 2-OGD4 | SEQ ID NO: 55 | SEQ ID NO: 64 |
| Csa3G135720.1 | 2-OGD5 | SEQ ID NO: 56 | SEQ ID NO: 65 |
| Csa3G135730.1 | 2-OGD6 | SEQ ID NO: 57 | SEQ ID NO: 66 |
| Csa3G135740.1 | 2-OGD7 | SEQ ID NO: 58 | SEQ ID NO: 67 |
| Csa3G135750.1 | 2-OGD8 | SEQ ID NO: 59 | SEQ ID NO: 68 |
| Csa3G135760.1 | HR-like | SEQ ID NO: 60 | SEQ ID NO: 69 |

The sequence of the QTL3.1 region, comprising the 9 genes, was compared between the resistant (MYCR3 donor) and the reference genome (cucumber Chinese Long V2) and the duplicated QTL3.1 sequences were compared to each other. The proteins encoded were all identical. However the genomic sequence of the MYCR3 donor and the reference genome contain a low level of sequence variation, which was not measured but the sequence of the reference genome provided herein (SEQ ID NO: 72 and 73) was estimated to be at least 97%, most likely at least 98% or 99% identical to the actual sequence of the MYCR3 donor.

The homozygous resistant plant, containing a duplication of QTL3.1, thus contained four copies of each gene (two copies on each chromosome 3), while the homozygous susceptible plant contained 2 copies of each gene, one on each chromosome 3. The plant thus contained 32 copies of a gene encoding a 2-oxoglutarate dependent dioxygenase and four copies of a gene encoding a HR-like protein.

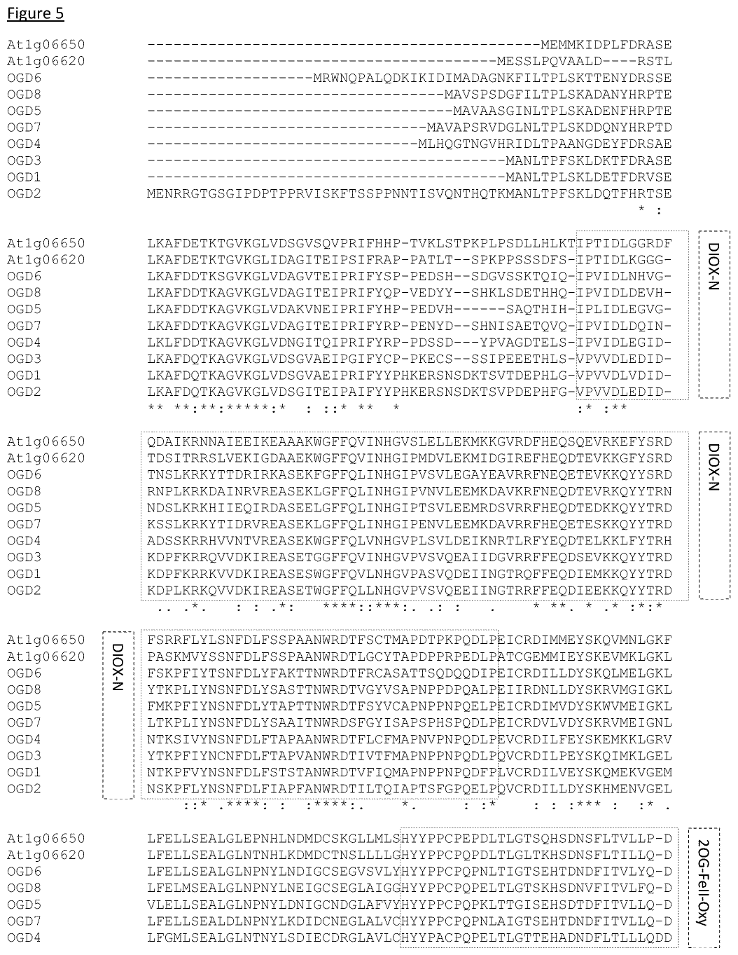

A multiple sequence alignment of the eight CsOGD proteins and the two Arabidopsis 2OGD proteins is shown in FIG. 5. The proteins all contain the conserved Pfam domain named “DIOX-N” (Pfam14226) and the conserved Pfam domain 2-OG-Fell-Oxy (Pfam 03171), indicated in the figure.

As the (homozygous) resistant plant contained four copies of QTL3.1 (two on each chromosome), and thus four copies of each of OGD1 to OGD8 and four copies of the HR-like protein, it was decided to look at (and quantify) the expression of these nine genes and to compare the expression between resistant plants and susceptible plants.

Total RNA of leaf tissue and ovary tissue was extracted using a plants RNA extraction kit (RNeasy Plant Mini Kit, Qiagen), and the transcriptome was analyzed using RNA-Seq (Illumina dye sequencing, Illumina) and gene expression quantified by divided the average raw count of each gene within a library are by the geometric mean of all counts in all samples and the median of the quotients is calculated per library. Each raw count is then divided through the library-specific median value, according to Anders and Huber (2010, Genome Biology 2010, 11: R106). The displayed number represents the average of the normalized values of all replicates (n=3) to determine the gene expression of the nine genes. RNA-Seq as well the quantification were carried out by a service company GenXpro.

TABLE 7

| | | | | | | Increase in |
|---|---|---|---|---|---|---|
| | | Resistant | Susceptible | Resistant | Susceptible | resistant plant |
| | | Leaf | leaf | Ovary | Ovary | (ovary) relative |
| | gene | Expression* | Expression* | Expression* | Expression* | to susceptible |
| Csa3G135680.1 | 2-OGD1 | 2.772 | 2.163 | 14.341 | 5.614 | 2.55 times |
| Csa3G135690.1 | 2-OGD2 | 29.345 | 15.079 | 73.035 | 35.016 | 2.08 times |
| Csa3G135700.1 | 2-OGD3 | 18.35 | 12.597 | 46.295 | 24.25 | 1.90 times |
| Csa3G135710.1 | 2-OGD4 | 0.356 | 0.269 | 1.838 | 1.314 | 1.39 times |
| Csa3G135720.1 | 2-OGD5 | 9.384 | 4.754 | 1.229 | 0.708 | 1.73 times |
| Csa3G135730.1 | 2-OGD6 | 25.495 | 14.371 | 18.057 | 9.411 | 1.91 times |
| Csa3G135740.1 | 2-OGD7 | 5.063 | 3.468 | 1.27 | 0.595 | 2.13 times |
| Csa3G135750.1 | 2-OGD8 | 4.53 | 2.013 | 19.74 | 10.652 | 1.85 times |
| Average of 8 | OGD1 | 11.91 | 6.84 | 21.97 | 10.95 | 2 times |
| OGD genes | to | | | | | |
| | OGD8 | | | | | |
| Csa3G135760.1 | HR-like | 15.012 | 10.345 | 13.019 | 7.358 | 1.76 times |

*average of the normalized values of all replicates (n = 3)

The results showed that in the resistant plant gene expression (mRNA levels) of all nine genes was indeed approximately 1.3 times to 2.6 times higher than in the susceptible plant, both in leaves and in the ovaries. It was, therefore, concluded that the increased gene expression of these nine genes, due to gene duplication, caused the resistance against internal fruit rot.

The eight OGD genes had an average increase in expression in resistant tissue of 1.74 times in leaf and 2 times the expressions in ovary tissue.

Based on the information above, it is thought that the in vivo function of 2-OGD1 to 2-OGD8 is the same, meaning that the same effect can be achieved by any one or any subset of the eight 2-oxoglutarate Fe(II)-dependent oxygenases, as long as the amount of functional 2-oxoglutarate Fe(II)-dependent oxygenase protein is sufficient to prevent DB infection of the fruits (ovaries). Thus, an increase in the average gene expression of all eight OGD genes will result in more OGD enzyme to be available in the tissue, thereby reducing infection of the fruits.

This can of course be tested experimentally by e.g. transforming a susceptible plant with one or more of the genes. Similarly, knocking out individual genes in the resistant plant should reduce resistance against DB, as the amount of functional 2-oxoglutarate Fe(II)-dependent oxygenase protein decreases. The role of the HR-like protein in resistance against DB fruit infection can also be tested by knocking out the gene or silencing the gene in the resistant plant, or overexpressing the gene in susceptible plants.

In one aspect, the functional effect of the duplication of QTL3.1 (i.e. increasing resistance to DB fruit infection) can therefore be not only achieved by duplication of the gene complex of the 9 genes, but can similarly be achieved by enhancing gene expression and protein production of any one or several of the eight 2-oxoglutarate Fe(II)-dependent oxygenases and/or of the HR-like protein.

In one aspect the average expression of the eight OGD genes is significantly increased, e.g. to such an extent that it is at least 1.3 times, 1.4 times, 1.5 times, 1.6 times 1.7 times, 1.8 times, 1.9 times, 2.0 times, 2.2 times, 2.5 times, 3.0 times the average expression of the eight OGD genes in the susceptible plant (e.g. in ovary and/or leaf tissue).

### Example 7-Fine mapping of QTL5.1

QTL5.1 was fine mapped and the QTL region was found to be in-between SNP_12 and SNP_15. Additional SNP markers are included in this region, as shown in FIG. 6 and Table 8 below.

| | | Genotype | | | |
|---|---|---|---|---|---|
| | Physical position | of | Genotype | | |
| | of SNP (base | intro- | of | | |
| | number) in | gression | recurrent | | |
| | genome of | fragment | parent | | |
| | Chinese Long V2 | (homozygous | lacking | Donor SNP | |
| SNP | (cucurbitgenomics. | for | intro- | Haplotype | Genomic sequence |
| marker | org) | QTL5.1) | gression | of QTL5.1 | comprising SNP |
| SNP_12 | 3823864 | GG | AA | G | AAAAACTTGTACATATT |
| | | | | | AATATAGTCTTAACTCT |
| | | | | | TAACCATCACAACATC |
| | | | | | [A/G]TTCAAAAGATGCTC |
| | | | | | CTTTTAACACCATTTCT |
| | | | | | CCAAAACGAAGCTGCA |
| | | | | | GTT |
| | | | | | SEQ ID NO: 12 |
| SNP_45 | 3828984 | AA | GG | A | GAATCTTCTTGGAAAAT |
| | | | | | TCAGTCTGCAATGAAAG |
| | | | | | AAAAACAAAGAATGTT |
| | | | | | [G/A]ATGCAAATGAAAT |
| | | | | | TAAATGCCAAGTGGTTT |
| | | | | | TTGCTTTCTTACCTTTTA |
| | | | | | CA |
| | | | | | SEQ ID NO: 45 |
| SNP_13 | 3832094 | AA | GG | A | ATTGAATATTTGAGAAG |
| | | | | | TTCAATGAAGATATTTA |
| | | | | | AAGGAAGAAAGAAAAA |
| | | | | | [A/G]GAAACAGAGATAC |
| | | | | | ATAGGTTTGTATTGGTA |
| | | | | | ACATAAAAAATGCAAA |
| | | | | | AAGA |
| | | | | | SEQ ID NO: 13 |
| SNP_14 | 3843862 | GG | AA | G | TAGCTATGTTATTGTAT |
| | | | | | GTATTGAAAATTAATAT |
| | | | | | AGCCATTGTTTAATTT[A/ |
| | | | | | G]TTGCAATGATTATGA |
| | | | | | TATATTGCATGAGTCGT |
| | | | | | GCAAGTTATGAACAACT |
| | | | | | T |
| | | | | | SEQ ID NO: 14 |
| SNP_46 | 3844051 | TT | CC | T | CAAACTTTATAAATACA |
| | | | | | TAACACGnTCKTATACT |
| | | | | | ATGTTTGTCTTGATAG[C/ |
| | | | | | T]TTTGTATCTCTTCAC |
| | | | | | ATGGCCATTTAGTTACT |
| | | | | | TGCATTGCAATGTAGAT |
| | | | | | A |
| | | | | | SEQ ID NO: 46 |
| SNP_47 | 3862941 | TT | CC | T | CATCGACCTCGCTTCGA |
| | | | | | ACCCAGCCATTCCCATC |
| | | | | | CTGGCACGCCACTTCC |
| | | | | | [C/T]CGAAACCTCGCATC |
| | | | | | TTTCCACTCGGTCTCTA |
| | | | | | CCCTCACAAGCTAAGG |
| | | | | | AAT |
| | | | | | SEQ ID NO: 47 |
| SNP_48 | 3877105 | CC | AA | C | TTATTTTACACACCACT |
| | | | | | AAAGTAATATTAACATA |
| | | | | | AATGGCAATGTTGATT |
| | | | | | [A/C]TTCATTAAACTAAT |
| | | | | | TTTGGCTTTAAATTTAG |
| | | | | | AAGAAAAAGAAAAGGA |
| | | | | | TGA |
| | | | | | SEQ ID NO: 48 |
| SNP_49 | 3878745 | GG | TT | G | ACATTTAAAATGCTAGA |
| | | | | | AAATTTATCGAAAATGA |
| | | | | | TACATTTATAATTGTT[T/ |
| | | | | | G]GAAGGAGTAGGGTT |
| | | | | | TAGTAGATTTTTGAAGT |
| | | | | | GATTGTTCTTTTATTAC |
| | | | | | AT |
| | | | | | SEQ ID NO: 49 |
| SNP_50 | 3941874 | TT | AA | T | AATCAAGGATTTGGATT |
| | | | | | TGGATTTGGATTTTGTT |
| | | | | | GGATTGGATGCTTGGT |
| | | | | | [A/T]TGATTTGGTGCTTT |
| | | | | | TGTCCWTTTTTGTGAGA |
| | | | | | AAAARATGCAATAAAA |
| | | | | | GTA |
| | | | | | SEQ ID NO: 50 |
| SNP_51 | 3948683 | AA | GC | A | ATTTCAAAAACTTAGCT |
| | | | | | TGCCTTCTAAGCTTTTA |
| | | | | | CTATATATTTTTGTTC[G/ |
| | | | | | A]AGTTCAATTTTACTT |
| | | | | | GTTTCTAAATGTAGGAC |
| | | | | | TAGAATGACTCGAGGTT |
| | | | | | T |
| | | | | | SEQ ID NO: 51 |
| SNP_15 | 3967955 | GG | TT | G | TTGGATTGTTGCATTTT |
| | | | | | GAGCATTCAATTATTAA |
| | | | | | GTCATAACAGAAGAAC |
| | | | | | [T/G]ATCTCTTTAAATAA |
| | | | | | TTTTTAAAAGGTTATGT |
| | | | | | TCTCAATAATTGATCTC |
| | | | | | AA |
| | | | | | SEQ ID NO: 15 |

Further fine mapping or sequencing will be done.

1. A Cucumis sativus var. sativus plant or plant part or seed comprising an increased resistance against internal fruit rot symptoms caused by Didymella bryoniae, wherein said plant, plant part or seed comprises an introgression fragment from a wild cucumber on chromosome 5, comprising Quantitative Trait Locus QTL5.1, wherein the introgression fragment comprising QTL5.1 comprises a SNP haplotype of at least 3 markers selected from:
1 a Guanine for SNP_12 at nucleotide 51 of SEQ ID NO: 12 or at nucleotide 51 of a sequence comprising at least 97% identity to SEQ ID NO: 12,
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 or at nucleotide 51 of a sequence comprising at least 97% identity to SEQ ID NO: 13,
a Guanine for SNP_14 at nucleotide 51 of SEQ ID NO: 14 or at nucleotide 51 of a sequence comprising at least 97% identity to SEQ ID NO: 14,
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 or at nucleotide 51 of a sequence comprising at least 97% identity to SEQ ID NO: 15.

2. The plant, plant part or seed according to claim 1, wherein said QTL5.1 is located in-between SNP_12 at nucleotide 51 in SEQ ID NO: 12 and SNP_15 at nucleotide 51 in SEQ ID NO: 15.

3. The plant, plant part or seed according to claim 1, wherein said QTL5.1 is located on chromosome 5 in-between SNP_12 at nucleotide 3823864 of chromosome 5 and SNP_15 at nucleotide 3967955.

4. The plant, plant part or seed according to claim 1, wherein said QTL5.1 is located physically in the region starting at 3.81 Mb and ending at 3.97 Mb of chromosome 5.

5. The plant, plant part or seed according to claim 1, wherein said QTL5.1 is present in heterozygous form or in homozygous form in the genome.

6. The plant, plant part or seed according to claim 1, wherein said QTL5.1 is obtainable from seeds deposited under accession number NCIMB43530.

7. The plant, plant part or seed according to claim 1, wherein the plant is one of the following cucumber types: slicing cucumber, long cucumber, European greenhouse cucumber.

8. The plant, plant part or seed according to claim 1, wherein the plant is a single cross F1 hybrid or an inbred line.

9. The plant, plant part or seed according to claim 1, wherein the plant produces seedless fruits without pollination.

10. A cucumber fruit harvested from a plant according to claim 1, wherein said fruit comprises QTL5.1.

11. A cucumber cell, cell-culture, tissue culture, vegetative plant tissue, flower, pollen or embryo of a plant according to claim 1, wherein said cell, cell-culture, tissue culture, vegetative plant tissue, flower, pollen or embryo comprises QTL5.1.

12. The plant according to claim 1, wherein the plant is a vegetatively propagated plant.

13. The plant according to claim 1, wherein the plant comprises said SNP haplotype of at least 9 of said SNP markers.

14. A method for identifying or detecting a cultivated C. sativus var. sativus plant or plant part comprising an introgression fragment on chromosome 5 comprising:
a) providing a cultivated C. sativus var. sativus plant or plant parts or DNA of such plant or plant part,
b) screening said plant, plant part or DNA using a molecular marker assay which detects at least one SNP marker selected from the group consisting of SNP_12 to SNP_15 for detecting the introgression fragment on chromosome 5; and
c) identifying and/or selecting a plant comprising:
i) at least 1, 2, 3, 4, 5, 6, 7, 8, 9, 10 or more of the SNP markers of SNP_12 to SNP_15, for detecting the introgression fragment on chromosome 5; or
ii) at least 2, 3, 4 5, 6, 7, 8, 9, 10 or more consecutive markers selected from SNP_12 to SNP_15, for detecting the introgression fragment on chromosome 5,
wherein said SNP markers are selected from:
a Guanine for SNP_12 at nucleotide 51 of SEQ ID NO: 12 or at nucleotide 51 of a sequence comprising at least 97% identity to SEQ ID NO: 12,
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 or at nucleotide 51 of a sequence comprising at least 97% identity to SEQ ID NO: 13,
a Guanine for SNP_14 at nucleotide 51 of SEQ ID NO: 14 or at nucleotide 51 of a sequence comprising at least 97% identity to SEQ ID NO: 14,
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 or at nucleotide 51 of a sequence comprising at least 97% identity to SEQ ID NO: 15.

15. A method of producing C. sativus F1 hybrid plants comprising an introgression fragment conferring enhanced internal fruit rot resistance is provided comprising:
a) providing a first inbred cucumber plant comprising a recombinant chromosome 5 in homozygous form having an introgression fragment comprising an introgression fragment from a wild cucumber on chromosome 5, said introgression fragment comprises Quantitative Trait Locus QTL5.1 and a SNP haplotype of at least 3 markers selected from:
a Guanine for SNP_12 at nucleotide 51 of SEQ ID NO: 12 or at nucleotide 51 of a sequence comprising at least 97% identity to SEQ ID NO: 12,
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 or at nucleotide 51 of a sequence comprising at least 97% identity to SEQ ID NO: 13,
a Guanine for SNP_14 at nucleotide 51 of SEQ ID NO: 14 or at nucleotide 51 of a sequence comprising at least 97% identity to SEQ ID NO: 14,
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 or at nucleotide 51 of a sequence comprising at least 97% identity to SEQ ID NO: 15,
b) providing a second inbred cucumber plant,
c) crossing said cucumber plant of a) with said cucumber plant of b),
d) collecting F1 hybrid seeds from said cross.

16. A method for transferring the recombinant chromosome 5 comprising the QTL5.1 from one cultivated cucumber plant into another cultivated cucumber plant is provided, said method comprises the steps of:
a) providing a first inbred cucumber plant comprising a recombinant chromosome 5 having an introgression fragment from a wild cucumber in homozygous form, said introgression fragment comprises Quantitative Trait Locus QTL5.1 and a SNP haplotype of at least 3 markers selected from:
a Guanine for SNP_12 at nucleotide 51 of SEQ ID NO: 12 or at nucleotide 51 of a sequence comprising at least 97% identity to SEQ ID NO: 12,
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 or at nucleotide 51 of a sequence comprising at least 97% identity to SEQ ID NO: 13,
a Guanine for SNP_14 at nucleotide 51 of SEQ ID NO: 14 or at nucleotide 51 of a sequence comprising at least 97% identity to SEQ ID NO: 14,
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 or at nucleotide 51 of a sequence comprising at least 97% identity to SEQ ID NO: 15,
b) providing a second cultivated cucumber plant,
c) crossing said cucumber plant of a) with said cucumber plant of b),
d) collecting F1 hybrid seeds from said cross, and
e) optionally selfing the plant grown from said F1 hybrid seeds to produce F2 seeds or further selfing generations, and optionally selecting the F2 seeds or further selfing generation seeds having the recombinant chromosome 5, and
f) optionally breeding further with plants grown from said F1 or F2 or further generation selfing seeds to produce a cucumber plant having good agronomic characteristics and comprising the introgression fragment in homozygous or heterozygous form.

17. A method for identifying a wild cucumber comprising a fruit rot resistance QTL on chromosome 5, said method comprising:
a) providing a wild cucumber accession or several accessions;
b) screening said accessions using a molecular marker assay which detects at least 1, 2, 3, 4, 5, 6, 7, 8, 9, 10 or more SNP marker selected from SNP_12, SNP_45, SNP_13, SNP_14, SNP_46 to SNP_51 and SNP_15;
c) identifying and/or selecting an accession from B) comprising a marker genotype or haplotype for at least 1, 2, 3, or more of the following markers selected from:
a Guanine for SNP_12 at nucleotide 51 of SEQ ID NO: 12 or at nucleotide 51 of a sequence comprising at least 97% identity to SEQ ID NO: 12,
an Adenine for SNP_45 at nucleotide 51 of SEQ ID NO: 45 or at nucleotide 51 of a sequence comprising at least 97% identity to SEQ ID NO: 45,
an Adenine for SNP_13 at nucleotide 51 of SEQ ID NO: 13 or at nucleotide 51 of a sequence comprising at least 97% identity to SEQ ID NO: 13,
a Guanine for SNP_14 at nucleotide 51 of SEQ ID NO: 14 or at nucleotide 51 of a sequence comprising at least 97% identity to SEQ ID NO: 14,
a Thymine for SNP_46 at nucleotide 51 of SEQ ID NO: 46 or at nucleotide 51 of a sequence comprising at least 97% identity to SEQ ID NO: 46,
a Thymine for SNP_47 at nucleotide 51 of SEQ ID NO: 47 or at nucleotide 51 of a sequence comprising at least 97% identity to SEQ ID NO: 47,
a Cytosine for SNP_48 at nucleotide 51 of SEQ ID NO: 48 or at nucleotide 51 of a sequence comprising at least 97% identity to SEQ ID NO: 48,
a Guanine for SNP_49 at nucleotide 51 of SEQ ID NO: 49 or at nucleotide 51 of a sequence comprising at least 97% identity to SEQ ID NO: 49,
a Thymine for SNP_50 at nucleotide 51 of SEQ ID NO: 50 or at nucleotide 51 of a sequence comprising at least 97% identity to SEQ ID NO: 50,
an Adenine for SNP_51 at nucleotide 51 of SEQ ID NO: 51 or at nucleotide 51 of a sequence comprising at least 97% identity to SEQ ID NO: 51,
a Guanine for SNP_15 at nucleotide 51 of SEQ ID NO: 15 or at nucleotide 51 of a sequence comprising at least 97% identity to SEQ ID NO: 15;
and optionally
d) introgressing said QTL from said wild accession into cultivated cucumber.
